(19) United States
(12) Patent Application Publication
(10) Pub. No.: US 2025/0057813 A1
(43) Pub. Date: Feb. 20, 2025

(54) APOPTOSIS RELATED PROTEIN IN THE TGF-BETA SIGNALING PATHWAY (ARTS) MIMETIC COMPOUNDS, COMPOSITIONS, METHODS AND USES THEREOF IN INDUCTION OF APOPTOSIS

(71) Applicant: Carmel-Haifa University Economic Corporation Ltd., Haifa (IL)

(72) Inventor: Sarit LARISCH, Zichron Yaakov (IL)

(21) Appl. No.: 18/814,135

(22) Filed: Aug. 23, 2024

Related U.S. Application Data
(63) Continuation-in-part of application No. PCT/IL2023/050198, filed on Feb. 24, 2023.
(63) Continuation-in-part of application No. PCT/IL2023/050212, filed on Mar. 1, 2023.
(60) Provisional application No. 63/313,789, filed on Feb. 25, 2022.
(60) Provisional application No. 63/315,209, filed on Mar. 1, 2022.

(51) Int. Cl. A61K 31/4164

(52) U.S. Cl. CPC A61K 31/4164

(57) ABSTRACT

The present invention provides ARTS mimetic compounds that act as novel antagonists for XIAP and Bcl-2. Moreover, the novel ARTS mimetic compounds of the invention induce apoptosis in premalignant and malignant cells. The invention thus provides compositions, methods and uses of said ARTS mimetic compounds in the treatment of cancer and premalignant conditions.

### CROSS-REFERENCE TO RELATED APPLICATIONS

The present application is a continuation-in-part of PCT/IL2023/050198 filed on Feb. 24, 2023, claiming priority from U.S. Application No. 63/313,789 filed on Feb. 25, 2022, and it is a continuation-in-part of PCT/IL2023/050212 filed on Mar. 1, 2023 claiming priority from U.S. Application 63/315,209 filed on Mar. 1, 2022. The entire contents of each of said two international applications and said two provisional applications are hereby incorporated herein by reference in their entirety for all purposes.

The Sequence Listing in ASCII text file format of 74,228 bytes in size, created Aug. 23, 2024, with the file name “2024-08-23seqlst-LARISCH16,” filed in the U.S. Patent and Trademark Office on even date herewith, is hereby incorporated herein by reference.

### FIELD OF THE INVENTION

The invention relates to the field of cancer therapy. More particularly, the invention relates to novel ARTS mimetic compounds that induce apoptosis, specifically, in malignant cells. The invention further provides compositions comprising the ARTS mimetic compounds, methods and uses thereof in treating proliferative disorders.

### PRIOR ART

References considered to be relevant as background to the presently disclosed subject matter are listed below:
Fuchs, Y., and H. Steller. Cell. 147:1-17 (2011).
Gottfried, Y., A. et al., EMBO J. 23:1627-35 (2004).
Larisch, S., et al., Nat Cell Biol. 2:915-21 (2000).
Edison, N., D. et al. Cell Death Differ. 19:356-68 (2012b).
Bornstein, B., Y. et al., Apoptosis 16:869-881 (2011).
Reingewertz, T. H., et al., PLoS One. 6:e24655 (2011).
Adams, J. M., and S. Cory. Trends Biochem Sci. 26:61-6 (2001).
Youle, R. J., and A. Strasser. Nat Rev Mol Cell Biol. 9:47-59 (2008).
Happo, L., A. et al., J Cell Sci. 125:1081-7 (2012).
Edison, N., T. H. et al., Clin Cancer Res (2012a).
Bornstein, B. N., et al., IntJBiochem Cell Biol. 44:489-95 (2012).
Barkan D. et al., Cancer Research 68 (15): 6241-50 (2008).
Debnath et al., Methods 30 (3): 256-68 (2003)
Muthuswamy et al., Nature Cell Biology. 3(9): p. 785. (2001)
Edison, N. et al., Cell Reports 21, 442-454 (2017)
   Acknowledgement of the above references herein is not to be inferred as meaning that these are in any way relevant to the patentability of the presently disclosed subject matter.

### BACKGROUND OF THE INVENTION

Apoptosis is a process of programmed cell death that plays a major role in tissue development, tissue homeostasis, and as a defense mechanism against unwanted and potentially dangerous cells.

Apoptosis is controlled by a diverse range of cell signals which can originate either from extrinsic inducers thus activating the extrinsic, apoptotic signaling pathway or from intrinsic inducers, which activate the intrinsic, mitochondrial signaling pathway.

The control of apoptosis is achieved through the activity of pro- and anti-apoptotic proteins. For example, caspases, are a family of cysteine proteases that play a central executioners of apoptosis and the action of activators and inhibitors of caspases affect apoptosis. Inhibition of the caspases activity was reported to occur through the action of the inhibitor of apoptosis (IAP) proteins. Apoptosis has been reported to have a critical role in a variety of diseases. It has been shown that deregulation of the apoptosis pathway can result in various pathologic conditions, including cancer (Fuchs and Steller, 2011). Involvement of an abnormal ratio of pro- and anti-apoptotic proteins has been also associated with neurodegenerative diseases such as schizophrenia as well as in immune-related disorders.

To potentiate apoptosis, the function of IAPs needs to be overcome. This is achieved by IAP-antagonists such as Smac/Diablo, Omi/HtrA2 and ARTS (Gottfried et al., 2004; Larisch et al., 2000).

ARTS is localized at mitochondrial outer membrane (MOM) (Edison et al., 2012b). Upon induction of apoptosis, ARTS translocate from the mitochondria to the cytosol, directly binds and antagonizes XIAP, causing activation of caspases and cell death (Bornstein et al., 2011; Edison et al., 2012b; Reingewertz et al., 2011). XIAP, the best studied IAP, can directly bind and inhibit caspases 3, 7 and 9 via its three Baculoviral IAP Repeats (BIR) domains.

The inventors have further shown that upon induction of apoptosis, ARTS binds directly to both XIAP and Bcl-2, acting as a scaffold to bring these proteins together. This binding leads to a UPS mediated degradation of Bcl-2. Moreover, the inventors have found that ARTS comprise a BH3-like domain. These data indicate that ARTS functions as a Bcl-2 antagonist and as such, allows degradation of Bcl-2 and inhibits the anti-apoptotic activity of Bcl-2 (WO 2013/121428, Edison, N. et al., Cell Reports 21, 442-454 (2017)).

The intrinsic pathway of apoptosis is regulated by Bcl-2 family members (Adams and Cory, 2001). This family is composed of pro- and anti-apoptotic proteins that share up to four conserved Bcl-2 homology (BH) domains (Youle and Strasser, 2008). The pro-apoptotic members can be separated into the “multidomain” proteins and to “BH3 only” proteins. Bax and Bak “multidomain” proteins which share three BH regions and structurally similar to the antiapoptotic proteins. The “BH3-only” proteins, which include Bnip3, Nix/Bnip3L, Bid, Noxa, Puma, and Bad, share only the BH3 domain and are structurally diverse (Happo et al., 2012).

The inventors have further shown that upon induction of apoptosis, ARTS binds directly to both XIAP and Bcl-2, acting as a scaffold to bring these proteins together. This binding leads to a UPS mediated degradation of Bcl-2. Moreover, the inventors have found that ARTS comprise a BH3-like domain. These data indicate that ARTS functions as a novel Bcl-2 antagonist and as such, inhibits the anti-apoptotic activity of Bcl-2 (WO 2013/121428).

There is need for novel compounds that target and inhibit both classes of pro-apoptotic proteins, the XIAP and Bcl-2. As ARTS functions in a distinct way, different from all known IAP-antagonists (Gottfried et al, 2004, Edison et al, 2012a, Bornstein et al, 2011, Bornstein et al, 2012) and as a novel Bcl-2 antagonist, it offers a unique approach for anti-cancer therapies targeting the two major anti-apoptotic proteins. These compounds would be better and more effective inducers of apoptosis in malignant pathologies.

### SUMMARY OF THE INVENTION

In a first aspect, the present disclosure provides an apoptosis related protein in the TGF-beta signaling pathway (ARTS) mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for inducing apoptosis in a cell, wherein formula (I) is:

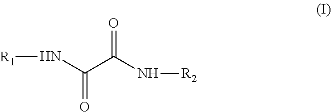

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

Another aspect of the present disclosure relates to an ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for treating, preventing, inhibiting, reducing, eliminating, protecting or delaying the onset of a pathological disorder in a subject in need thereof, wherein formula (I) is:

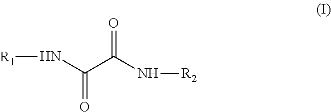

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

A further aspect of the preset disclosure relates to a method for inducing apoptosis in a cell, wherein said method comprises the step of contacting said cell with an effective amount of at least one ARTS mimetic compound, any combination thereof or any composition comprising the same, wherein said ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same. In some embodiments, Formula (I) is:

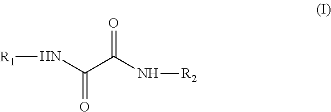

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
   L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—, each optionally substituted by one or more of C₁-C₅alkoxy, C₁-C₅ carboxylic acid, —(CH₂)ₘ—OH, —(CH₂)ₘ SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5.

A further aspect of the present disclosure relates to a method for treating, preventing, inhibiting, reducing, eliminating, protecting or delaying the onset of a pathological disorder in a subject in need thereof. The disclosed method comprises administering to said subject a therapeutically effective amount of at least one ARTS mimetic compound or of a composition comprising the same, said ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same; wherein said Formula (I) is:

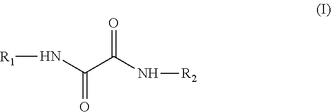

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—, each optionally substituted by one or more of C₁-C₅alkoxy, C₁-C₅ carboxylic acid, —(CH₂)ₘ—OH, —(CH₂)ₘ SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5.

Another aspect of the preset disclosure relates to an ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, wherein formula (I) is:

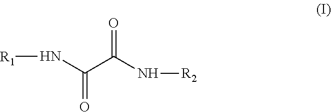

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

A further aspect of the present disclosure relates to a combined composition comprising an effective amount of at least one ARTS mimetic compound and at least one and at least one B-cell lymphoma 2 (Bcl-2) Homology 3 (BH3) mimetic compound, wherein said ARTS mimetic compound is having the general formula (I) or a pharmaceutically acceptable salt, solvate, hydrate, any stereoisomer thereof or physiologically functional derivative thereof, and wherein formula (I) is:

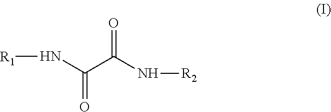

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5 each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

A further aspect of the present disclosure relates to a kit comprising: at least one ARTS mimetic compound having the general formula (I) or a pharmaceutically acceptable salt, solvate, hydrate, any stereoisomer thereof or physiologically functional derivative thereof, wherein formula (I) is:

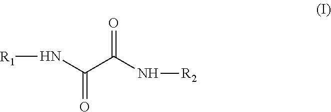

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5 each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5; and
(b) at least one BH3 mimetic compound.

A further aspect of the present disclosure relates to a method for inducing apoptosis in a cell comprising the step of contacting said cell with an effective amount of at least one ARTS mimetic compound as defined by the present disclosure, and of at least one BH3 mimetic compound, with a combined composition as defined by the present disclosure, or with any kit as defined by the present disclosure.

In some embodiments of the disclosed methods, the BH3 mimetic compound is 4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

A further aspect of the present disclosure relates to a method for treating, preventing, inhibiting, reducing, eliminating, protecting or delaying the onset of a proliferative disorder in a subject in need thereof, the method comprises administering to said subject a therapeutically effective amount of at least one ARTS mimetic compound as defined by the present disclosure, and of at least one BH3 mimetic compound, or of any composition or kit comprising the BH3 mimetic compound and said ARTS mimetic compound.

A further aspect of the present disclosure relates to an effective amount of at least one of apoptosis related protein in the TGF-beta signaling pathway (ARTS), any fragments thereof, or at least one mimetic compound thereof or any vehicle, matrix, nano- or micro-particle comprising the same, for use in a method for upregulating p53 levels in a cell.

Another aspect of the present disclosure relates to a composition comprising effective amount of at least one of ARTS, any fragments thereof, at least one mimetic compound thereof or any vehicle, matrix, nano- or micro-particle comprising the same, for use in a method for upregulating p53 levels in a cell. More specifically, the composition optionally further comprises at least one of pharmaceutically acceptable carrier/s, excipient/s, auxiliaries, and/or diluent/s.

Another aspect of the present disclosure relates to methods for upregulating the levels of p53 in a cell. More specifically, the disclosed method comprises the step of contacting the cell with an effective amount of ARTS, any fragments thereof, or at least one mimetic compound thereof or any vehicle, matrix, nano- or micro-particle comprising the same, or any composition thereof.

Another aspect of the present disclosure relates to a method for treating, preventing, inhibiting, reducing, eliminating, protecting or delaying the onset of a pathological disorder in a subject in need thereof, by upregulating p53 levels in at least one cell of the subject. The method comprising administering to the subject a therapeutically effective amount of at least one of ARTS, any fragments thereof, or at least one mimetic compound thereof or any vehicle, matrix, nano- or micro-particle thereof or of a composition comprising the same.

Another aspect of the present disclosure relates to a kit comprising: In one component (a), ARTS, or any fragments thereof, or at least one mimetic compound thereof, optionally, in first dosage form. It should be noted that the ARTS mimetic compound of the disclosed kits may have the general formula (I) or a pharmaceutically acceptable salt, solvate, hydrate, any stereoisomer thereof or physiologically functional derivative thereof, wherein formula (I) is:

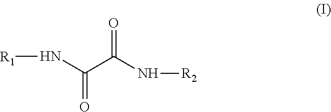

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5; and

In another component (b), the disclosed kits further comprise at least one therapeutic compound, optionally, in a second dosage form.

These and other aspect of the invention will become apparent by the hand of the following drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1. Docking site of ARTS mimetic compounds within BIR3/XIAP

Figure illustrates an “in silico” screen for ARTS mimetic small molecules that fit into the binding site of ARTS unique C-terminus and its distinct binding sequence in the BIR3 domain of XIAP.

FIG. 2. Binding of B3 to BIR3-XIAP

Figure illustrates the molecular structure of the B3 small molecule (in bold-nude) and its interaction with the ARTS binding site within the XIAP molecule. Favorable interactions of said candidate molecule with BIR3-XIAP are shown in green, where negative interaction is shown in red.

FIG. 3. The ARTS mimetic B3 small molecule

Figure shows the B3 chemical structure indicating residues Thr271, Thr274, Tyr277 and Gly293 of the BIR3/XIAP that interact with the B3 molecule.

FIG. 4. XIAP and Bcl-2 levels in A375 human melanoma cells treated with different ARTS mimetic small molecules

A375 cells were treated with 20 uM of each of the indicated molecules for 24 hrs. SDS PAGE and Western blot analysis were performed with the indicated Abs.

FIG. 5. The ARTS mimetic small molecule B3 induces apoptosis of MCF10AT1K (M2) cells

MCF10A (M1) and M2 cells were cultured using the 3D BME system. M2 organoids at day 4 were either untreated or treated for additional 7 days with increasing concentrations of the B3 small molecule. B3 molecule was re-supplemented every 4 days. The figure shows representative light images, magnification ×20.

FIG. 6. The ARTS mimetic small molecule B3 enhances apoptosis in premalignant cells

The extent of apoptosis is shown by representative bright field images (upper panel magnification ×10) and digital zooming of confocal images (lower panel, magnification ×4). M2 cells were cultured in the 3D BME system for 4 days followed by treatment with B3 for 24 h. The cells were stained for apoptosis using TUNEL assay. TUNEL positive cells are in red and nuclei (stained with Dapi) are in blue. Bar=20 μm.

FIG. 7. IC50 of ARTS mimetic B3 small molecule on different cancer cell lines vs. normal control

Viability assay results examining the effect of B3 on 94 different cancer cell lines. A-375 cells are considered relatively resistant to Bx as they displayed a high GI50 score of 14.78 uM.

FIG. 8(I)-8(II). IC50 of ARTS mimetic B3 small molecule on different cancer cell lines vs. normal control

IC50 of ARTS mimetic B3 small molecule (was calculated for several cancer cell lines indicated therein. PBMC were used as normal cell control. FIG. 8-I shows cell lines resistant to B3 and FIG. 8-I shows sensitive cell lines

FIG. 9A-9C. ABT-199 upregulates the endogenous ARTS levels in Melanoma

FIG. 9A. Western Blot analysis of A-375 μMelanoma cell line treated for 24 hrs with different concentrations of ABT-199 with and without the small molecule B3. Combined treatment of ABT-199 and B3 increased cleavage of apoptotic markers PARP and Caspase3. Actin is used as a reference for overall protein level in the sample.

FIG. 9B. Histogram presenting the cleaved PARP/Actin ratio.

FIG. 9C. Histogram presenting the cleaved Caspase3/Actin ratio.

FIG. 10A-10B. ABT-199 upregulates Bcl-2 levels in Melanoma

FIG. 10A. Western Blot analysis of A-375 μMelanoma cell line treated for 24 hrs with different concentrations of ABT-199 with and without the small molecule B3, showing Bcl-2 levels in the cells. Actin is used as a reference. Treatment of ABT199 alone upregulated Bcl-2 expression while combined treatment ABT199+B3 significantly decrease Bcl-2 levels.

FIG. 10B. Histogram presenting the Bcl-2/Actin ratio.

FIG. 11A-11B. Combined treatment of B3 with ABT-199 increases sensitivity of resistant CCRF-CEM to ABT-199

FIG. 11A. Western Blot analysis of CCRF-CEM (acute lymphoblastic leukemia) cell line treated for 24 hrs with ABT-199 with and without the small molecule B3 and another small molecule A4, showing cCaspase3 levels in the cells. Actin is used as a reference for overall protein level in the sample.

FIG. 11B. Histogram presenting the cleaved Caspase3/Actin ratio.

FIG. 12A-12F. Endogenous ARTS restricts the killing effect of ABT-199

FIG. 12A. Western Blot analysis of A375 melanoma cell line. Cells expressing low endogenous ARTS protein, were treated for 24 hrs with ABT-199 with and without the small molecules B3 and A4, showing cPARP and cCaspase3 levels. Actin is used as a reference for overall protein level in the sample. A374 cells with low levels of endogenous ARTS show a significant apoptotic response to simultaneous treatment of ABT199+B3.

FIG. 12B. Western Blot analysis of WT HeLa and shARTS HeLa (that were KD for ARTS). Cells with different amounts of endogenous ARTS protein treated for 24 hrs with ABT-199 with and without the small molecules B3 and A4, showing cPARP and cCaspase3 levels. Actin is used as a reference for overall protein level in the sample.

Actin is used as a reference for overall protein level in the sample. HeLa cells with Knockdown of ARTS show high apoptotic response to combined treatment of ABT199+B3 than WT HeLa cells.

FIG. 12C. Western Blot analysis of WT Mefs and Sept4/ARTS KO MEFs. Cells with different amounts of endogenous ARTS protein treated for 24 hrs with ABT-199 with and without the small molecules B3 and A4, showed different cPARP and cCaspase3 levels. Actin is used as a reference for overall protein level in the sample. Sept4/ARTS KO MEFs cells showed high apoptotic response to combined treatment of ABT199+B3 than WT Mefs cells.

FIG. 12D. Histogram presenting the cleaved PARP/Actin ratio in A-375 cells.

FIG. 12E. Histogram presenting the cleaved PARP/Actin ratio in WT HeLa and shARTS HeLa cells.

FIG. 12F. Histogram presenting the cleaved PARP/Actin ratio, in Sept4/ARTS KO MEFs and WT MEFs cells.

FIG. 13A-13D. Combined treatment of B3 with ABT-199 decreases expression of Bcl2 after 6 hrs in A-375 cell line

FIG. 13A. Western Blot analysis of A-375 μMelanoma cell line treated for 2,6 and 24 hrs with ABT-199 with and without the small molecule B3, showing cPARP, BCL-2 and XIAP levels in the cells. BCL-2 levels were upregulated upon 2 h and 6 h of treatment with ABT-199 alone. While BCL-2 levels downregulated after 6 hrs treatment with B3.

The combined treatment of ABT-199 with B3 decreased XIAP levels.

FIG. 13B. shows one-way ANOVA data presentation of the cPARP/Actin ratio.

FIG. 13C. shows one-way ANOVA data presentation of the XIAP/Actin ratio.

FIG. 13D. shows one-way ANOVA data presentation of the BCL-2/Actin ratio. FIG. 14. Combined treatment of B3 with ABT-199 increase cell death

FIG. 14. Histogram presenting cell death as defined by XTT viability assay in A-375 (melanoma), MALME (melanoma) and DU145 (prostate cancer) cell lines treated with increasing concentrations of ABT-199 with and without 20 μM small molecule B3 for 24 hrs.

FIG. 15. Dose dependent effect of B3 on cell death of MG-63 osteosarcoma cell line

Dose response of cell death with B3 treatment relative to DMSO treatment.

FIG. 16A(I-II)-16C. 20 uM B3 combined with ABT-199 showed the maximal apoptotic effect in A-375 cells

FIG. 16A(I). Western Blot analysis of A-375 cells treated with different concentrations of ABT-199 and B3. Rising concentrations of ABT-199 increased cCaspase 3 expression, while the combination of 1 μM ABT199 with 20 μM B3 showed synergistic effect on Apoptosis in A-375cells.

FIG. 16A(II). Histogram presenting cCasp3/Actin ratio.

FIG. 16B. Cell death measured using XTT assay in A-375 and MALME cell lines.

Different concentrations of ABT199 treatment together with 20 uM B3 showed the strongest apoptotic effect.

FIG. 16C. Cell Titer-Glo® Viability Assay with of rising concentrations of treatment of A-375 cells, the strongest apoptotic effect was recorded with 5 and 10 uM ABT-199 with 20 uM B3.

FIG. 17A-17B. Combined treatment of B3 with ABT-199 enhances the apoptotic effect of ABT-199

FIG. 17A. Western Blot analysis of A-375 cells treated with ABT-199 and B3. Combined treatment of ABT-199+20 uM B3 increased cCaspase3 levels relative to treatment with ABT-199 alone.

FIG. 17B. Shows one-way ANOVA data presentation of cCasp3.Statistical One way ANOVA statistical analysis of two-three biological independent experiments with Dunnett's multiple comparisons test, p<0.05 (*), p<0.01 (**), p<0.001 (***).

FIG. 18A(I-II)-18D. Both B3 and ABT-199 promote the binding of XIAP to Bcl-2

FIG. 18A(I). Nano-DSF method (Crelux) binding assay of B3 binding to XIAP in a dose response manner.

FIG. 18A(II). MST binding assay of B3 binding to Bcl-2. in a dose response manner.

FIG. 18B. Immunoprecipitation (IP) assay of HCT 116 XIAP KO cells transfected with GFP-XIAP and Flag-Bcl-2, and treated with 20 uM B3. B3 treatment promotes the binding of Bcl-2 to GFP-XIAP.

FIG. 18C(I). Histogram presenting the BiFC assay in A375 melanoma cells treated with ABT-199 and B3. Cells were transfected with Bcl-2-VC and XIAP-VN for 24 hours. Just 20 μM B3 slightly increased binding of XIAP to Bcl-2 after 24 hours. Statistical One-way ANOVA statistical analysis of three-four biological independent experiments, p<0.05 (*).

FIG. 18C(II). B3 increase binding between XIAP and Bcl2 in a dose dependent manner. A-375 Cells were transfected with Bcl-2 and XIAP fused to parts of Venus fluorescent protein (VN 1-173 and VC 1-155, respectively) for 24 hours and treated with 10 μM and 20 μM of ABT199 or B3. pdsRED plasmid was used as a transfection efficiency marker. The experiment was done in duplicates. FACS results were normalized to the readings of transfection efficiency reporter (pdsRED) relative to Untreated cells. Just 20 μM B3 slightly increased binding of XIAP to Bcl-2 after 24 hours Shows one-way ANOVA data presentation of BiFC assay p<0.001 (***)

FIG. 18D. Administering ABT-199 and B3 results in a increase in cell death that is higher than what each of them achieves alone.

FIG. 19. B3 and ABT199 increase interaction between ARTS and Bcl2 during early Apoptosis

Histogram presenting BiFC assay in A-375 cells treated with ABT-199 and B3. Hela wt cells were transfected with the following pairs of plasmids Bcl2 and ARTS fused to parts of Venus fluorescent protein (VN 1-173 and VC 1-155, respectively). 36 h post-transfection cells were exposed to the following components 20 μM ABT199, 20 μM B3, B3+ABT199 20 μM each or DMSO for 3 and 24 hours. pdsRED plasmid was used as a transfection efficiency marker. The experiment was done in duplicates. FACS results were normalized to the readings of transfection efficiency reporter (pdsRED) relative to Untreated cells. B3 and ABT199 increase interaction between ARTS and Bcl2 at early stages of apoptosis and had no effect on the interaction at later stages of apoptosis (N=2).

FIG. 20. Proposed model of ABT-199 and B3 mechanism of action through the regulation of Bcl-2

Treatment with ABT-199 and with B3 induces upregulation of ARTS which promotes the formation of protein complex between Bcl-2-ARTS and XIAP. This results in high levels of Bcl-2. Treatment with B3 or the combined treatment leads to elevated levels of endogenous ARTS and prominent auto-ubiquitylation and degradation of XIAP and Bcl-2. In the combined treatments of ABT-199 and B3, the binding of B3 to XIAP leads to the activation of the E3-ligase function of XIAP. This results in enhanced degradation of Bcl-2 and substantial increase in the apoptotic effect.

FIG. 21A-21B. B3 induces cell death Jurkat cells as efficient as the strong pro-apoptotic chemotherapeutic agent Etoposide

FIG. 21A. PrestoBlue viability assay of different small molecules in A-375 and Jurkat cell lines. Cells were incubated with molecules and apoptotic agents for 24 h. Blue line indicates the threshold of Etoposide treated cells alone.

FIG. 21B. The effect of 20 μM B3 and 12.5 μM Etoposide on cell death of Jurkat cell lines.

Average of three independent experiments.

FIG. 22. The effect of small-molecule B3 in A375 and HCT wt cells

A375 (melanoma) cell line was treated with 20 uM of each of the indicated molecules for 24 hrs. SDS PAGE and Western blot analysis were performed with the indicated Abs. Both cell lines showed increase in cleavage of early apoptotic marker Caspase 9 and decrease in XIAP levels upon treatment with B3 small molecule. A-375 cell line also showed decrease in Bcl2 level and significant increase in cCaspase3.

FIGS. 23A-23F. The effect of small-molecule B3 in resistant and sensitive to ABT199 Leukemia cell lines

CCRF_CEM—resistant to ABT-199 (FIGS. 23B, 23E, 23F) and HL-60 sensitive to ABT199 leukemia (FIGS. 23A, 23C, 23D) cell lines were treated with 10 μM or 1 μM ABT199 respectively with or without 20 uM of each of the indicated molecules for 24 hrs. SDS PAGE and Western blot analysis were performed with the indicated Abs. Apoptotic marker proteins cleaved PARP (FIGS. 23D, 23F) and cleaved Caspase 3 (FIGS. 23C, 23E) Apoptotic marker proteins cleaved PARP and cleaved Caspase 3 are upregulated upon treatment with 10M ABT-199 in CCRF-CEM and 1 μM ABT199 in HL-60 cell line. B3 promoted apoptosis alone. A combination treatment of ABT-199 with B3 increased significantly expression of cPARP and cCasp3 in ABT199 resistant CCRF-CEM cell line. While in ABT199 sensitive HL-60 cell line no synergistic effect was observed. Experiment repeated twice.

FIGS. 24A-24B(I)—B(IV). Combined treatment of 20 μM B3 with 10 μm ABT-199 significantly increase Apoptosis in A-375 cell line

A-375 cells were treated with various concentrations of ABT 199 and B3 for 24 h. Western blot analysis was conducted with the indicated antibodies (FIG. 24A). Cells treated with 10 μM or 20 μM ABT199 together with 20 μM B3 showed a synergistic effect on apoptosis. Significant increase in cCaspase 3 and cPARP levels, and decrease in Bcl2 and XIAP levels in comparison to untreated cells or cells treated with each compound separately (FIG. 24B(I) cCaspase; 24B(II) cPARP; 24B(III) Bcl-2; and 24B(IV) XIAP) (N=3).

FIG. 25A-25H. B3 apoptotic effect on different cell lines

FIG. 25 A-25E. Three melanoma cell lines: SKMEL-5, A-375, UACC257, Hela wt (cervical cancer), Mefs (mouse embryonic fibroblasts) were treated with 20 μM from each molecule, 20 μM ABT199 or both for 24 h. Cells were lysed and Western blot analysis was conducted with the indicated antibodies. B3 molecules induced cleavage of apoptotic markers Caspase3 and PARP. All Cells when exposed to combined treatment of ABT-199 with B3 showed synergistic effect on Apoptosis.

FIG. 25F. One-way ANOVA data presentation of the cPARP/Actin ratio in wt HeLa cells.

FIG. 25G. One-way ANOVA data presentation of the XIAP/Actin ratio in wt MEF cells.

FIG. 25H. One-way ANOVA data presentation of the BCL-2/Actin ratio in A-375cells.

FIG. 26A-26D. ARTS is a transcriptional target of p53

FIG. 26A. Scheme of the putative p53 binding site in ARTS promoter sequences. Scheme of Sept4 μgene. The sept4 μgene encodes two main splice variants, Sept4_i1 (PNUTL2, H5) and Sept4_i2 (ARTS). Only the ARTS protein can promote apoptosis. The scheme shows the location of a putative p53 binding site at the ARTS promoter located between nucleotides −391 to −351 upstream of the ARTS TSS.

FIG. 26B. p53 binds to promoter sequences of ARTS rapidly upon induction of apoptosis. Chromatin IP (ChIP) assay was performed on WT HCT116 cells. The binding of p53 is shown after 5 minutes of UV treatment. These results suggest that the ARTS promoter contains specific binding sequences for p53. Collectively, these results suggest that p53 acts as a transcriptional factor inducing upregulation of ARTS upon induction of DNA damage and apoptosis.

FIG. 26C. UV irradiation induces p53-dependent upregulation of ARTS RNA. Real-Time PCR results. WT HCT116 and p53 KO HCT116 cells were induced for apoptosis with UV for the indicated time points. Real-Time PCR was performed, and cDNAs were normalized to GPI (housekeeping gene). Error bars represent +/− SER of three biologically independent experiments. (*p-value<0.05, **p-value<0.01, ***p-value<0.001).

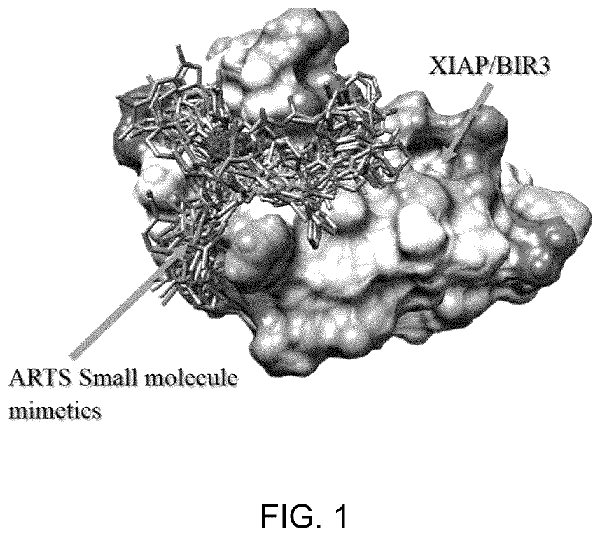

FIG. 26D(i)-26D(ii). RT-PCR of ARTS mRNA levels in HCT116 or HCT116 p53 knockout cells treated with 200 μM Etoposide for up to 24 hours FIG. 1D(i).

Densitometry analysis is shown in FIG. 1D(ii).

FIG. 27A-27D. ARTS regulates p53 protein levels through proteasome-mediated degradation

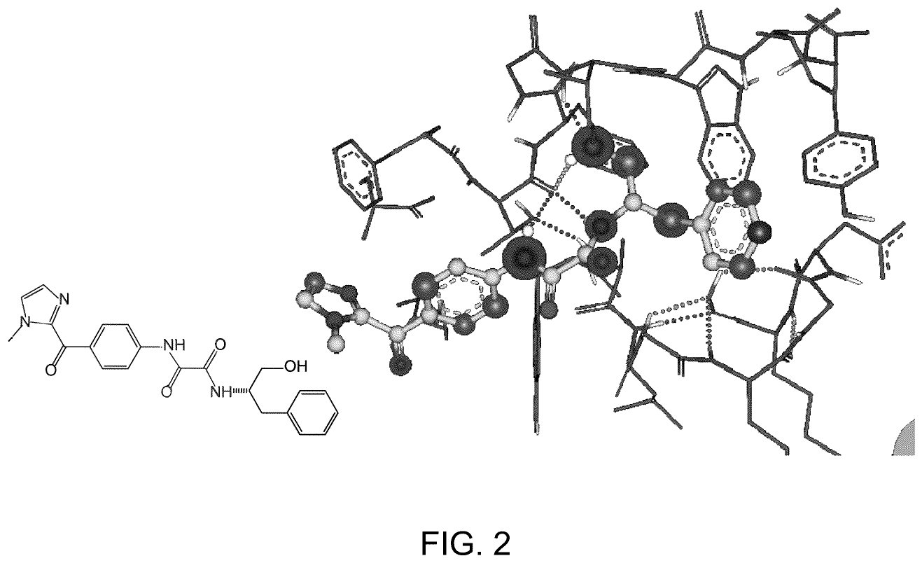

FIG. 27A(i)-27A(ii). Proteins were isolated from Sept4/ARTS KO MEFs cells, and a Western blot was performed. Treatment with DMSO alone served as a negative control (FIG. 2A(i)). Densitometry analysis of four biologically independent repeats (FIG. 27A(ii)).

FIG. 27B(i)-27B(ii). MEFs cells and Sept4/ARTS KO MEFs cells were treated with MG132 and induced for apoptosis with UV for 10 minutes. Western blots were performed (FIG. 2B(i)) using p53, ARTS, XIAP and MDM2 antibodies. Actin serves as a control. Densitometry analysis of three biologically independent repeats (FIG. 27B(ii)).

FIG. 27C(i)-27C(ii). Sept4/ARTS KO MEFs were treated with 200 ng/ml Nocodazole (NOC) for 1 hour following 40 hours of ARTS overexpression (FIG. 27C(i)). Densitometry analysis of four biologically independent repeats (FIG. 27C(ii)). One Way ANOVA statistical analysis was done using GraphPad Prism version 8, Mean+/−SD; **p-value<0.001; *p-value<0.05.

FIG. 27D. Bimolecular fluorescence complementary assay (BiFC). Etoposide promotes the formation of p53-ARTS complex. Bimolecular fluorescence complementation (BiFC) assay was performed on WT MEFs that were treated with 200 μM Etoposide for the indicated time points. Cells were transfected with VN-p53 and VC-ARTS plasmids for 36 hours. Fluorescence-activated cell sorting (FACS) analyses revealed an increase in the formation of ARTS-p53 complex upon Etoposide treatment.

FIG. 28A-28C. ARTS is important for the localization of p53 to the nucleus FIG. 28A(i)-28A(ii). MEFs cells (FIG. 28A(i)) and Sept4/ARTS KO MEFs cells (FIG. 28A(ii)) were induced for apoptosis with UV for the indicated time points. Nuclei and cytosol fractions were dissected from the cells and Western blots were performed using p53, ARTS, Lamin, Tubulin and Her2 antibodies.

FIG. 28B(i)-28B(ii). MEFs cells and Sept4/ARTS KO MEFs cells were induced for apoptosis with UV for 10 minutes. Cells were immobilized and fluorescently stained for p53, mitotracker (a mitochondrial marker) and DAPI (a nuclei marker) (FIG. 28B(i)). The graphs represent the numbers of cells displaying cytoplasmic/nuclear staining as a percent of total counted cells (mean±S.E, n¼ 300) (FIG. 28B(ii)).

FIG. 28C(i)-28C(ii). MEFs cells and Sept4/ARTS KO MEFs cells were induced for apoptosis with UV for 10 minutes. Cells were immobilized and fluorescently stained for p53, mitotracker (a mitochondrial marker) and DAPI (a nuclei marker) (FIG. 28C(i)). The graphs represent the numbers of cells displaying cytoplasmic/nuclear staining as a percent of total counted cells (mean±S.E, n¼ 300) (FIG. 28C(ii)).

FIG. 29A-29E. XIAP acts as an E3 ligase for p53

FIG. 29A(i)-29A(ii). Western blot analysis of WT MEFs and XIAP KO MEFs (FIG. 29A(i)). Densitometry analysis represents three biologically independent repeats (FIG. 29A(ii)).

FIG. 29B. XIAP binds p53. Immunoprecipitation assay of XIAP shows that XIAP and p53 bind each other under normal conditions and to a lesser extent under apoptotic induction. Apoptosis was induced using 200 μM Etoposide (ETP) for 3 hours.

FIG. 29C(i)-29C(ii). XIAP serves as an E3 ligase of p53. In vitro ubiquitylation assay was performed using recombinant XIAP and p53 proteins, and UbcH5b as E2. Western blot (FIG. 29C(i)). Densitometry analysis of the non-ubiquitinated p53 (FIG. 29C(ii)).

FIG. 29D. p53 half-life in HCT 116 WT and XIAP KO HCP 116 cells treated with Cycloheximide (CHX) 200 μM for the indicated time points.

FIG. 29E(i)-29E(ii). ARTS inhibits p53 ubiquitylation by antagonizing XIAP. In vitro ubiquitylation assay was performed by incubating 0.2 μg of recombinant GST-XIAP and 0.2 μg of recombinant p53 together for 1 hour at 37° C. Different doses of ARTS (0.2, 0.4, and 0.8 μg) were added to the mix (FIG. 29E(i)). Densitometry analysis represents three biologically independent repeats (FIG. 29E(ii)).

FIG. 30. Small-molecule ARTS mimetics can bind XIAP

MST (microscale thermophoresis) analysis of B3 binding to fluorescently labelled recombinant XIAP revealed a direct binding to XIAP-BIR3 with Kd of 36 μM+/−11 μM.

FIG. 31A-31H. Small-molecule ARTS mimetics can reduce XIAP and increase p53 levels to promote apoptosis

FIG. 31A. Western Blot analysis of WT MEFs and Sept4/ARTS KO MEFs cells that were induced for apoptosis with 20 or 40 μM of either A4 or B3 ARTS mimetic compounds for 24 hours, showing that B3 can restore elevation of p53.

FIG. 31B. Co-immuno-precipitation between ARTS and p53. Western Blot analysis of lysates and immunoprecipitation of myc-ARTS using Sept4/ARTS KO MEFs cells. Cells were treated with 20M of B3 for 24 hrs.

FIG. 31C(i)-31C(vi). Western Blot analysis of A375 cells that were induced for apoptosis with 20, 30, 40 μM B3 for 24 hours (31C(i)). Densitometry analysis of three biologically independent repeats (31C(ii)-31C(vi)).

FIG. 31D(i)-31D(v). Western Blot analysis of HCT116 WT cells that were induced for apoptosis with 10, 20, 30 μM B3 for 24 hours (FIG. 31D(i)). Densitometry analysis of three biologically independent repeats (FIG. 31D(ii)-31D(v)).

FIG. 31E(i)-31E(viii). Western Blot analysis of MEF cells (WT and XIAP KO cells) that were induced for apoptosis with 20 μM B3 for 2, 4, 6, 16 hours (FIG. 31E(i)). Densitometry analysis of three biologically independent repeats (FIG. 31E(ii)-31E(viii)), FIGS. 31E(ii), (iv), (vi) and (viii) show the WT, and FIGS. 31E(iii), (v), and (vii) show the KO cells.

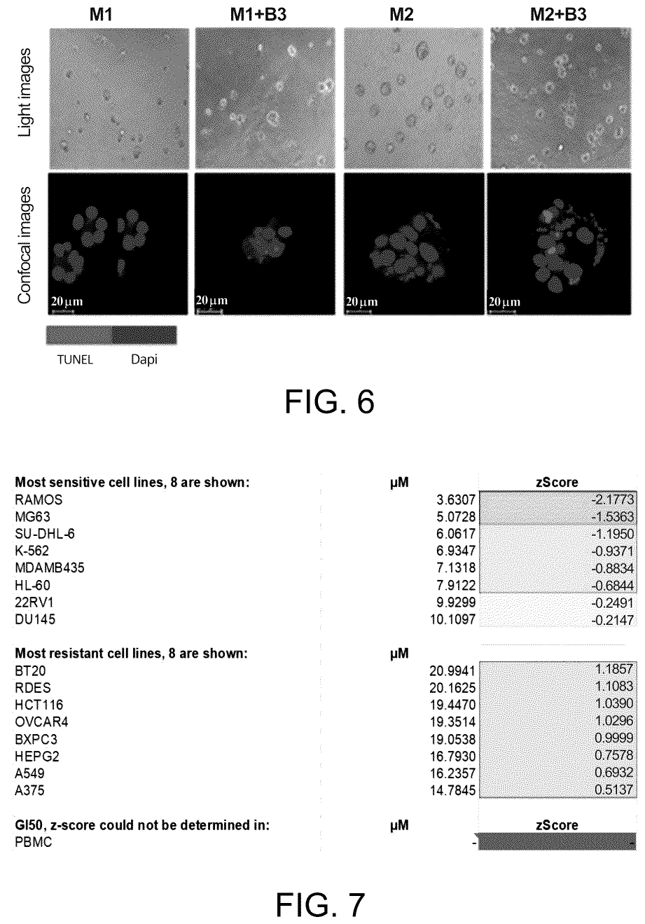

FIG. 31F(i)-31F(ii). in vivo ubiquitylation assay of p53 was performed using HCT 116 WT and HCT 116 XIAP KO cells, followed by Western blot analysis (FIG. 6F(i)). Densitometry analysis represents three biologically independent repeats (FIG. 6F(ii)).

FIG. 31G(i)-31G(ii). B3 inhibits p53 ubiquitylation by XIAP. In vitro ubiquitylation assay was performed by incubating 0.2 μg of recombinant GST-XIAP and 0.2 μg of recombinant p53 with 20 and 40 μM of B3 for 1 hour at 37C (FIG. 6G(i)). Densitometry analysis represents three biologically independent repeats (FIG. 6G(ii)).

FIG. 31H. B3 disrupts the binding between XIAP and p53. Bimolecular fluorescence complementation (BiFC) assay was performed on WT MEFs that were transfected with BiFC pairs of plasmids: VN-p53 and VC-XIAP or VN-p53 and VC-ARTS. 24 h hours post-transfection cells were treated with 20 μM B3 for 18 hours. The fluorescent signal indicating the proximity of each pair of proteins was measured by flow cytometry. MFI, mean fluorescence intensity. Fluorescence-activated cell sorting (FACS) results were normalized to the readings of the transfection efficiency reporter (pdsRED).

One Way ANOVA statistical analysis was done using GraphPad Prism version 8, Mean +/−SD; **p-value<0.001; *p-value<0.05.

### DETAILED DESCRIPTION OF THE INVENTION

The apoptotic pathway is an ordered process of programmed cell death that is often altered in various pathologic conditions associated with either increased or decreased apoptosis.

Modulating apoptosis by external means provides an important and promising approach that paves the way for a variety of therapeutically opportunities. For example, cancer is a condition associated with deregulated apoptosis, resulting in cells displaying increased survival. Thus, inducing apoptosis is valuable as a defense mechanism against hyper proliferating cells. It was shown that Bcl-2 proteins that are anti-apoptotic proteins govern the pro-survival pathway and are over expressed in a variety of tumor types such small cell lung cancer, melanoma, prostate and breast cancer.

Cancer treatment is among others aimed in restoring the apoptotic capabilities of cancer cells. Further, inhibitors of Bcl-2 and XIAP anti-apoptotic proteins are needed in order to revert to normal apoptotic processes and thus trigger tumor cell death.

As indicated above, the inventors have previously found that upon induction of apoptosis, ARTS binds directly to both XIAP and Bcl-2, acting as a scaffold to bring these proteins together, leading to a UPS mediated degradation of Bcl-2.

The inventors have now developed novel ARTS mimetic compounds that target the ARTS-binding site within the XIAP BIR3 domain. These compounds act as ARTS mimetic compound mimicking ARTS unique C-terminal domain and binding thereof to distinct binding sequences in XIAP BIR3 domain. Functional assays revealed that the ARTS mimetic compounds of the invention induce apoptosis.

The inventors found that compounds having at least one amine group and at least one carbonyl group act as ARTS mimetics compounds. Specifically, the inventors found that 1,2 and 1,5 di-carbonyl compounds act as ARTS mimetics.

In a first aspect, the present disclosure provides an apoptosis related protein in the TGF-beta signaling pathway (ARTS) mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for inducing apoptosis in a cell, wherein formula (I) is:

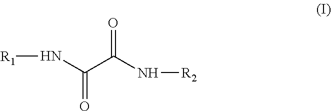

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In some embodiments, of the disclosed compound or composition, the L1′, L1″, L2′ and L2″ are independently from each other selected from —(CH₂)ₙ—, C(═O), optionally —(CH₂)ₙ— substituted with —(CH₂)ₘ—OH and wherein n is an integer selected from 1, 2, or 3 and m is an integer selected from 1, 2 or 3.

In yet some further embodiments of the compound or composition for use of the present disclosure, each one of R₃′, R₃″, R₄′, R₄″ is absent or may be independently from each other an aromatic or a heteroaromatic ring, each is independently from each other optionally substituted with OH, alkyl, halogen, CF₃, NO₂, C(═O).

Still further in some embodiments of the compound or composition for use in accordance with the present disclosure, having the general formula (II)

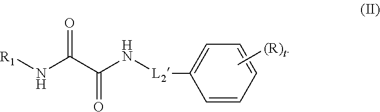

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some further embodiments of the disclosed compound or composition for use according to the present disclosure, wherein L2′ is (CH₂)ₙ—, or C(═O), optionally substituted by one or more of C₁-C₅alkoxy, C₁-C₅ carboxylic acid, —(CH₂)ₘ—OH, —(CH₂)ₘ SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In yet some further embodiments of the disclosed compound or composition for use, the compounds having the general formula (IIb):

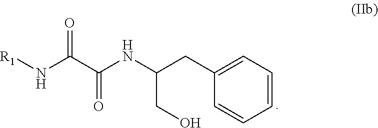

In certain embodiments of the compound or composition for use in accordance with the present disclosure, wherein R1 is L1′-R₃′-L1″-R₃″.

In some further embodiments of the compound or composition for use of the present disclosure, wherein R₁ is at least one of
(i) L1′, L1″ and R₃″ are each absent and R₃′ is an optionally substituted

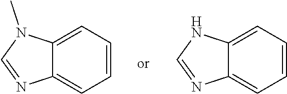

(ii) L1′, L1″ and R₃″ are each absent and R₃′ is:

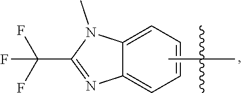

(iii) L1′ is absent R₃′ is an optionally substituted phenyl, R₃″ is an optionally substituted:

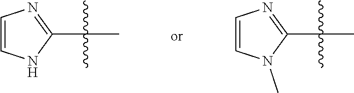

and L1″ is C(═O).

In some further embodiments of the compound or composition for use of the present disclosure, the compound is having the general formula (IIIa) or (IIIb):

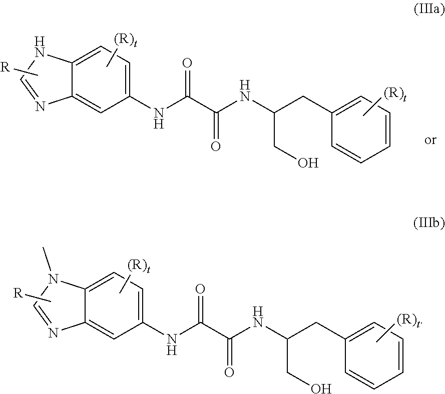

wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5. In yet some further embodiments of the compound or composition for use of the present disclosure, the compound having the general formula (IIIc), (Id) or (IIIe):

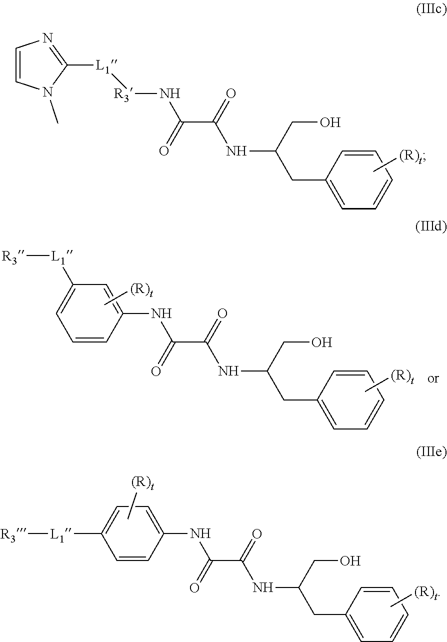

wherein L1″ and R₃″ are each as defined above, wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments, the compound or composition for use according to the present disclosure, wherein the compound is having the general formula (IIIc), or (IIIe):

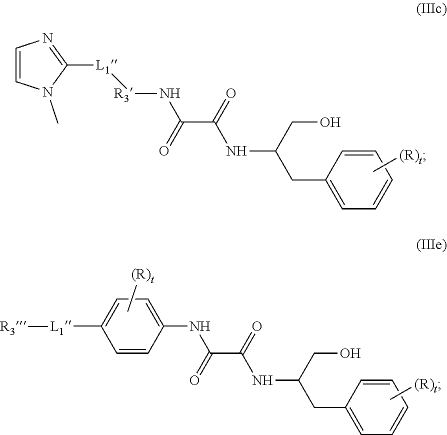

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some further embodiments of the compound or composition for use of the present disclosure, the compound is having the formula (3.1), (3.2), (3.3);

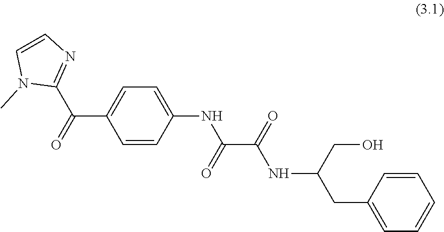 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

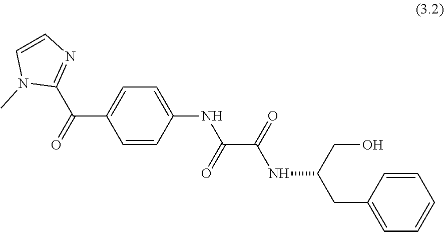

### (S)-N1-(1-hydroxy-3-phenylpropan-2-yl)-N2-(4-(1-methyl-1H-imidazole-2-carbonyl)phenyl)oxalamide (denoted herein as “B3”)

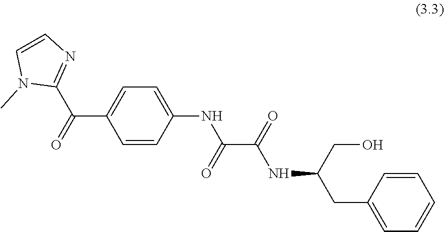 [SMILES: Cn1ccnc1C(=O)c1ccc(NC(=O)C(=O)N[C@@H](CO)Cc2ccccc2)cc1]

### (R)-N1-(1-hydroxy-3-phenylpropan-2-yl)-N2-(4-(1-methyl-1H-imidazole-2-carbonyl)phenyl)oxalamide

In some further embodiments of the compound or composition for use in accordance with the present disclosure, wherein the ARTS mimetic compound leads to ubiquitin proteasome system (UPS) mediated degradation of at least one of B-cell lymphoma 2 (Bcl-2) and X-linked-Inhibitor of Apoptosis (XIAP) in a cell.

Still further, in some further embodiments of the compound or composition for use of the present disclosure, wherein said ARTS mimetic compound leads to elevation in at least one of c-caspase and c-PARP levels in a cell.

In some further embodiments of the compound or composition for use of the present disclosure, wherein the ARTS mimetic compound induces apoptosis in a premalignant and/or a malignant cell.

In some further embodiments of the compound or composition for use in accordance with the present disclosure, the cell is at least one of an epithelial carcinoma cell, a melanoma cell, a sarcoma cell, and hematological cancer cell.

Still further, in some further embodiments of the compound or composition for use of the present disclosure, wherein the cell is of a subject suffering from at least one proliferative disorder.

In some further embodiments of the compound or composition for use according to the present disclosure, the method is for inducing apoptosis in at least one cell in a subject suffering from at least one pathologic disorder, and wherein the method comprising administering to said subject a therapeutically effective amount of said compound.

In yet some further embodiments of the compound or composition for use of the present disclosure, is for use in a method for treating, preventing, inhibiting, reducing, eliminating, protecting or delaying the onset of a pathologic disorder in a subject in need thereof.

Another aspect of the present disclosure relates to an ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for treating, preventing, inhibiting, reducing, eliminating, protecting or delaying the onset of a pathological disorder in a subject in need thereof, wherein formula (I) is:

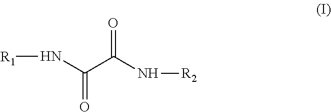

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In some embodiments of the compound or composition for use of the present disclosure, the compound is having the general formula (II)

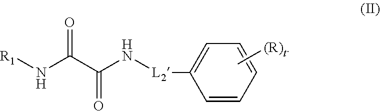

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the compound or composition for use of the present disclosure, the compound is having the general formula (IIIc), or (IIIe):

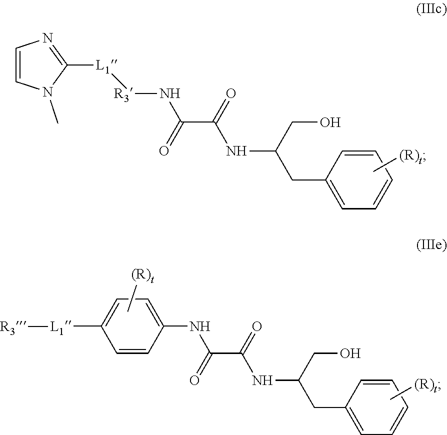

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In yet some further embodiments of the compound or composition for use in accordance with the present disclosure, the compound is having the formula (3.1), (3.2), (3.3);

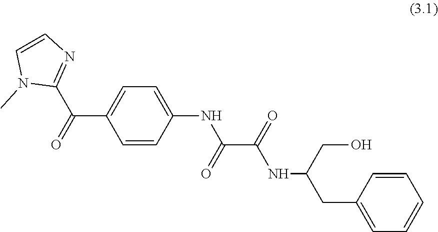 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

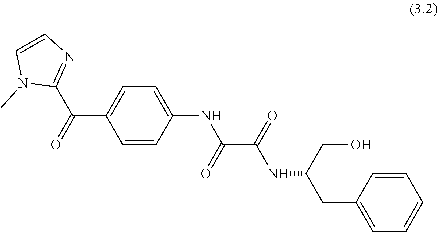

### (S)—N1-(1-hydroxy-3-phenylpropan-2-yl)-N2-(4-(1-methyl-1H-imidazole-2-carbonyl)phenyl)oxalamide (denoted herein as “B33”)

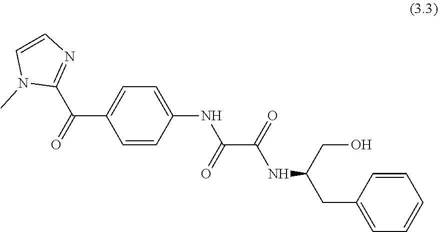 [SMILES: Cn1ccnc1C(=O)c1ccc(NC(=O)C(=O)N[C@@H](CO)Cc2ccccc2)cc1]

### (R)—N1-(1-hydroxy-3-phenylpropan-2-yl)-N2-(4-(1-methyl-1H-imidazole-2-carbonyl)phenyl)oxalamide

In some embodiments of the compound or composition for use of the present disclosure,
wherein the pathologic disorder is characterized by at least one of:
(a) over expression of Bcl-2;
(b) over expression of XIAP; and
(c) low or no expression of ARTS.

Still further, in some embodiments of the compound or composition for use of the present disclosure, the subject is a subject suffering from at least one proliferative disorder, optionally, said proliferative disorder is at least one of carcinoma, melanoma, sarcoma and/or at least one hematological disorder.

A further aspect of the preset disclosure relates to a method for inducing apoptosis in a cell, wherein said method comprises the step of contacting said cell with an effective amount of at least one ARTS mimetic compound, any combination thereof or any composition comprising the same, wherein said ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same. In some embodiments. Formula (I) is:

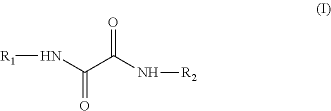

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—, each optionally substituted by one or more of C₁-C₅alkoxy, C₁-C₅ carboxylic acid, —(CH₂)ₘ—OH, —(CH₂)ₘ SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5.

In some embodiments of the disclosed methods, the ARTS mimetic compound is having the general formula (II)

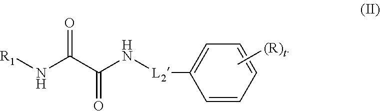

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the methods of the present disclosure, the ARTS mimetic compound is having the general formula (IIIc), or (IIIe):

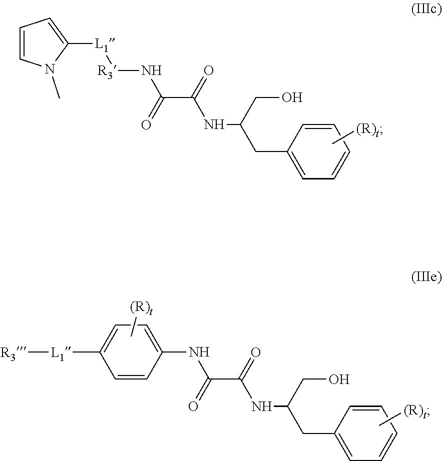

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the methods of the present disclosure, the ARTS mimetic compound is having the formula (3.1), (3.2), (3.3);

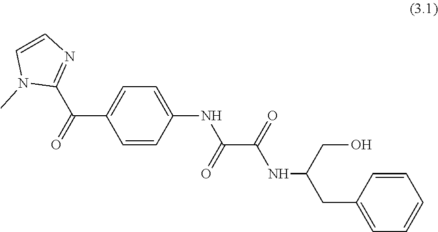 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

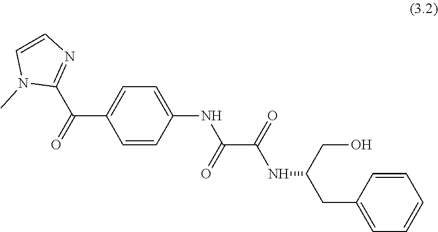

### (S)-N1-(1-hydroxy-3-phenylpropan-2-yl)-N2-(4-(1-methyl-1H-imidazole-2-carbonyl)phenyl)oxalamide (denoted herein as “B3”)

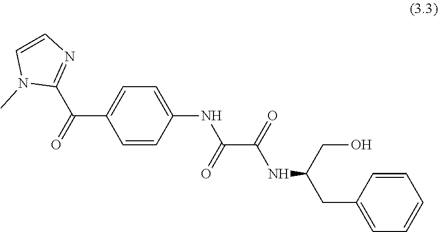 [SMILES: Cn1ccnc1C(=O)c1ccc(NC(=O)C(=O)N[C@@H](CO)Cc2ccccc2)cc1]

### (R)-N1-(1-hydroxy-3-phenylpropan-2-yl)-N2-(4-(1-methyl-1H-imidazole-2-carbonyl)phenyl)oxalamide

In some embodiments of the methods of the present disclosure, the ARTS mimetic compound leads to at least one of UPS mediated degradation of at least one of Bcl-2 and XIAP and elevation in at least one of c-caspase and c-PARP levels.

In some embodiments, the methods of the present disclosure are for inducing apoptosis in at least one of pre-malignant and malignant cell/s.

In some embodiments of the methods of the present disclosure, the cell is at least one of an epithelial carcinoma cell, a sarcoma cell, a melanoma cell and hematological malignant cell.

In some embodiments of the methods of the present disclosure, the cell is characterized by at least one of: (a) over expression of Bcl-2; (b) over expression of XIAP; and (c) low or no expression of ARTS.

In some embodiments, the methods of the present disclosure are for inducing apoptosis of at least one cell in a subject in need thereof, wherein contacting said cell with an effective amount of at least one ARTS mimetic compound comprises administering to said subject an effective amount of said compound or of any composition thereof.

A further aspect of the present disclosure relates to a method for treating, preventing, inhibiting, reducing, eliminating, protecting or delaying the onset of a pathological disorder in a subject in need thereof. The disclosed method comprises administering to said subject a therapeutically effective amount of at least one ARTS mimetic compound or of a composition comprising the same, said ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same; wherein said Formula (I) is:

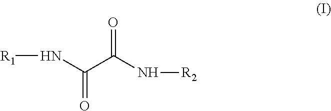

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—, each optionally substituted by one or more of C₁-C₅alkoxy, C₁-C₅ carboxylic acid, —(CH₂)ₘ—OH, —(CH₂)ₘ SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5.

In some embodiments of the methods of the present disclosure, the L1′, L1″, L2′ and L2″ are independently from each other selected from —(CH₂)ₙ—, C(═O) optionally —(CH₂)ₙ— substituted with —(CH₂)ₘ—OH and wherein n is an integer selected from 1, 2, or 3 and m is an integer selected from 1, 2 or 3.

In some embodiments of the methods of the present disclosure, each one of R₃′, R₃″, R₄′, R₄″ is absent or may be independently from each other an aromatic or a heteroaromatic ring, each is independently from each other optionally substituted with OH, alkyl, halogen, CF₃, NO₂, C(═O).

In some embodiments of the methods of the present disclosure, the compound used is having the general formula (II)

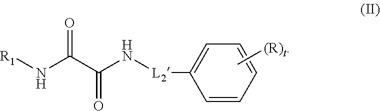

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the methods of the present disclosure, the L₂′ is (CH₂)ₙ—, or C(═O), optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some embodiments of the methods of the present disclosure, the compound used is having the general formula (IIb):

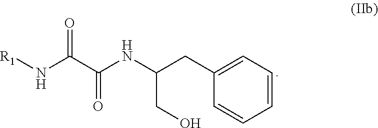

In some embodiments of the methods of the present disclosure, the R1 is L1′-R₃′-L1″-R₃″.

In some embodiments of the methods of the present disclosure, the R₁ is at least one of
(iii) L1′, L1″ and R₃″ are each absent and R₃′ is an optionally substituted

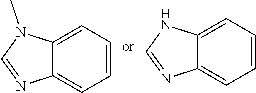

(iv) L1′, L1″ and R₃″ are each absent and R₃′ is:

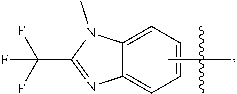

(iii) L1′ is absent R₃′ is an optionally substituted phenyl, R₃″ is an optionally substituted:

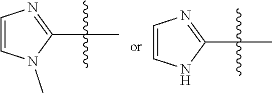

### and L1″ is C(═O).

In some embodiments of the methods of the present disclosure, the compound is having the general formula (IIIa) or (IIIb):

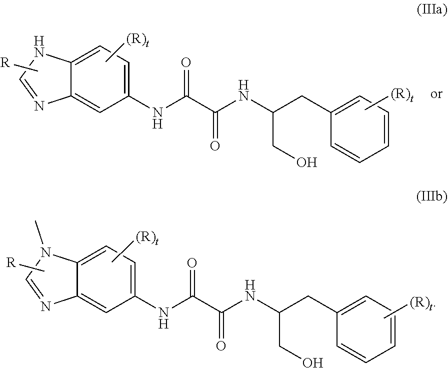

wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the methods of the present disclosure, the compound is having the general formula (IIIc), (IIId) or (IIIe):

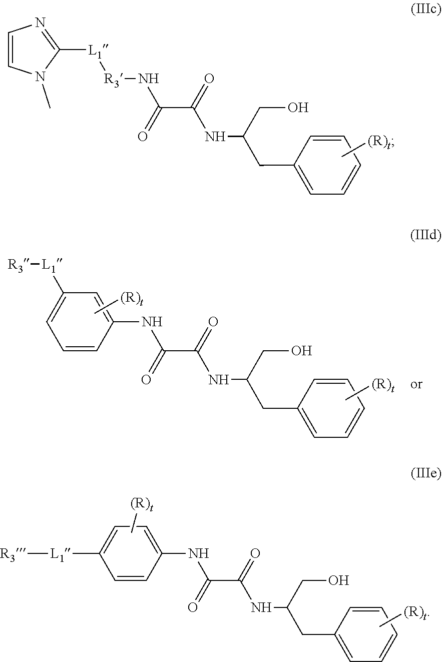

wherein L1″ and R3″ are each as defined above, wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the methods of the present disclosure, the compound is having the general formula (IIIc), or (IIIe):

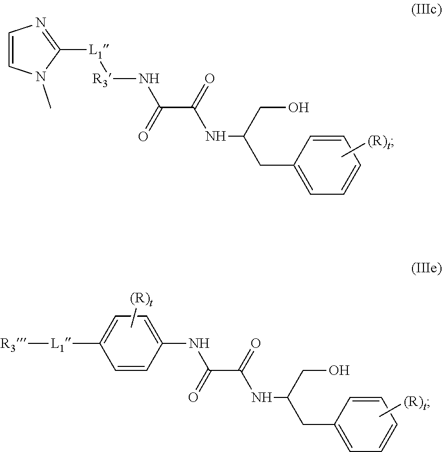

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the methods of the present disclosure, the compound is having the formula (3.1), (3.2), (3.3);

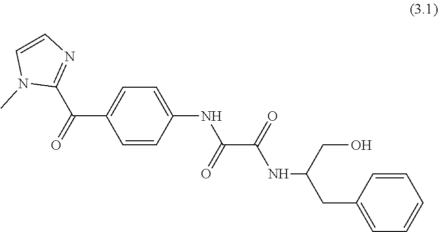 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

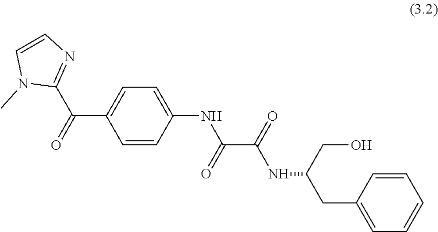

### (S)-N1-(1-hydroxy-3-phenylpropan-2-yl)-N2-(4-(1-methyl-1H-imidazole-2-carbonyl)phenyl)oxalamide (denoted herein as “B3”)

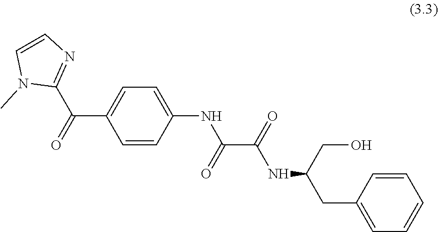 [SMILES: Cn1ccnc1C(=O)c1ccc(NC(=O)C(=O)N[C@@H](CO)Cc2ccccc2)cc1]

### (R)-N1-(1-hydroxy-3-phenylpropan-2-yl)-N2-(4-(1-methyl-1H-imidazole-2-carbonyl)phenyl)oxalamide

In some embodiments of the methods of the present disclosure, the subject is suffering from a pathologic disorder characterized by at least one of: (a) over expression of Bcl-2; (b) over expression of XIAP; and (c) low or no expression of ARTS.

In some embodiments of the methods of the present disclosure, the subject is a subject suffering from at least one proliferative disorder.

In some embodiments of the methods of the present disclosure, the subject is a subject suffering from at least one of carcinoma, melanoma, sarcoma and/or at least one hematological disorder.

Another aspect of the preset disclosure relates to an ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, wherein formula (I) is:

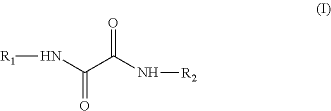

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In some embodiments of the disclosed compound or composition, the L₁′, L₁″, L₂′ and L₂″ are independently from each other selected from —(CH₂)ₙ—, C(═O) optionally-(CH₂)ₙ-substituted with —(CH₂)ₘ—OH and wherein n is an integer selected from 1, 2, or 3 and m is an integer selected from 1, 2 or 3.

In some embodiments of the disclosed compound or composition, each one of R₃′, R₃″, R₄′, R₄″ is absent or may be independently from each other an aromatic or a heteroaromatic ring, each is independently from each other optionally substituted with OH, alkyl, halogen, CF₃, NO₂, C(═O).

In some embodiments of the disclosed compound or composition, the compound is having the general formula (II)

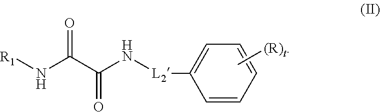

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

The compound or composition claim 53, L₂′ is (CH₂)ₙ—, or C(═O), each optionally substituted by one or more of C₁-C₅alkoxy, C₁-C₅ carboxylic acid, —(CH₂)ₘ—OH, —(CH₂)ₘ SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some embodiments of the disclosed compound or composition, the compound is having the general formula (IIb):

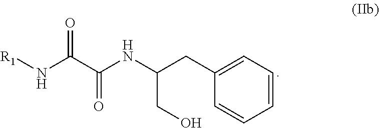

In some embodiments of the disclosed compound or composition, the R1 is L1′-R₃′-L1″-R₃″.

In some embodiments of the disclosed compound or composition, wherein R1 is at least one of
(v) L1′, L1″ and R₃″ are each absent and R₃′ is an optionally substituted

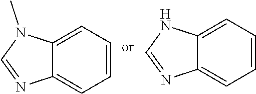

(vi) L1′, L1″ and R₃″ are each absent and R₃′ is:

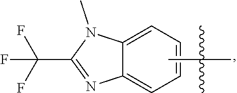

(iii) L₁′ is absent R₃′ is an optionally substituted phenyl, R₃″ is an optionally substituted:

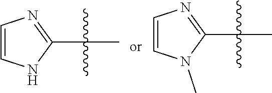

and L1″ is C(═O).

In some embodiments of the disclosed compound or composition, the compound is having the general formula (IIIa) or (IIIb):

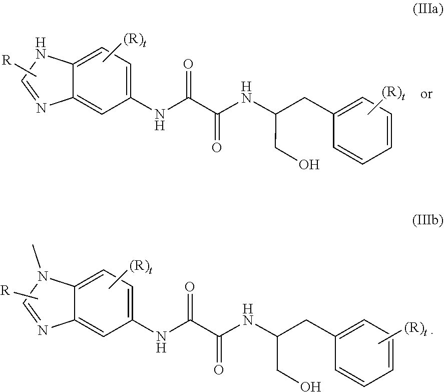

wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the disclosed compound or composition, the compound is having the general formula (IIIc), (IIId) or (IIIe):

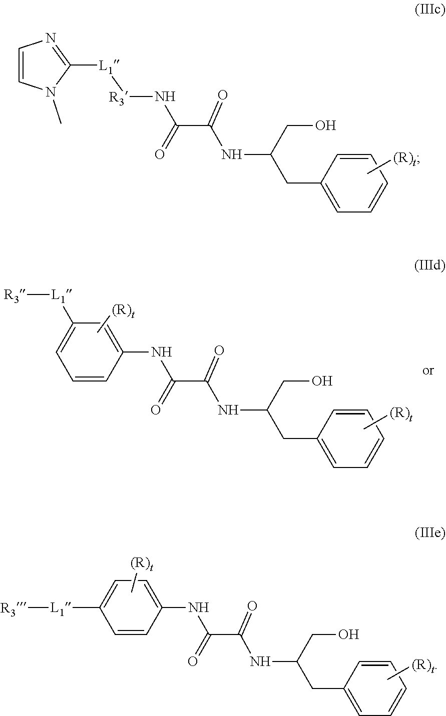

wherein L1″ and R3″ are each as defined above, wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the disclosed compound or composition, the compound is having the general formula (IIIc), or (IIIe):

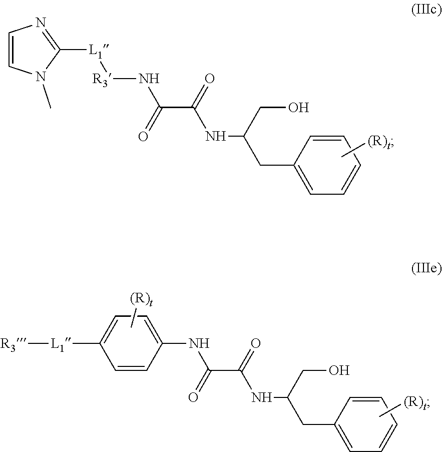

wherein R3′ and R3″ is each independently from the other, an optionally substituted aryl or an optionally substituted heteroaryl, and L1″ is C(═O), wherein R is one or more substituents as defined above, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other.

In some embodiments of the disclosed compound or composition, the compound having the formula (3.1), (3.2), (3.3);

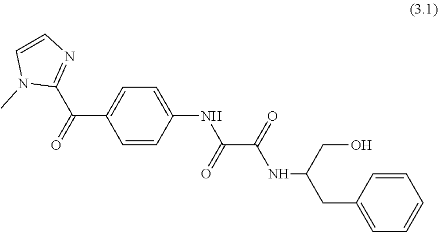 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

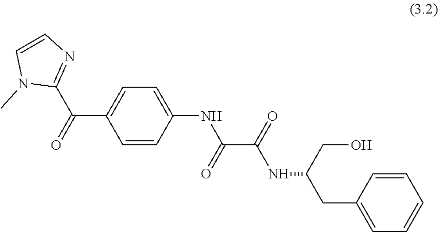

### (S)-N1-(1-hydroxy-3-phenylpropan-2-yl)-N2-(4-(1-methyl-1H-imidazole-2-carbonyl)phenyl)oxalamide (denoted herein as “B3”)

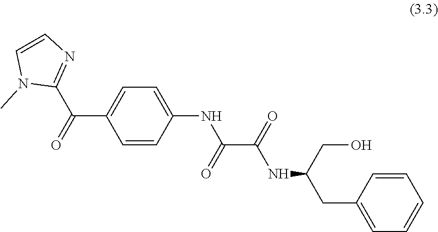 [SMILES: Cn1ccnc1C(=O)c1ccc(NC(=O)C(=O)N[C@@H](CO)Cc2ccccc2)cc1]

### (R)-N1-(1-hydroxy-3-phenylpropan-2-yl)-N2-(4-(1-methyl-1H-imidazole-2-carbonyl)phenyl)oxalamide

In some embodiments of the disclosed compound or composition, the ARTS mimetic compound leads to at least one of UPS mediated degradation of at least one of Bcl-2, XIAP, and/or elevation in at least one of c-caspase 3 and c-PARP levels in a cell.

It should be appreciated that in some embodiments, the present disclosure provides and encompasses any of the compounds disclosed herein.

In some specific and non-limiting embodiments compound is any of the disclosed compounds with the proviso that the compound is not the compound of formula (3.1), specifically;

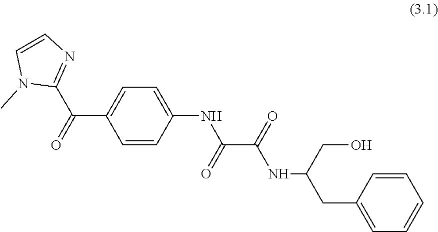 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

In some specific and non-limiting embodiments compound is any of the disclosed compounds with the proviso that the compound is not the compound of formula (3.2), specifically;

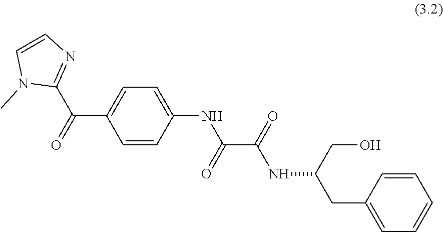 [SMILES: Cn1ccnc1C(=O)c1ccc(NC(=O)C(=O)N[C@H](CO)Cc2ccccc2)cc1]

### (S)-N1-(1-hydroxy-3-phenylpropan-2-yl)-N2-(4-(1-methyl-1H-imidazole-2-carbonyl)phenyl)oxalamide (denoted herein as “B3”)

In some specific and non-limiting embodiments compound is any of the disclosed compounds with the proviso that the compound is not the compound of formula (3.3), specifically:

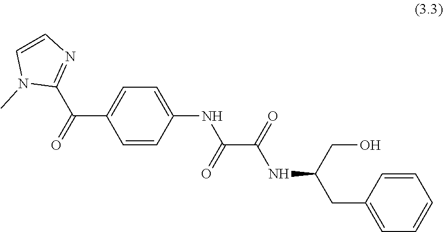 [SMILES: Cn1ccnc1C(=O)c1ccc(NC(=O)C(=O)N[C@@H](CO)Cc2ccccc2)cc1]

### (R)—N1-(1-hydroxy-3-phenylpropan-2-yl)-N2-(4-(1-methyl-1H-imidazole-2-carbonyl)phenyl)oxalamide

A further aspect of the present disclosure relates to a combined composition comprising an effective amount of at least one ARTS mimetic compound and at least one and at least one B-cell lymphoma 2 (Bcl-2) Homology 3 (BH3) mimetic compound, wherein said ARTS mimetic compound is having the general formula (I) or a pharmaceutically acceptable salt, solvate, hydrate, any stereoisomer thereof or physiologically functional derivative thereof, and wherein formula (I) is:

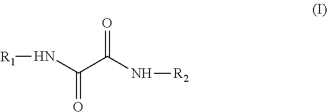

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5 each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In some embodiments of the combined composition of the present disclosure, the wherein said at least one ARTS mimetic compound is any of the compounds defined and disclosed by the present disclosure.

In some embodiments of the combined composition of the present disclosure, the BH3 mimetic compound is 4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

A further aspect of the present disclosure relates to a kit comprising: at least one ARTS mimetic compound having the general formula (I) or a pharmaceutically acceptable salt, solvate, hydrate, any stereoisomer thereof or physiologically functional derivative thereof, wherein formula (I) is:

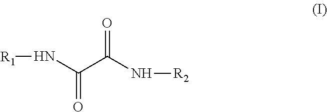

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5 each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5; and
(b) at least one BH3 mimetic compound.

In some embodiments of the kits of the preset disclosure, the ARTS mimetic compound is as defined and disclosed by the present disclosure.

Still further, in some embodiments of the kits of the present disclosure, the ARTS mimetic compound is having the formula (3.1), (3.2), (3.3);

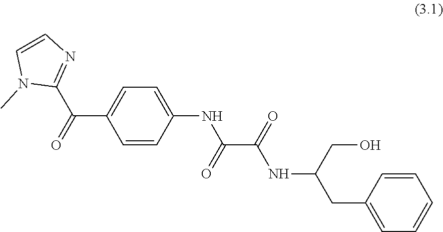 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

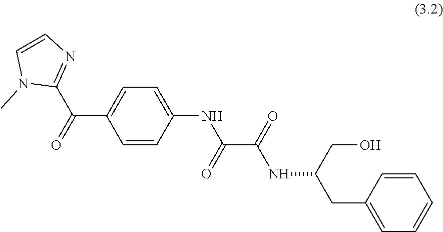 [SMILES: Cn1ccnc1C(=O)c1ccc(NC(=O)C(=O)N[C@H](CO)Cc2ccccc2)cc1]

### (S)-N1-(1-hydroxy-3-phenylpropan-2-yl)-N2-(4-(1-methyl-1H-imidazole-2-carbonyl)phenyl)oxalamide (Denoted Herein as “B3”)

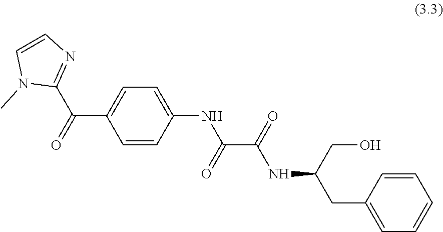 [SMILES: Cn1ccnc1C(=O)c1ccc(NC(=O)C(=O)N[C@@H](CO)Cc2ccccc2)cc1]

### (R)—N1-(1-hydroxy-3-phenylpropan-2-yl)-N2-(4-(1-methyl-1H-imidazole-2-carbonyl)phenyl)oxalamide

In yet some further embodiments of the disclosed kits, the BH3 mimetic compound is 4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

A further aspect of the present disclosure relates to a method for inducing apoptosis in a cell comprising the step of contacting said cell with an effective amount of at least one ARTS mimetic compound as defined by the present disclosure, and of at least one BH3 mimetic compound, with a combined composition as defined by the present disclosure, or with any kit as defined by the present disclosure.

In some embodiments of the disclosed methods, the BH3 mimetic compound is 4-[4-[[2-(4-Chlorophenyl)-4,4-dimethyl-1l-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

A further aspect of the present disclosure relates to a method for treating, preventing, inhibiting, reducing, eliminating, protecting or delaying the onset of a proliferative disorder in a subject in need thereof, the method comprises administering to said subject a therapeutically effective amount of at least one ARTS mimetic compound as defined by the present disclosure, and of at least one BH3 mimetic compound, or of any composition or kit comprising the BH3 mimetic compound and said ARTS mimetic compound.

In some embodiments of the disclosed methods, the BH3 mimetic compound is 4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

In accordance with one aspect, the present disclosure provides 1,2-di-carbonyl compounds. In accordance with this aspect, the present disclosure provides a compound comprising at least one oxalamide moiety. The present disclosure also encompasses pharmaceutically acceptable salt, solvate, hydrate or any stereoisomer of the compounds described herein.

In accordance with this aspect, the present disclosure provides a compound having the general formula (I):

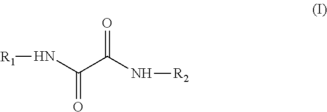

or a pharmaceutically acceptable salt, solvate, hydrate, any stereoisomer thereof or physiologically functional derivative thereof;
wherein
R₁, R₂ are each, independently from each other, absent or H, alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, or —S—(CH₂)ₙ—, each optionally substituted; n is an integer selected from any one of 0, 1, 2, 3, 4, 5 each may be independently from each other, optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen and m is an integer selected from 0, 1, 2, 3, 4, 5.

In the following text, when referring to a compound (such as ARTS mimetic compound) it is to be understood as also referring to the composition, uses (compound for use, composition for use), methods, combined therapies and kits disclosed herein. Thus, whenever providing a feature with reference to the compound, it is to be understood as defining the same feature with respect to the composition, uses (compound for use, composition for use), methods, combined therapies and kits, mutatis mutandis.

In some embodiments, each one of L1′, L1″, L2′ and L2″ is each independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen and m is an integer selected from 0, 1, 2, 3, 4, 5.

In some examples, R₁, R₂ are each, independently from each other, a ring system containing five to twelve atoms.

In some embodiments, a ring system containing five to twelve atoms may be substituted with one or more substituents, in certain embodiments one, two, three or four substituents as further described herein.

In some examples, R₁, R₂ are each, independently from each other, a monocyclic or polycyclic ring system containing five to twelve atoms, optionally substituted with one or more substitutes.

In some embodiments, each one of R₃′, R₃″, R₄′ and R₄″ may be independently from each other a ring system containing five to twelve atoms, each optionally substituted.

In some examples, each one of R₃′, R₃″, R₄′, R₄″ is independently from each other, a monocyclic or polycyclic ring system containing five to twelve atoms, optionally substituted with one or more substitutes.

In some other embodiments, the ring system of each one of R₃′, R₃″, R₄′, R₄″ may be independently from each other an aryl, heteroaryl or aliphatic ring (non-aromatic ring).

In some further embodiments, each one of R₃′, R₃″, R₄′, R₄″ may be independently from each other C₅-C₁₂ saturated cycloalkyl, C₅-C₁₂ saturated cycloalkylene, C₅-C₁₂ aryl, C₅-C₁₂ heteroaryl or C₅-C₁₂ arylene.

In some further embodiments, the ring system of each one of R₃′, R₃″, R₄′, R₄″ may independently from each other contain at least two carbon atoms and may include at least one heteroatom ring.

In some further embodiments, each one of R₃′, R₃″, R₄′, R₄″ may be independently from each other heteroaryl, heteroarylene, heterocycloalkylene or heterocycloalkyl.

In some further embodiments, each one of R₃′, R₃″, R₄′, R₄″ may be independently from each other C₂-C₁₂ heterocycloalkyl ring, C₂-C₁₂ heteroaryl or C₂-C₁₂ heteroarylene.

In some embodiments, the heteroatom in a heteroaryl ring may be N, O, S.

In yet some further embodiments, the heteroatom in a heteroaryl ring may be N, O.

In some other embodiments, the heteroatom in a heteroaryl ring may be N.

In some embodiments, each one of R₃′, R₃″, R₄′, R₄″ is absent, H, an optionally substituted aryl or an optionally substituted heteroaryl.

In some embodiments, each one of R₃′, R₃″, R₄′, R₄″ is absent, an optionally substituted aryl or an optionally substituted heteroaryl.

In some further embodiments, each one of R₃′, R₃″, R₄′, R₄″ is absent or may be independently from each other H, an optionally substituted aromatic ring or an optionally substituted heteroaromatic ring.

In some further embodiments, each one of R₃′, R₃″, R₄′, R₄″ is absent or may be independently from each other an aromatic or heteroaromatic five to eleven membered ring.

In some other embodiments, each one of R₃′, R₃″, R₄′, R₄″ is absent or may be independently from each other H, phenyl, 1-naphthyl, 2-naphthyl, and 4-biphenyl, pyridyl, pyrimidinyl, pyrazinyl, pyridazinyl, triazinyl, furyl (furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, 1,2,3,-oxadiazoyl, 1,2,4,-oxadiazoyl, 1,2,5,-oxadiazoyl, 1,3,4,-oxadiazoyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, and 1,1-dioxo thiomorpholinyl, 1-methyl-1H-benzo[d]imidazole, benzoimidazole, pyridine, pyrrole, 1-methyl-1H-imidazole or 1H-imidazole, each optionally substituted.

In some other embodiments, each one of R₃′, R₃″, R₄′, R₄″ is absent or may be independently from each other H, phenyl, 1-methyl-1H-benzo[d]imidazole, benzoimidazole, pyridine, pyrrole, 1-methyl-1H-imidazole or 1H-imidazole.

In some embodiments, each one of R₃′, R₃″, R₄′, R₄″ is absent or may be independently from each other an aromatic or a heteroaromatic ring, each is independently from each other optionally substituted with one or more of OH, CF₃, halogen, C(═O), —COOH, —NH₂, alkyl, alkylene, C₁-C₁₂ alkoxy, a ring system containing five to twelve atoms, C₁-C₅ carboxyl, halogen.

In some embodiments, each one of R₃′, R₃″, R₄′, R₄″ is absent or may be independently from each other an aromatic or a heteroaromatic ring, each is independently from each other optionally substituted with OH, alkyl, halogen, CF₃, NO₂, C(═O).

In some embodiments, each one L1′, L1″, L2′ and L2″ may be substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen; In some embodiments, each one of L1′, L1″, L2′ and L2″ may be absent or independently from each other selected from —(CH₂)ₙ—, C(═O).

In some further embodiments, each one of L2′, L2″ is absent or may be —(CH₂)ₙ—.

In some further embodiments, each one of L2′, L2″ may be optionally substituted with —(CH₂)ₘ—OH.

In some embodiments, n may be 0 to 3, at times n may be 1 to 3, at times n may be 2 to 3 and m may be 1 to 3.

In some other embodiments, each one of L1′ or L1″ is absent or may be C(═O).

In some examples in which R1 is L1′-R₃′-L1″-R₃″, L₁′ and L1″ are each absent, R₃′ is an optionally substituted aromatic or heteroaromatic five to eleven membered ring and R₃″ is absent.

In some examples in which R1 is L1′-R₃′-L1″-R₃″, L₁′ and L1″ are each absent, R₃′ is an optionally substituted aryl or optionally substituted heteroaryl and R₃″ is absent.

In some examples, in which R1 is L1′-R₃′-L1″-R₃″, L₁′ and L₁″ are each absent, R₃′ is an optionally substituted phenyl, benzoimidazole, 1-methyl-1H-benzo[d]imidazole, pyridine, pyrrole, 1-methyl-1H-imidazole or 1H-imidazole and R₃″ is absent.

In some embodiments, in which R1 is L1′-R₃′-L1″-R₃″, L₁′ and L₁″ are each absent, R₃′ is phenyl, benzoimidazole, 1-methyl-1H-benzo[d]imidazole, pyridine, pyrrole, 1-methyl-1H-imidazole or 1H-imidazole optionally substituted with one or more of OH, CF₃, halogen, C(═O), —COOH, —NH₂, alkyl, alkylene, C₁-C₁₂ alkoxy, a ring system containing five to twelve atoms, C₁-C₅ carboxyl, halogen and R₃″ is absent.

In some embodiments, in which R1 is L1′-R₃′-L1″-R₃″, L₁′ and L₁″ are each absent, R₃′ is phenyl, benzoimidazole, 1-methyl-1H-benzo[d]imidazole, pyridine, pyrrole, 1-methyl-1H-imidazole or 1H-imidazole optionally substituted with OH, alkyl, halogen, CF₃, NO₂, C(═O) and R₃″ is absent.

In some embodiments, in which R1 is L1′-R₃′-L1″-R₃″, L₁′ and L₁″ are each absent, R₃′ is phenyl, benzoimidazole, 1-methyl-1H-benzo[d]imidazole, optionally substituted with OH, alkyl, halogen, CF₃, NO₂, or C(═O) and R₃″ is absent.

In some embodiments, in which R1 is L1′-R₃′-L1″-R₃″, L₁′ and L1″ are each absent, R₃′ is 1-methyl-1H-benzo[d]imidazole, optionally substituted with OH, alkyl, halogen, CF₃, NO₂, or C(═O) and R₃″ is absent.

In some embodiments, in which R1 is L1′-R₃′-L1″-R₃″, L1′ and L1″ are each absent, R₃′ is 1-methyl-1H-benzo[d]imidazole, optionally substituted with halogen, or CF₃, and R₃″ is absent.

In some embodiments, in which R1 is L1′-R₃′-L1″-R₃″, L1′ and L1″ are each absent, R₃′ is 1-methyl-1H-benzo[d]imidazole, optionally substituted with, CF₃, and R₃″ is absent.

In some examples, in which R1 is L1′-R₃′-L1″-R₃″, L₁′ is absent, R₃′ and R₃″ are each independently from the other, an optionally substituted aromatic or heteroaromatic five to eleven membered ring and L₁″ is —(CH₂)ₙ, —NH—(CH₂)ₙ—, C(═O), C(═O)—(CH₂)ₙ—, —O—, —SO₂—, —S—, —S—S—, or —S—(CH₂)ₙ—, each optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen, n is an integer selected from any one of 0, 1, 2, 3, 4, 5 and m is an integer selected from 0, 1, 2, 3, 4, 5.

In some examples in which R1 is L1′-R₃′-L1″-R₃″, L₁′ is absent, R₃′ and R₃″ are each independently from the other, an optionally substituted aryl or an optionally substituted heteroaryl and L₁″ is —(CH₂)ₙ—, —NH—(CH₂)ₙ—, C(═O), C(═O)—(CH₂)ₙ—, —O—, —SO₂—, —S—, —S—S—, or —S—(CH₂)ₙ—, each optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen, n is an integer selected from any one of 0, 1, 2, 3, 4, 5 and m is an integer selected from 0, 1, 2, 3, 4, 5.

In some examples in which R1 is L1′-R₃′-L1″-R₃″, L₁′ is absent, R₃′ and R₃″ are each independently from the other, an optionally substituted aryl or an optionally substituted heteroaryl and L₁″ is —(CH₂)ₙ—, or C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ halogen, n is an integer selected from any one of 0, 1, 2, 3, 4, 5 and m is an integer selected from 0, 1, 2, 3, 4, 5.

In some examples in which R1 is L1′-R₃′-L1″-R₃″, L₁′ is absent, R₃′ and R₃″ are each independently from the other, an optionally substituted aryl or an optionally substituted heteroaryl and L1″ is C(═O).

In some examples in which R1 is L1′-R₃′-L1″-R₃″, L₁′ is absent, R₃′ and R₃″ are each independently from the other, an optionally substituted phenyl, benzoimidazole, 1-methyl-1H-benzo[d]imidazole, pyridine, pyrrole, 1-methyl-1H-imidazole or 1H-imidazole and L1″ is —(CH₂)ₙ—, or C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some examples in which R1 is L1′-R₃′-L1″-R₃″, L₁′ is absent, R₃′ and R₃″ are each independently from the other, an optionally substituted phenyl, benzoimidazole, 1-methyl-1H-benzo[d]imidazole, pyridine, pyrrole, 1-methyl-1H-imidazole or 1H-imidazole and L1″ is C(═O).

In some examples in which R1 is L1′-R₃′-L1″-R₃″, L₁′ is absent, R₃′ and R₃″ are each independently from the other, an optionally substituted phenyl or 1-methyl-1H-imidazole or 1H-imidazole and L1″ is —(CH₂)ₙ—, C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ halogen, n is an integer selected from any one of 0, 1, 2, 3, 4, 5 and m is an integer selected from 0, 1, 2, 3, 4, 5.

In some examples in which R1 is L1′-R₃′-L1″-R₃″, L₁′ is absent, R₃′ is phenyl, R₃″ is 1-methyl-1H-imidazole and L1″ is C(═O).

In some embodiments, the compound of the present disclosure having general formula (I) have the general formula (Ia), (Ib), (Ic), (Id), (Ie), (If), (Ig), (Ih), (Ii), (Ij) (Ik) or (II):

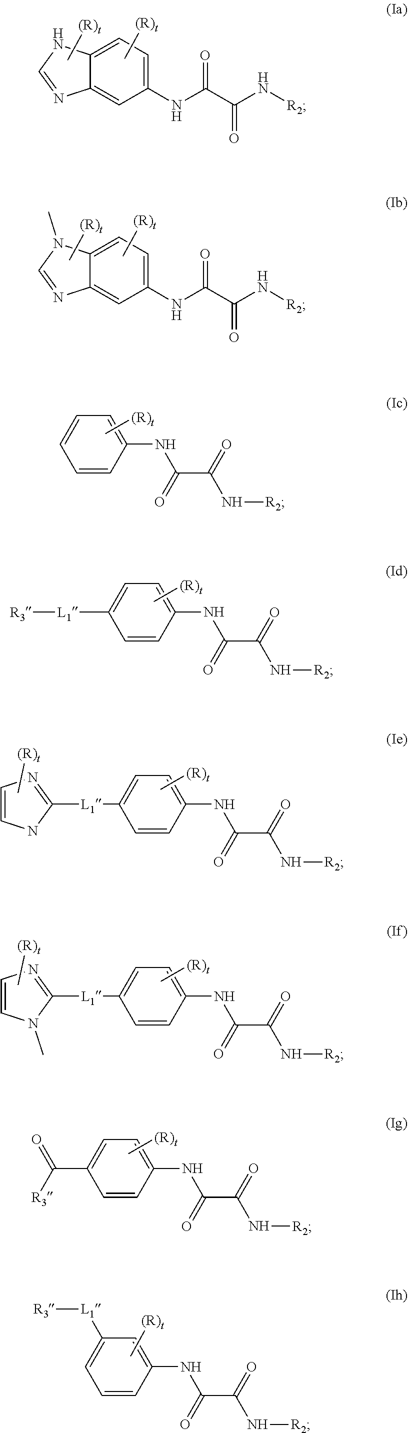

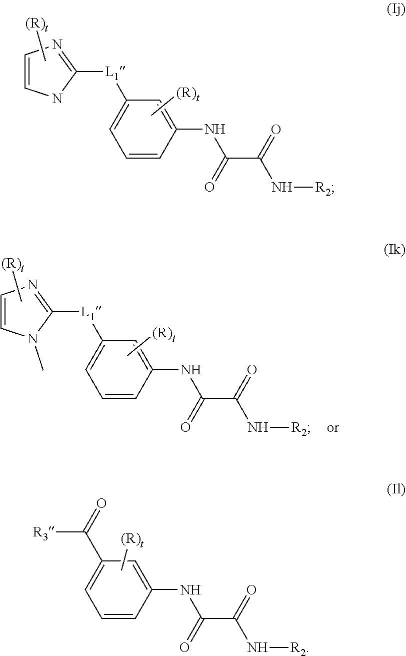

wherein L1″, R3″ are as defined above and wherein R is one or more substituents, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other; t represents the number of substituents and may vary within the same compound being one or more identical substituents (for example one or more H) or different substituents.

In some examples, in compounds of Formula (I), (Id), (Ie), (If), (Ig), (Ih), (Ii), (Ij) (Ik) or (II), L1″ is —(CH₂)ₙ—, —NH—(CH₂)ₙ—, C(═O), C(═O)—(CH₂)ₙ—, —O—, —SO₂—, —S—, —S—S—, or —S—(CH₂)ₙ—, each optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen, n is an integer selected from any one of 0, 1, 2, 3, 4, 5 and m is an integer selected from 0, 1, 2, 3, 4, 5.

In some examples, in compounds of Formula (I), (Id), (Ie), (If), (Ig), (Ih), (Ii), (Ij) (Ik) or (II), L1″ is —(CH₂)ₙ—, or C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some examples, in compounds of Formula (I), (Id), (Ie), (If), (Ig), (Ih), (Ii), (Ij) (Ik) or (II), L1′″ is C(═O).

In some examples, in compounds of Formula (I), (Id), (Ie), (If), (Ig), (Ih), (Ii), (Ij) (Ik) or (II), R3″ is an optionally substituted aryl or an optionally substituted heteroaryl.

In some examples, in compounds of Formula (I), (Id), (Ie), (If), (Ig), (Ih), (Ii), (Ij) (Ik) or (II), R3″ is an optionally substituted 1-methyl-1H-imidazole.

In some examples, in compounds of Formula (I), (Id), (Ie), (If), (Ig), (Ih), (Ii), (Ij) (Ik) or (II), R3″ is 1-methyl-1H-imidazole.

In some examples, in compounds of Formula (I), (Id), (Ie), (If), (Ig), (Ih), (Ii), (Ij) (Ik) or (II), L1″ is C(═O) and R₃″ is 1-methyl-1H-imidazole.

In some examples, in compounds of Formula (I), (Ia), (Ib), (Ic), (Id), (Ie), (If), (Ig), (Ih), (Ii), (Ij) (Ik) or (II), R₂ is L2′-R₄′-L2″-R₄″.

In some examples in which R₂ is L2′-R₄′-L2″-R₄″, L2′ is (CH₂)ₙ—, or C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5, R4′ is an optionally substituted aromatic or heteroaromatic five to eleven membered ring and L2″ and R₄″ are each absent.

In some examples in which R₂ is L2′-R₄′-L₂″-R₄″, L₂′ is an optionally substituted —(CH₂)ₙ—or C(═O), n is an integer selected from 1, 2, 3, 4, 5, R4′ is an optionally substituted aromatic or heteroaromatic five to eleven membered ring and L2″ and R₄″ are each absent.

In some examples in which R₂ is L2′-R₄′-L₂″-R₄″, L₂′ is an optionally substituted —(CH₂)ₙ—or C(═O), n is an integer selected from 1, 2, 3, 4, 5, R4′ is an optionally substituted aryl or an optionally substituted heteroaryl and L2″ and R₄″ are each absent.

In some examples in which R₂ is L2′-R₄′-L₂″-R₄″, L₂′ is an optionally substituted —(CH₂)ₙ, n is an integer selected from 1, 2, 3, 4, 5, R4′ is an optionally substituted aryl or an optionally substituted heteroaryl and L2″ and R₄″ are each absent.

In some examples in which R₂ is L2′-R₄′-L₂″-R₄″, L₂′ is an optionally substituted —(CH₂)ₙ, n is an integer selected from 1, 2, 3, 4, 5, R4′ is an optionally substituted phenyl and L2″ and R₄″ are each absent.

In some examples in which R₂ is L2′-R₄′-L₂″-R₄″, L₂′ is an optionally substituted —(CH₂)₂, R4′ is an optionally substituted aryl or an optionally substituted heteroaryl and L2″ and R₄″ are each absent.

In some examples in which R₂ is L2′-R₄′-L₂″-R₄″, L₂′ is an optionally substituted —(CH₂)₂, R4′ is an optionally substituted phenyl and L2″ and R₄″ are each absent.

In some examples in which R₂ is L2′-R₄′-L₂″-R₄″, L₂′ is —(CH₂)₂ substituted with OH, R4′ is an optionally substituted aryl or an optionally substituted heteroaryl and L2″ and R₄″ are each absent.

In some examples in which R₂ is L2′-R₄′-L₂″-R₄″, L₂′ is —(CH₂)₂ substituted with OH, R4′ is an optionally substituted phenyl and L2″ and R₄″ are each absent.

In some examples in which R₂ is L2′-R₄′-L₂″-R₄″, L₂′, L2″ and R₄″ are each absent, R₄′ is an aryl optionally substituted with OH, alkyl, halogen, CF₃, NO₂, C(═O).

In some examples in which R₂ is L2′-R₄′-L₂″-R₄″, L₂′, L2″ and R₄″ are each absent, R₄′ is a phenyl optionally substituted with OH, alkyl, halogen, CF₃, NO₂, C(═O).

In some examples in which R₂ is L2′-R₄′-L₂″-R₄″, L₂′, L2″ and R₄″ are each absent, R₄′ is a phenyl optionally substituted with OH.

In some embodiments with this aspect, a compound of the invention has the general formula (II)

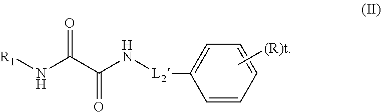

wherein R1 and L2′ is as defined above and wherein R is one or more substituents as defined above, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is as defined above.

In some embodiments, a compound of the invention has the general formula (II), L2′ is (CH₂)ₙ—, or C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some embodiments, a compound of the invention has the general formula (II), L2′ is (CH₂)ₙ—, optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some embodiments, a compound of the invention has the general formula (II), L2′ is (CH₂)ₙ—, substituted by OH, n is an integer selected from any one of 0, 1, 2, 3, 4, 5.

In some embodiments, a compound of the invention has the general formula (II), L2′ is (CH₂)ₙ—, substituted by OH, n is 2.

In some embodiments with this aspect, a compound of the invention has the general formula (IIa) or (IIb):

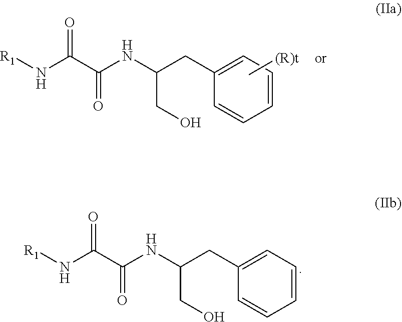

In some examples in the compounds of Formula (II) (IIa), or (IIb), R1 is L1′-R₃′-L₁″-R₃″ as defined above.

In some examples, in the compounds of the disclosure having formula (I), (II), (IIa), or (IIb), R1 is L1′-R₃′-L1″-R₃″, L1′, L1″ and R₃″ are each absent and R₃′ is an optionally substituted

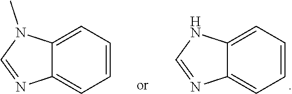

In some examples, in the compounds of the disclosure having formula (I), (II), (IIa), or (IIb), R1 is L1′-R₃′-L1″-R₃″, L1′, L1″ and R₃″ are each absent and R₃′ is an optionally substituted:

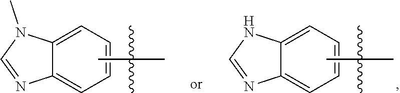

the wavy line represents that the ring linked to formula (I), (II), (IIa), or (IIb).

In some examples, in the compounds of the disclosure having formula (I), (II), (IIa), or (IIb), R1 is L1′-R₃′-L1″-R₃″, L₁′, L₁″ and R₃″ are each absent and R₃′ is:

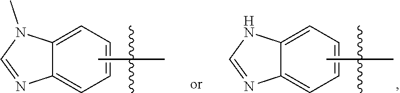

or, optionally substituted with OH, alkyl, halogen, CF₃, NO₂, C(═O), the wavy line indicate bond to formula (I), (II), (IIa), or (IIb).

In some examples, in the compounds of the disclosure having formula (I), (II), (IIa), or (IIb), R1 is L1′-R₃′-L1″-R₃″, L₁′, L₁″ and R₃″ are each absent and R₃′ is:

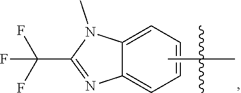

the wavy line indicate bond to formula (I), (II), (IIa), or (IIb).

In some examples, in the compounds of the disclosure having formula (I), (II), (IIa), or (IIb), R1 is L1′-R₃′-L1″-R₃″, L₁′ is absent R₃′ and R₃″ is each independently from the other optionally substituted aryl or an optionally substituted heteroaryl, and L₁″ is —(CH₂)ₙ—, C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5, at times 1, 2, 3, 4, 5 and m is an integer selected from 0, 1, 2, 3, 4, 5.

In some examples, in the compounds of the disclosure having formula (I), (II), (IIa), or (IIb), R1 is L1′-R₃′-L1″-R₃″, R₃′ is an optionally substituted phenyl and R₃″ is an optionally substituted:

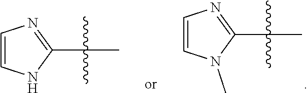

In some examples, in the compounds of the disclosure having formula (I), (II), (IIa), or (IIb), R1 is L1′-R₃′-L1″-R₃″, L₁′ is absent R₃′ is an optionally substituted phenyl, R₃″ is an optionally substituted:

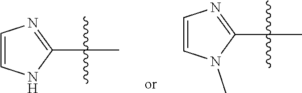

### and L1″ is C(═O).

In some examples, the compound of the present disclosure having general formula (IIIa) or (IIIb):

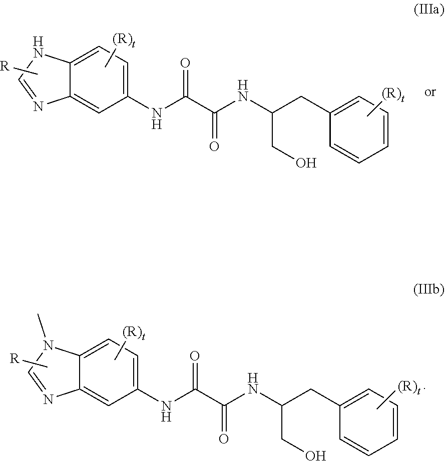

wherein R is one or more substituents as defined above, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other; represents the number of substituents and may vary within the same compound being one or more identical substituents (for example one or more H) or different substituents.

In some examples, the compound of the present disclosure having the general formula (IIIc):

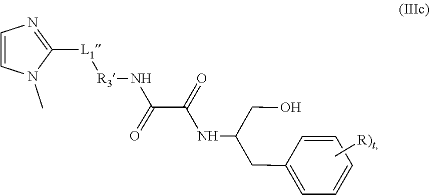

wherein L1″ and R3′ are each as defined above, R is one or more substituents as defined above, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other; t represents the number of substituents and may vary within the same compound being one or more identical substituents (for example one or more H) or different substituents.

In some examples, the compound of the present disclosure having the general formula (IIId):

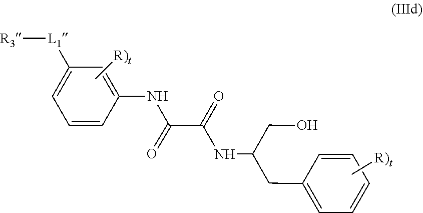

wherein L1″ and R3″ are each as defined above, R is one or more substituents as defined above, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other; t represents the number of substituents and may vary within the same compound being one or more identical substituents (for example one or more H) or different substituents.

In some examples, the compound of the present disclosure having the general formula (IIIe)

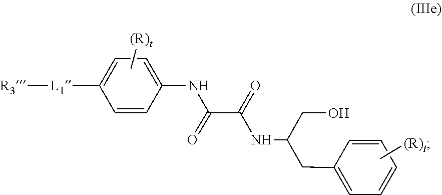

wherein L1″ and R3″ are each as defined above, wherein R is one or more substituents as defined above, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other; t represents the number of substituents and may vary within the same compound being one or more identical substituents (for example one or more H) or different substituents.

In some embodiments, specific examples of compounds or pharmaceutically acceptable salts or hydrates of the compounds of Formula I, II, include, without limitation:

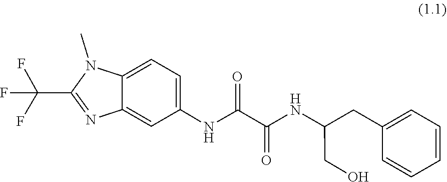 [SMILES: Cn1c(C(F)(F)F)nc2cc(NC(=O)C(=O)NC(CO)Cc3ccccc3)ccc21]

### N1-(1-hydroxy-3-phenylpropan-2-yl)-N2-(1-methyl-2-(trifluoromethyl)-1H-benzo[d]imidazol-5-yl)oxalamide

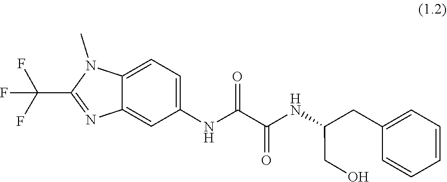 [SMILES: Cn1c(C(F)(F)F)nc2cc(NC(=O)C(=O)N[C@@H](CO)Cc3ccccc3)ccc21]

### (R)-N1-(1-hydroxy-3-phenylpropan-2-yl)-N2-(1-methyl-2-(trifluoromethyl)-1H-benzo[d]imidazol-5-yl)oxalamide

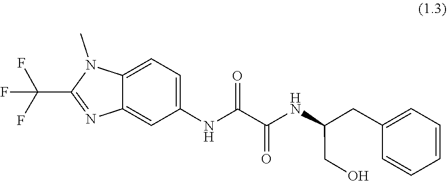 [SMILES: Cn1c(C(F)(F)F)nc2cc(NC(=O)C(=O)N[C@H](CO)Cc3ccccc3)ccc21]

### (S)-N1-(1-hydroxy-3-phenylpropan-2-yl)-N2-(1-methyl-2-(trifluoromethyl)-1H-benzo[d]imidazol-5-yl)oxalamide

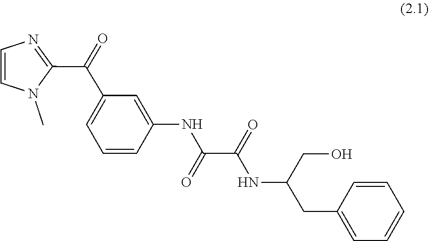 [SMILES: Cn1ccnc1C(=O)c1cccc(NC(=O)C(=O)NC(CO)Cc2ccccc2)c1]

### N1-(1-hydroxy-3-phenylpropan-2-yl)-N2-(3-(1-methyl-1H-imidazole-2-carbonyl)phenyl)oxalamide

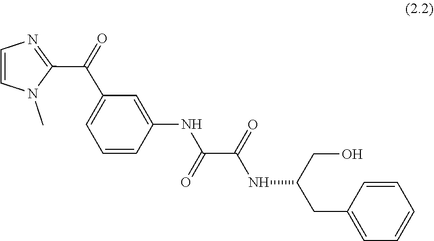 [SMILES: Cn1ccnc1C(=O)c1cccc(NC(=O)C(=O)N[C@H](CO)Cc2ccccc2)c1]

### (S)-N1-(1-hydroxy-3-phenylpropan-2-yl)-N2-(3-(1-methyl-1H-imidazole-2-carbonyl)phenyl)oxalamide

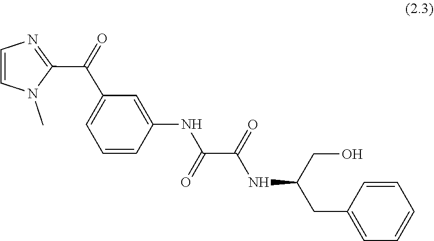 [SMILES: Cn1ccnc1C(=O)c1cccc(NC(=O)C(=O)N[C@@H](CO)Cc2ccccc2)c1]

### (R)-N1-(1-hydroxy-3-phenylpropan-2-yl)-N2-(3-(1-methyl-1H-imidazole-2-carbonyl)phenyl)oxalamide

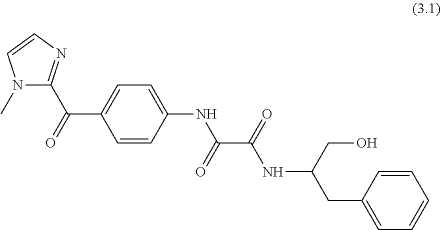 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

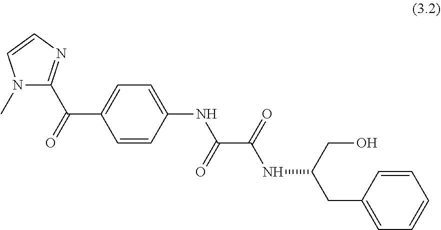

### (S)-N1-(1-hydroxy-3-phenylpropan-2-yl)-N2-(4-(1-methyl-1H-imidazole-2-carbonyl)phenyl)oxalamide (denoted herein as “B3”)

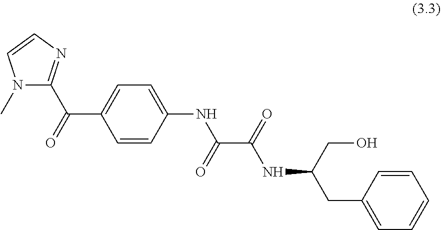 [SMILES: Cn1ccnc1C(=O)c1ccc(NC(=O)C(=O)N[C@@H](CO)Cc2ccccc2)cc1]

### (R)—N1-(1-hydroxy-3-phenylpropan-2-yl)-N2-(4-(1-methyl-1H-imidazole-2-carbonyl)phenyl)oxalamide

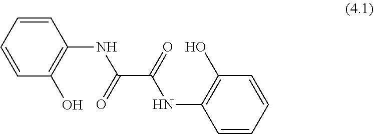 [SMILES: O=C(Nc1ccccc1O)C(=O)Nc1ccccc1O]

### N1,N2-bis(2-hydroxyphenyl)oxalamide

In accordance with the second aspect, the present disclosure provides 1,5-di-carbonyl compounds. In accordance with this aspect, the compounds contain one or more nitrogen atoms. In some other embodiments, the compounds contain at least one ring structure. In some other embodiments, the ring structure containing at least one nitrogen atom. In some further embodiments, the compounds contain a heterocyclic ring structure containing five to twelve atoms, the ring structure containing at least two carbon atoms and at least one heteroatom being N, O or S.

The present disclosure provides in accordance with the second aspect, a compound having the general formula (VIII):

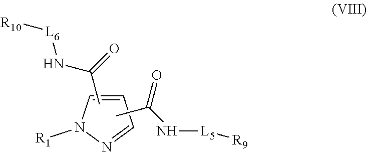

or a pharmaceutically acceptable salt, solvate, hydrate, any stereoisomer thereof or physiologically functional derivative thereof;
wherein
R₉ and R₁₀ may be the same or may be different and may be independently selected from each other from a ring system containing five to twelve atoms, each optionally substituted;
R₁₁ may be independently selected from H, straight or branched C₁-C₁₂ alkyl, straight or branched C₂-C₁₂ alkenyl, straight or branched C₂-C₁₂alkynyl;
L₅ and L₆, are each independently of each other, are absent or may be selected independently from —(CH₂)ₚ—; —NH—C(═O)—(CH₂)ₚ—, —C(═O)—NH—(CH₂)ₚ—; —S—S—(CH₂)ₚ—; —O—(CH₂)ₚ—; —NH—(CH₂)ₚ—; C(═O)—(CH₂)ₚ—; —S—(CH₂)ₚ—; —NH—S(═O)ₚ—(CH₂)ₚ—;
each p, being independently, may be 0 to 5;

In some embodiments, each one of R₉ and R₁₀ being a ring system containing five to twelve atoms may be substituted with one or more substituents, in certain embodiments one, two, three or four substituents, In some further embodiments, the substituents may be selected from OH, CF₃, halogen, C(═O), —COOH, —NH₂, CN, C(═O)—alkyl, alkyl, alkenyl, alkynyl, alkylene, alkenylene, alkynylene, C₁-C₁₂ alkoxy, C₁-C₅ carboxyl, halogen, aromatic or heteroaromatic ring.

In some embodiments, R₉ and R₁₀ may be a ring system containing five to twelve atoms. In some further embodiments, the ring system of R₉ and R₁₀ may contain at least two carbon atoms. In some other embodiments, the ring system of R₉ and R₁₀ may be an aromatic ring, non-aromatic ring, fused ring or the like. In some further embodiments, R₉ and R₁₀ may be C₅-C₁₂ saturated cycloalkyl, C₅-C₁₂ saturated cycloalkylene, C₅-C₁₂ aryl or C₅-C₁₂ arylene. In some other embodiments, the ring system of R₉ and R₁₀ may include at least one heteroatom ring. In some further embodiments, R₉ and R₁₀ may be heteroaryl or heterocycloalkyl. In some further embodiments, R₉ and R₁₀ may be C₂-C₁₂ hetero cycloalkyl or C₂-C₁₂ hetero aromatic ring (aryl or arylene). It should be noted that according with some embodiments, R₉ and R₁₀ may be independently from each other contain different carbon atoms. In some embodiments, the heteroatom may be N, O, S. In some embodiments, R₉ and R₁₀ may be independently from each other selected from C₅-C₁₂ aryl. In some embodiments, R₉ and R₁₀ may be C₆ aryl, optionally substituted.

In some embodiments, L5 and L6, may be —(CH₂)ₚ—. According to these embodiments, the group —(CH₂)ₚ—wherein p=1 to 5, may encompasses an alkyl, alkylene, alkenyl, alkenylene and alkynyl as defined herein having at most five carbon atoms. In some embodiments, each of p may be 0. At times, when p=O, L₅ and L₆ are each a bond.

In some embodiments, specific examples of compounds or pharmaceutically acceptable salts or hydrates of the compounds of Formula VIII include, without limitation:

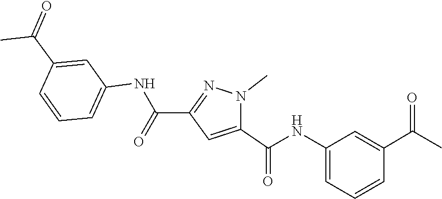 [SMILES: CC(=O)c1cccc(NC(=O)c2cc(C(=O)Nc3cccc(C(C)=O)c3)n(C)n2)c1]

### N3,N5-bis(3-acetylphenyl)-1-methyl-1H-pyrazole-3,5-dicarboxamide (5.1)

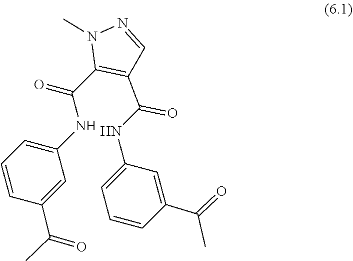 [SMILES: CC(=O)c1cccc(NC(=O)c2cnn(C)c2C(=O)Nc2cccc(C(C)=O)c2)c1]

### N4,N5-bis(3-acetylphenyl)-1-methyl-1H-pyrazole-4,5-dicarboxamide

In accordance with the third aspect, the present disclosure provides compounds containing one or more nitrogen atoms. In some other embodiments, the compounds contain at least one ring structure. In some other embodiments, the ring structure containing at least one nitrogen atom. In some further embodiments, the compounds contain a heterocyclic ring structure containing five to twelve atoms, the ring structure containing at least two carbon atoms and at least one heteroatom being N, O or S. in some other embodiments, the compounds contain an amide group.

In accordance with the third aspect, the present disclosure provides a compound having a general formula:

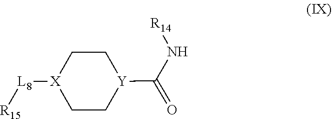

or a pharmaceutically acceptable salt or hydrate thereof;
wherein:
X, Y independently may be each independently selected from each other from NH, CH;
R₁₄ and R₁₅ may be the same or may be different and may be independently from each other selected from a ring system containing five to twelve atoms, each optionally substituted;
L8 may be selected independently from —(CH₂)_{q}—; —NH—C(═O)—(CH₂)_{q}—, —C(═O)—NH—(CH₂)_{q}—; —S—S—(CH₂)_{q}—; —O—(CH₂)_{q}—; —NH—(CH₂)_{q}—; C(═O)—(CH₂)_{q}—; —S—(CH₂)_{q}—; —NH—S(═O)_{q}—(CH₂)_{q}—; each q, being independently, may be 0 to 5;

In some embodiments, R₁₄ and R₁₅ may be a ring system containing five to twelve atoms. In some further embodiments, the ring system of R₁₄ and R₁₅ may contain at least two carbon atoms. In some other embodiments, the ring system of R₁₄ and R₁₅ may be an aromatic ring or a non-aromatic ring. In some further embodiments, R₁₄ and R₁₅ may be C₅-C₁₂ saturated cycloalkyl, C₅-C₁₂ saturated cycloalkylene, C₅-C₁₂ aryl or C₅-C₁₂ arylene. In some other embodiments, the ring system of R₁₄ and R₁₅ may include at least one heteroatom ring. In some further embodiments, R₁₄ and R₁₅ may be C₂-C₁₂ hetero cycloalkyl or C₂-C₁₂ hetero aromatic ring. It should be noted that according with some embodiments, R₁₄ and R₁₅ may be independently from each other contain different carbon atoms. In some embodiments, the heteroatom may be N, O, S. In some embodiments, R₁₄ and R₁₅ may be independently from each other selected from C₅-C₁₂ aryl optionally substituted. In some embodiments, R₁₄ and R₁₅ may be independently from each other selected from isoquinoline or phyel each independently from the other optionally substituted.

In some embodiments, L8, may be —(CH₂)_{q}—. According to these embodiments, the group —(CH₂)_{q}—wherein q=0 to 5, may encompasses an alkyl, alkenyl and alkynyl as defined herein having at most five carbon atoms. In some embodiments, each of q may be 0. At times, when q=0, L₈ may be a bond.

In some embodiments, a ring system containing five to twelve atoms may be substituted with one or more substituents, in certain embodiments one, two, three or four substituents. In some further embodiments, the substituents may be selected from OH, CF₃, halogen, NO₂, C(═O), —COOH, —NH₂, CN, C(═O)—C₁-C₁₂ alkyl, straight or branched C₁-C₁₂ alkyl, straight or branched C₂-C₁₂ alkenyl, straight or branched C₂-C₁₂ alkynyl, straight or branched C₁-C₁₂ alkylene, straight or branched C₂-C₁₂ alkenylene, straight or branched C₂-C₁₂ alkynylene, C₁-C₁₂ alkoxy, C₁-C₅ carboxyl, aromatic or heteroaromatic ring.

In some embodiments, specific examples of compounds or pharmaceutically acceptable salts or hydrates of the compounds of Formula IX include, without limitation:

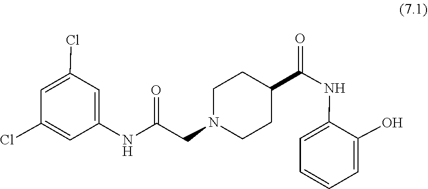

### 1-[(3,5-Dichloro-phenylcarbamoyl)-methyl]-piperidine-4-carboxylic acid (2-hydroxy-phenyl)-amide

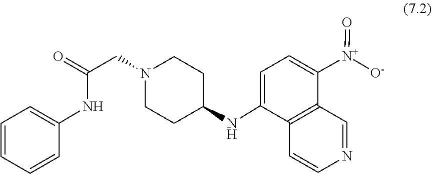

### 2-[4-(8-Nitro-isoquinolin-5-ylamino)-piperazin-1-yl]-N-phenyl-acetamide

For compounds of the disclosure in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the disclosure, which are, for clarity, described in the context of separate embodiments, can also be provided in combination in a single embodiment/example. Conversely, various features of the disclosure which are, for brevity, described in the context of a single embodiment, can also be provided separately or in any suitable subcombination.

The term “alkyl” by itself or as part of another substituent, means, unless otherwise stated, as used herein refers to a linear (straight), branched saturated hydrocarbon and can have a number of carbon atoms optionally designated (i.e., C₁-C₆ means one to six carbons). The term “C₁-C₁₂ alkyl” or “C₁-C₁₂ alkylene” refers to a linear (straight), branched saturated hydrocarbon having from 1 to 12 carbon atoms, in some embodiments, contain from 2 to 8 carbons, in yet some embodiments from 2 to 5 carbons, in yet some further embodiments, from 1 to 3 carbon atoms. It should be noted that alkyl refers to an alkyl end chain and alkylene refers to a middle chain alkyl. Representative C₁-C₁₂ alkyl groups include, but are not limited to, methyl, ethyl, propyl, n-propyl, isopropyl, cyclopropyl, n-butyl, butyl, sec-butyl, iso-butyl, tert-butyl, cyclobutyl, n-pentyl, pentyl, iso-pentyl, neo-pentyl, tert-pentyl, cyclopentyl, hexyl, cyclohexyl, heptyl, cycloheptyl, octyl, sec-octyl (1-methylheptyl), and cyclooctyl as well as homologs and isomers of, for example, n-pentyl, n-hexyl, and the like.

The term “haloalkyl” as used herein can include alkyl structures that are substituted with one or more halo groups or with combinations thereof, for example, “C₁-C₁₂ haloalkyl” refers to a C₁-C₁₂ alkyl as defined above, with one or more hydrogens substituted by halogen atoms.

The term “alkenyl” as used herein refers to a linear (straight), branched unsaturated hydrocarbon having from 2 to 20 carbon atoms and at least one carbon-carbon double bond. The term “C₂-C₁₂ alkenyl” or “C₂-C₁₂ alkenylene” as used herein refers to a linear, branched unsaturated hydrocarbon having from 2 to 12 carbon atoms and at least one carbon-carbon double bond, in some embodiments from 3 to 8 carbons, in yet some further embodiments, from 3 to 5 carbon atoms and at least one double bond. It should be noted that alkenyl refers to an alkyl end chain and alkenylene refers to a middle chain alkyl. Examples of alkenyl groups, include, but are not limited to, groups such as ethenyl (i.e., vinyl), prop-1-enyl (i.e., allyl), but-1-enyl, pent-1-enyl, penta-1,4-dienyl, and the like.

The term “C₂-C₁₂ haloalkenyl” as used herein refers to a C₂-C₁₂alkenyl as defined above, with one or more hydrogens substituted by halogen atoms.

The term “alkynyl” as used herein refers to a linear (straight), branched unsaturated hydrocarbon having from 2 to 20 carbon atoms and at least one carbon-carbon triple bond. The term “C₂-C₁₂ alkynyl” or “C₂-C₁₂ alkynylene” as used herein refers to a linear, branched unsaturated hydrocarbon having from 2 to 12 carbon atoms in certain embodiments, from 3 to 8 carbons, and at least one triple bond (at least one carbon-carbon triple bond). It should be noted that alkynyl refers to an alkyl end chain and alkynylene refers to a middle chain alkyl. Examples of alkynyl groups, include, but are not limited to, groups such as ethynyl, propynyl, butynyl, pentynyl, hexynyl, and the like.

The term “C₂-C₁₂ haloalkynyl” as used herein refers to a C₂-C₁₂ alkynyl as defined above, with one or more hydrogens substituted by halogen atoms.

As used herein “alkoxy” refers to an alkyl group bonded to an oxygen atom. Similarly, the term “C₁-C₁₂ alkoxyl” as used herein refers to a C₁-C₁₂ alkyl group linked to an oxygen. At times, the alkyl group may include one to twelve carbon atoms, at times between one to eight carbon atoms, at times one to five carbon atoms and at times one to three carbon atoms. Examples of alkoxy groups, include, but are not limited to, groups such as methoxy, ethoxy, propyloxy, isopropyloxy, butyloxy, isobutyloxy, tert-butyloxy, pentyloxy, or hexyloxy, and the like.

The term “halo” or “halogen” (halide) independently or as part of another substituent, mean, unless otherwise stated, a fluorine, chlorine, bromine, or iodine atom. The term “halide” by itself or as part of another substituent, refers to a fluoride, chloride, bromide, or iodide atom.

As used herein, a ring system containing five to twelve atoms refers to a mono- or multi-cyclic ring system having 5 to 12 atoms. The ring system containing five to twelve atoms may be saturated, unsaturated or aromatic rings and the like including for example cycloalkyl, heterocycloalkyl, aryl, arylene, aromatic, heteroaromatic rings. A ring system containing five to twelve atoms may contain two rings (bicyclic, etc.), for example aromatic rings and in such case the aromatic rings of the aryl group may be joined at a single point (e.g., biphenyl), or fused (e.g., naphthyl). In some embodiments, a ring system containing five to twelve atoms is a carbocyclic ring or heterocyclic ring. The term “carbocyclic ring” refers to cyclic compounds containing only carbon atoms. The carbocyclic ring may be optionally substituted by one or more substituents, and may be saturated, unsaturated or aromatic. The term “heterocyclic ring” refers to cyclic compounds where one or more carbons are substituted by heteroatoms. Exemplary heteroatoms include, but not limited to, nitrogen, sulfur, and oxygen. The heterocyclic ring may be optionally substituted, and may be saturated, unsaturated or aromatic. The term “saturated” as used herein means that the compound does not contain double or triple bonds. The term “unsaturated” as used herein means that the compound contains at least one double or triple bond. The term “aromatic” as used herein means that the compound contains alternating double and single bonds.

As used herein, “aryl” refers to polyunsaturated, aromatic ring systems having between 5 to 12 atoms which can be a single ring or multiple rings (e.g., 1 to 2 rings) which are fused together or linked covalently. Unless otherwise specifically defined, the term “aryl” refers to cyclic, aromatic hydrocarbon groups that have 1 to 2 aromatic rings, including monocyclic or bicyclic groups having between 5 to 12 atoms. Non-limiting examples include phenyl, biphenyl or naphthyl. The aryl group may be optionally substituted by one or more substituents, e.g., 1 to 5 substituents, at any point of attachment. The substituents can themselves be optionally substituted. As used herein, “C₅-C₂ aromatic” refers to aromatic ring systems having 5 to 12 carbon atoms, such as phenyl, naphthalene and the like.

As used herein, the term “heteroaryl” refers to aryls as defined above where one or more carbons are substituted by heteroatoms. Exemplary heteroatoms include, but not limited to, nitrogen, sulfur, and oxygen. As used herein, “heteroaromatic” refers to refers to a monocyclic or multi-cyclic (fused) aromatic ring system, where one or more of the atoms in the ring system is a heteroatom, that is, an element other than carbon, including but not limited to, nitrogen, oxygen or sulfur. The term “heteroaromatic” used interchangeably with the term “heteroaryl” denotes a heterocyclic aromatic ring systems containing 5 to 12 atoms, with at least one, preferably two carbon atoms and one or more heteroatoms selected from nitrogen, oxygen and sulfur. Non-limiting examples include furan, thipohene, pyrrole, oxazole, thiazole, imidazole, pyrazole, isoxazole, thiazolem benzofurna, indole, benzothiophene, benzoimidazole, indazole, benzoxazole, benzoisoxazole, benzothiazole, isobenzfuran, isoidole, purine, pyridine, pyrazine, pyrimidine, pyrisazine, quinoline, quinozaline, quinazoline, isoquinoline, furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzothiazolyl, benzoisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl, 1-methyl-1H-benzo[d]imidazole, 1H-benzo[d]imidazole and the like.

As used herein, “C₅-C₂ saturated cycloalkyl” refers to a saturated mono- or multi-cyclic ring system having 5 to 12 carbon atoms, preferably having 5 to 7 carbon atoms. Example of “C₅-C₂ cycloalkyl” groups include, but are not limited to cyclopentyl, cyclohexyl and cycloheptyl.

As used herein, “heterocycloalkyl” or “heterocyclyl” or the term “heterocyclic” refers to a monocyclic or multi-cyclic non-aromatic ring system having 5 to 12 members, preferably having 5 to 7 carbon atoms, where one or more, in certain embodiments, 1 to 3, of the atoms in the ring system is a heteroatom, that is, an element other than carbon, including but not limited to, nitrogen, oxygen or sulfur. Examples of “heteroalkyl” include, but are not limited to, pyran, 1,4-dioxane, 1,3-dioxane, piperidine, pyrrolidine, morpholine, tetrahydrothiopyran, tetrahydrothiophene, and the like. The term heterocycloalkyl” also encompasses non-aromatic ring being unsaturated or having one or more degrees of unsaturation containing one or more heteroatomic substitutions selected from S, SO, SO₂, O, or N. “heterocyclic” ring(s) or cycloalkyl ring(s). Examples of “heterocyclic” include, but are not limited to, tetrahydrofuran, pyran, 1,4-dioxane, 1,3-dioxane, piperidine, pyrrolidine, morpholine, tetrahydrothiopyran, tetrahydrothiophene, and the like.

The term “N-containing group” is used herein a chemical group containing a nitrogen atom for example as amino group. The term “amino” as used herein encompass primary, secondary, tertiary or quaternary amines where the point of attachment is through the nitrogen atom which is substituted. For example, the “N-containing group” include N, NH, NH₂, tertiary amine (tertiary alkyl amine), quaternary ammonium (quaternary alkyl ammonium). The nitrogen atom may be substituted with alkyl. In case of a tertiary amine or quaternary amines, the substituent may be the same or may be different.

The term “bond” as used herein denotes a covalent bond. The bond may be between two similar atoms or between different atoms. Non-limiting examples include C—C, C—S, C—O, C—N. S—O, S—N, N—O and the like. It should be noted that a bond as defined above, for example, C—S encompasses both C—S and S—C and this holds for the bonds as defined herein.

The term “optionally substituted” refers to substitution with the named substituent or substituents, multiple degrees of substitution being allowed unless otherwise stated. The term substituted as used herein means that the compounds may contain one or more substituents, including, but not limited to, optionally substituted OH, CF₃, halogen, C(═O), —COOH, —NH₂, CN, alkyl, alkenyl, alkynyl, alkylene, straight alkenylene, alkynylene, haloalkyl, haloalkenyl, haloalkynyl, alkoxy, carboxyl, halogen, ring system including five to twelve atoms, aromatic or heteroaromatic ring, C(═O)—alkyl, alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂,
—N(R^{a})C(O)OR^{a}, —N(R^{a})C(O)R^{a}, —N(R^{a})C(O)N(R^{a})₂, N(R^{a})C(NR^{a})N(R^{a})₂,
—N(R^{a})S(O)_{w}R^{a} (where w is 1 or 2), —S(O)_{w}OR^{a}
(where w is 1 or 2), —S(O)_{w}N(R^{a})₂ (where w is 1 or 2), or PO₃(R^{a})₂, wherein each R^{a} is independently hydrogen or alkyl.

It should be noted that the carbon number, as used herein, refers to the carbon backbone and carbon branching, but does not include carbon atoms of the substituents, such as alkoxy substitutions and the like.

The invention also embraces solvates, pharmaceutically acceptable prodrugs, pharmaceutically active metabolites, and pharmaceutically acceptable salts of compounds of the formula (I) or any variations detailed herein.

The present disclosure also includes any or all of the stereochemical forms, including any enantiomeric or diastereomeric forms, and any tautomers or other forms of the compounds described.

The term “solvate” refers to an aggregate of a molecule with one or more solvent molecules, such as hydrate, alcoholate (aggregate or adduct with alcohol), and the like.

As used herein the term “pharmaceutically acceptable salt” refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound described herein having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di- or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a salt of a compound described herein.

The term “hydrate” refers to a compound formed by the addition of water. The hydrates may be obtained by any known method in the art by dissolving the compounds in water and recrystallizing them to incorporate water into the crystalline structure.

The compounds of the present invention, as defined above, may have the ability to crystallize in more than one form, a characteristic, which is known as polymorphism, and it is understood that such polymorphic forms (“polymorphs”) are within the scope of formulae (I). Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

Certain of the compounds described herein may contain one or more chiral atoms, or may otherwise be capable of existing as two enantiomers or as two or more diastereomers. Accordingly, the compounds of this invention include mixtures of enantiomers as well as purified enantiomers or enantiomerically enriched mixtures. Furthermore, the compounds of this invention include mixtures of diastereomers, as well as purified stereoisomers or diastereomerically enriched mixtures. Also included within the scope of the invention are the individual isomers of the compounds of the invention, as defined above, as well as any wholly or partially mixtures thereof. The present invention also covers the individual isomers of the compounds represented by the formulas above as mixtures with isomers thereof in which one or more chiral centers are inverted.

The term “stereoisomer” as used herein is meant to encompass an isomer that possess identical constitution as a corresponding stereoisomer, but which differs in the arrangement of its atoms in space from the corresponding stereoisomer. For example, stereoisomers may be enantiomers, diastereomers and/or cis-trans (E/Z) isomers. It should be understood that a composition comprising a fatty acid amide of the invention may comprise single enantiomers, single diastereomers as well as mixtures thereof at any ratio (for example racemic mixtures, non racemic mixtures, mixtures of at least two diastereomers and so forth). Furthermore, the invention encompasses any stereoisomer of a fatty acid amide of the invention achieved through in vivo or in vitro metabolism, or by any type of synthetic rout.

Certain of the compounds described herein may contain one or more chiral atoms, or may otherwise be capable of existing as two enantiomers or as two or more diastereomers. Accordingly, the compounds of this invention include mixtures of enantiomers as well as purified enantiomers or enantiomerically enriched mixtures. Furthermore, the compounds of this invention include mixtures of diastereomers, as well as purified stereoisomers or diastereomerically enriched mixtures. Also included within the scope of the invention are the individual isomers of the compounds of the invention, as defined above, as well as any wholly or partially mixtures thereof. The present invention also covers the individual isomers of the compounds represented by the formulas above as mixtures with isomers thereof in which one or more chiral centers are inverted.

It is also noted that the compounds of the present invention may form tautomers. It is understood that all tautomers and mixtures of tautomers of the compounds of the present invention, are included within the scope of the compounds of the present invention. The term “solvate” refers to an aggregate of a molecule with one or more solvent molecules, such as hydrate, alcoholate (aggregate or adduct with alcohol), and the like.

The term “physiologically functional derivative” used herein relates to any physiologically acceptable derivative of a compound as described herein.

The physiologically functional derivatives also include prodrugs of the compounds of the invention. Such prodrugs may be metabolized in vivo to a compound of the invention.

These prodrugs may or may not be active themselves and are also an object of the present invention.

A “pharmaceutically acceptable prodrug” is a compound that may be converted under physiological conditions to the specified compound or to a pharmaceutically acceptable salt of such compound.

A “pharmaceutically active metabolite” is a pharmacologically active product produced through metabolism in the body of a specified compound or salt thereof. Metabolites of a compound may be identified using routine techniques known in the art and their activities determined using tests such as those described herein. Prodrugs and active metabolites of a compound may be identified using routine techniques known in the art.

It should be appreciated that the present disclosure provides in some aspects thereof, any of the ARTS mimetic compound as disclosed herein above, specifically the compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for inducing apoptosis in a cell.

Still further aspects of the present disclosure relate to any of the ARTS mimetic compound as disclosed herein above, specifically the compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for treating, preventing, inhibiting, reducing, eliminating, protecting or delaying the onset of a pathological disorder in a subject in need thereof.

A further aspect of the invention relates to a composition comprising an effective amount of at least one apoptosis related protein in the TGF-beta signaling pathway (ARTS) mimetic compound having the general formula (I) or a pharmaceutically acceptable salt, solvate, hydrate, any stereoisomer thereof or physiologically functional derivative thereof

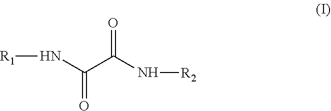

or a pharmaceutically acceptable salt, solvate, hydrate, any stereoisomer thereof or physiologically functional derivative thereof;
wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—, each may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5, said composition optionally further comprises at least one pharmaceutically acceptable carrier/s, excipient/s, auxiliaries, and/or diluent/s.

In some embodiments, the ARTS mimetic compound/s comprised within the composition of the invention may be any of the compounds defined by the invention.

In more specific embodiments, the composition of the invention comprises an effective amount of the ARTS mimetic compound having the formula (3.1) or a pharmaceutically acceptable salt, solvate, hydrate, any stereoisomer thereof or physiologically functional derivative thereof;

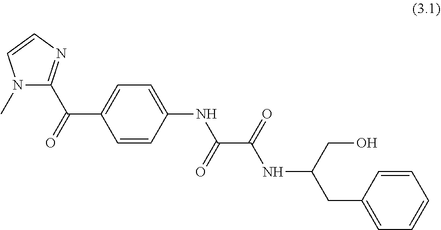 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

In more specific embodiments, the composition of the invention comprises an effective amount of the ARTS mimetic compound having the formula (3.2) or a pharmaceutically acceptable salt, solvate, hydrate, any stereoisomer thereof or physiologically functional derivative thereof;

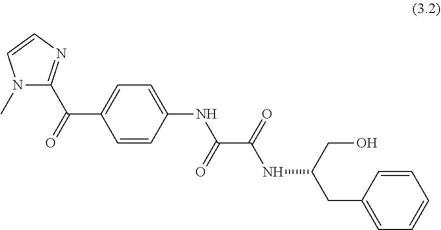

### (S)-N1-(1-hydroxy-3-phenylpropan-2-yl)-N2-(4-(1-methyl-1H-imidazole-2-carbonyl)phenyl)oxalamide

In more specific embodiments, the composition of the invention comprises an effective amount of the ARTS mimetic compound having the formula (3.3) or a pharmaceutically acceptable salt, solvate, hydrate, any stereoisomer thereof or physiologically functional derivative thereof;

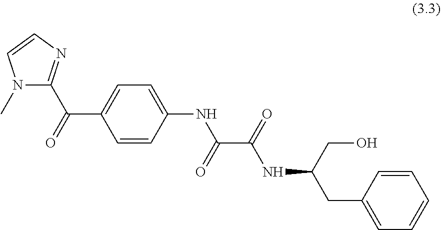 [SMILES: Cn1ccnc1C(=O)c1ccc(NC(=O)C(=O)N[C@@H](CO)Cc2ccccc2)cc1]

### (R)-N1-(1-hydroxy-3-phenylpropan-2-yl)-N2-(4-(1-methyl-1H-imidazole-2-carbonyl)phenyl)oxalamide

It should be appreciated that the present disclosure provides in some aspects thereof, any of the compositions as disclosed herein above, specifically the compositions comprising the compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for inducing apoptosis in a cell.

Still further aspects of the present disclosure relate to any of the compositions as disclosed herein above, specifically the compositions comprising the compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, for use in a method for treating, preventing, inhibiting, reducing, eliminating, protecting or delaying the onset of a pathological disorder in a subject in need thereof.

It should be noted that in some embodiments, the composition of the invention may comprise a compound having the structure of formula (3.2). In some embodiments, this compound (as well as derivatives thereof may be referred to herein as “B3” or “B3 ARTS mimetic compound”.

The invention provides different ARTS mimetic compounds that specifically mimic the C″ domain of ARTS, specifically in binding thereof to its binding site within the BIR3 domain of XIAP.

As used herein “ARTS” (apoptosis-related protein in the TGF-β signaling pathway) is a septin-like mitochondrial protein derived from alternative splicing of the H5/PNUTL2/hCDCre12a/2b gene. ARTS acts as a tumor suppressor protein that functions as an antagonist of XIAP and thereby promotes apoptosis.

It should be appreciated that in certain embodiments, as used herein in the specification and in the claim section below, ARTS protein refers to the human ARTS (as denoted by SEQ ID NO. 1). More specifically, the human ARTS protein comprises an amino acid sequence of 274 amino acid residues as denoted by GenBank Accession No. AF176379, encoded by a nucleic acid sequence of SEQ ID NO. 2.

In some specific embodiments, the ARTS mimetic compound/s of the invention and any compositions thereof may lead to ubiquitin proteasome system (UPS) mediated degradation of at least one of B-cell lymphoma 2 (Bcl-2) and X-linked-Inhibitor of Apoptosis (XIAP) in a cell.

More specifically, as shown by the Examples, the ARTS mimetic compounds of the invention act as XIAP and/or Bcl-2 antagonists, leading to UPS mediated degradation of at least one of XIAP and Bcl2. The invention thus provides a novel antagonist for Bcl-2 protein. As used herein the term Bcl-2 pro-survival protein refers to a proto-oncogenic protein known as an apoptosis inhibitor. The Bcl-2 protein forms the basis of a growing family of related proteins collectively denoted herein as Bcl-2 family of proteins. These proteins are known to control apoptotic cell death by the mitochondrial pathway.

As appreciated in the art, the members of the Bcl-2 family are either pro-survival or pro-apoptotic but regardless of their activity, they all share significant sequence and structural homology. Specifically, the Bcl-2 family of proteins is characterized by up to four regions of sequence homology, known as the Bcl-2 homology (BH) domains.

As previously described in the art, the Bcl-2 family of proteins includes three different groups of proteins: the first group is a pro-survival or anti-apoptotic group denoted herein as “Bcl-2 pro-survival proteins”, the second group is a pro-apoptotic group including BAX and BAK; and a third group denoted herein as BH3-only proteins that exhibit a pro-apoptotic activity.

The ARTS mimetic compound/s of the invention antagonizes the anti-apoptotic activity of the pro-survival Bcl-2 protein leading to enhanced apoptosis of the cells. The “Bcl-2 pro-survival proteins” or “anti-apoptotic” or “Bcl-2 like” as used herein denotes a group of proteins responsible for protecting cells from apoptotic stimuli and are sequentially characterized by containing all four BH domains.

Bcl-2 (B-cell CLL/lymphoma 2) as used herein, is an integral outer mitochondrial membrane protein that blocks the apoptotic death of some cells such as lymphocytes. Bcl-2 suppresses apoptosis in a variety of cell systems including factor-dependent lympho-hematopoietic and neural cells. It regulates cell death by controlling the mitochondrial membrane permeability. Bcl-2 appears to function in a feedback loop system with caspases, it inhibits caspase activity either by preventing the release of cytochrome c from the mitochondria and/or by binding to the apoptosis-activating factor (APAF-1). It should be noted that in certain embodiments, the invention refers to the human Bcl-2 protein as denoted by GenBank Accession No. NP_000624 and SEQ ID NO: 3 and NP_000648 of SEQ ID NO:4), encoded by the Bcl-2 μgene of GenBank Accession No. NM_000633 of SEQ ID NO: 5 and NM_000657 of SEQ ID NO:6.

As recently shown by the inventors, ARTS binds to XIAP through a domain comprising 27 residues covering the C-terminus of ARTS. This interaction induces auto degradation of XIAP. The ARTS mimetic compound/s of the invention target BIR3 domain of XIAP mimicking the ability of ARTS to enhance XIAP degradation. Thus, the ARTS mimetic compounds of the invention act as and therefore may be used as XIAP antagonists. In yet some further embodiments, the ARTS mimetic compounds of the invention may act as dual antagonists of Bcl-2 and XIAP.

As used herein the term “IAPs” denotes a family of proteins that harbor between one to three copies of a baculovirus IAP repeat (BIR) domain that enable interaction with activated caspases. It was previously suggested that the BIR domains of certain IAPs, in particular XIAP, have the ability to directly inhibit caspase activity in vitro.

X-linked inhibitor of apoptosis protein (XIAP), also known as inhibitor of apoptosis protein 3 (IAP3) and baculoviral IAP repeat-containing protein 4 (BIRC) denotes a protein known to stop an apoptotic process and thus inhibit cell death. In human, XIAP is produced by a gene named XIAP gene located on the X chromosome. XIAP is also called human IAP-like Protein (hILP), because it is not as well conserved as the human IAPS: hIAP-1 and hIAP-2-XIAP are the most potent human IAP proteins currently identified.

XIAP belongs to a family of apoptotic suppressor proteins. Members of this family share a conserved motif termed, baculovirus IAP repeat (BIR domain), which is necessary for their anti-apoptotic function. XIAP acts as a direct caspase inhibitor by directly binding to the active site pocket of CASP3 and CASP7 and obstructing substrate entry. It further inactivates CASP9 by keeping it in a monomeric, inactive state.

It should be noted that in certain embodiments, the invention relates to the human XIAP protein (GenBank Accession Nos. NP_001158, NP_001191330, as denoted by SEQ ID NO. 9) encoded by the XIAP gene (GenBank Accession Nos. NM_001167, NM_001204401, as denoted by SEQ ID NO. 10).

In yet another embodiment, the ARTS mimetic compounds of the invention bind XIAP thereby leading to UPS mediated degradation of Bcl-2. As such, they may further act on other Bcl-2 family members. Thus, in some embodiments the ARTS mimetic compounds of the invention may antagonize Bcl-xL. B-cell lymphoma-extra large (Bcl-xL) as used herein, is a transmembrane molecule in the mitochondria. It is a member of the Bcl-2 family of proteins and acts as a pro-survival protein by preventing the release of mitochondrial contents such as cytochrome c, which would lead to caspase activation. In certain embodiments the invention relates to the human Bcl-xL protein (GenBank Accession No. CAA80661 SEQ ID NO: 7), encoded by the Bcl-xL gene as denoted by GenBank Accession No. Z23115 and SEQ ID NO: 8.

In yet another embodiment, the ARTS mimetic compounds of the invention may antagonize any one of the human Bcl-2 pro-survival proteins Mel-1, Bcl-w, A1/Bfl-1 and Bcl-B/Bcl2L10 as denoted by accession number: AAF64255, AAB09055, NP_033872 and NP_065129, respectively.

As indicated above, the present invention relates to the ARTS mimetic compounds of the invention that act as antagonist/s of XIAP and Bcl-2. An antagonist is a compound that competes with a specific protein, a ligand for example, on binding to another protein, a receptor for example. Such binding usually, induces a specific biological response or action that is blocked by the competing antagonist. Antagonists have affinity but no efficacy for their cognate binding protein and binding will disrupt the interaction and inhibit the function of such cognate protein. Antagonists mediate their effects by binding to the active (orthosteric=right place) site or to allosteric (=other place) sites on any cognate protein, in this case, XIAP (or receptor, in case applicable), or they may interact at unique binding sites not normally involved in the biological regulation of the cognate protein.

As shown in the Examples, down regulation of XIAP and Bcl-2 protein levels was observed in the presence of the ARTS mimetic compounds of the invention. This suggests that the down-regulation of Bcl-2 levels observed may be mediated by the ubiquitin—proteasome machinery (UPS).

Thus, in certain embodiments, the ARTS mimetic compounds of the invention, mediate ubiquitin proteasome system (UPS) degradation of XIAP anti-apoptotic protein and Bcl-2 prosurvival protein, thereby reducing survival of the cells.

As used herein the term “ubiquitin proteasome system” denotes a multi component system that identifies and degrades unneeded, damaged or misfolded proteins by breaking peptide bonds (proteolysis) of the protein in the cytoplasm of cells. As appreciated in the art, degradation of a protein via the UPS involves two discrete and successive steps. In the first step, proteins are tagged for degradation with a small protein called ubiquitin. The tagging reaction is catalyzed by enzymes called ubiquitin ligases. Once a protein is tagged with a single ubiquitin molecule, this is a signal to other ligases to attach additional ubiquitin molecules.

More specifically, conjugation of ubiquitin, a highly evolutionarily conserved 76 amino acid residue polypeptide, to the protein substrate proceeds via a three-step cascade mechanism involving E1, E2 and E3 enzymes. By successively adding activated ubiquitin moieties to internal lysine residues on the previously conjugated ubiquitin molecule, a polyubiquitin chain is synthesized that is subsequently recognized by the downstream 26S proteasome complex.

In the second step, degradation of polyubiquitinated substrates is carried out by a large, protease complex, referred to as the 26S proteasome that does not recognize nonmodified substrates. The proteasomes are multicatalytic protease protein complexes found in all cells that degrades polyubiquitinated proteins to short peptides by breaking peptide bonds (proteolysis). Following degradation of the substrate, short peptides derived from the substrate are released, along with reusable ubiquitin.

It should be noted that the ubiquitin-proteasome system (UPS) plays a central and complex role in regulating apoptosis by directly targeting key cell death proteins, including caspases.

It should be noted that by inducing XIAP and Bcl-2 degradation, the ARTS mimetic compound/s of the invention, inhibit the pro-survival or anti-apoptotic effect of Bcl-2 protein. The terms “inhibition”, “moderation” or “attenuation” as referred to herein, relate to the retardation, restraining or reduction of the anti-apoptotic activity of a Bcl-2 pro-survival protein. Such inhibition may be of about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95%, 99.9% or 100%.

It should be further noted that by inhibiting the anti-apoptotic action of Bcl-2 proteins, ARTS mimetic compounds of the invention induce or enhances apoptosis. More specifically, the ARTS mimetic compounds of the invention, specifically, as well as any of the compositions and methods of the invention described herein after, may lead to an increase, enhancement, induction or elevation in apoptosis of treated cells, said increase, induction or elevation of apoptosis may be an increase by about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%. More specifically, an increase of about 5%, 10%, 15%, 20%, 25%, 30%, 35%, 40%, 45%, 50%, 55%, 60%, 65%, 70%, 75%, 80%, 85%, 90%, 95%, 99% OR 100% as compared to untreated control.

With regards to the above, it is to be understood that, where provided, percentage values such as, for example, 10%, 50%, 120%, 500%, etc., are interchangeable with “fold change” values, i.e., 0.1, 0.5, 1.2, 5, 10, 20, 30, 40, 50, 60, 70, 80, 90, 100 etc., respectively.

Moreover, in certain embodiments, the ARTS mimetic compound/s of the invention may lead to elevation in at least one of c-caspase and c-PARP levels in a cell. In some specific embodiments, the ARTS mimetic compound/s of the invention may lead to elevation in cleaved caspase, specifically, cleaved caspase 3 and/or caspase 9.

In further embodiments, the ARTS mimetic compound/s of the invention and compositions thereof may induce programmed cell death, or apoptosis.

The term “apoptosis” refers to a regulated network of biochemical events which lead to a selective form of cell suicide and is characterized by readily observable morphological and biochemical phenomena. Cells undergoing apoptosis show characteristic morphological and biochemical features. These features include chromatin aggregation or condensation, DNA fragmentation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial dysfunction accompanying apoptosis.

As indicated above, apoptosis is a tightly controlled form of active cell death that is necessary for development and organismal homeostasis. Death by the apoptotic pathway is achieved among others, by the activation of a family of highly potent and specific proteases, termed caspases (for cysteine-aspartate protease).

The activity of caspases is tightly regulated, and the cell maintains several “checkpoints” to control their activity. The first level of regulation is intrinsic to caspases themselves. Caspases are initially transcribed as weakly active zymogens, which only upon proper stimulation are cleaved to form the active enzyme.

The second level of caspase regulation is achieved by inhibitors, namely the family of proteins called IAPs (Inhibitor of Apoptosis Protein).

Still further, the ARTS mimetic compound/s of the invention and compositions thereof may induce apoptosis in at least one of a premalignant and a malignant cell.

In some embodiments, such cell may be an epithelial carcinoma cell.

In more specific embodiments, such cell may be an epithelial breast carcinoma cell.

In yet some further embodiments, the cell may be a cervical carcinoma cell.

Still further, in some embodiments, the cell may be at least one melanoma cell.

In yet some further embodiments, the cell may be at least one sarcoma cell. Still further in some embodiments, the cell may be an osteosarcoma cell.

Still further in some embodiments, the cell may be a hematological malignancy cell.

In some particular embodiments, the ARTS mimetic compound/s of the invention may induce apoptosis in a cell characterized by at least one of: (a) over expression of Bcl-2; (b) over expression of XIAP; and (c) low or no expression of ARTS.

In some particular embodiments, the ARTS mimetic compound/s of the invention may induce apoptosis in a cell over expressing Bcl-2.

In yet some further embodiments, the ARTS mimetic compound/s of the invention may induce apoptosis in a cell over expressing XIAP.

In yet some further embodiments, the ARTS mimetic compound/s of the invention may induce apoptosis in a cell over expressing XIAP and Bcl-2.

In further embodiments, the ARTS mimetic compound/s of the invention may induce apoptosis in a cell that express low levels of ARTS, or in a cell that show no expression of ARTS. As ARTS mediates the degradation of both, XIAP and Bcl-2, cells that do not express ARTS or show low levels of expression of ARTS, may in some embodiments display overexpression of at least one of XIAP and Bcl-2.

In yet some further embodiments, the ARTS mimetic compound/s of the invention as well as any composition/s thereof may be applicable in inducing programmed cell death in premalignant or malignant epithelial cells in a subject in need thereof.

The present invention therefore further provides pharmaceutical compositions.

In certain embodiments, the compositions of the present invention can be administered for prophylactic and/or therapeutic treatments. In therapeutic application, compositions are administered to a patient already affected by a proliferative disorder (e.g., carcinoma, specifically breast carcinoma) in an amount sufficient to cure or at least partially arrest the condition and its complications. An amount adequate to accomplish this is defined as a “therapeutically effective dose.” Amounts effective for this use will depend upon the severity of the condition and the general state of the patient's own immune system, but generally range from about 0.001 to about 1000 mg/Kg. Single or multiple administrations on a daily, weekly or monthly schedule can be carried out with dose levels and pattern being selected by the treating physician. Additionally, the administration of the compositions of the invention, may be periodic, for example, the periodic administration may be affected twice daily, three time daily, or at least one daily for at least about three days to three months. The advantages of lower doses are evident to those of skill in the art. These include, inter alia, a lower risk of side effects, especially in long-term use, and a lower risk of the patients becoming desensitized to the treatment. In another embodiment, treatment using the compositions of the invention, may be affected following at least 1, 2, 3, 4, 5, 6, 7, 8, 9, 10, 14, 30, 60, 90 days of treatment, and proceeding on to treatment for life.

In some embodiments, the effective amount of the disclosed ARTS mimetic compounds, and specifically of the B3 compound, may range from about 0.1 μM to about 100 μM. Specifically, from 0.5 μM to about 100 μM, from 1 μM to about 100 μM, 1 μM to 90, 1 μM to 80 μM, 1 μM to 70 μM, 1 μM to 80 μM, 1 μM to 70 μM, 1 μM to 60 μM, 1 μM to 50 μM, 1 μM to 40 μM, 1 μM to 30 μM, 1 μM to 20 μM, 1 μM to 10 μM, specifically, 1 μM or less, 2, 3, 4, 5, 6, 7, 8, 9, 10, 11, 12, 13, 14, 15, 16, 17, 18, 19, 20 μM or more. In some embodiments, an effective amount of the “B3” compound may be 20 μM.

It should be noted that the treatment of different proliferative conditions may indicate the use of different doses or different time periods, these will be evident to the skilled medical practitioner.

For prophylactic applications, the compositions of the invention may include a prophylactic effective amount of the active ingredient. The term “prophylactically effective amount” is intended to mean that amount of a pharmaceutical composition that will prevent or reduce the risk of occurrence or recurrence of the biological or medical event that is sought to be prevented in a tissue, a system, animal or human by a researcher, veterinarian, medical doctor or other clinician. In prophylactic applications, the compositions of the invention are administered to a patient who is at risk of developing the disease state to enhance the patient's resistance. Such an amount is defined to be a “prophylactically effective dose”. In this use, the precise amounts again depend upon the patient's state of health and general level of immunity, but generally range from 0.001 to 1000 mg per dose.

It should be appreciated that the ARTS mimetic compounds of the present disclosure, specifically, the B3 compound, may be formulated in any vehicle, matrix, nano- or micro-particle, or composition. Of particular relevance are formulations of the ARTS mimetic compounds adapted for use as a nano- or micro-particles. Nanoscale drug delivery systems using micellar formulations, liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as micelles, liposomes or polyelectrolyte multilayer capsules, incorporate nanoparticle (NP) actuators. More specifically, Controlled drug delivery systems (DDS) have several advantages compared to the traditional forms of drugs. It should be therefore understood that the present disclosure further encompasses the use of various nanostructures, including micellar formulations, liposomes, polymers, dendrimers, silicon or carbon materials, polymeric nanoparticles and magnetic nanoparticles, as carriers in drug delivery systems. The term “nanostructure” or “nanoparticle” is used herein to denote any microscopic particle smaller than about 100 nm in diameter. In some other embodiments, the carrier is an organized collection of lipids. When referring to the structure forming lipids, specifically, micellar formulations or liposomes, it is to be understood to mean any biocompatible lipid that can assemble into an organized collection of lipids (organized structure). In some embodiments, the lipid may be natural, semi-synthetic or fully synthetic lipid, as well as electrically neutral, negatively or positively charged lipid. In some embodiments, the lipid may be a naturally occurring phospholipid.

It should be appreciated that the at least one ARTS mimetic compounds disclosed herein may be associated with any of the nanostructures described above, specifically, any of the micellar formulations, liposomes, polymers, dendrimers, silicon or carbon materials, polymeric nanoparticles and magnetic nanoparticles disclosed herein above. The term “association” may be used interchangeably with the term “entrapped”, “attachment”, “linked”, “embedded”, “absorbed” and the like, and contemplates any manner by which the at least one ARTS mimetic compounds of the disclosure is held.

As mentioned herein before, the compositions provided by the invention optionally further comprise at least one pharmaceutically acceptable excipient or carrier. As used herein “pharmaceutically acceptable carrier” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

The pharmaceutical composition of the invention can be administered and dosed by the methods of the invention, in accordance with good medical practice. More specifically, the compositions used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

The phrases “systemic administration,” “administered systemically,” “peripheral administration” and “administered peripherally” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation is vacuum-drying and freeze drying techniques which yield a powder of the active ingredient plus any additional desired ingredient from a previously sterile-filtered solution thereof.

Pharmaceutical compositions used to treat subjects in need thereof according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Local administration to the area in need of treatment may be achieved by, for example, local infusion during surgery, topical application, direct injection into the specific organ, etc.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

In particular embodiments, the unit dosage formulations are those containing a daily dose or sub-dose, as herein above recited, or an appropriate fraction thereof, of an active ingredient.

A further aspect of the invention relates to a method for inducing apoptosis in a cell. In more specific embodiments, the method comprises the step of contacting the cell with an effective amount of at least one ARTS mimetic compound, any combination thereof or any composition comprising the same. In further embodiments, the ARTS mimetic compound having the general formula (I) or a pharmaceutically acceptable salt, solvate, hydrate, any stereoisomer thereof or physiologically functional derivative thereof;

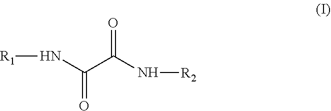

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—, each may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5.

It should be noted that in certain embodiments, the method/s of the invention may use any of the ARTS mimetic compound/s as defined by the invention.

In more particular embodiments, the method/s of the invention may use an ARTS mimetic compound having the formula (3.1) or a pharmaceutically acceptable salt, solvate, hydrate, any stereoisomer thereof or physiologically functional derivative thereof;

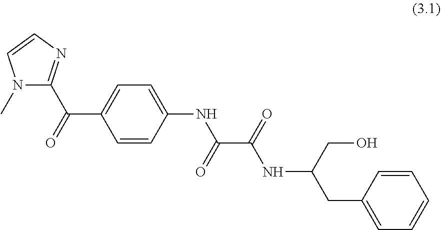 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

In more particular embodiments, the method/s of the invention may use an ARTS mimetic compound having the formula (3.2) or a pharmaceutically acceptable salt, solvate, hydrate, any stereoisomer thereof or physiologically functional derivative thereof;

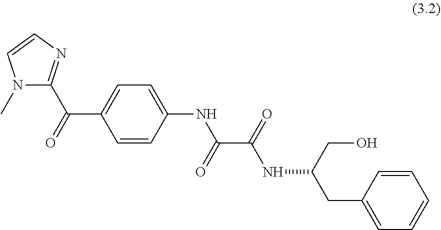

### (S)-N1-(1-hydroxy-3-phenylpropan-2-yl)-N2-(4-(1-methyl-1H-imidazole-2-carbonyl)phenyl)oxalamide

In more particular embodiments, the method/s of the invention may use an ARTS mimetic compound having the formula (3.3) or a pharmaceutically acceptable salt, solvate, hydrate, any stereoisomer thereof or physiologically functional derivative thereof;

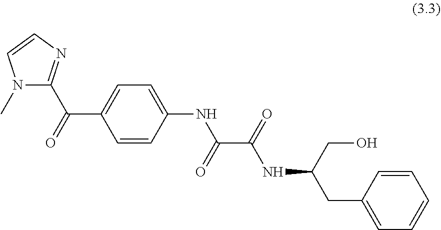 [SMILES: Cn1ccnc1C(=O)c1ccc(NC(=O)C(=O)N[C@@H](CO)Cc2ccccc2)cc1]

### (R)-N1-(1-hydroxy-3-phenylpropan-2-yl)-N2-(4-(1-methyl-1H-imidazole-2-carbonyl)phenyl)oxalamide

Still further, in certain embodiments, the ARTS mimetic compound/s used by the methods of the invention may lead to at least one of UPS mediated degradation of at least one of Bcl-2 and XIAP and elevation in at least one of c-caspase and c-PARP levels. In yet some specific embodiments, the ARTS mimetic compound/s used by the methods of the invention may lead to at least one of elevation in at least one of c-caspase-3, c-caspase-9 and c-PARP levels.

In yet some further embodiments, the ARTS mimetic compound/s used by the methods of the invention may induce apoptosis in at least one of pre-malignant and malignant cell/s.

In more specific embodiments, the cell may be an epithelial carcinoma cell or a premalignant cell. More specifically, the cell may be an epithelial breast carcinoma cell or a pre-malignant epithelial breast cell.

In yet some further embodiments, the cell may be a cervical carcinoma cell. Still further, in some embodiments, the cell may be at least one melanoma cell. In yet some further embodiments, the cell may be at least one sarcoma cell. Still further in some embodiments, the cell may be an osteosarcoma cell. Still further in some embodiments, the cell may be a hematological malignancy cell. In some embodiments, the ARTS mimetic compound/s of the invention may induce apoptosis in a cell characterized by at least one of: (a) over expression of Bcl-2; (b) over expression of XIAP; and (c) low or no expression of ARTS.

In certain embodiments, the ARTS mimetic compound/s of the invention may induce apoptosis in a cell over expressing Bcl-2.

In yet some further embodiments, the ARTS mimetic compound/s of the invention may induce apoptosis in a cell over expressing XIAP.

In yet some further embodiments, the ARTS mimetic compound/s of the invention may induce apoptosis in a cell over expressing XIAP and Bcl-2.

In further embodiments, the ARTS mimetic compound/s of the invention may induce apoptosis in a cell that express low levels of ARTS, or in a cell that show no expression of ARTS.

In yet some further embodiments, the invention provides a method for inducing apoptosis of pre-malignant or malignant epithelial cells in a subject in need thereof.

Another aspect of the invention relates to a method for treating, preventing, inhibiting, reducing, eliminating, protecting or delaying the onset of a proliferative disorder in a subject in need thereof. More specifically, the method comprises administering to the subject a therapeutically effective amount of at least one ARTS mimetic compound or of a composition comprising the same. More specifically, the ARTS mimetic compound used by the method of the invention may have the general formula (I) or a pharmaceutically acceptable salt, solvate, hydrate, any stereoisomer thereof or physiologically functional derivative thereof

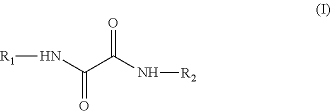

or a pharmaceutically acceptable salt, solvate, hydrate, any stereoisomer thereof or physiologically functional derivative thereof;
wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—. each may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5.

In more specific embodiments, the method of the invention may use any of the ARTS mimetic compound/s defined by the invention or any of the compositions of the invention.

As used herein, “proliferative disorder” is a disorder displaying hyper proliferation. This term means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper proliferative disease” or “hyper proliferative disorder.” It should be noted that the term “proliferative disorder”, “cancer”, “tumor” and “malignancy” all relate equivalently to a hyperplasia of a tissue or organ. In yet some further embodiments, the methods of the invention may be applicable for treating malignant, pre-malignant disorders and/or cancer. In general, the compositions and methods of the present invention may be used in the treatment of non-solid and solid tumors.

In certain embodiments, the therapeutic method of the invention may be particularly effective for a subject suffering from any one of a pre-malignant condition and carcinoma.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges. In terms of solid tumors, this group of cancers may include, among others, carcinomas of the breast, lung, bladder as well as gastric, colorectal, ovarian and uterine carcinomas. The term “carcinoma” refers herein to any tumor tissue derived from putative epithelial cells, or cells of endodermal or ectodermal germ layer during embryogenesis, that become transformed and begin to exhibit abnormal malignant properties.

In more specific embodiments, the therapeutic methods of the invention may be applicable for subjects suffering from a breast carcinoma.

Breast cancer is one of the leading causes of cancer death in women in the Western world. Though current therapies are effective, a considerable population will relapse, rendering the essential need for improved and new avenues of targeted therapies. Gene expression profiling can be used to distinguish breast cancers into distinct molecular subtypes with prognostic significance, based upon phenotypic diversity in biological factors such as histological grade, estrogen receptor (ER) status, progesterone receptor (PgR) status, and HER2/neu expression (HER2).

When presently referring to breast cancer, is meant any type of cancer originating from breast tissue, including ductal and lobular carcinomas. The present context also encompasses genetic or hereditary breast cancers (5-10% of all cases) developing from predisposing mutations in BRCA1 and BRCA2 μgenes and also other relevant mutations in p53 (Li-Fraumeni syndrome), PTEN (Cowden syndrome), and STK11 (Peutz-Jeghers syndrome), CHEK2, ATM, BRTP1, and PALB2 μgenes. The present context also encompasses all breast cancer classifications, including those using histopathology (e.g. mammary ductal carcinoma, carcinoma in situ, invasive carcinoma or inflammatory breast cancer), grade (e.g. well differentiated/low grade, moderately differentiated/intermediate grade and poorly differentiated/high grade), stage (0=pre-cancerous, 1-3=regional, 4=metastatic), receptor status (relating to the expression of estrogen receptor ER, PR progesterone receptor and/or HER2/ERBB2 receptor), DNA and protein based classification (using specific mutations or gene expression profiles), and other classification approaches.

According to another embodiment, as leading to degradation of at least one of Bcl-2 and XIAP, the ARTS mimetic compound/s, compositions and methods of the invention may be further applicable for pathological disorders characterized by at least one of: (a) over expression of Bcl-2; (b) over expression of XIAP; and (c) low or no expression of ARTS. In yet some further embodiments, such disorders are characterized by overexpression of XIAP, Bcl-2 and low or no expression of ARTS.

According to other embodiments, as leading to degradation of Bcl-2, the ARTS mimetic compound/s, compositions and methods of the invention may be further applicable for Bcl-2 over-expressing pathological disorders. More specifically, the invention thus further provides compositions for treating, inhibiting, preventing, ameliorating or delaying the onset of a Bcl-2 over-expressing pathological disorder. Such composition optionally may further comprise at least one pharmaceutically acceptable carrier, diluent or excipient.

The phrases “Bcl-2-over-expressing-disorder” and “Bcl-2-mediated disorder” refer to pathological and disease conditions in which a Bcl-2 protein is over-expressed as indicated herein above. Moreover, this term also encompasses conditions in which Bcl-2 plays a role. Such roles can be directly related to the pathological condition or can be indirectly related to the condition. The feature common to this class of conditions is that they can be ameliorated by inhibiting the expression of, activity of, function of, or association with Bcl-2 proteins.

The term “over expressed” refers to an increase in the measurable expression level of Bcl-2 μgene as measured by the amount of RNA and/or the amount of protein in a sample as compared with the measurable expression level of Bcl-2 μgene in a second sample, specifically, a control sample. “Over expressed Bcl-2” can be measured and evaluated using the ratio of the level of expression of Bcl-2 in a sample as compared with the mean expression level of Bcl-2 of a control sample wherein the ratio is not equal and specifically, is above 1.0. When determining over expression on the basis of the ratio, an RNA or protein is over expressed if the ratio of the level of expression in a first sample as compared with a second sample is greater than 1.0. For example, a ratio of greater than 1.2, 1.5, 1.7, 2, 3, 4, 5, 6, 7, 8, 9, 10, 20, 30, 40, 50, 60, 70, 80, 90, 100 or more.

More specifically, disorders displaying “over or increased expression” or “up regulation” of Bcl-2 refer to disorders which demonstrate at least 10% or more, for example, 20%, 30%, 40%, or 50%, 60%, 70%, 80%, 90%, 95%, 100% or more, or 1.1 fold, 1.2 fold, 1.4 fold, 1.6 fold, 1.8, 1.9, 2, 3, 4, 5, 6, 7, 8, 9, 10, 20, 30, 40, 50, 60, 70, 80, 90, 100 fold, or more increase in Bcl-2 expression (as measured by RNA expression or protein expression), relative to a control sample.

Thus, a Bcl-2 over-expressing pathological disorder is meant a disorder characterized by over-expression of Bcl-2 in said subject or in a diseased tissue of said subject as compared to a healthy subject or a healthy tissue of the same subject.

It should be noted that the Bcl-2 over-expressing disorder may be caused by chromosomal translocation, hypo-methylation and down regulation of the microRNAs that target Bcl-2.

In yet another embodiment, the pharmaceutical composition of the invention is specifically applicable for treating Bcl-2 over-expressing proliferative disorders.

According to another embodiment, as leading to degradation of XIAP, the ARTS mimetic compound/s, compositions and methods of the invention may be further applicable for XIAP over-expressing pathological disorders. More specifically, the invention thus further provides compositions for treating, inhibiting, preventing, ameliorating or delaying the onset of a XIAP over-expressing pathological disorder. Such composition optionally may further comprise at least one pharmaceutically acceptable carrier, diluent or excipient.

The phrases “XIAP-over-expressing-disorder“and” XIAP-mediated disorder” refer to pathological and disease conditions in which a XIAP protein is over-expressed as indicated herein above. Moreover, this term also encompasses conditions in which XIAP plays a role. Such roles can be directly related to the pathological condition or can be indirectly related to the condition. The feature common to this class of conditions is that they can be ameliorated by inhibiting the expression of, activity of, function of, or association with XIAP proteins.

The term “over expressed” refers to an increase in the measurable expression level of XIAP gene as measured by the amount of RNA and/or the amount of protein in a sample as compared with the measurable expression level of XIAP gene in a second sample, specifically, a control sample. “Over expressed XIAP” can be measured and evaluated using the ratio of the level of expression of XIAP in a sample as compared with the mean expression level of XIAP of a control sample wherein the ratio is not equal and specifically, is above 1.0. When determining over expression on the basis of the ratio, an RNA or protein is over expressed if the ratio of the level of expression in a first sample as compared with a second sample is greater than 1.0. For example, a ratio of greater than 1.2, 1.5, 1.7, 2, 3, 4, 5, 6, 7, 8, 9, 10, 20, 30, 40, 50, 60, 70, 80, 90, 100 or more.

More specifically, disorders displaying “over or increased expression” or “up regulation” of XIAP refer to disorders which demonstrate at least 10% or more, for example, 20%, 30%, 40%, or 50%, 60%, 70%, 80%, 90%, 95%, 100% or more, or 1.1 fold, 1.2 fold, 1.4 fold, 1.6 fold, 1.8, 1.9, 2, 3, 4, 5, 6, 7, 8, 9, 10, 20, 30, 40, 50, 60, 70, 80, 90, 100 fold, or more increase in XIAP expression (as measured by RNA expression or protein expression), relative to a control sample.

Thus, a XIAP over-expressing pathological disorder is meant a disorder characterized by over-expression of XIAP in said subject or in a diseased tissue of said subject as compared to a healthy subject or a healthy tissue of the same subject.

In yet another embodiment, the pharmaceutical composition of the invention is specifically applicable for treating XIAP over-expressing proliferative disorders.

In yet some further embodiments, said disorders are characterized by low expression or no expression of ARTS.

Still further, it must be appreciated that in some embodiments, selection or identification of a patient or population of patients that may be suitably treated by the ARTS mimetic compounds of the invention may involve a diagnostic step of measuring the expression levels of at least one of ARTS, XIAP and Bcl-2. Patients that display at least one of (a) over expression of Bcl-2; (b) over expression of XIAP; and (c) low or no expression of ARTS, are to be treated by the ARTS mimetic compounds of the invention.

Still further, malignancy, as contemplated in the present invention may be any one of lymphomas, leukemias, carcinomas, melanomas, myeloma and sarcomas. Therefore, in certain embodiments, the ARTS mimetic compound/s, compositions and methods of the invention may be further relevant for other malignancies such as lymphomas, leukemia, melanomas, myeloma and sarcomas.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Melanoma as used herein is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Further malignancies that may find utility in the present invention can comprise but are not limited to hematological malignancies (including lymphoma, leukemia and myeloproliferative disorders), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. More particularly, the malignant disorder may be lymphoma. Non-limiting examples of cancers treatable according to the invention include hematopoietic malignancies such as all types of lymphomas, leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. Still further the invention relates to any neurological tumor, for example, neuroblastoma, astrocytoma, CNS lymphoma, neuroma, glioma, Chordoma, medulloblastoma, Oligodendroglioma, Craniopharyngioma, and any mixed neurological tumor.

The methods provided herein involve administration of the ARTS mimetic compound/s of the invention in a therapeutically effective amount. The term “effective amount” as used herein is that determined by such considerations as are known to the man of skill in the art. The amount must be sufficient to prevent or ameliorate tissue damage caused by proliferative disorders. Dosing is dependent on the severity of the symptoms and on the responsiveness of the subject to the active drug, specifically, the antagonist of the invention. Medically trained professionals can easily determine the optimum dosage, dosing methodology and repetition rates. In any case, the attending physician, taking into consideration the age, sex, weight and state of the disease of the subject to be treated, will determine the dose. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence times and concentrations of the compositions and combined composition of the invention in bodily fluids or tissues. Following successful treatment, it may be desirable to have the patient undergo maintenance therapy to prevent the recurrence of the disease state, wherein the ARTS mimetic compound used by the method of the invention is administered in maintenance doses, once or more daily. As use herein “therapeutically effective amount” means an amount of the ARTS mimetic compound/s, a composition comprising the same which provides a medical benefit as noted by the clinician or other qualified observer. Regression of a tumor in a patient is typically measured with reference to the diameter of a tumor. Decrease in the diameter of a tumor indicates regression. Complete regression is also indicated by failure of tumors to reoccur after treatment has stopped.

The present invention provides methods for treating proliferative disorder. The term “treatment or prevention” refers to the complete range of therapeutically positive effects of administrating to a subject including inhibition, reduction of, alleviation of, and relief from, proliferative disorder symptoms or undesired side effects of such proliferative disorder related disorders. More specifically, treatment or prevention includes the prevention or postponement of development of the disease, prevention or postponement of development of symptoms and/or a reduction in the severity of such symptoms that will or are expected to develop. These further include ameliorating existing symptoms, preventing—additional symptoms and ameliorating or preventing the underlying metabolic causes of symptoms.

As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the treatment methods herein described are desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the methods and compositions of the invention are intended for mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. It should be further noted that particularly in case of human subject, administering of the compositions of the invention to the patient includes both self-administration and administration to the patient by another person.

The invention provides methods for treating proliferative disorders, and further relates to disorders associated or related to cancer. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

The invention further provides the use of an effective amount of at least one ARTS mimetic compound and any combination thereof in the preparation of a composition for treating, preventing, inhibiting, reducing, eliminating, protecting or delaying the onset of a proliferative disorder in a subject in need thereof.

The invention further provides for the use of an effective amount of at least one ARTS mimetic compound/s as defined by the invention, and any combination thereof in the preparation of a composition for treating, preventing, inhibiting, reducing, eliminating, protecting or delaying the onset of a proliferative disorder in a subject in need thereof.

Still further, the invention provides an effective amount of at least one ARTS mimetic compound/s according to the invention, any combination thereof or any composition comprising the same for use in a method for inducing programmed cell death.

The invention further provides an effective amount of at least one ARTS mimetic compound/s as defined by the invention, any combination thereof or any composition comprising the same for use in a method for inducing apoptosis in a subject in need thereof.

Still further, the invention relates to an effective amount of at least one ARTS mimetic compound/s as defined herein, any combination thereof or any composition comprising the same for use in a method for treating, preventing, inhibiting, reducing, eliminating, protecting or delaying the onset of a proliferative disorder in a subject in need thereof.

As shown by Example 4, the combined treatment of ABT-199 with B3 significantly reduced Bcl-2 expression, and moreover, B3 synergistically restores the apoptotic effect of ABT-199. Thus, the invention further provides a combined composition comprising an effective amount of at least one ARTS mimetic compound as defined by the invention, specifically, the B3 mimetic compound, and at least one B-cell lymphoma 2 (Bcl-2) Homology 3 (BH3) mimetic compound. In some embodiments, such BH3 mimetic compound is 4-[4-[[2-(4-Chlorophenyl)-4,4-dimethyl-1-cyclohexen-1-yl]methyl]-1-piperazinyl]-N—[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

In yet some further aspects, the invention provides a method for inducing apoptosis in a cell comprising the step of contacting said cell with an effective amount of at least one ARTS mimetic compound as defined by the invention, specifically, the B3 compound, and of at least one BH3 mimetic compound. In more specific embodiments, the BH3 mimetic compound is 4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

As indicated above, the Bcl-2 proteins can be broadly categorized as acting in either a pro-apoptotic or anti-apoptotic manner. Whilst these groups act directly in driving or diminishing apoptosis, a third group of proteins, which are functionally and structurally unique, and when over-expressed can sensitize cells to biochemical cues that induce apoptosis, are the BH3-only proteins (or sensitizer proteins). The Bcl-2 proteins are composed of conserved BH1-4 domains and in some instances a transmembrane domain. The key structural component of intrinsic importance, which is present in all of the pro-apoptotic Bcl-2 family protein members, is unquestionably the BH3 domain, which is a structure composed of about 15 amino acids from α-helix 2, and which interacts with the hydrophobic pocket structure formed by α-helices 2-5 of the anti-apoptosis proteins, such as Bcl-2 protein.

The principles of BH3-mimetics are mechanistically founded on disrupting the interaction of the pro-apoptotic BH3 domain with the hydrophobic pocket of the anti-apoptotic Bcl-2 proteins (such as Bcl-2, Bcl-xL or Mc11), thus permitting oligomerized BAX (or BAK) to form the MCP, thereby leading to apoptosis. To name but few, BH-3 mimetic compounds applicable in the present disclosure, specifically for the kits and combined compositions, include, but are not limited to ABT-199, ABT-263 (Navitoclax), WEHI-539, BXI-61, BXI-72, GX15-070 (Obatoclax), s1, JY-1-106, Apogossypolone (ApoG2), BI97C1 (sabutoclax), TW-37, MIM1, MS1 (MCL-specific peptide), BH3I-1 and its structural derivatives, UMI-77, Marinopyrrole A (Martioclax).

ABT-199 (also known as Venetoclax, RG7601, CDC-0199), was developed through rational design approaches as a high-affinity antagonist for Bcl-2 and much lower affinity binding for Bcl-xL, to help overcome thrombocytopenia side effects derived from off-target Bcl-xL inhibition, and a common feature associated with ABT-737 and Navitoclax treatments. As used herein, ABT-199, is denoted by the following formula:

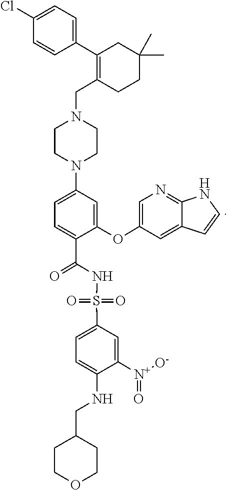 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

The invention further provides a method for treating, preventing, inhibiting, reducing, eliminating, protecting or delaying the onset of a proliferative disorder in a subject in need thereof, said method comprises administering to said subject a therapeutically effective amount of at least one ARTS mimetic compound as defined by the invention, specifically, the B3 compound, and of at least one BH3 mimetic compound, or of any composition comprising said BH3 mimetic compound and said ARTS mimetic compound. In more specific embodiments, the BH3 mimetic compound is 4-[4-[[2-(4-Chlorophenyl)-4,4-dimethyl-1-cyclohexen-1-yl]methyl]-1-piperazinyl]-N—[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof.

A further aspect of the invention relates to a kit comprising: (a) at least one ARTS mimetic compound as defined by the invention, specifically, the B3 compound; and (b) at least one BH3 mimetic compound. More specifically, the BH3 mimetic compound used for the kit of the invention is 4-[4-[[2-(4-Chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)benzamide (ABT-199), and any derivatives thereof. ABT-199 (venetoclax, RG7601, GDC-0199) represents the first-in-class, selective, oral BCL-2 inhibitor sparing platelets. It showed sub-nanomolar affinity to BCL-2 (K i<0.010 nM) with antitumor activity against non-Hodgkin's lymphoma (NHL), CLL, and acute leukemias in vitro. In vivo mouse xenograft studies showed activity against aggressive (Myc+) lymphomas as well as acute leukemia.

The present disclosure further describes the identification of XIAP as a novel E3-ligase of p53 and that ARTS upregulates p53 by antagonizing XIAP, indicating that ARTS and p53 regulate each other in a positive feedback loop manner. Moreover, the present disclosure presents the small molecule ARTS mimetics, B3, that binds directly to the unique sequence of ARTS in XIAP-BIR3. B3, just like ARTS, promoted apoptosis by simultaneously downregulating XIAP levels and upregulating p53 protein levels. The apoptotic pathway is an ordered process of programmed cell death that is often altered in various pathologic conditions associated with either increased or decreased apoptosis.

Modulating apoptosis by external means provides an important and promising approach that paves the way for a variety of therapeutically opportunities. For example, cancer is a condition associated with deregulated apoptosis, resulting in cells displaying increased survival. Thus, inducing apoptosis is valuable as a defense mechanism against hyper proliferating cells. It was shown that the anti-apoptotic proteins of the Bcl-2 family govern the pro-survival pathway and are over expressed in a variety of tumor types such as small cell lung cancer, melanoma, prostate and breast cancer.

Cancer treatment is among others aimed in restoring the apoptotic capabilities of cancer cells. Further, inhibitors of Bcl-2 and XIAP anti-apoptotic proteins are needed in order to revert to norm al apoptotic processes and thus trigger tumor cell death.

As indicated above, the inventors have previously found that upon induction of apoptosis, ARTS binds directly to both XIAP and Bcl-2, acting as a scaffold to bring these proteins together, leading to a UPS mediated degradation of Bcl-2.

The inventors now identified new players to regulate p53 levels. XIAP was revealed as a new E3-ligase to regulate p53 levels through the UPS (FIG. 29C). Also, based on the presented results, p53 and ARTS regulate each other in a positive feedback loop manner: p53 activates ARTS transcription, and in turn, ARTS upregulates p53 protein levels by antagonizing XIAP and preventing its degradation through the UPS. Significantly, discovering these new players to regulate p53 levels assisted in developing a novel class of dual targeting compounds to promote apoptosis by antagonizing XIAP and upregulating p53 (FIGS. 1, 3, 30 and FIG. 31). Due to the high frequency of p53 mutations, it became an appealing target for therapeutic strategies (Duffy et al. 2022 Cancers (Basel) 14; Hassin and Oren 2022 Nat Rev Drug Discov 1-18; Hu et al. 2021 J Hematol Oncol 14 157). In cancers containing low levels of WT-p53, inhibiting its E3-ligase MDM2 can reactivate p53 anti-tumor activity (Duffy et al. 2022 Cancers (Basel) 14; Hassin and Oren 2022 Nat Rev Drug Discov 1-18; Hu et al. 2021 J Hematol Oncol 14 157). Unfortunately, this approach was met with a significant challenge as it caused widespread activation of WT-p53 also in normal tissue (Hu et al. 2021 J Hematol Oncol 14 157). In addition, monotherapy of MDM2 antagonists is insufficient to suppress tumor progression. Thus, many researchers are focusing in identifying promising drug combination (Hassin and Oren 2022 Nat Rev Drug Discov 1-18; Hu et al. 2021 J Hematol Oncol 14 157). Importantly, p53 is a major player in promoting apoptosis in response to DNA damage induced by chemotherapy (Paek et al. 2016 Cell 165 631-642). However, many cancers develop chemotherapy resistance because of a limited increase in the p53 levels, this is due to the upregulation of the IAP family, mainly XIAP (Paek et al. 2016 Cell 165 631-642). Accordingly, the ARTS mimetic B3 compound disclosed herein (specifically, (S)-N1-(1-hydroxy-3-phenylpropan-2-yl)-N2-(4-(1-methyl-1H-imidazole-2-carbonyl)phenyl)oxalamide) could provide a potential solution for limiting the effect of p53 through developing XIAP-dependent chemotherapy resistant. Collectively, the results of the present disclosure provide an understanding of a new dynamic to regulate the levels of p53 protein. Both tumor suppressors, ARTS and p53 regulate each other under cellular stress conditions. Discovering the potent E3-ligase XIAP to regulate p53 levels indicates the importance of regulating p53 levels under normal conditions. Unfortunately, cancers can manipulate this p53 check-point by upregulating its negative regulators, especially XIAP. Consequently this led to reduction in p53 levels, thereby resulting in resistance to chemotherapy. The identification of the small molecule ARTS mimetics, B3 a novel class of dual-targeting compounds, regulate XIAP and p53 levels through the UPS. Hence, XIAP downregulation and degradation via the UPS thereby upregulating p53 levels by preventing its degradation through the UPS.

A first aspect of the present disclosure relates to an effective amount of at least one of apoptosis related protein in the TGF-beta signaling pathway (ARTS), any fragments thereof, or at least one mimetic compound thereof or any vehicle, matrix, nano- or micro-particle comprising the same, for use in a method for upregulating p53 levels in a cell.

Tumor suppression is the main function of p53 protein, which is encoded by the TP53 μgene on human chromosome 17. The p53 protein is posited to inhibit the phenotypic and genomic alterations associated with cancer development through a complex interplay with several signaling pathways known to play critical roles in essential cellular processes such as cell division, maintenance of genomic stability, apoptosis, autophagy, immune response, and regulation of tumor microenvironment. Approximately 50% of human tumors harbor mutations in the TP53 μgene, while the remaining malignancies expressing WT p53 display functional inactivation of the p53 pathway by alternative mechanisms implicating viral oncoproteins or negative regulators of p53 such as MDM2 or MDM4. In most if not all human malignancies, inactivation of the TP53 μgene usually occurs through the acquisition of loss of function mutations or negative regulation of wild-type p53 proteins. Inactivation of the TP53 μgene drives invasion, proliferation, and cell survival, thereby facilitating cancer progression and metastasis. More than 75% of TP53 μgene mutations result in loss of wild-type p53's activities. Mutated p53 proteins might act either as dominant negative over wild-type p53 action, or gain novel tumorigenic properties counteracting the protective function of wild-type p53. p53 acts mainly as a transcription factor that is activated in response to multiple stressors to regulate the expression of genes controlling proliferation, DNA repair, senescence and cell death. More specifically, binding of wild-type p53 protein to specific DNA response elements induces the expression of a wide array of genes that ultimately guard against cancer development and progression. Under physiological conditions, exposure of cells to different stress signals activates the p53 signaling pathway, allowing the cells to activate several transcriptional programs including cell cycle arrest, DNA repair, senescence, and apoptosis leading to suppression of tumor growth.

In some embodiments, “p53” as used by the present disclosure refers to the human p53 as denoted by Genebank Accession No. NC_000017.11. Still further, in some embodiments, p53 as used herein is the human p53 that comprises the amino acid sequence as denoted by NP_000537.3. Still further, in some embodiments, p53 as used herein comprises the amino acid sequence as denoted by SEQ ID NO: 19, or any variants and derivatives thereof. In yet some further embodiments, p53 as used herein is the human p53, encoded by the nucleic acid sequence comprising the nucleic acids sequence of SEQ ID NO: 20, or any homologs or variants thereof.

In some embodiments, the disclosed methods may upregulate the Wild type p53, specifically, p53 that display no mutations. In yet some further embodiments, p53 as indicated herein may alternatively refer to p53 that comprise mutations that do not affect its function. In some embodiments, upregulation of p53 by the disclosed ARTS and/or fragments and/or mimetics thereof, restores p53 function.

In certain embodiments, the present disclosure further encompasses the use of the ARTS polypeptide and/or any fragments or peptides thereof. Thus, in such specific embodiments, the present disclosure provides uses of the ARTS and fragments thereof in methods for upregulating p53 levels in cells, or as disclosed herein after, in a subject in need thereof. According to the disclosed uses, the methods may comprise the step of contacting said cells with an effective amount of ARTS, any fragments thereof or any composition comprising the same.

As used herein “ARTS” (apoptosis-related protein in the TGF-β signaling pathway) is a septin-like mitochondrial protein derived from alternative splicing of the H5/PNUTL2/hCDCre12a/2b gene. ARTS acts as a tumor suppressor protein that functions as an antagonist of XIAP and thereby promotes apoptosis.

It should be appreciated that in certain embodiments, as used herein in the specification and in the claim section below, ARTS protein refers to the human ARTS (as denoted by SEQ ID NO. 1). More specifically, the human ARTS protein comprises an amino acid sequence of 274 amino acid residues as denoted by GenBank Accession No. AF176379, encoded by a nucleic acid sequence of SEQ ID NO. 2.

As indicated above, any fragment of ARTS, specifically, any functional fragment that may lead directly or indirectly to upregulation in p53 levels, may be used. In certain embodiments, any ARTS fragment that leads to reduction in the levels of at least one E3 ligase, for example, XIAP, and/or MDM2, may be applicable in the disclosed uses. In some specific embodiments, peptides derived from ARTS C′-terminal domain, more specifically, peptides derived from 27 amino acid sequence of ARTS C′-terminus may be applicable in the disclosed uses and methods. In more specific embodiments, the ARTS C′ terminus peptide applicable in the disclosed uses and methods, may comprise the amino acid sequence of: YGPSLRLLAPPGAVKGTGQEHQGQGCH, as denoted by SEQ ID NO. 24. In some particular embodiments, such ARTS fragments or peptides may comprise any peptide derived from the ARTS C′ terminal 27 amino acid residues. In more specific embodiments, such peptides applicable in the disclosed uses and methods, may comprise the amino acid sequence of any one of YGPSLRLLA, as denoted by SEQ ID NO. 25, PPGAVKGTG, as denoted by SEQ ID NO. 26, and QEHQGQGCH, as denoted by SEQ ID NO. 27.

In yet some further embodiments, ARTS fragments derived from its N-terminus, may be used in the disclosed uses and methods, as described above. In more specific embodiments, such ARTS fragments may include ARTS BH3-like domain. In some particular embodiments, such BH3-like domain ARTS fragments may comprise the amino acid sequence of residues 1-128, 1-148, 106-148, 106-133, 106-128, 112-148, 112-133 and 112-128 of ARTS N′-terminus, as denoted by any one of SEQ ID NO. 28 to 35, respectively.

In some embodiments of the effective amount of ARTS or at least one mimetic compound thereof for use as disclosed herein, the upregulation of p53 levels may comprise reduction of p53 ubiquitylation by at least one E3 ligase.

More specifically, as firstly shown herein, XIAP serves as an E3 ligase for p53, thereby leading to ubiquitylation and UPS mediated degradation thereof. As further shown by the present disclosure, ARTS, fragments thereof, as well as any mimetic compounds thereof, specifically, the “B3” and the “A4” compounds of the present disclosure, lead to UPS degradation of XIAP, and in some embodiments, the UPS mediated degradation of other E3 ligases, thereby inhibiting and attenuating ubiquitylation of p53, that results in upregulation of p53 levels. More specifically, ARTS, fragments thereof and/or any mimetic compounds thereof, specifically, the “B3 compound”, are provided in the present disclosure for use in methods, compositions and kits for upregulating p53 levels. In some embodiments, the upregulation is a result of UPS-mediated induction of degradation of E3 ligases such as XIAP and/or MDM2. The down regulation in E3 ligases results in a clear decrease in p53 ubiquitylation, thereby upregulating the levels of p53.

As used herein the term “ubiquitin proteasome system” (UPS) denotes a multi component system that identifies and degrades unneeded, damaged or misfolded proteins by breaking peptide bonds (proteolysis) of the protein in the cytoplasm of cells. As appreciated in the art, degradation of a protein via the UPS involves two discrete and successive steps. In the first step, proteins are tagged for degradation with a small protein called ubiquitin. The tagging reaction is catalyzed by enzymes called ubiquitin ligases. Once a protein is tagged with a single ubiquitin molecule, this is a signal to other ligases to attach additional ubiquitin molecules.

More specifically, conjugation of ubiquitin, a highly evolutionarily conserved 76 amino acid residue polypeptide, to the protein substrate proceeds via a three-step cascade mechanism involving E1, E2 and E3 enzymes. By successively adding activated ubiquitin moieties to internal lysine residues on the previously conjugated ubiquitin molecule, a polyubiquitin chain is synthesized that is subsequently recognized by the downstream 26S proteasome complex.

In the second step, degradation of polyubiquitinated substrates is carried out by a large, protease complex, referred to as the 26S proteasome that does not recognize nonmodified substrates. The proteasomes are multicatalytic protease protein complexes found in all cells that degrades polyubiquitinated proteins to short peptides by breaking peptide bonds (proteolysis). Following degradation of the substrate, short peptides derived from the substrate are released, along with reusable ubiquitin.

It should be noted that the ubiquitin-proteasome system (UPS) plays a central and complex role in regulating apoptosis by directly targeting key cell death proteins, including caspases.

E3 ligases are the largest and most studied group of the Ubiquitin Proteasome System (UPS), which is responsible for the regulated degradation of intracellular proteins.

Ubiquitylation is the post-translational conjugation of a ubiquitin protein, which tags proteins destined for degradation via the 26S proteasome The ubiquitylation cascade requires a ubiquitin activating enzyme (E1), ubiquitin conjugating enzymes (E2) and the ubiquitin ligases (E3). The E1 enzyme forms an ATP-dependent thioester linkage with the carboxyl-terminus of ubiquitin; E1 is not substrate-specific. The E2 enzyme receives the activated ubiquitin from E1, which in turn transfers the ubiquitin to the E3 ligase. Different E2 enzymes can regulate a single E3 ligase. The E3 ligases are substrate-specific and are essential for the final transfer of the activated ubiquitin from the E2 enzyme to the lysine residue onto the target protein.

E3 ligases can be classified into three major groups: (1) the RING E3 ligase family (largest group), (2) the HECT family (homologous to Human Papilloma virus E6 Carboxyl Terminal domain), and (3) the RBR (RING between RING fingers) E3 ligase family.

In some embodiments, such E3 ligase may be XIAP. In some embodiments, by down-regulating the levels of XIAP, specifically, increasing the UPS mediated degradation of XIAP, the ARTS, fragments thereof and the ARTS mimetic compounds, specifically the “B3” compound as disclosed herein after, used by the present disclosure, lead to upregulation of p53. Alternatively, due to the observed direct interaction of ARTS/ARTS mimetics with p53 (FIG. 27D and FIG. 31B), the inventors assume that ARTS, ARTS fragments and the ARTS mimetic compounds disclosed herein may affect p53 activity also by translocating p53 into the nucleus (FIG. 28) and/or by other p53-dependent signaling pathway means.

As previously shown by the inventors, ARTS binds to XIAP through a domain comprising 27 residues covering the C-terminus of ARTS. This interaction induces auto degradation of XIAP. The ARTS mimetic compound/s of the invention target BIR3 domain of XIAP mimicking the ability of ARTS to enhance XIAP degradation.

As used herein the term “IAPs” denotes a family of proteins that harbor between one to three copies of a baculovirus IAP repeat (BIR) domain that enable interaction with activated caspases. It was previously suggested that the BIR domains of certain IAPs, in particular XIAP, have the ability to directly inhibit caspase activity in vitro.

X-linked inhibitor of apoptosis protein (XIAP), also known as inhibitor of apoptosis protein 3 (IAP3) and baculoviral IAP repeat-containing protein 4 (BIRC) denotes a protein known to stop an apoptotic process and thus inhibit cell death. In human, XIAP is produced by a gene named XIAP gene located on the X chromosome. XIAP is also called human IAP-like Protein (hILP), because it is not as well conserved as the human IAPS: hIAP-1 and hIAP-2-XIAP are the most potent human IAP proteins currently identified.

XIAP belongs to a family of apoptotic suppressor proteins. Members of this family share a conserved motif termed, baculovirus IAP repeat (BIR domain), which is necessary for their anti-apoptotic function. XTAP acts as a direct caspase inhibitor by directly binding to the active site pocket of CASP3 and CASP7 and obstructing substrate entry. It further inactivates CASP9 by keeping it in a monomeric, inactive state.

It should be noted that in certain embodiments, the invention relates to the human XIAP protein (GenBank Accession Nos. NP_001158, NP_001191330, as denoted by SEQ ID NO. 9) encoded by the XIAP gene (GenBank Accession Nos. NM_001167, NM_001204401, as denoted by SEQ ID NO. 10).

In yet some further embodiments, as shown by the following Examples, the ARTS, fragments thereof and the ARTS mimetic compounds, specifically the “B3” compound, used by the present disclosure, may lead to down-regulation of MDM2 levels, specifically, via UPS, thereby leading to upregulation of p53 levels. In some embodiments, MDM2, as used herein, refers to the human MDM2, that is an E3 ligase that acts on p53. In some embodiments MDM2 is the human MDM2, that comprises the amino acid sequence as denoted by UNIPROT accession number Q00987. In yet some further embodiments, MDM2, as used herein may be the human MDM2, encoded by a nucleic acid sequence comprising the sequence as denoted by GeneBank accession number NM_001145337.3. In yet some further embodiments, the MDM2 as disclosed herein may comprise the amino acid sequence as denoted by SEQ ID NO. 22, or any variants or derivatives thereof. In some further embodiments, the human MDM2 as disclosed herein may be encoded by a nucleic acid sequence comprising the nucleic aid sequence as denoted by SEQ ID NO. 23.

As indicated above, the present invention relates to the ARTS mimetic compounds of the invention that act as antagonist/s of XIAP and other E3 ligases (e.g., MDM2). An antagonist is a compound that competes with a specific protein, a ligand for example, on binding to another protein, a receptor for example. Such binding usually, induces a specific biological response or action that is blocked by the competing antagonist. Antagonists have affinity but no efficacy for their cognate binding protein and binding will disrupt the interaction and inhibit the function of such cognate protein. Antagonists mediate their effects by binding to the active (orthosteric=right place) site or to allosteric (=other place) sites on any cognate protein, in this case, XIAP (or receptor, in case applicable), or they may interact at unique binding sites not normally involved in the biological regulation of the cognate protein.

It should be noted that by inducing XIAP degradation (and optionally, any other relevant E3 ligase), ARTS, fragments thereof and the ARTS mimetic compound/s of the present disclosure, inhibit the anti-apoptotic effect of XIAP protein. The terms “inhibition”, “moderation” or “attenuation” as referred to herein, relate to the retardation, restraining or reduction of the anti-apoptotic activity of a the XIAP protein, and specifically, the E3 ligase activity thereof on p53. Such inhibition may be of about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95%, 99.9% or 100%.

It should be further noted that by decreasing the levels of E3 ligases such as XIAP, and/or MDM2, the present disclosure provides a powerful approach leading to elevation in the levels of p53. Elevation of p53 levels results in the inhibition of the anti-apoptotic action of XIAP, and optionally of MDM2. Thus, in some embodiments ARTS, fragments thereof and/or the ARTS mimetic compounds of the present disclosure upregulate p53, and/or induce or enhances apoptosis. More specifically, the ARTS, fragments thereof and/or the ARTS mimetic compounds of the present disclosure, specifically, B3, as well as any of the compositions and methods of the invention described herein after, may lead to upregulation, an increase, enhancement, induction or elevation in p53 levels in the treated cells, and/or in apoptosis of treated cells, the upregulation, increase, induction or elevation of apoptosis may be an increase by about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%. More specifically, an increase of about 5%, 10%, 15%, 20%, 25%, 30%, 35%, 40%, 45%, 50%, 55%, 60%, 65%, 70%, 75%, 80%, 85%, 90%, 95%, 99% or 100% as compared to untreated control, specifically, cells that were not treated with ARTS, fragments thereof and/or the ARTS mimetic compounds of the present disclosure.

With regards to the above, it is to be understood that, where provided, percentage values such as, for example, 10%, 50%, 120%, 500%, etc., are interchangeable with “fold change” values, i.e., 0.1, 0.5, 1.2, 5, 10, 20, 30, 40, 50, 60, 70, 80, 90, 100 etc., respectively.

In yet some further embodiments, the ARTS mimetic compound for use in the preset disclosure, is a compound having the general formula (I) or a pharmaceutically acceptable salt, solvate, hydrate, any stereoisomer thereof or physiologically functional derivative thereof, wherein formula (I) is:

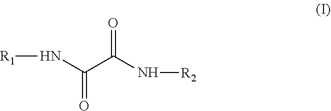

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In some embodiments of the ARTS mimetic compound for use in the preset disclosure, the L1′, L1″, L2′ and L2″ are independently from each other selected from —(CH₂)ₙ—, C(═O), optionally —(CH₂)ₙ— substituted with —(CH₂)ₘ—OH and wherein n is an integer selected from 1, 2, or 3 and m is an integer selected from 1, 2 or 3.

In yet some further embodiments, the ARTS mimetic compound for use in accordance with the present disclosure, each one of R₃′, R₃″, R₄′, R₄″ is absent or may be independently from each other an aromatic or a heteroaromatic ring, each is independently from each other optionally substituted with OH, alkyl, halogen, CF₃, NO₂, C(═O).

In some further embodiments of the present disclosure, the ARTS mimetic compound for use, is having the general formula (II)

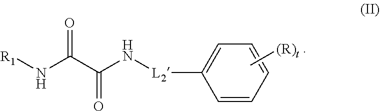

Wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments of the ARTS mimetic compound for use in the preset disclosure, the L2′ is (CH₂)ₙ—, or C(═O), optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some further embodiments, the ARTS mimetic compound for use in accordance with the present disclosure, is a compound having the general formula (IIb):

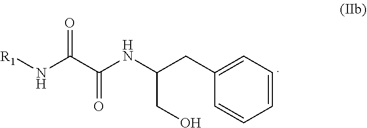

In some further embodiments of disclosed ARTS mimetic compound for use, the R1 is L1′-R₃′-L1″-R₃″.

In yet some further embodiments of disclosed ARTS mimetic compound for use, the R1 is at least one of:
(i) L1′, L1″ and R3″ are each absent and R₃′ is an optionally substituted

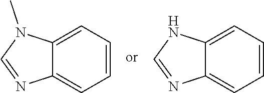

(ii) L1′, L1″ and R₃″ are each absent and R₃′ is:

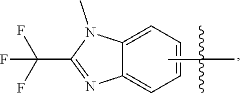

or
(iii) L1′ is absent R₃′ is an optionally substituted phenyl, R₃″ is an optionally substituted:

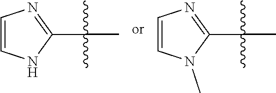

and L1″ is C(═O).

In some embodiments, the ARTS mimetic compound for use, according to the present disclosure, is a compound having the general formula (IIIa) or (IIIb):

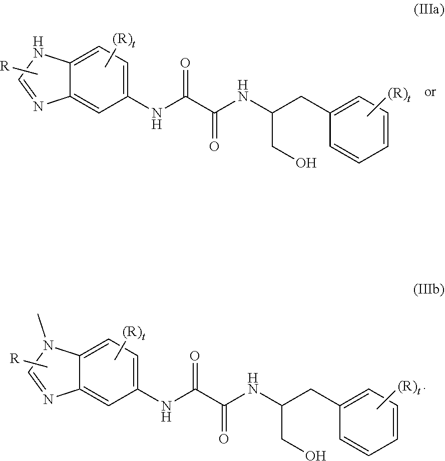

wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments, the ARTS mimetic compound for use of the present disclosure, is a compound having the general formula (IIIc), (Id) or (IIIe):

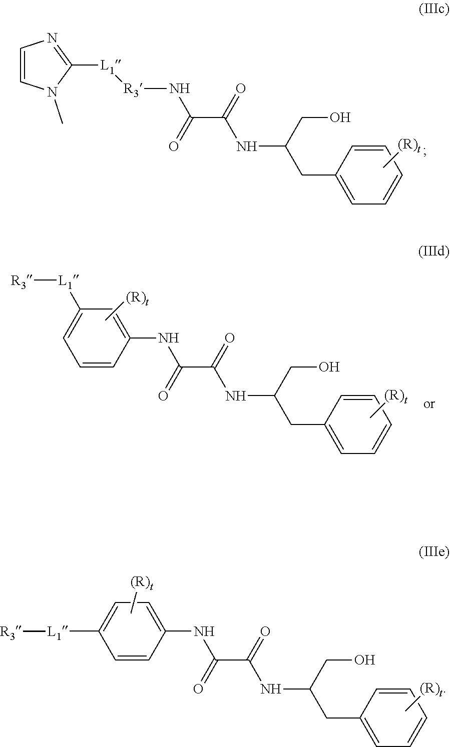

wherein L1″ and R3″ are each as defined above, wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments, the ARTS mimetic compound for use in accordance with the present disclosure is a compound having the general formula (IIIc), or (IIIe):

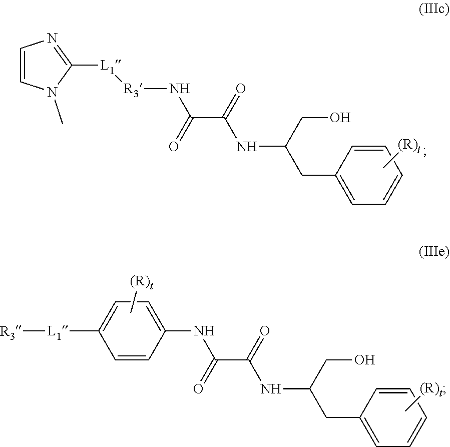

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In yet some further embodiments, the ARTS mimetic compound for use in accordance with the present disclosure, is a compound having the formula (3.1), (3.2), (3.3);

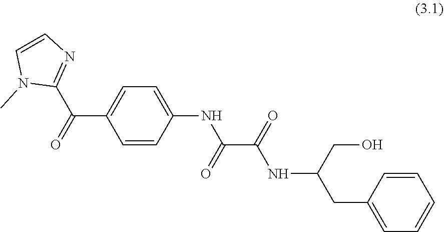 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

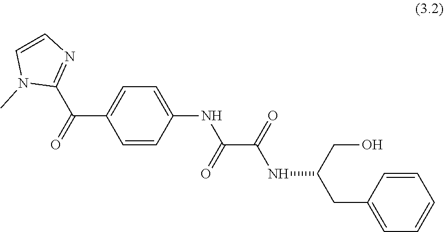

### (S)—N1-(1-hydroxy-3-phenylpropan-2-yl)-N2-(4-(1-methyl-1H-imidazole-2-carbonyl)phenyl)oxalamide (denoted herein as “B3”)

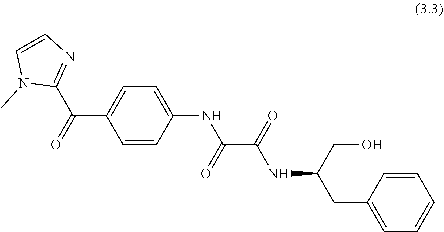 [SMILES: Cn1ccnc1C(=O)c1ccc(NC(=O)C(=O)N[C@@H](CO)Cc2ccccc2)cc1]

### (R)-N1-(1-hydroxy-3-phenylpropan-2-yl)-N2-(4-(1-methyl-1H-imidazole-2-carbonyl)phenyl)oxalamide

More specifically, the present disclosure provides the use of 1,2-di-carbonyl compounds that serve herein as ARTS mimetics, in upregulating p53 levels. In accordance with this aspect, the present disclosure provides a compound comprising at least one oxalamide moiety. The present disclosure also encompasses pharmaceutically acceptable salt, solvate, hydrate or any stereoisomer of the compounds described herein.

In accordance with this aspect, the present disclosure provides the use of a compound having the general formula (I):

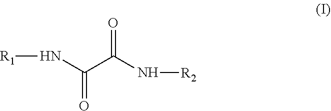

or a pharmaceutically acceptable salt, solvate, hydrate, any stereoisomer thereof or physiologically functional derivative thereof;
wherein
R₁, R₂ are each, independently from each other, absent or H, alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, or —S—(CH₂)ₙ—, each optionally substituted; n is an integer selected from any one of 0, 1, 2, 3, 4, 5 each may be independently from each other, optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ-halogen and m is an integer selected from 0, 1, 2, 3, 4, 5.

It should be noted that in some embodiments, the ARTS mimetic compound used in the methods, composition and kits of the present disclosure may comprise a compound having the structure of formula (3.2). In some embodiments, this compound (as well as derivatives thereof may be referred to herein as “B3” or “B3 ARTS mimetic compound”. This compound has been described in more detail in connection with previous aspect of the present disclosure that are all encompassed in connection with the following aspects as well.

A indicated herein above, the present disclosure provides the uses, compositions and methods of any ARTS mimetic compound. Specifically, the present disclosure provides the uses of different ARTS mimetic compounds that specifically mimic the C′ domain of ARTS, specifically in binding thereof to its binding site within the BIR3 domain of XIAP. In some specific embodiments, the ARTS mimetic compound used in the methods, kits and compositions disclosed herein may be a compound having the Formula (X), or any analogs or derivative thereof including any stereoisomer or salt thereof.

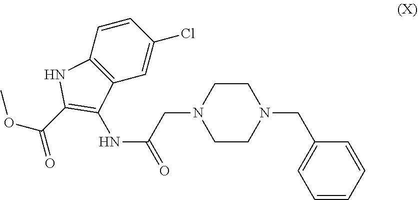 [SMILES: COC(=O)c1[nH]c2ccc(Cl)cc2c1NC(=O)CN1CCN(Cc2ccccc2)CC1]

### 3-[2-(4-Benzyl-piperazin-1-yl)-acetylamino]-5-chloro-1H-indole-2-carboxylic acid methyl ester

It should be appreciated that such compound may be referred to herein by the present disclosure as “A4” or “ARTS mimetic A4 small molecule” or the like. It should be also noted that when referring to A4, the compound may include any stereoisomer or salt thereof, for example the stereoisomer having the structure:

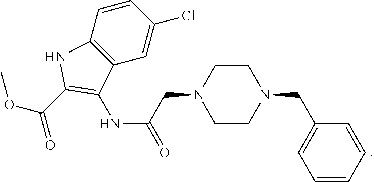

In some embodiments, the ARTS or at least one mimetic compound as disclosed herein, is for use in a method for upregulating p53 levels in at least one cell of a subject suffering from at least one pathologic disorder.

In some further embodiments, the present disclosure provides ARTS or at least one mimetic compound for use in a method for upregulating p53 levels in at least one cell of a subject suffering from at least one p53-associated disorder.

In yet some further embodiments of the ARTS or at least one mimetic compound thereof for use in accordance with the preset disclosure, the p53-associated disorder comprises at least one of: at least one proliferative disorder and at least one metabolic disorder.

In some embodiments, the ARTS or at least one mimetic compound thereof for use in a method as disclosed herein, the proliferative disorder is at least one neoplastic malignant disorder.

In some further embodiments, the present disclosure provides ARTS or at least one mimetic compound thereof for use in a method for upregulating the levels of p53 in at least one cell in a subject suffering from at least one pathologic disorder. In some embodiments, the method comprising administering to the subject a therapeutically effective amount of the ARTS or at least one mimetic compound thereof.

As shown herein in the following Examples, the use of ARTS, fragments thereof and/or mimetic compounds thereof, lead to upregulation of p53 levels, and to increased apoptosis, as also reflected by elevation in apoptotic markers such as cleaved caspases and PARP. The term “apoptosis” refers to a regulated network of biochemical events which lead to a selective form of cell suicide and is characterized by readily observable morphological and biochemical phenomena. Cells undergoing apoptosis show characteristic morphological and biochemical features. These features include chromatin aggregation or condensation, DNA fragmentation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial dysfunction accompanying apoptosis.

As indicated above, apoptosis is a tightly controlled form of active cell death that is necessary for development and organismal homeostasis. Death by the apoptotic pathway is achieved among others, by the activation of a family of highly potent and specific proteases, termed caspases (for cysteine-aspartate protease).

The activity of caspases is tightly regulated and the cell maintains several “checkpoints” to control their activity. The first level of regulation is intrinsic to caspases themselves. Caspases are initially transcribed as weakly active zymogens, which only upon proper stimulation are cleaved to form the active enzyme. The second level of caspase regulation is achieved by inhibitors, namely the family of proteins called IAPs (Inhibitor of Apoptosis Protein) as described above.

As indicated above, the preset disclosure encompasses uses of ARTS and any fragments and peptides thereof, in methods for upregulating p53 levels. The present disclosure thus provides peptides and polypeptides, as well as any derivatives and variants thereof. The term “polypeptide” as used herein refers to amino acid residues, connected by peptide bonds. A polypeptide sequence is generally reported from the N-terminal end containing free amino group to the C-terminal end containing free carboxyl group.

More specifically, “Amino acid molecule”, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

Amino acids, as used herein refer to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

It should be noted that in addition to any of the ARTS derived fragments or peptides described herein, the present disclosure further encompasses any derivatives, analogues, variants or homologues of any of the peptides. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present disclosure.

It should be further noted that the polypeptides according to the disclosure can be produced synthetically, or by recombinant DNA technology. Methods for producing polypeptides peptides are well known in the art.

In some embodiments, derivatives include, but are not limited to, polypeptides that differ in one or more amino acids in their overall sequence from the polypeptides defined herein (either the ARTS protein or any fragment or peptide derived therefrom according to the present disclosure), polypeptides that have deletions, substitutions, inversions or additions.

In some embodiments, derivatives refer to polypeptides, which differ from the polypeptides specifically defined in the present disclosure by insertions of amino acid residues. It should be appreciated that by the terms “insertions” or “deletions”, as used herein it is meant any addition or deletion, respectively, of amino acid residues to the polypeptides used by the present disclosure, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the present disclosure may occur in any position of the modified peptide, as well as in any of the N‘ or C’ termini thereof.

The peptides of the present disclosure may all be positively charged, negatively charged or neutral. In addition, they may be in the form of a dimer, a multimer or in a constrained conformation, which can be attained by internal bridges, short-range cyclizations, extension or other chemical modifications.

The polypeptides of the present disclosure can be coupled (conjugated) through any of their residues to another peptide or agent. For example, the polypeptides of the present disclosure can be coupled through their N-terminus to a lauryl-cysteine (LC) residue and/or through their C-terminus to a cysteine (C) residue.

Further, the peptides may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residue/s, which may be naturally occurring or synthetic amino acid residue/s, for example, a specific aromatic amino acid residue may be tryptophan. The peptides may be extended at the N-terminus and/or C-terminus thereof with various identical or different organic moieties, which are not naturally occurring or synthetic amino acids. As an example for such extension, the peptide may be extended at the N-terminus and/or C—terminus thereof with an N-acetyl group.

For every single peptide sequence defined by the present disclosure and disclosed herein, this disclosure includes the corresponding retro-inverse sequence wherein the direction of the peptide chain has been inverted and wherein all the amino acids belong to the D-series.

The present disclosure also encompasses any homologues of the polypeptides (either the ARTS protein or any fragments or peptides thereof) specifically defined by their amino acid sequence according to the present disclosure. The term “homologues” is used to define amino acid sequences (polypeptide) which maintain a minimal homology to the amino acid sequences defined by the present disclosure, e.g. preferably have at least about 65%, more preferably at least about 75%, even more preferably at least about 85%, most preferably at least about 95% overall sequence homology with the amino acid sequence of any of the polypeptide as structurally defined above, e.g. of a specified sequence, more specifically, an amino acid sequence of the polypeptides as denoted by any one of SEQ ID NO:1 and SEQ ID Nos: 28 to 35.

More specifically, “Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions or deletions shall be construed as reducing identity or homology. Methods and computer programs for the alignment are well known in the art.

In some embodiments, the present disclosure also encompasses polypeptides which are variants of, or analogues to, the polypeptides specifically defined in the present disclosure by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art. Such conservatively modified variants are in addition to and do not exclude polymorphic variants, interspecies homologues, and alleles and analogous peptides of the present disclosure.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

In certain embodiments the peptide compounds of the present disclosure may comprise one or more amino acid residue surrogate. An “amino acid residue surrogate” as herein defined is an amino acid residue or peptide employed to produce mimetics of critical function domains of peptides.

Examples of amino acid surrogate include, but are not limited to chemical modifications and derivatives of amino acids, stereoisomers and modifications of naturally occurring amino acids, non-protein amino acids, post-translationally modified amino acids, enzymatically modified amino acids, and the like. Examples also include dimers or multimers of peptides. An amino acid surrogate may also include any modification made in a side chain moiety of an amino acid. This thus includes the side chain moiety present in naturally occurring amino acids, side chain moieties in modified naturally occurring amino acids, such as glycosylated amino acids. It further includes side chain moieties in stereoisomers and modifications of naturally occurring protein amino acids, non-protein amino acids, post-translationally modified amino acids, enzymatically synthesized amino acids, derivatized amino acids, constructs or structures designed to mimic amino acids, and the like.

It should be appreciated that the present disclosure further encompass any of the peptides of the present disclosure any serogates thereof, any salt, base, ester or amide thereof, any enantiomer, stereoisomer or disterioisomer thereof, or any combination or mixture thereof. Pharmaceutically acceptable salts include salts of acidic or basic groups present in compounds of the present disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the present disclosure can form pharmaceutically acceptable salts with various amino acids. Suitable base salts include, but are not limited to, aluminum, calcium, lithium, magnesium, potassium, sodium, zinc, and diethanolamine salts.

It should be noted that the present disclosure further encompasses any peptidomimetic compound mimicking the C′-terminus derived peptides used by the present disclosure. When referring to peptidomimetics, what is meant is a compound that mimics the conformation and desirable features of a particular natural peptide but avoids the undesirable features, e.g., flexibility and bond breakdown. From chemical point of view, peptidomimetics can have a structure without any peptide bonds, nevertheless, the compound is peptidomimetic due to its chemical properties and not due to chemical structure. Peptidoinimetics (both peptide and non-peptidyl analogues) may have improved properties (e.g., decreased proteolysis, increased retention or increased bioavailability). It should be noted that peptidomimetics may or may not have similar two-dimensional chemical structures, but share common three-dimensional structural features and geometry. Each peptidomimetic may further have one or more unique additional binding elements.

Another aspect of the present disclosure relates to a composition comprising effective amount of at least one of ARTS, any fragments thereof, at least one mimetic compound thereof or any vehicle, matrix, nano- or micro-particle comprising the same, for use in a method for upregulating p53 levels in a cell. More specifically, the composition optionally further comprises at least one of pharmaceutically acceptable carrier/s, excipient/s, auxiliaries, and/or diluent/s.

In some embodiments, the present disclosure provides a composition for use in a method for upregulating p53 levels in a cell. In some embodiments, the upregulating p53 levels comprises reduction of p53 ubiquitylation by at least one E3 ligase.

In some embodiments, the ARTS mimetic compound of the composition for use in accordance with the present disclosure, is a compound having the general formula (I), wherein formula (I) is:

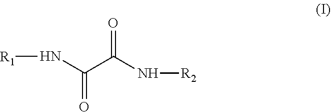

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In a further embodiment, the ARTS mimetic compound of the of the composition for use in accordance with the preset disclosure, is a compound having the general formula (II)

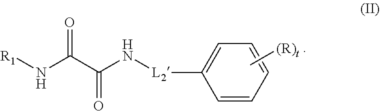

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In yet some further embodiment, the ARTS mimetic compound of the composition for use, as disclosed herein, is having the general formula (IIIc), or (IIIe):

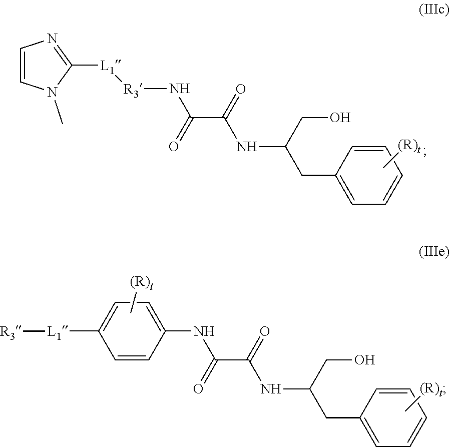

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In further embodiments of the composition for use as disclosed herein, the ARTS mimetic compound is having the formula (3.1), (3.2), (3.3);

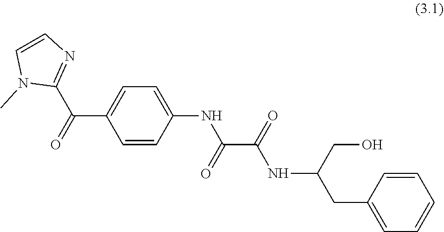 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

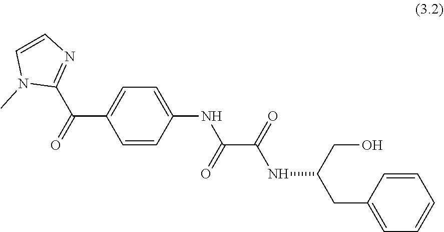

### (S)-N1-(1-hydroxy-3-phenylpropan-2-yl)-N2-(4-(1-methyl-1H-imidazole-2-carbonyl)phenyl)oxalamide (denoted herein as “B3”)

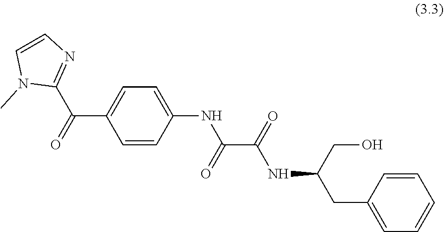 [SMILES: Cn1ccnc1C(=O)c1ccc(NC(=O)C(=O)N[C@@H](CO)Cc2ccccc2)cc1]

### (R)—N1-(1-hydroxy-3-phenylpropan-2-yl)-N2-(4-(1-methyl-1H-imidazole-2-carbonyl)phenyl)oxalamide

In certain embodiments, the compositions of the present invention can be administered for prophylactic and/or therapeutic treatments. In therapeutic application, compositions are administered to a patient already affected by any p53-associated disorder, for example, a proliferative disorder (e.g., carcinoma, specifically breast carcinoma), and/or any metabolic disorder in an amount sufficient to cure or at least partially arrest the condition and its complications. An amount adequate to accomplish this is defined as a “therapeutically effective dose.” Amounts effective for this use will depend upon the severity of the condition and the general state of the patient's own immune system, but generally range from about 0.001 to about 1000 mg/Kg. Single or multiple administrations on a daily, weekly or monthly schedule can be carried out with dose levels and pattern being selected by the treating physician. Additionally, the administration of the compositions of the invention, may be periodic, for example, the periodic administration may be affected twice daily, three time daily, or at least one daily for at least about three days to three months. The advantages of lower doses are evident to those of skill in the art. These include, inter alia, a lower risk of side effects, especially in long-term use, and a lower risk of the patients becoming desensitized to the treatment. In another embodiment, treatment using the compositions of the present disclosure, may be affected following at least 1, 2, 3, 4, 5, 6, 7, 8, 9, 10, 14, 30, 60, 90 days of treatment, and proceeding on to treatment for life.

In some embodiments, the effective amount of the disclosed ARTS mimetic compounds, and specifically of the B3 compound, may range from about 0.1 μM to about 100 μM. Specifically, from 0.5 μM to about 100 μM, from 1 μM to about 100 μM, 1 μM to 90, 1 μM to 80 μM, 1 μM to 70 μM, 1 μM to 80 μM, 1 μM to 70 μM, 1 μM to 60 μM, 1 μM to 50 μM, 1 μM to 40 μM, 1 μM to 30 μM, 1 μM to 20 μM, 1 μM to 10 μM, specifically, 1 μM or less, 2, 3, 4, 5, 6, 7, 8, 9, 10, 11, 12, 13, 14, 15, 16, 17, 18, 19, 20 μM or more. In some embodiments, an effective amount of the “B3” compound may be 20 μM.

It should be noted that the treatment of different p53-associated disorders, e.g., proliferative conditions and/or metabolic disorders may indicate the use of different doses or different time periods, these will be evident to the skilled medical practitioner.

For prophylactic applications, the compositions of the invention may include a prophylactic effective amount of the active ingredient. The term “prophylactically effective amount” is intended to mean that amount of a pharmaceutical composition that will prevent or reduce the risk of occurrence or recurrence of the biological or medical event that is sought to be prevented in a tissue, a system, animal or human by a researcher, veterinarian, medical doctor or other clinician. In prophylactic applications, the compositions of the invention are administered to a patient who is at risk of developing the disease state to enhance the patient's resistance. Such an amount is defined to be a “prophylactically effective dose”. In this use, the precise amounts again depend upon the patient's state of health and general level of immunity, but generally range from 0.001 to 1000 mg per dose.

It should be appreciated that the ARTS, functional fragments thereof, and ARTS mimetic compounds of the present disclosure, specifically, the B3 compound, may be formulated in any vehicle, matrix, nano- or micro-particle, or composition. Of particular relevance are formulations of the ARTS mimetic compounds adapted for use as a nano- or micro-particles. Nanoscale drug delivery systems using micellar formulations, liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as micelles, liposomes or polyelectrolyte multilayer capsules, incorporate nanoparticle (NP) actuators. More specifically, Controlled drug delivery systems (DDS) have several advantages compared to the traditional forms of drugs. It should be therefore understood that the present disclosure further encompasses the use of various nanostructures, including micellar formulations, liposomes, polymers, dendrimers, silicon or carbon materials, polymeric nanoparticles and magnetic nanoparticles, as carriers in drug delivery systems. The term “nanostructure” or “nanoparticle” is used herein to denote any microscopic particle smaller than about 100 nm in diameter. In some other embodiments, the carrier is an organized collection of lipids. When referring to the structure forming lipids, specifically, micellar formulations or liposomes, it is to be understood to mean any biocompatible lipid that can assemble into an organized collection of lipids (organized structure). In some embodiments, the lipid may be natural, semi-synthetic or fully synthetic lipid, as well as electrically neutral, negatively or positively charged lipid. In some embodiments, the lipid may be a naturally occurring phospholipid.

It should be appreciated that the ARTS, functional fragments thereof, and ARTS mimetic compounds of the present disclosure may be associated with any of the nanostructures described above, specifically, any of the micellar formulations, liposomes, polymers, dendrimers, silicon or carbon materials, polymeric nanoparticles and magnetic nanoparticles disclosed herein above. The term “association” may be used interchangeably with the term “entrapped”, “attachment”, “linked”, “embedded”, “absorbed” and the like, and contemplates any manner by which the at least one ARTS mimetic compounds of the disclosure is held.

As mentioned herein before, the compositions provided by the present disclosure optionally further comprise at least one pharmaceutically acceptable excipient or carrier. As used herein “pharmaceutically acceptable carrier” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

The pharmaceutical composition of the present disclosure can be administered and dosed by the methods of the invention, in accordance with good medical practice. More specifically, the compositions used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient.

As mentioned herein before, the compositions provided by the disclosure optionally further comprise at least one pharmaceutically acceptable excipient or carrier. As used herein “pharmaceutically acceptable carrier” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

As mentioned above, the compositions provided by the disclosure comprise an effective amount of any of the ARTS, functional fragments thereof (e.g. any of the peptides of SEQ ID NO: 24 to 35), and any mimetic compounds thereof, specifically, the B3 compound of the disclosure, specifically, the A4 compound including any stereoisomer or salt thereof, as well as any vehicle, matrix, nano- or micro-particle comprising the same.

The pharmaceutical composition of the disclosure can be administered and dosed by the methods of the disclosure, in accordance with good medical practice. More specifically, the compositions used in the methods and kits of the disclosure, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

The phrases “systemic administration,” “administered systemically,” “peripheral administration” and “administered peripherally” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Regardless of the route of administration selected, the compounds of the present disclosure, which may be used in a suitable hydrated form, and/or the pharmaceutical compositions of the present disclosure, are formulated into pharmaceutically-acceptable dosage forms by conventional methods known to those of skill in the art.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred methods of preparation are vacuum-drying and freeze drying techniques which yield a powder of the active ingredient plus any additional desired ingredient from a previously sterile-filtered solution thereof.

Pharmaceutical compositions used to treat subjects in need thereof according to the disclosure generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Local administration to the area in need of treatment may be achieved by, for example, local infusion during surgery, topical application, direct injection into the specific organ, etc.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions used to treat subjects in need thereof according to the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present disclosure also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

Another aspect of the present disclosure relates to methods for upregulating the levels of p53 in a cell. More specifically, the disclosed method comprises the step of contacting the cell with an effective amount of ARTS, any fragments thereof, or at least one mimetic compound thereof or any vehicle, matrix, nano- or micro-particle comprising the same, or any composition thereof.

In some embodiments of the disclosed methods, upregulating p53 levels comprises reduction of p53 ubiquitylation by at least one E3 ligase.

In some further embodiments of the disclosed methods, the ARTS mimetic compound is having the general formula (I), wherein formula (I) is:

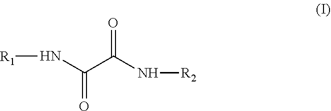

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L₁″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In some embodiments of the disclosed methods, the ARTS mimetic compound is having the general formula (II)

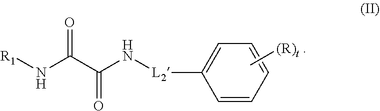

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some further embodiments of the disclosed methods, the ARTS mimetic compound is having the general formula (IIIc), or (IIIe):

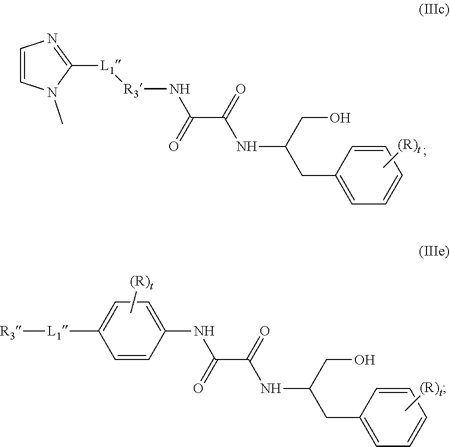

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In yet some further embodiments of the disclosed methods, the ARTS mimetic compound is having the formula (3.1), (3.2), (3.3);

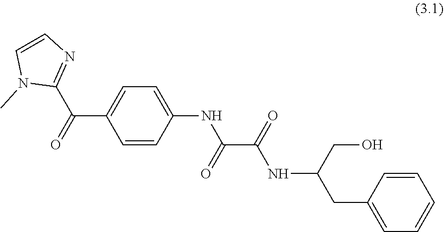 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

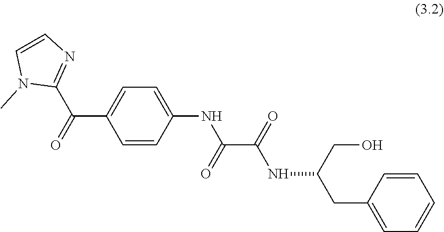

### (S)—N1-(1-hydroxy-3-phenylpropan-2-yl)-N2-(4-(1-methyl-1H-imidazole-2-carbonyl)phenyl)oxalamide (denoted herein as “B3”)

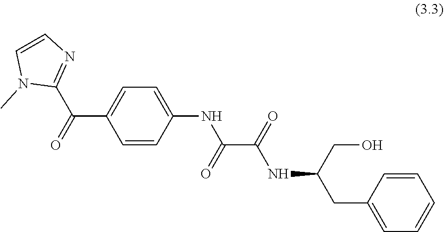 [SMILES: Cn1ccnc1C(=O)c1ccc(NC(=O)C(=O)N[C@@H](CO)Cc2ccccc2)cc1]

### (R)—N1-(1-hydroxy-3-phenylpropan-2-yl)-N2-(4-(1-methyl-1H-imidazole-2-carbonyl)phenyl)oxalamide

In yet some further embodiments, the disclosed methods upregulate p53 levels in a cell that may be of a subject suffering from at least one pathologic disorder.

In some embodiments of, the disclosed methods upregulate p53 levels in a cell that may be of a subject suffering from at least one p53-associated disorder.

In some embodiments of the disclosed methods, the p53-associated disorder may comprise at least one of: at least one proliferative disorder and at least one metabolic disorder.

In some further embodiments, the disclosed methods upregulate p53 levels in a cell that may be of a subject suffering from at least one proliferative disorder, in more specific embodiments, the disorder may be at least one neoplastic malignant disorder.

In some embodiments, the disclosed method is for upregulating the levels of p53 in at least one cell in a subject suffering from at least one pathologic disorder. Accordingly, the contacting step may comprise administering to the subject a therapeutically effective amount of said ARTS or at least one mimetic compound thereof.

Another aspect of the present disclosure relates to a method for treating, preventing, inhibiting, reducing, eliminating, protecting or delaying the onset of a pathological disorder in a subject in need thereof, by upregulating p53 levels in at least one cell of the subject. The method comprising administering to the subject a therapeutically effective amount of at least one of ARTS, any fragments thereof, or at least one mimetic compound thereof or any vehicle, matrix, nano- or micro-particle thereof or of a composition comprising the same.

In some embodiments of the therapeutic methods disclosed herein, the disorder is at least one p53-associated disorder.

The phrases “p53-associated disorder” and “p53-mediated disorder” refer to pathological and disease conditions in which a p53 protein is downregulated, not functioning, or display altered function. Moreover, this term also encompasses conditions in which p53 plays a role. Such roles can be directly related to the pathological condition or can be indirectly related to the condition. The feature common to this class of conditions is that they can be ameliorated by elevating, upregulating the expression of, activity of, function of, or association with p53.

The term “downregulated” refers to a decrease in the measurable level of p53 in a sample. More specifically, disorders displaying “downregulation” or “non-function” of p53 refer to disorders which demonstrate at least 10% or more, for example, 20%, 30%, 40%, or 50%, 60%, 70%, 80%, 90%, 95%, 100% or more, or 1.1 fold, 1.2 fold, 1.4 fold, 1.6 fold, 1.8, 1.9, 2, 3, 4, 5, 6, 7, 8, 9, 10, 20, 30, 40, 50, 60, 70, 80, 90, 100 fold, or more, reduction, downregulation, decrease in p53 levels and/or function, and/or stability, relative to a control sample.

Thus, a p53-associated pathological disorder is meant in some embodiments, a disorder characterized by low levels, loss of function, altered function of p53 in a subject or in a diseased tissue of a subject as compared to a healthy subject or a healthy tissue of the same subject.

The invention provides methods for treating disorders associated with, or related to p53. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

More specifically, converging evidence indicates that p53 also plays a major role in metabolism in both normal and cancer cells [Matthieu Lacroix et al. Mol Metab. 33: 2-22. 2020]. Wild type p53 associated metabolic functions in muscle cells include mitochondrial biogenesis and respiration, ATP production, reactive oxygen species (ROS) production, fatty acid oxidation (FAO), pentose phosphate pathway (PPP) and glucose utilization. The consequences of their deregulations on human disease beyond cancer may include for example cardiovascular diseases. In liver, the metabolic functions include amino-acid catabolism, gluconeogenesis, FAO, mitochondrial respiration, glucose utilization, anti-oxidant defenses, insulin signaling, cholesterol metabolism and ureagenesis and their deregulation may result in liver diseases such as steatosis and nonalcoholic steatohepatitis (NASH) as well as diabetes. In pancreas, p53 metabolic functions include insulin secretion and mitochondrial respiration and in adipose tissue include energy expenditure, fatty acid synthesis and storage and mitochondrial respiration, all of which may result in obesity in case of p53 deregulation. Thus, in some embodiments, as used herein, p53-associated and/or related- and/or mediated-disorders as used herein, encompass any disorder or condition affected by disfunction of p53, and/or altered function of p53, and/or reduced function of p53, loss-of function of p53, caused by at least one of degradation of p53, instability of p53, reduced expression of p53, mutations in p53, altered conformation of p53, reduced ratio between mutated and non-mutated p53, and any combinations thereof. In yet some further specific and non-limiting embodiments, the subject suffering from a p53-associated disorder is heterozygous for mutant p53.

In some embodiments, the p53-associated disorder treated by the therapeutic methods disclosed herein, may comprise at least one of: at least one proliferative disorder and at least one metabolic disorder.

In some further embodiments of the therapeutic methods disclosed herein, the proliferative disorder may be at least one neoplastic malignant disorder, as disclosed herein before in connection with previous aspects of the present disclosure.

In some further embodiments, the therapeutic methods disclosed herein may be applicable for at least one metabolic disorder. In more specific embodiments, such metabolic disorder may comprise at least one cardiovascular disorder, liver diseases, diabetes and metabolic syndrome.

Metabolic disease is any of the diseases or disorders that disrupt normal metabolism, the process of converting food to energy on a cellular level. Thousands of enzymes participating in numerous interdependent metabolic pathways carry out this process. Metabolic diseases negatively affect the ability of the cell to perform critical biochemical reactions that involve the processing or transport of proteins (amino acids), carbohydrates (sugars and starches), or lipids (fatty acids). Numerous molecular pathways and thus, several organs, can be affected. Many of the metabolic diseases are caused by genetic mutations or by a combination of genetic and environmental factors. Some of the metabolic diseases or diseases associated with metabolic disorders include for example: diabetes (or diabetes mellitus), a group of common endocrine diseases characterized by sustained high blood sugar levels affecting nearly every major bodily organ; non-alcoholic steatohepatitis (NASH), a liver inflammation and damage caused by a buildup of fat in the liver; steatosis (or fatty change), an abnormal retention of fat (lipids) within a cell or organ, most often affects the liver, but can also occur in other organs, including the kidneys, heart, and muscle; obesity, a medical condition, in which excess body fat has accumulated to such an extent that it may negatively affect health; dyslipidemia, is the imbalance of lipids such as cholesterol, low-density lipoprotein cholesterol, (LDL-C), triglycerides, and high-density lipoprotein (HDL); cardiovascular diseases, a group of disorders of the heart and blood vessels and include coronary heart disease, cerebrovascular disease, rheumatic heart disease and other conditions.

It should be noted that in some embodiments, the therapeutic methods disclosed herein involve upregulation of p53 in the treated subject. Accordingly, upregulating p53 levels may comprise reduction of p53 ubiquitylation by at least one E3 ligase.

In some embodiments, such E3 ligase may be XIAP. More specifically, as firstly shown by the present disclosure, XIAP serves as an E3 ligand for p53. Thus, by down-regulating the levels of XIAP, specifically, increasing the UPS mediated degradation of XIAP, the ARTS, fragments thereof and the ARTS mimetic compounds, specifically the “B3” compound, used by the therapeutic methods disclosed herein, lead to upregulation of p53. In yet some further embodiments, as shown by the following Examples, the ARTS, fragments thereof and the ARTS mimetic compounds, specifically the “B3” compound, used by the therapeutic methods disclosed herein, lead to down-regulation of MDM2 levels, specifically, via UPS, thereby leading to upregulation of p53 levels.

In some further embodiments, the ARTS mimetic compound of the therapeutic methods disclosed herein, is having the general formula (I) or a pharmaceutically acceptable salt, solvate, hydrate, any stereoisomer thereof or physiologically functional derivative thereof, wherein formula (I) is:

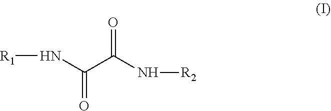

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

In some embodiments, of the ARTS mimetic compound of the therapeutic methods disclosed herein, the L1′, L1″, L2′ and L2″ are independently from each other selected from —(CH₂)ₙ—, C(═O), optionally —(CH₂)ₙ— substituted with —(CH₂)ₘ—OH and wherein n is an integer selected from 1, 2, or 3 and m is an integer selected from 1, 2 or 3.

In some embodiments, of the ARTS mimetic compound of the therapeutic methods disclosed herein, each one of R₃′, R₃″, R₄′, R₄″ is absent or may be independently from each other an aromatic or a heteroaromatic ring, each is independently from each other optionally substituted with OH, alkyl, halogen, CF₃, NO₂, C(═O).

In some further embodiments of the therapeutic methods disclosed herein, the ARTS mimetic compound is having the general formula (II)

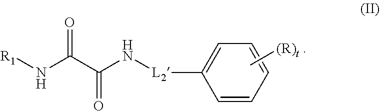

wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some embodiments, of the ARTS mimetic compound of the therapeutic methods disclosed herein, L₂′ is (CH₂)ₙ—, or C(═O), optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

In some further embodiments of the therapeutic methods disclosed herein, the ARTS mimetic compound is having the general formula (IIb):

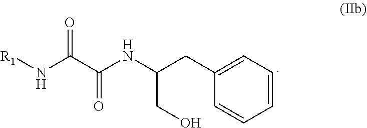

In some further embodiments of the ARTS mimetic compound of the therapeutic methods disclosed herein, the R1 is L1′-R₃′-L1″-R₃″.

In some embodiments of the ARTS mimetic compound of the therapeutic methods disclosed herein, the R1 is at least one of:
(iv) L1′, L1″ and R₃″ are each absent and R₃′ is an optionally substituted

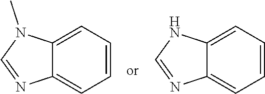

(v) L1′, L1″ and R₃″ are each absent and R₃′ is:

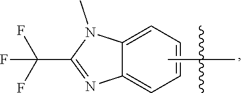

(vi) L1′ is absent R₃′ is an optionally substituted phenyl, R₃″ is an optionally substituted:

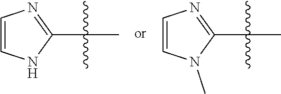

and L1″ is C(═O).

In yet some further embodiments, the ARTS mimetic compound of the therapeutic methods disclosed herein may have the general formula (IIIa) or (IIIb):

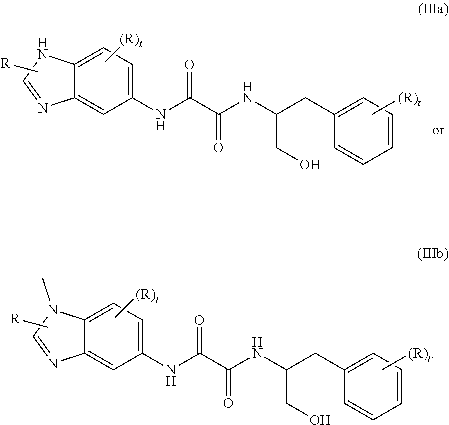

wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some further embodiments, the ARTS mimetic compound of the therapeutic methods disclosed herein, is having the general formula (IIIc), (IIId) or (IIIe):

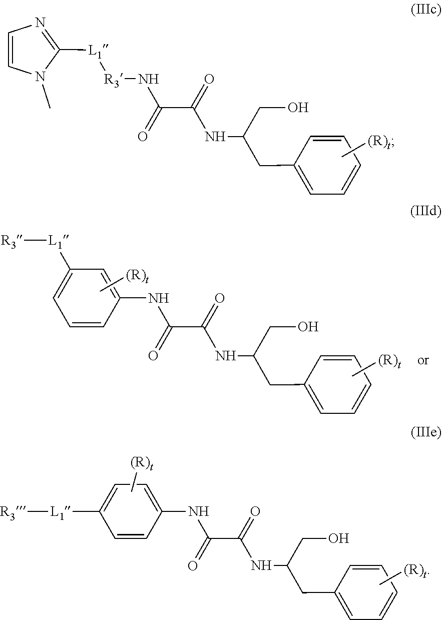

wherein L1″ and R3″ are each as defined above, wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some further embodiments, the ARTS mimetic compound of the therapeutic methods disclosed herein, is having the general formula (IIIc), or (IIIe):

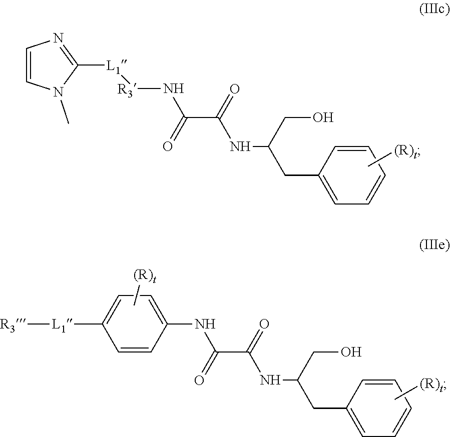

wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

In some further embodiments, the ARTS mimetic compound of the therapeutic methods disclosed herein, is having the formula (3.1), (3.2), (3.3);

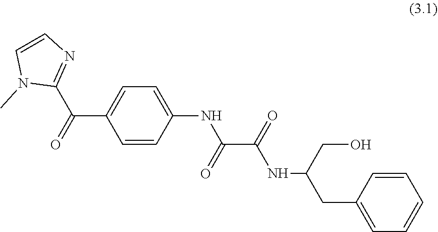 [SMILES: Cn1ccnc1C(=O)c1ccc(NC(=O)C(=O)NC(CO)Cc2ccccc2)cc1]

### N1-(1-hydroxy-3-phenylpropan-2-yl)-N2-(4-(1-methyl-1H-imidazole-2-carbonyl)phenyl)oxalamide

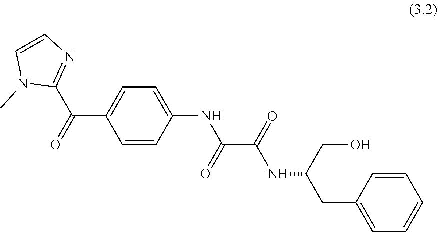

### (S)-N1-(1-hydroxy-3-phenylpropan-2-yl)-N2-(4-(1-methyl-1H-imidazole-2-carbonyl)phenyl)oxalamide (denoted herein as “B3”)

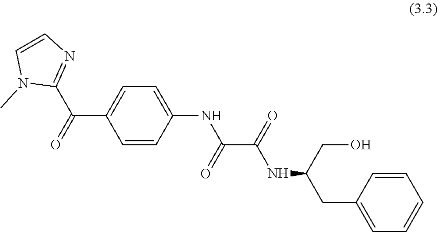 [SMILES: Cn1ccnc1C(=O)c1ccc(NC(=O)C(=O)N[C@@H](CO)Cc2ccccc2)cc1]

### (R)—N1-(1-hydroxy-3-phenylpropan-2-yl)-N2-(4-(1-methyl-1H-imidazole-2-carbonyl)phenyl)oxalamide

The methods provided herein involve administration of the ARTS mimetic compound/s of the invention in a therapeutically effective amount. The term “effective amount” as used herein is that determined by such considerations as are known to the man of skill in the art. The amount must be sufficient to prevent or ameliorate tissue damage caused by proliferative disorders and/or metabolic disorders. Dosing is dependent on the severity of the symptoms and on the responsiveness of the subject to the active drug, specifically, the antagonist of the invention. Medically trained professionals can easily determine the optimum dosage, dosing methodology and repetition rates. In any case, the attending physician, taking into consideration the age, sex, weight and state of the disease of the subject to be treated, will determine the dose. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence times and concentrations of the compositions and combined composition of the invention in bodily fluids or tissues. Following successful treatment, it may be desirable to have the patient undergo maintenance therapy to prevent the recurrence of the disease state, wherein the ARTS, functional fragments thereof, and/or ARTS mimetic compounds used by the method of the present disclosure is administered in maintenance doses, once or more daily. As use herein “therapeutically effective amount” means an amount of the ARTS, functional fragments thereof, and/or ARTS mimetic compounds, a composition comprising the same which provides a medical benefit as noted by the clinician or other qualified observer. Regression of a tumor in a patient is typically measured with reference to the diameter of a tumor. Decrease in the diameter of a tumor indicates regression. Complete regression is also indicated by failure of tumors to reoccur after treatment has stopped.

Another aspect of the present disclosure related to a kit comprising:

In one component (a), ARTS, or any fragments thereof, or at least one mimetic compound thereof, optionally, in first dosage form. It should be noted that the ARTS mimetic compound of the disclosed kits may have the general formula (I) or a pharmaceutically acceptable salt, solvate, hydrate, any stereoisomer thereof or physiologically functional derivative thereof, wherein formula (I) is:

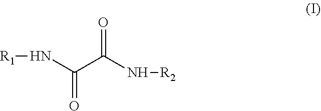

wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5; and

In another component (b), the disclosed kits further comprise at least one therapeutic compound, optionally, in a second dosage form.

The present disclosure thus provides in some aspects thereof combined therapy, combining the disclosed ARTS, fragments thereof or any mimetic thereof, specifically, the B3 mimetic, with any therapeutic compound, and specifically, with modulators of p53, that lead to upregulation of p53 levels.

The phrase “combination therapy” or “adjunct therapy” or in defining use of a compound described herein, specifically, the ARTS, and/or any functional fragments thereof (e.g. any of the peptides of SEQ ID NO: 24 to 35), and any mimetic compounds thereof, specifically, the B3 mimetic compound of the disclosure, and one or more other active pharmaceutical agents, specifically, any modulator of p53 (specifically, modulators that lead to upregulation of p53), is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent

As indicated herein, in some embodiments, the present disclosure provides kits, combined composition and combined therapeutic methods combining the disclosed ARTS, fragments thereof and/or mimetic compounds thereof, specifically, the B3 compound, with at least one therapeutic agent. The additional therapeutic agent or compound may be any compound used to treat the disclosed p53-associated condition. In some specific embodiments, the compound may be any compound that modulates p53 levels. Specifically, any compound that upregulates the levels of p53.

More specifically, p53-related drugs applicable in the combined therapeutic approach disclosed herein may include, but are not limited to small molecule compounds that are directed against any mutated p53, thereby restoring WTp53 conformation and function, for example, CP31398, PRIMA-1, MIRA-1, STIMA-1, pCAPs, ATO, ZMC1(=NSC319726), APR-246, ReACp53 or ADH-6, PhiKan083 and PC14586; drugs targeted at the WT p53 to reduce degradation thereof, e.g., by inhibiting or degrading negative regulators of p53 (specifically, MDM2, MDM4 and HPV E6), these compounds include for example RG7112, APG-115, ALRN-6924, AMG 232, siremadlin, milademetan, PROTACs, SAH-p53-8, ALRN-6924, RETRA or NSC59984, RITA, compound 12, XI-011 (NSC146109), BI-907828, CGM097, RG7388, DS-3032b and HDM201; and/or drugs targeted at the Truncated p53, for example, compounds that inhibit the recognition of premature termination codons (PTCs), enabling translational readthrough and synthesis of full-length p53 protein in cells that harbour truncating TP53 mutations. Such as: aminoglycoside antibiotic gentamicin and its derivatives, such as G418 and the new-generation synthetic derivative NB124, inhibition of the NMD process, as an example NMD14 which targets a structural pocket of SMG7, a key component of the NMD machinery.

In yet some further embodiments, proteasome inhibitors, such as Bortezomib (Velcade, PS341), as well as its second-generation derivative Carfilzomib (Kyprolis) and Ixazomib (MLN9708, Ninlaro) may be applicable in the combined therapy disclosed herein.

Still further, in some embodiments, the combined therapy disclosed by the present disclosure may combine the ARTS, fragments thereof, and/or any mimetics thereof, with gene therapy. For example, p53-based virus-like vectors, such as Gendicine, advexin and SCH-58500, Nanoparticles: selectively restore p53 expression in cancer cells, for example, SGT-53, a cationic liposome carrying wtp53-encoding DNA that homes selectively to tumour cells via an anti-transferrin single-chain antibody fragment and p53 mRNA-nanoparticle formulations.

In addition, the disclosed combinations may further use small interfering RNA (siRNA) oligonucleotides that target specific mutations within p53 mRNA and CRISPR-Cas9 base editing technologies.

In more specific embodiments, the present disclosure provides a combined therapy comprising ARTS, fragments thereof and/or mimetic compounds thereof, specifically, the B3 compound, with CP31398, that restores wild-type-like, P53 conformation and prevents its degradation through inhibition of its ubiquitylation.

In some other embodiments, the present disclosure provides a combined therapy comprising ARTS, fragments thereof and/or mimetic compounds thereof, specifically, the B3 compound, with PRIMA-1 (p53 reactivation with induction of massive apoptosis 1) -compound that restored wtp53 conformation and function upon binding to mutp53.

Still further, in some embodiments, the present disclosure provides a combined therapy comprising ARTS, fragments thereof and/or mimetic compounds thereof, specifically, the B3 compound, with MIRA-1 and STIMA-1, a 2-styrylquinazolin-4(3H)-one-related derivative. Like PRIMA-1, both MIRA-1 and STIMA-1 also possess Michael acceptor activity, and can potentially modify cysteines in the p53 protein to stabilize the wild-type conformation and prevent mutp53 unfolding.

In yet some further embodiments, the present disclosure provide a combined therapy comprising ARTS, fragments thereof and/or mimetic compounds thereof, specifically, the B3 compound, with mutp53-reactivating small peptides (pCAPs), that bind preferentially to the wtp53 conformation; when a mutp53 molecule assumes transiently a wild-type-like conformation, the peptide binds and stabilizes it, gradually shifting the dynamic equilibrium of the p53 population in that direction.

In some embodiments, the present disclosure provides a combined therapy comprising ARTS, fragments thereof and/or mimetic compounds thereof, specifically, the B3 compound, with arsenic trioxide (ATO), ZMC1(=NSC319726), that act predominantly on structural p53 mutants (such as p53(R175H)) to restore wtp53 conformation and induce p53 target gene expression.

In some further embodiments, the present disclosure provides a combined therapy comprising ARTS, fragments thereof and/or mimetic compounds thereof, specifically, the B3 compound, with APR-246 and pCAPs), that target a broad spectrum of p53 mutants to restore a wtp53-like structure, thus enabling p53 target gene activation.

In some additional embodiments, the present disclosure provides a combined therapy comprising ARTS, fragments thereof and/or mimetic compounds thereof, specifically, the B3 compound, with ReACp53 or ADH-6, that act by inhibiting mutp53 aggregation, restoring wtp53-like structure and activating p53 target gene.

Still further, in some embodiments, the present disclosure provides a combined therapy comprising ARTS, fragments thereof and/or mimetic compounds thereof, specifically, the B3 compound, with PhiKan083, that was found to bind the thermodynamically stabilize p53 (Y220C), shifting it towards a wtp53-like state.

In yet some further embodiments, the present disclosure provides a combined therapy comprising ARTS, fragments thereof and/or mimetic compounds thereof, specifically, the B3 compound, with RG7112, that was the first MDM2 inhibitor tested in clinical trials.

Still further, in some embodiments, the present disclosure provides a combined therapy comprising ARTS, fragments thereof and/or mimetic compounds thereof, specifically, the B3 compound, with APG-115, an orally bioavailable potent MDM2 inhibitor.

In some embodiments, the present disclosure provides a combined therapy comprising ARTS, fragments thereof and/or mimetic compounds thereof, specifically, the B3 compound, with ALRN-6924, that blocks both MDM2-p53 and MDM4-p53 interactions. In yet some further embodiments, the present disclosure provides a combined therapy comprising ARTS, fragments thereof and/or mimetic compounds thereof, specifically, the B3 compound, with AMG 232, that is another orally bioavailable MDM2 inhibitor, shown to promote wtp53 functionality and tumour regression in osteosarcoma cells.

Still further, in some embodiments, the present disclosure provides a combined therapy comprising ARTS, fragments thereof and/or mimetic compounds thereof, specifically, the B3 compound, with any additional MDM2 inhibitors, including siremadlin and milademetan.

In some further embodiments, the present disclosure provides a combined therapy comprising ARTS, fragments thereof and/or mimetic compounds thereof, specifically, the B3 compound, with p53-activating proteolysis targeting chimeras (PROTACs), that work by targeting MDM2 for ubiquitylation by particular E3 ligases, resulting in MDM2 degradation. Thus, in some embodiments, the present disclosure provides a combined therapy comprising ARTS, fragments thereof and/or mimetic compounds thereof, specifically, the B3 compound, with SAH-p53-8, that is capable of blocking the interactions of both MDM2 and MDM4 with p53.

In some further embodiments, the present disclosure provides a combined therapy comprising ARTS, fragments thereof and/or mimetic compounds thereof, specifically, the B3 compound, with ALRN-6924, that demonstrated high efficacy against multiple breast cancer cell lines with wtp53.

In yet some further embodiments, the present disclosure provides a combined therapy comprising ARTS, fragments thereof and/or mimetic compounds thereof, specifically, the B3 compound, with RETRA (reactivation of transcriptional reporter activity) and/or NSC59984, which inhibit the interaction of mutp53 with p73, unleash p73 and enable it to enter the nucleus and transactivate target genes that partly overlap with p53 target genes.

In some further embodiments, the present disclosure provides a combined therapy comprising ARTS, fragments thereof and/or mimetic compounds thereof, specifically, the B3 compound, with RITA, that display reactivation of p53 and induction of tumour cell apoptosis by blocking E6-P53 interaction.

In some further embodiments, the present disclosure provides a combined therapy comprising ARTS, fragments thereof and/or mimetic compounds thereof, specifically, the B3 compound, with compound 12—A new inhibitor of the E6-p53 interaction.

In some further embodiments, the present disclosure provides a combined therapy comprising ARTS, fragments thereof and/or mimetic compounds thereof, specifically, the B3 compound, with XI-011 (NSC146109), a small-molecule MDM4 inhibitor, exerted antiproliferative effects in HPV-positive cervical cancer cell lines, suggesting a potential utility of MDM4 inhibitors for treating HPV-positive cervical cancer.

In some further embodiments, the present disclosure provides a combined therapy comprising ARTS, fragments thereof and/or mimetic compounds thereof, specifically, the B3 compound, with aminoglycoside antibiotic gentamicin and its derivatives, such as G418 and the new-generation synthetic derivative NB124.

In some further embodiments, the present disclosure provides a combined therapy comprising ARTS, fragments thereof and/or mimetic compounds thereof, specifically, the B3 compound, with NMD14, which targets a structural pocket of SMG7, a key component of the NMD machinery.

In some further embodiments, the present disclosure provides a combined therapy comprising ARTS, fragments thereof and/or mimetic compounds thereof, specifically, the B3 compound, with ataluren.

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Reagents and Materials

### Reagents

Caspase inhibitors, ARTS mimetic molecules and apoptosis inducers

The caspase inhibitor Q-VD-OPh was purchased from Biovision and resuspended in DMSO as per the manufacturer's instructions.

The pan caspase inhibitor Z-VAD-FMK was purchased from Sigma Aldrich.

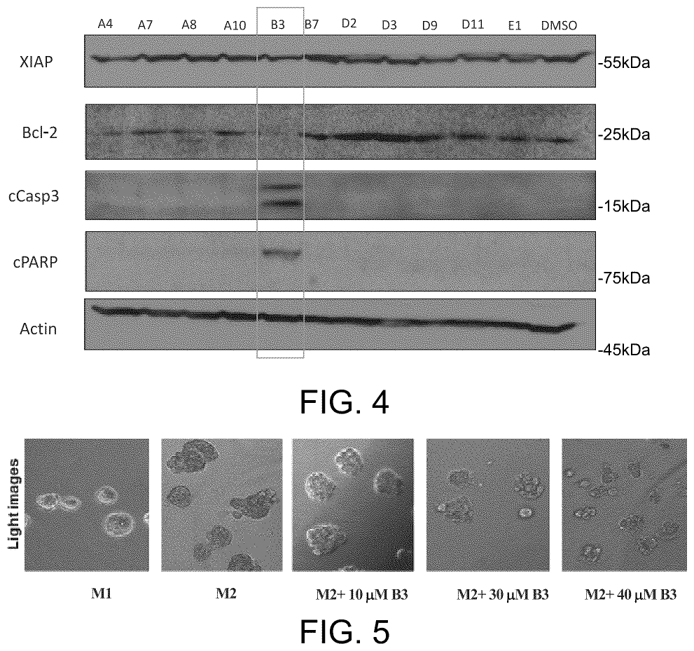

ARTS mimetics small molecules, specifically, B3 and all small molecules used for FIG. 4 were purchased from eMolecules, suspended in DMSO and resuspended in PBS as per the manufacturer's instructions.

The apoptotic induction was performed using 1.75 μM STS for the indicated times (Alexis Biochemicals).

BH3 mimetic compound ABT-199, was purchased from Selleckchem cat #8048.

### Materials

ARTS mimetics small molecules, specifically, B3 and A4, were purchased from eMolecules, suspended in DMSO and resuspended in PBS as per the manufacturer's instructions.

### Antibodies

Antibodies specific for the various proteins were purchased from the indicated companies, and used as instructed.

More specifically, the following antibodies were used:
monoclonal anti-ARTS antibody (Sigma, St. Louis);
antibodies specific for apoptotic proteins: Bcl2 (DBB DB-132); XIAP (Cat #610716, BD); Caspase-3 (Cat #9662, Cell Signaling), Anti-cleaved PARP (Cell signaling, Cat #5625).

Actin (Cat #69100, MP Biomedicals) and C-myc (Cell Signaling cat #9402) were also used.

ARTS, mouse monoclonal anti-ARTS antibody, specifically targeting the unique c-terminal sequence of ARTS (but not other Septin 4 splice variants) at a dilution of 1:1000 (Sigma A4471).

XIAP, mouse monoclonal anti-XIAP antibody (BD cat #610717) at a dilution of 1:4000.

XIAP, Rabbit monoclonal anti-XIAP antibody (Cell signaling cat #CS14334) at a dilution of 1:3000.

Actin, mouse monoclonal anti-actin antibody (ImmunoTM cat #08691002) at a dilution of 1:50,000.

Cleaved PARP (cl.PARP), rabbit monoclonal anti-cl.PARP antibody (Cell signaling cat #CS5625) at a dilution of 1:2000.

Cleaved Casp3, rabbit monoclonal anti cleaved Caspase3 antibody (cell signaling cat #cs9664).

Tubulin, monoclonal rat anti-tubulin (Abcam cat #YOL1/34) at a dilution of 1:6000.

p53, rabbit monoclonal anti-p53 antibody (Cell signaling cat #CS32532) at a dilution of 1:4000.

p53, mouse monoclonal anti-p53 antibody (Cell signaling cat #CS2524) for immunoprecipitation.

p53, mouse monoclonal anti-p53 antibody (Santacruz DO-1 cat #SC-126) at dilution of 1:1000.

p53, goat polyclonal anti-p53 antibody (R&D cat #AF1355).

MDM2, mouse monoclonal anti-MDM2 antibody (abeam cat #ab16895).

GAPDH, goat polyclonal anti-GAPDH antibody (abacm cat #ab9483).

### Transfections Reagents

The following reagents were used according to the manufacturer's instructions for transient transfections: Transit-X2 (Mirus) and PolyJet (SignaGen).

### Treatments and Reagents

Cells were pre-incubated with the following reagents:

Etoposide (abeam cat #ab120227)—200 μM, for three hours or as indicated.

Nocodazole (sigma cat #m1404)—200 ng/ml for 1 hour.

MG-132 (APExBIO cat #A2585)—20 μM for 6 hours.

Bortezomib (APExBTO cat #A2614)—20 μM for 6 hours.

Cycloheximide— (sigma cat #C7698) 200 μM was induced for up to 180 min.

B3—((S)-N1-(1-hydroxy-3-phenylpropan-2-yl)-N2-(4-(1-methyl-1H-imidazole-2-carbonyl)phenyl)oxalamide)—20 μM was added in the last 5 h of incubation with the proteasome inhibitor or as indicated.

### Experimental Procedures

### Cell Lines and Culture in 2D

Human breast cancer cell lines MCF-10A (M1) was received from Prof. Israel Vlodavsky (Technion, Israel), MCF10AT1K (M2) and MCF1OACAlh (M3) were received from Dr.

Fred Miller (Barbara Ann Karmanos Cancer Institute). M1 and M2 cells were maintained in DMEM/F12 supplemented with 5% donor horse serum (DHS), 1% sodium pyrovate, 1% L-glutamine, 0.02 μg/ml epidermal growth factor (EGF; Peprotech), 0.01 mg/ml insulin (Sigma), 0.5 μg/ml hydrocortisone (Sigma), 0.1 μg/ml cholera toxin (Sigma) and 1% penicillin-streptomycin at 37° C., 5% CO2 incubator. M3 cells were maintained in DMEM/F12 supplemented with 5% DHS and 1% penicillin-streptomycin at 37° C. and incubated in 5% CO₂ incubator.

### Cell Lines and Transfections

A-375, HeLa, SKMEL-5 cell lines and MEFs were grown in Dulbecco's modified Eagle's medium, supplemented with 10% fetal calf serum, penicillin (100 units/ml), streptomycin (100 μg/ml), and glutamine (2 mM) at 37° C. in 5% CO₂ atmosphere.

### Three-Dimensional Cell Cultures

Cells were harvested from their growth plates using 0.25% trypsin EDTA. Collected cells were cultured in Cultrex® growth factor reduced Basement Membrane Extract (BME: Trevigen, Inc) as follows: An 8 chamber glass slide system (Lab-TEK® II, Naperville, IL) was coated with 60 μl BME [Barkan D. et al., Cancer Research (2008)](protein concentration between 15 mg/ml; thickness-1-2 mm). 5×10³ cells per well were re-suspended in DMEM/F12 supplemented with 2.5% DHS and 2% BME and cultured on the coated slides. Slides were incubated at 37° C., 5% CO₂ incubator. Cells were re-fed every 4 days. Cell morphology was monitored by light microscopy. Immunofluorescence images were captured by Nikon Al—R confocal laser scanning microscope (Haifa University, Haifa, Israel).

### Semi Quantitative RT-PCR

RNA was extracted from cells using Total RNA Mini Kit (Bio-Rad) according to the manufacturer's instructions. Equal amounts of total RNA (1 μg) were used as template for first-strand synthesis with oligo dT primers (High Capacity RNA-to-cDNA Kit; Applied Biosystems) in 20 μl volume and the resulting first-strand cDNA was used for qPCR reactions.

qPCR Reaction

A reaction mixture containing 300 ng cDNA, 10 μl PCR Dream Taq Mix and 4 μl of 5 μM primers (F+R) was assayed in a Gradient Thermal Cycler PCR system (MJ Mini™).

The 20 μl reaction mixtures were heated to 95° C. for 3 minutes, and 36 PCR cycles were carried out as follows: Denaturation at 95° C. for 30 seconds, annealing at 58° C. for 30 seconds, and extension at 72° C. for 30 seconds. The reaction was heated at 72° C. for 10 minutes and subsequently cooled to 4° C. indefinitely. Electrophoresis of the samples was carried out on a 1.5% agarose gel.

The following PCR primers were used: For ARTS:
Forward: 5′-GAGACGAGAGTGGCCTGAACCGA-3′, as denoted by SEQ ID NO. 11;
Reverse, 5′-AACAGGAACCTGTGACCACCTGC-3′ as denoted by SEQ ID NO. 12;

For human GAPDH:
Forward, 5′-ATGGGGAAGGTGAAGGTCG-3′, as denoted by SEQ ID NO. 13;
Reverse, 5′-GGGGTCATTGATGGCAACAATA-3′, as denoted by SEQ ID NO. 14;

### Transient Transfections of MCF-10 A Cell Lines

The cells were transfected with either 6-MycTag-ARTS in pCS2 or sport-ARTS in pCMV plasmid, that was produced in Sarit Larisch lab as described previously (Larisch & Yi 0.2000). For the transfection the Turbofect (Fermentas) was used with concentration of 0.1 μg/μl DNA for 6-MycTag-ARTS and sport-ARTS plasmid.

### Western Blot Analysis

The cells were lysed in WCE (whole-cell extract) buffer [25 mM Hepes, pH 7.7, 0.3M NaCl, 1.5 mM MgCl2, 0.2 mM EDTA, 0.1% Triton X-100, 100[μg/ml PMSF and protease inhibitor cocktail (Roche, 1:100 dilution)]. 100 μg of total cell protein, measured with the Bio-Rad Protein Assay kit, were separated by SDS-PAGE (12%) followed by transfer for 2 h on to a nitrocellulose membrane. The membrane was blocked with 5% (w/v) non-fat dried skimmed milk powder in PBS supplemented with 0.05% Tween20 (PBS-T) for 1 hour at room temperature (R.T). Membrane was then probed with primary antibody at 4° C. overnight. Next, the membrane was incubated with the appropriate HRP-conjugated secondary antibody, for 1 hour at R.T. and washed 15 min ×3 with PBS-T. WesternBright ECL (Advansta) was added to the membrane for 2 min and analyzed using ImageQuant LAS-4000 analyzer (GE Healthcare Life Sciences) & “ImageQuant LAS-4000” software (GE Healthcare Life Sciences). Densitometry analysis was performed using ImageQuant total lab 7 (GE Healthcare Life Sciences), image analysis software.

### Co-Immunoprecipitation

Protein extracts were prepared with lysis buffer containing 150 mM NaCl, 50 mM Tris-HCl (pH 8), 1% NP-40, 0.5% deoxycholate acid with protease inhibitors (mini complete, Roche). Protein levels were determined and equal amounts were used for each sample. Lysates were pre-cleared with 1 mg mouse IgG (Sigma) coupled with protein A/G sepharose mix (Amersham Biosciences). Complexes were incubated overnight at 4° C., followed by low-speed centrifugation. Supernatants were immunoprecipitated using 5 ml of monoclonal anti-Bcl-2 antibodies (BD) overnight. Protein A/G sepharose beads were added to immunoprecipitate complex for 4 hours, collected and washed four times with PBS and then responded and heated in Sample buffer ×2 for 10 minutes.

### Cell Lysates

In all of the experiments cell lysates were prepared from floating dead cells and adherent cells harvested together. 40 h after the transfection, the cells were treated with different reagents according to the indicated assay. The cells were harvested by scraping the plate, washed twice with ice-cold 1× PBS, and lysed using radioimmune precipitation assay buffer (150 mM NaCl, 50 mM Tris-HCl (pH 8), 1% Nonidet P-40, 0.1% SDS, 0.5% sodium deoxycholate containing protease inhibitors) (mini complete, Roche Applied Science). The cells were allowed to remain on ice for 30 min followed by four cycles of freeze and thaw. The cell extract was centrifuged for 20 min at maximum speed (13,000 rpm) 4° C., and the supernatant was collected. Protein concentration was determined using the BCA kit (Promega).

### Immunofluorescence Staining in 3D Culture

The following two protocols were used for cells staining:

a. Laminin5 immunofluorescence staining: was performed as described by Barkan et al. (2008). Briefly, cells cultured in 8 well chamber glass slides in 3D BME, as described above. The cultured cells were fixed and permebalized for 5 minutes with 4% Paraformaldehyde (PFA) containing 5% sucrose and 0.2% Triton X-100, and re-fixed for an additional 25 minutes with 4% PFA containing 5% sucrose. The cells were washed for 10 minutes with PBS and an additional 10 minutes with PBS containing 0.05% Tween 20 (Sigma). Fixed cells were blocked with 3% BSA in PBS for 1 hour and incubated overnight at 4° C. with primary antibody (Rabbit polyclonal antibody to Laminin-5 (1:200)). The cells were washed three times with PBS for 15 minutes each, and incubated for 1 hour with rabbit conjugated to Alexa Fluor®647 (Invitrogen), washed as above and mounted with VECTASHIELD mounting medium with 4′, 6-diamidino-2-phenylindole (DAPI). For F-actin staining cells were incubated over night with Alexa-Fluor®488 Phalloidin (1:40) (Molecular Probes) washed three times with PBS for 15 minutes each, and mounted with VECTASHIELD mounting medium with DAPI. The slides were imaged using Nikon Al—R confocal laser scanning microscope (Haifa University, Haifa, Israel).

b. cis-Golgi protein GM130 immunofluorescence staining; was performed as a modification of Muthuswamy et al. (2001). Briefly, M1 and M2 cells grown in 3D culture, as described above, were fixed for 25 minutes at room temperature with 4% PFA containing 5% sucrose. Fixed structures were washed three times in PBS:Glycine (130 mM NaCl, 7 mM Na₂HPO₄, 3.5 mM NaH₂PO₄, 100 mM glycine) for 15 minutes each and permeabalized with 0.5% Triton X-100 in PBS for 5 minutes at room temperature and washed three times in IF buffer (130 mM NaCl, 7 mM Na₂HPO4, 3.5 mM NaH₂PO4, 7.7 mM NaN3, 0.1% BSA, 0.2% Triton X-100, 0.05% Tween 20) for 10 minutes each. The washed structures were blocked in IF buffer plus 10% donkey serum for 1 hour at room temperature. Primary antibody (GM 130; Abcam) was diluted in blocking buffer (1:500) and incubated overnight at 4° C. with primary antibody. Unbound primary antibody was removed by washing three times in IF buffer for 20 minutes each. Donkey anti rabbit secondary antibodies coupled with Alexa Fluor®647 (Invitrogen) was diluted in IF buffer containing 10% donkey serum and incubated for 1 hour. Unbound secondary antibody was washed as described above. Sides were mounted with VECTASHIELD mounting medium with DAPI. The slides were imaged using Nikon Al—R confocal laser scanning microscope (Haifa University, Haifa, Israel).

c. Immunofluorescence (IF) of A-375 Cells

Cells were seeded in 24 well plates. Following apoptotic induction (ABT-199/Bx), cells were fixed with 4% paraformaldehyde. Nuclei were stained with DAPI (157574, MP Biomedicals), mitochondria were visualized by MitoTracker (M-7512, Thermo Fisher Scientific), and immunofluorescence staining was performed with anti-Bcl-2 or anti-XIAP antibodies, followed by fluorescent-conjugated secondary antibody (Alexa fluor 488, Rhodomin RedX). Image analysis was carried out using a fluorescence microscope (Nikon 50i). 300 cells from each sample were analyzed for cellular localization of Bcl-2 or XIAP. The co-localization Pearson's coefficient was determined using ImageJ analyzing tool.

### In Situ Cell Death Detection Assay for Apoptosis—TUNEL

The cells were cultured as described above in 8 well chamber glass slides, fixed according to Barkan's immunofluorescence staining protocol and incubated for 1 hour with a mixture of TUNEL reaction mix according to the manufacturer's protocol (In Situ Cell death Detection Kit, TMR Red; Roche, Cat #12-156-792-910) covered with aluminum foil and placed in the 37° C., 5% CO₂ incubator. Following incubation, slides were washed three times with PBS for 5 minutes each, and mounted with VECTASHIELD mounting medium with DAPI. DAPI stain was used to assess total cell number. Ratio of TUNEL-positive cells out of total cells represented the ratio of apoptotic cells. The analysis was carried out using Nikon Al—R confocal laser scanning microscope (Haifa University, Haifa, Israel).

### Proliferation Assay

The CellTiter 96 AqueousOne Solution of cell proliferation assay kit (Promega) was added to the wells for 2 hours to measure cell proliferation according the manufacturer's instructions. The absorbance was recorded at 490 nm.

Viability Assays (IC50 Calculation) Performed with B3 on Different Cancer Cell Lines

The assay was performed at six tenfold serial dilutions of the B3 compounds: 10 mM (stock), 1 mM, 100 μM, 10 μM, 1 μM and 0.1 μM. The screen was performed on 93 cancer cell lines and PBMC as normal cell control. The B3 compound was incubated with the cells for 72 hours. The following cancer cell lines were screened:

JAR, JEG3, SKMEL5, MDAMB435, SKMEL28, A375, A431, EJ28, UMUC3, 5637, T24, CLS439, J82, MDAMB468, MT3, MCF7, SKBR3, MDAMB231, HS578T, JIMT1, MDAMB436, BT20, HEK293, 7860, CAKIl, U031, ACHN, HT1080, DU145, 22RV1, PC3, MG63, SAOS2, U2OS, MHHES1, RDES, RAMOS, MINO, SU—DHL-6, K-562, WSU-NHL, L-363, HL-60, GRANTA-519, MV4-11, KASUMI-1, THP-1, SKHEP1, HEPG2, PLCPRF5, CALU6, NCIH82, NCIH460, IMR90, A549, NCIH358M, NCIH292, A204, TE671, HS729, RD, A673, SKNSH, SKNAS, SF295, SF268, SNB75, U87MG, MIAPACA2, PANC1005, PANC1, BXPC3, ASPC1, HELA, C₃₃A, CASKI, A2780, OVCAR3, OVCAR4, IGROVI, EFO21, SKOV3, SKLMS1, LOVO, HCT15, SW620, DLD1, HT29, COLO205, HCT116, CACO2 and COL0678.

The Sulforhodamine B (SRB) assay was used as a screening method. Briefly, the method relies on the property of SRB, to bind stoichiometrically to proteins under mild acidic conditions and to be extracted under basic conditions. Thus, the amount of bound dye can be used as a proxy for cell mass, which can then be extrapolated to measure cell proliferation. The protocol can be divided into four main steps: preparation of treatment, incubation of cells with treatment of choice, cell fixation and SRB staining and absorbance measurement.

### XTT

XTT Cell Proliferation Kit (Biological Industries cat #20-300-1000) were used according to the manufacturer's instructions. Cells (50000 per well) were seeded into white 96-well plates Clear Flat Bottom TC-treated Culture Microplate (#353072) overnight. The next day the treatment was added with dilutions of drugs alone or in combination. Cell viability was assessed after 24 hours treatment using the following incubation at 37° C. with XTT or reagent for 2 h. The intensity of the color was measured using BioTeK ELISA synergyHT microplate reader (450 nm excitation and 630 nm emission). We determined the cell viability by normalizing the results in the treated cells to cells treated with DMSO (fold change to DMSO).

### Bimolecular Fluorescence Complementation (BiFC)

The split-Venus BiFC system was used to evaluate close proximity indicating possible direct binding between pairs of proteins. The proteins were fused either to the N-terminal part of the Venus-YFP (yellow fluorescence protein) (VN) or the C-terminal part (VC). All Venus fragments were fused to the C-terminal sequences of these proteins. The Jun and Fos pair was used as a positive control (P.C.), and the Jun and FosdeltaZIP pair was used as a negative control (N.C.). A vector encoding dsRed was used as a transfection efficiency marker. Cells were seeded in 12 well plates overnight. Cells were then transfected with (Bcl-2-VC, ARTS-VN, XIAP-VN and XIAP-VC) for 24-36 hours. Cells were then treated with apoptotic inducing reagents for 24 hrs.

### MicroScaleThermophoresis (MST)

Microscale thermophoresis (MST) binding assays were performed by CreLux, a WuXi AppTech company in Germany, using recombinant ARTS, XIAP, Bcl-2 and cIAP1 proteins. Specifically, for performing experiments with untagged XIAP, a fluorescent label (NT650) was covalently attached to the protein (Maleimide coupling). The labelling was performed in a buffer containing 50 mM HEPES pH 7.0, 150 mM NaCl and 0.005% Tween-20.

### Statistical Analysis

Densitometry analyses of the western blot results were performed using Image Studio Lite graphic software. At least 300 cells were counted for each immunofluorescence sample. For analysis of the results from all the different methods, GraphPad Prism software was used on two-six biological repeats by One-Way ANOVA with Scheffé post-hoc test, Pearson correlation coefficient or Dunnette's multiple comparison test. P-values were considered statistically significant when p-value<0.05 (*), p-value<0.01 (**), p-value<0.001 (***).

### Cell Line Culture

HCT 116 (WT, p53 KO and XIAP KO) cells were grown in complete McCoy's medium (1% sodium pyruvate, 1% L-glutamate, 1% Pen-strep, and 10% fetal bovine/calf serum). MEFs (WT, Sept4/ARTS KO, and XIAP KO) cells and A375 cells were grown in a complete DMEM medium (1% sodium pyruvate, 1% L-glutamate, 1% non-essential amino acids, 1% Pen-strep, 10% fetal bovine/calf serum, and 0.1% β-mercaptoethanol). A549 cells were grown in DMEM/F12 complete medium (1% sodium pyruvate, 1% L-glutamate, 1% Pen-strep, 10% fetal bovine/calf serum).

All cell lines were checked for mycoplasma and kept under passage 10.

### SDS-PAGE and Western Blot Analysis

Cells were lysed in whole-cell extract buffer [25 mM HEPES, pH 7.7, 0.3 μM NaCl, 1.5 mM MgCl2, 0.2 mM EDTA, 0.1% Triton X-100, 100 μg/ml phenylmethylsulfonyl fluoride (PMSF) and protease inhibitor cocktail (Roche, 1:100 dilution)] and placed on ice for 30 min (vortexing once after 15 min). After 30 minutes, the samples were centrifuged at 13,000×g for 10 minutes at 4° C. The supernatants containing total protein were measured for protein concentration using Bio-Rad Protein Assay Dye Reagent Concentrate Kit. 40 to 100 g protein was separated by sodium dodecyl sulfate-polyacrylamide gel electrophoresis (SDS-PAGE) (12% or 7.5%), followed by transfer to a nitrocellulose membrane. The membranes were blocked with 5% (w/v) non-fat dried skimmed milk powder in PBS supplemented with 0.05% Tween-20 (PBS-T) for 1 h at RT. Next, primary antibodies were added at 4° C. overnight or for 2 h at room temperature. Membranes were then incubated with secondary antibody for 1 h at RT and washed three times for 15 min each with PBS-T. Western Bright ECL (Advansta) was added to the membrane for 30-60 s and analyzed using Image Quant LAS-4000 (GE Healthcare Life Sciences) and Image Quant LAS-4000 software (GE Healthcare Life Sciences).

### Co-Immunoprecipitation

Cells were harvested and lysed with radioimmunoprecipitation assay (RIPA) buffer (Tris-HCl pH 7.5 50 mM, NaCl 150 mM, NP-40 (Igepal) 0.3%) containing protease inhibitor cocktail (Complete, Roche) and 100 μg/ml PMSF. Antibodies were used at 5 μg per 1000 g protein and incubated overnight, rotating at 4° C. The next day, agarose beads conjugated to protein A/G (Santa Cruz Biotechnology) were added for 4 h with rotation at 4° C. Samples were centrifuged at 4° C. for 5 min and washed five times with RIPA buffer. Proteins were eluted from beads by 10 min of boiling in sample buffer and separated on 12% SDS-PAGE gel, followed by Western blot analysis.

### In Vivo Ubiquitylation Assay

All cells were transiently transfected with a Ub-HA (ubiquitin-tagged with HA) construct and treated with a proteasome inhibitor (Bortezomib or MG-132, at 20 μM for 6 h). after 1 h of proteasome inhibitor incubation, B3 (20 μM) or DMSO was added to the medium for an additional 5 h. After 6 h of Bortezomib/MG-132 along with 5 h of B3 treatments, the cells were harvested and lysed using RIPA buffer (Tris-HCl pH 7.5 50 mM, NaCl 150 mM, NP-40 (Igepal) 0.3%) containing protease inhibitor cocktail (Complete, Roche), 100 g/ml PMSF, 5 mM N-ethylmaleimide, and 5 mM iodoacetamide to preserve ubiquitin chains. Following 15 min of centrifugation (10,000×g, 4° C.), the supernatant was transferred into a clean Eppendorf tube. A ubiquitination assay using immunoprecipitation with anti-p53 antibody (CS2524) (endogenous p53 was pulled down) was performed as described above with 1:500 antibody per 1000 μg protein. Poly-ubiquitylated forms of p53 were detected using an anti-p53 antibody (DO-1, sc-126).

### In Vitro Ubiquitylation Assay

Bacterially expressed His-ARTS was purified using fast protein liquid chromatography (FPLC), and bacterially expressed GST-XIAP was purified using glutathione beads. P53 recombinant protein was purchased from R&D (cat #SP-450). Recombinant p53 and GST-XIAP were incubated. E1, UbcH5b, ubiquitin (Ub), and the appropriate E3 in conjugation buffer (20 mM Tris-Cl [pH 7.6], 100 mM KCl, 5 mM MgCl2, and 1 mM DTT) containing 2 mM ATP at 37C for 1 hour.

### Chromatin Immunoprecipitation (ChIP)

The assay was done using Chromata ChIP Kit (Novus Bio cat #NBP1-71709). In brief, cells were exposed to UV irradiation for 5, 10, and 30 min. Cross-linking was done using 1% formaldehyde for 10 min. The reaction was stopped by incubation with 125 nM Glycine for 10 min at RT. Cells were washed twice with PBS and lysed on ice for 30 min with SDS-Lysis Buffer containing PI and PMSF. To achieve the appropriate length of DNA fragments (between 1000 and 200 bp) the samples were sonicated for 5 min (30 sec on/off). Samples were centrifuged for 10 min at 15000 rpm at 4° C., and the supernatant was transferred into a fresh tube. To determine DNA concentration and fragment sizes 50 μl was taken from each sonicated sample reverse cross-linked, cleaned, and run on 1% agarose gel. The samples were pre-cleared with 20 μl/sample of A/G agarose beads for 1 h at 4° C. 10% of pre-cleared lysate was taken from all the samples as an INPUT control and stored at −20° C. The remained sample was split into three aliquots. Each aliquot was incubated with a different antibody: p21 (sc-51689), IgG (ab18421), and p53 (ab1101 ChIP grade). p21 sample was used as a positive control, and the IgG sample was used as a negative control. All the samples were gently rotated at 4° C. for 2 h. A/G agarose beads (40 μl/sample) were washed, re-suspended in IP buffer, and added to the samples for overnight rotation at 4° C. The cells were washed twice with IP washing buffer, then washed twice with Lithium buffer and TE buffers. Protein-DNA complexes were eluted with Elution buffer. The samples were then reverse cross-linked with 1 μM NaCL for 4 h at 65° C., 1 μg/ml RNase A for 30 min at 37° C., and 20 μg/ml of Proteinase K for over night at 55° C. The samples were cleaned with a PCR purification kit. The samples were checked for the presence of the DNA fragments containing the Sept4/ARTS gene using PCR reaction with the following primers:
-′5GAGACGAGAGTGGCCTGAACCGA-3′ (Forwards), for forward sequencing of ARTS, as denoted by SEQ ID NO: 15; -5′ AACAGGAACCTGTGACCACCTGC-3′ (Reverse), for reverse sequencing of ARTS as denoted by SEQ ID NO: 16;
The samples were also checked for the presence of DNA fragments containing p21 μgene (positive control) using the following primers:
-′5GTGGCTCTGATTGGCTTTCTG-3′ (Forwards), for forward sequencing of p21, as denoted by SEQ ID NO: 17;
-5′CTGAAAACAGGCAGCCCAAG-3′ (Reverse), for reverse sequencing of p21, as denoted by SEQ ID NO: 18.

### Bimolecular Fluorescence Complementary Assay (BiFC)

The split-Venus BiFC system was used to evaluate close proximity indicating possible direct binding between pairs of proteins. The proteins were fused either to the N-terminal part of the VenusYFP (yellow fluorescence protein) (VN) or the C-terminal domain (VC). All Venus fragments were fused to the C-terminal sequences of these proteins. The Jun and bFos pair was used as a positive control (p.c.), and the Jun and bFosdeltaZTP pair was used as a negative control (n.c.). A vector encoding dsRed was used as a transfection efficiency marker.

### MST Binding Assays

Microscale thermophoresis (MST) binding assays were performed by CreLux, a WuXi AppTech company in Germany, using recombinant ARTS and XIAP proteins. Specifically, for performing experiments with untagged XIAP, a fluorescent label (NT650) was covalently attached to the protein (Maleimide coupling). The labeling was performed in a buffer containing 50 mM HEPES pH 7.0, 150 mM NaCl, and 0.005% Tween-20. A detailed description is provided in the Supplementary Materials and methods.

### Computational Screen

Three hundred thousand commercially available molecules were selected from a set of about 3 million and were screened using LeadIT and SeeSAR software suits from BioSolveIT. This computational screen-identified compound with predicted binding affinities in the micro-molar to the Nano-molar range, as assessed by the HYDE scoring function. The 100 top-ranked molecules exhibiting the best docking scores were determined. The ARTS unique binding site in XIAP-BIR3 was extrapolated by analyzing XIAP-SMAC crystal structures from the PDB and our data, described by Bornstein et al.

### Preparation of B3 Stock and Work Solution

The B3 small molecule (MW 406.43 μgr/mol as powder, SMILES: (CC22H22N4O4) was purchased from eMolecules, Inc., eMolecule ID: 30500827 (Supplier InterBioScreen STOCK 6S-95262). B3 was dissolved in dimethyl sulfoxide (DMSO) to a stock solution of 30 mM, followed by intensive pipetting and centrifugation at 300×g for 30 s. Next, the B3 suspension was incubated in a 37° C. bath for 1 min, mixed thoroughly by pipetting, and spun down again. B3 stock solution was aliquoted in Eppendorf tubes (7-10 μl/tube) and stored at −80° C. Aliquots were only used once to avoid freeze and thaw of the compound. Before use B3 aliquot was thawed, spun down (same settings), and mixed by tapping gently at the lower part of the Eppendorf tube. Next, the compound solution was diluted 1:100 in a warm complete medium in a 15 ml conical tube to a concentration of 0.3 mM and mixed well by tilting the closed vial up and down (do not vortex). The diluted B3 solution was then diluted again to the desired final concentration (5-40 μM) and added to the cells.

### Statistical Analysis

All graphs were made using the PRISM software. Significance was evaluated using PRISM's one-way ANOVA analysis or two-way ANOVA analysis. p<0.05 was considered significant.

### Example 1

### Screening for ARTS Mimetic Small Molecules

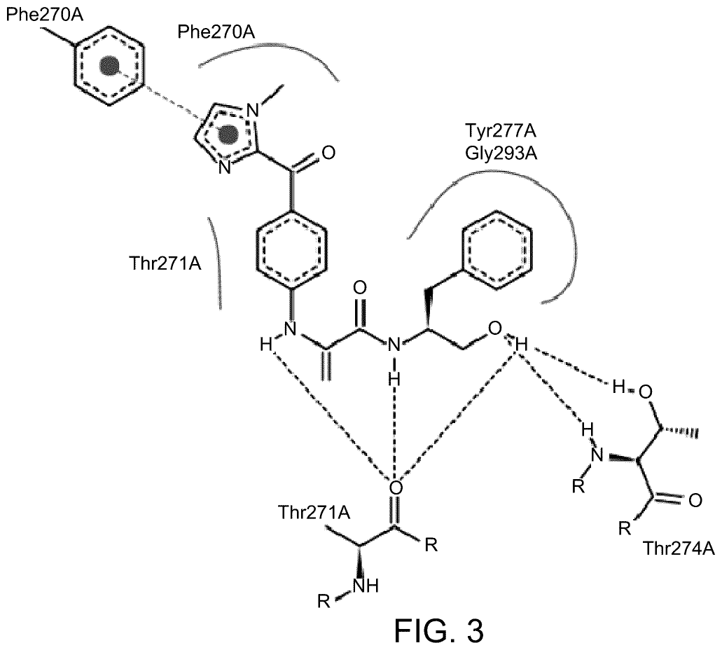

The inventors have previously shown that ARTS induces apoptosis by interaction of its unique C-terminal domain with distinct binding sequence within BIR3/XIAP. In an attempt to screen for further small molecules for cancer therapy, an “in silico” screen was done by “BioSolvit” to look for ARTS mimetic small molecules that fit into ARTS binding site within the XIAP molecule. As illustrated by FIG. 1, about 100 candidate molecules were revealed. FIG. 2 and FIG. 3 present the structure of one of the candidate molecules, B3 located within the binding site and the interactions of said candidate compound with residues Thr271, Thr274, Tyr277 and Gly293 of the BIR3/XIAP.

The candidate molecules were next subjected to functional assays, examining their ability to induce apoptosis, thereby increasing cell death, in melanoma (A375) and leukemia (Jurkat) cell lines FIGS. 21A and 21B. Further, the expression of several apoptotic markers was examined in A375 human melanoma cells exposed to 20 μM of each of the candidate compounds. As shown in FIG. 4, the B3 small molecule increased the cleavage of apoptotic markers Caspase 3 and PARP, as well of Caspase 9 (as disclosed in Example 4 below). In addition, B3 treatment clearly reduced the levels of the anti-apoptotic proteins XIAP and Bcl-2 in the melanoma cell lines. Similar results were also demonstrated for different time exposure (2-24 hrs) of the effective candidate B3 demonstrating reduction in anti-apoptotic markers in HeLa cells (derived from cervical cancer cells) exposed to 30 μM of ARTS mimetic small molecule B3. It is also important to note that B3 promotes a specific degradation of XIAP, but does not affect cIAP1 As indicated herein below in Example 4, the same apoptotic effect was observed in different cell lines from different origins.

Taken together, these findings indicate that B3 clearly antagonize anti-apoptotic proteins (e.g., Bcl-2 and XIAP), elevates pro-apoptotic proteins and thereby has an apparent pro-apoptotic effect on malignant and pre-malignant cells.

### Example 2

### The ARTs Mimetic Small Molecule B3 Promotes Apoptosis in Premalignant Cells

The effect of ARTS mimetic B3 molecule was examined in Breast Cancer model. To further characterize the apoptotic effect of the ARTS mimetic B3 molecule on premalignant cells, the 3D system has been used. More specifically, MCF10A (M1) (normal breast cancer cells) and MCF10AT1K (M2) (premalignant breast cancer cell line) cells were cultured in the 3D system constituted from growth factor reduced basement membrane (BME), a model to study normal and aberrant morphogenesis of the mammary gland. As shown in FIG. 5, M2 organoids at day 4 in the 3D BME system were either untreated or treated with increasing concentrations of B3 molecule for additional 7 days. B3 molecule was re-supplemented every 4 days. Light microscopy images indicate that B3 molecule induced cellular death depicted by blebbing of the cells and cell shrinkage in dose a dependent manner.

To further evaluate the pro-apoptotic effect of the ARTS mimetic B3 molecule, TUNEL assay has been used. More specifically, M2 cells were cultured in the 3D BME system and at day 4 the cells were either untreated or treated with B3 (40 μM) molecule for 24 h. Light microscopy images indicate that B3 molecule induced cellular death depicted by blebbing of the cells and cell shrinkage (FIG. 6A). Furthermore, B3 treated cells displayed a significant increase in the number of cells positive for TUNEL staining as shown in FIG. 6B.

### Example 3

Viability Assay Performed with ARTS Mimetic Small Molecule B3 on Different Cancer Cell Lines

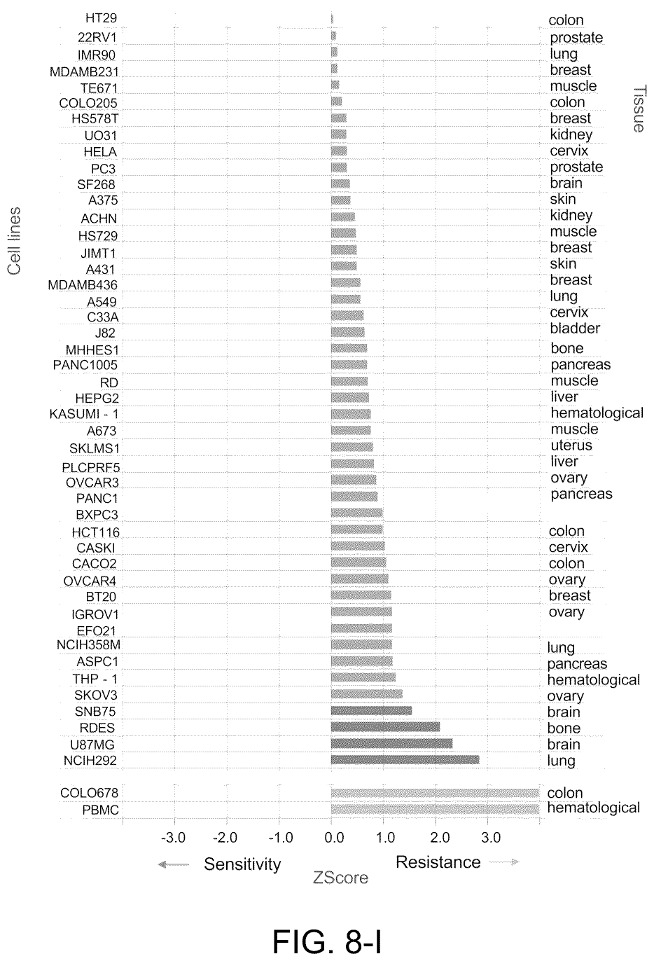

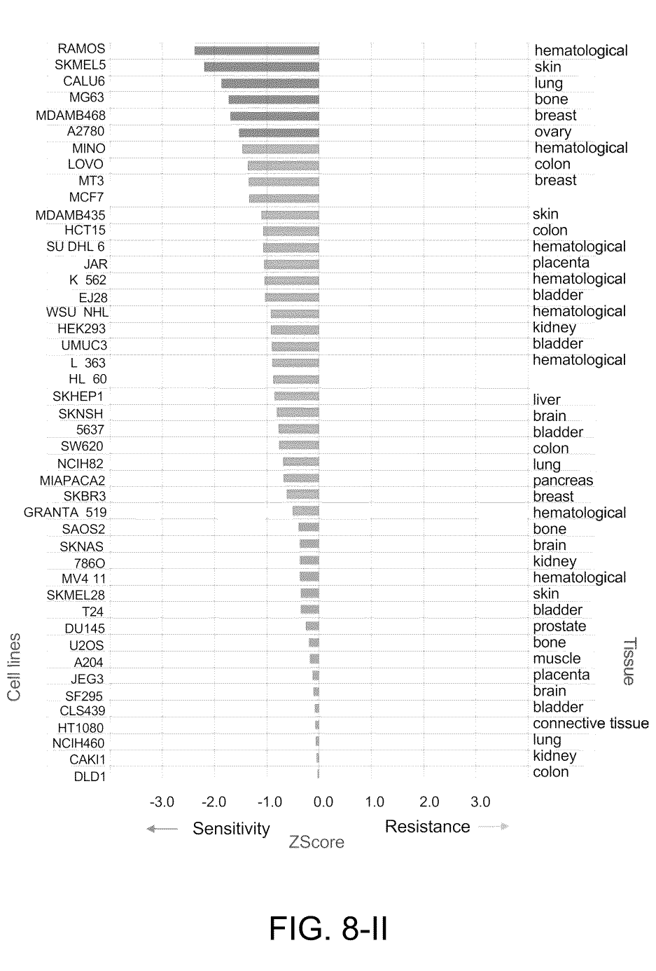

To further establish the feasibility of using the B3 small molecule of the invention as a specific anti-cancerous compound, viability of variety of cancer cell lines exposed to the B3 compound of the invention was next evaluated. As demonstrated by FIGS. 7 and 8(I)-8(II), IC50 of ARTS mimetic small molecule B3 was calculated following treatment of several cancer cell lines for 72 hrs with B3 concentrations ranging from 10 mM to 0.1 μM. Human peripheral blood mononuclear cells (PBMC) were used as normal cell control. As shown by FIG. 8II, a wide variety of cancer cell lines appear to be sensitive to B3 small molecule. Most sensitive cell lins are disclosed in FIG. 7. Specifically, cancer cells originating from placenta, skin, bladder, breast, kidney, bone and blood. However, human PBMCs that serve as normal cell control, appear to be resistant to B3 small molecule. The inventors specifically show the selectivity of B3 in osteosarcoma cells (MG-63) as compared to normal cells (Peripheral blood mononuclear cells (PBMC), and further demonstrate its activity by showing a dose-dependent death of these cells in response to increasing doses of B3 (FIG. 15). These results clearly demonstrate the specificity and selectivity of the B3 compound of the invention to malignant and pre-malignant cells. Moreover, the results demonstrate the feasibility of using the ARTS mimetic molecule B3 as a specific potent inducer of apoptosis in malignant cells.

### Example 4

Combined Treatment of ABT-199 with B3 Rescues the Effect of ABT-199 on Resistant Cancer Cells and Restores their Sensitivity to this Drug

Bcl-2 (B-cell lymphoma 2) protein functions as a potent inhibitor of apoptosis. Bcl-2 is highly expressed in many types of cancers. Therefore, Bcl-2 is a major target for developing anti-cancer drugs. Bcl-2 contains a BH3 binding domain which enables it to interact and neutralize other pro-apoptotic Bcl-2 family members resulting in inhibition of apoptosis. ABT-199 (Venclexta®) is a BH3 mimetic drug, approved by the FDA for treatment of CLL (Chronic Lymphocytic Leukemia) patients. ABT-199 acts by binding to Bcl-2 and neutralizing its anti-apoptotic effect, leading to death of the treated cancer cells. It is reported however, that patients treated with ABT-199 develop resistance to the drug by upregulating Bcl-2 and MCL-1 levels. As shown by FIG. 4, the B3 small molecule clearly reduced the levels of the anti-apoptotic proteins XIAP, c-IAP1 and Bcl-2, while clear increase in c-PARP was observed. Therefore, the inventors next examined whether the B3 compound may restore the ABT-199 activity in resistant cells.

Cancer cell lines which are relatively resistant to ABT-199 (A375 melanoma cells and CCRF-CEM, leukemia cells) apoptotic effect, were used. Treatment of A375 cells with ABT-199 resulted in increased levels of the anti-apoptotic protein Bcl-2.

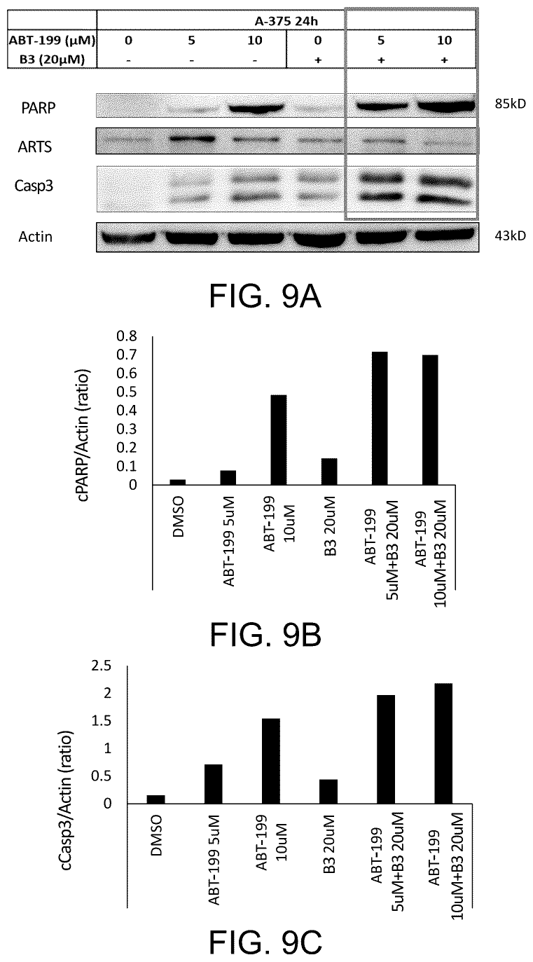

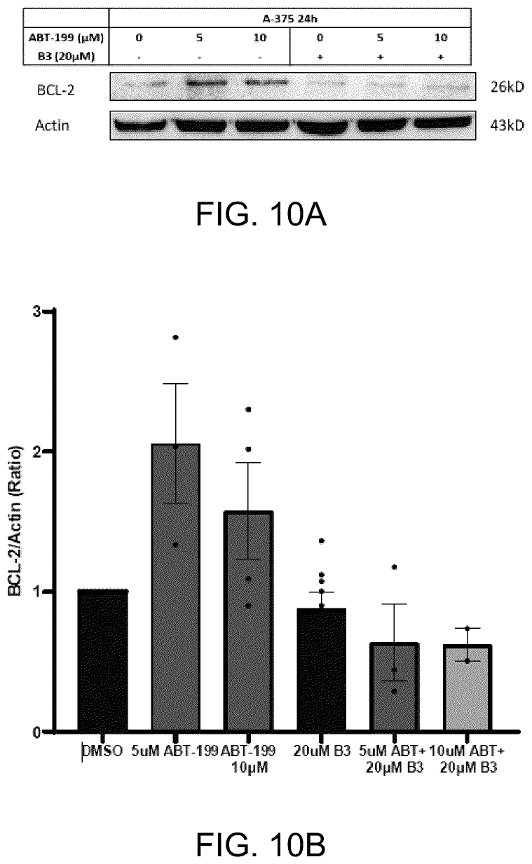

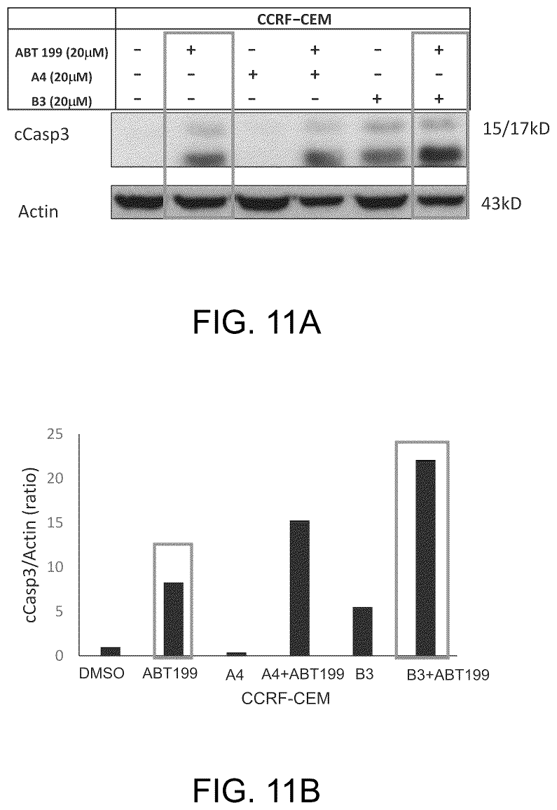

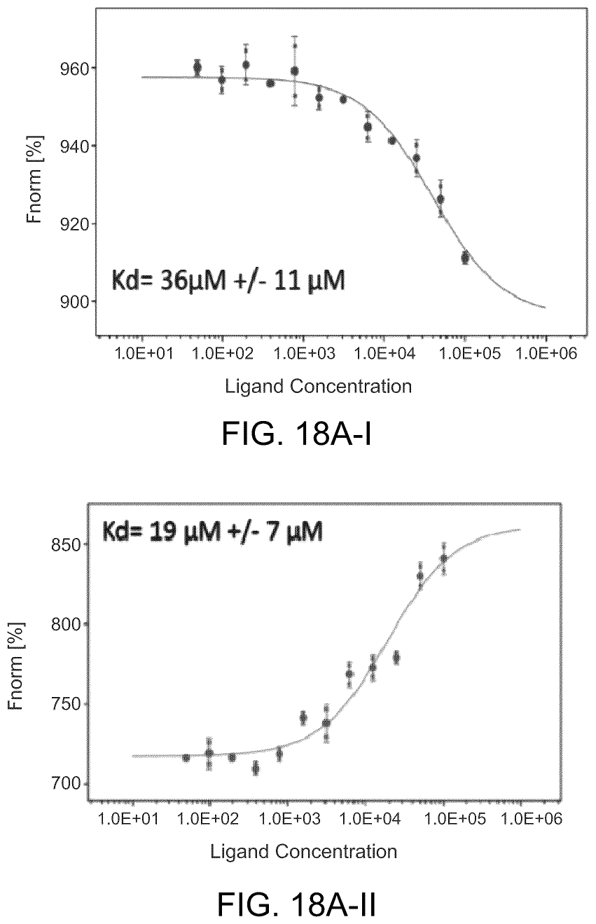

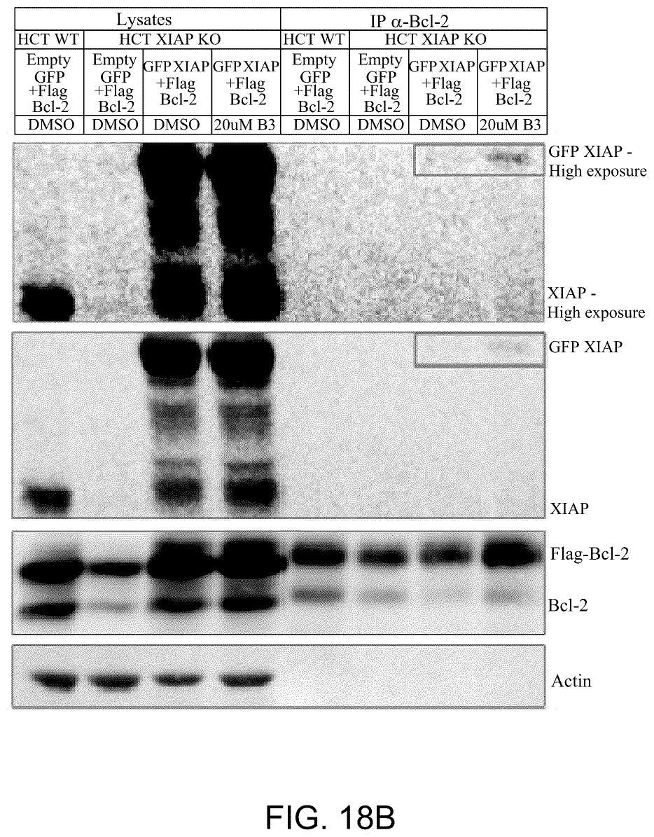

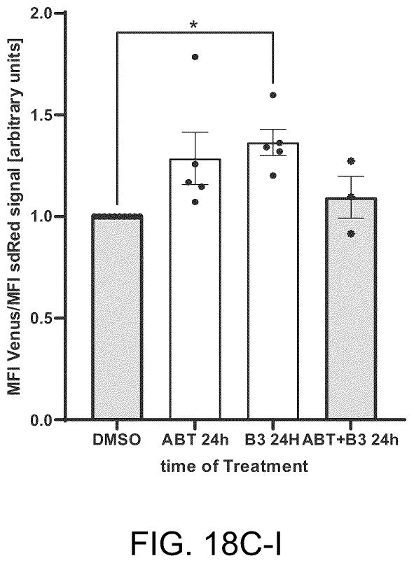

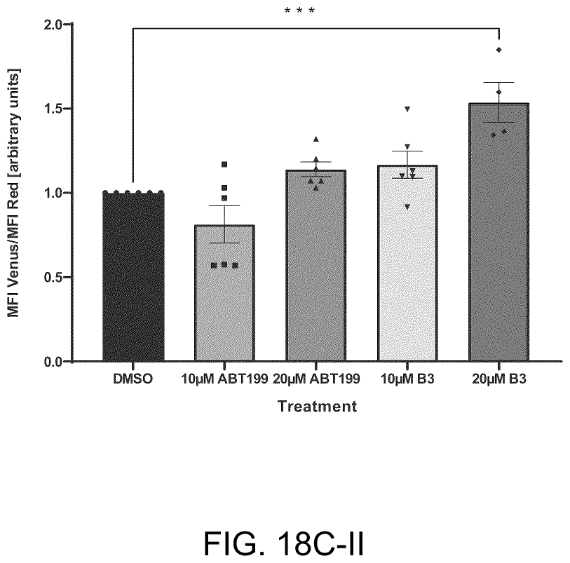

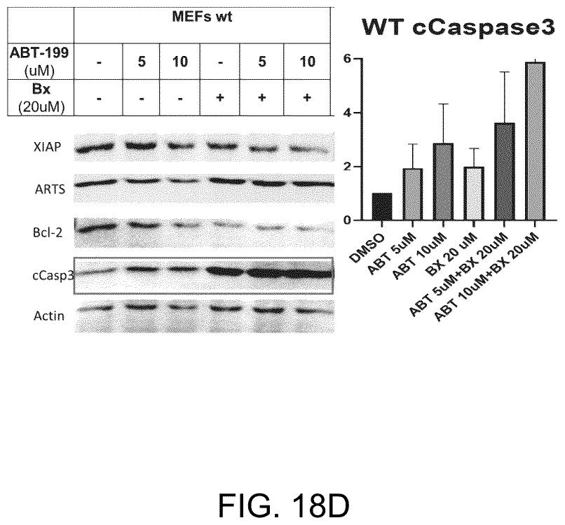

More specifically, as shown by the western blot of FIG. 9A, A-375 melanoma cell line treated for 24 hrs with different concentrations of ABT-199 with and without the small molecule B3. ARTS levels were upregulated upon treatment with 5 μM ABT-199 in addition to apoptotic markers, cleaved PARP and cleaved Caspase 3 (FIGS. 9B, 9C, respectively). The combined treatment with ABT-199 and B3 increased the apoptotic effect, as ARTS was downregulated by B3. FIG. 10A-10B and FIG. 22 shows that BCL-2 levels in these cells were upregulated upon treatment with 10 μM ABT-199. The combined treatment of ABT-199 with B3 decreased BCL-2 levels in both concentrations. Similarly, Analysis of CCRF-CEM (acute lymphoblastic leukemia) cell line treated for 24 hrs with ABT-199 with and without the small molecule A4 and B3, presented in FIG. 11A-11B, shows that apoptotic marker proteins cleaved Caspase 3 are increased upon treatment with ABT-199 alone. Importantly, treatment of B3 alone is sufficient to induce apoptosis in these cells. A combination treatment of ABT-199 with B3 increased cCasp3 expression indicating elevated cells death levels with the combined treatment. Interestingly the effect of B3 on ABT-199 sensitive cells is different. Similar results with a clear dose response, are also shown by FIG. 18D. FIG. 23 compares the effect of several molecules on HL-60 leukemia cell line and CCRF-CEM cell line. While a combination treatment of ABT-199 with B3 had a synergistic effect on cPARP and cCasp3 levels in CCRF-CEM cell line (FIG. 23B), a milder effect was observed in HL-60 cell line (FIG. 23A). Experiment repeated twice.

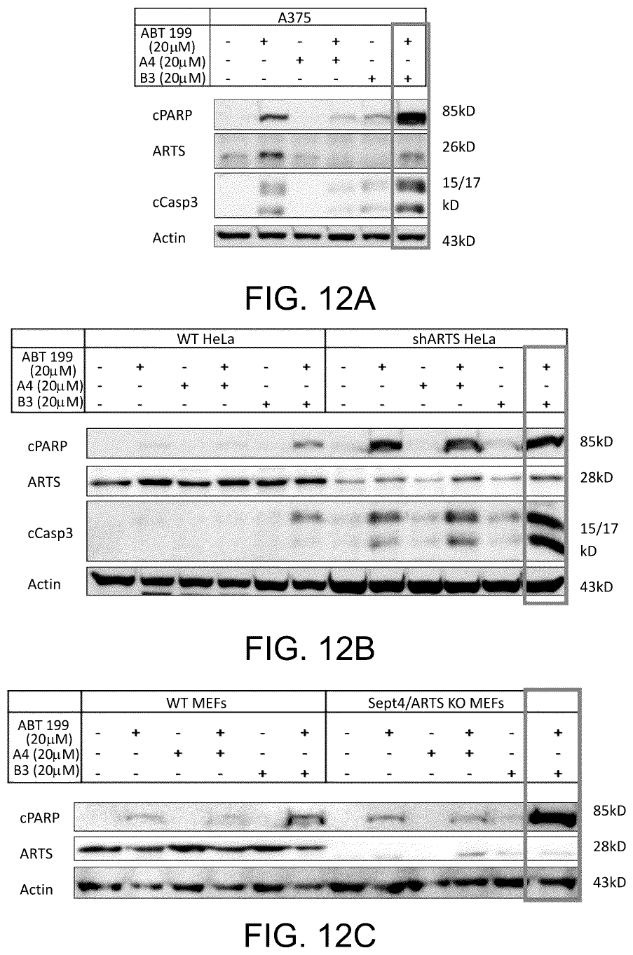

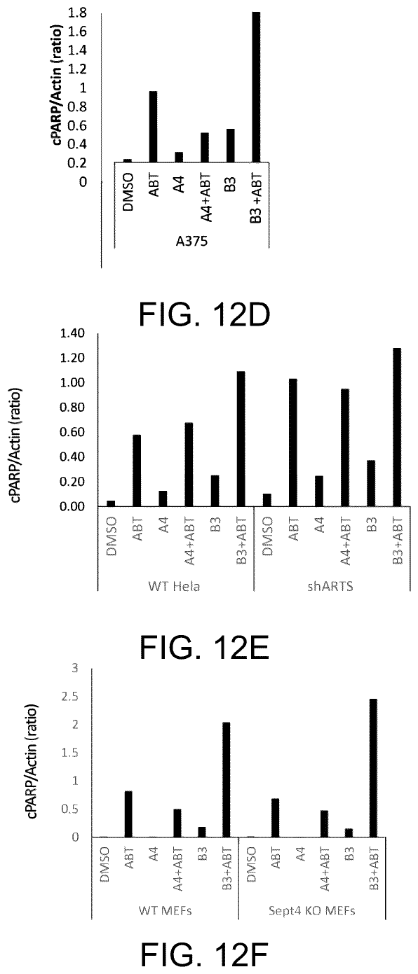

The effect of ARTS expression on the B3 effect was next examined. FIG. 12A-12F shows analysis of different cell lines with different amounts of endogenous ARTS protein treated for 24 hrs with ABT-199 with and without the small molecules A4 and B3. ARTS levels were upregulated upon treatment with ABT-199 alone. Cells with low or no ARTS expression (Sept4/ARTS KO MEFs, shARTS HeLa and A-375) show a better killing response to ABT-199 when compared to cells with high levels of endogenous ARTS (WT MEFs, WT HeLa). Combined treatment of ABT-199 with B3 increased the killing effect in all the presented cell lines.

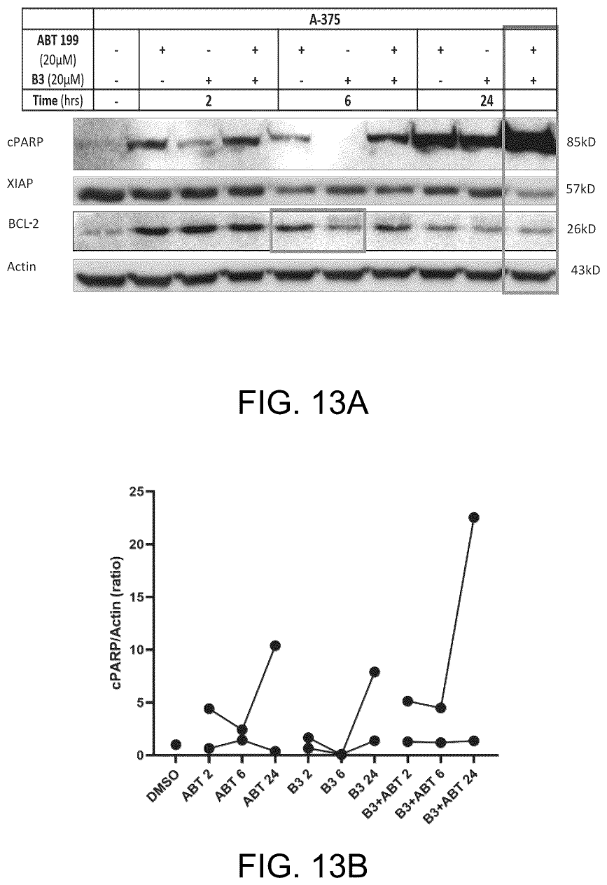

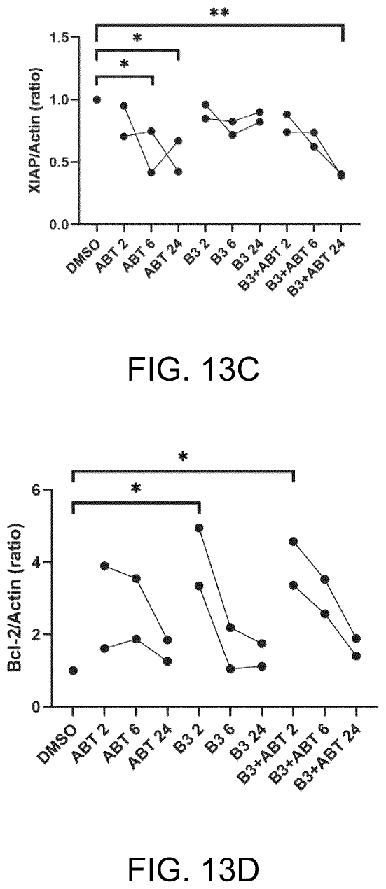

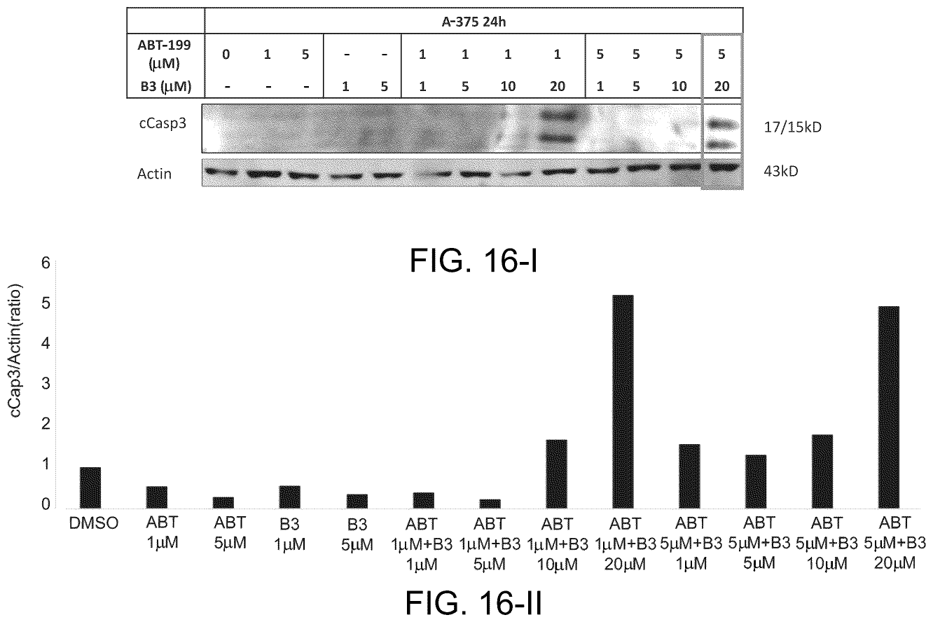

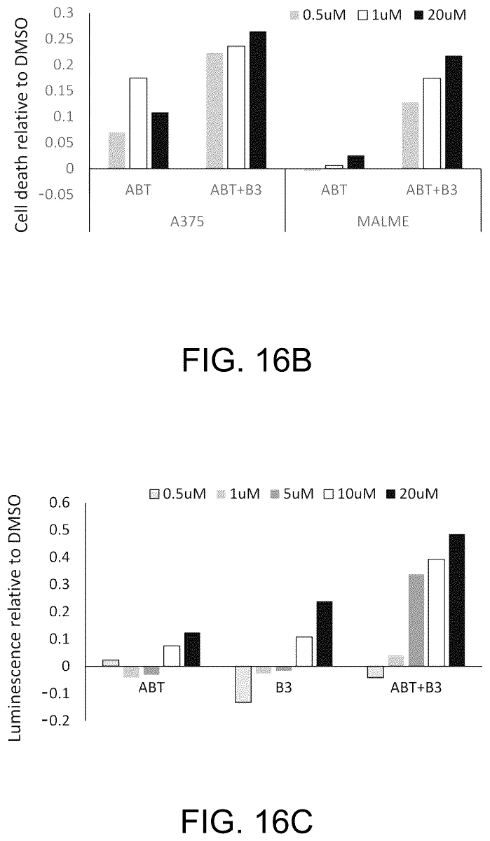

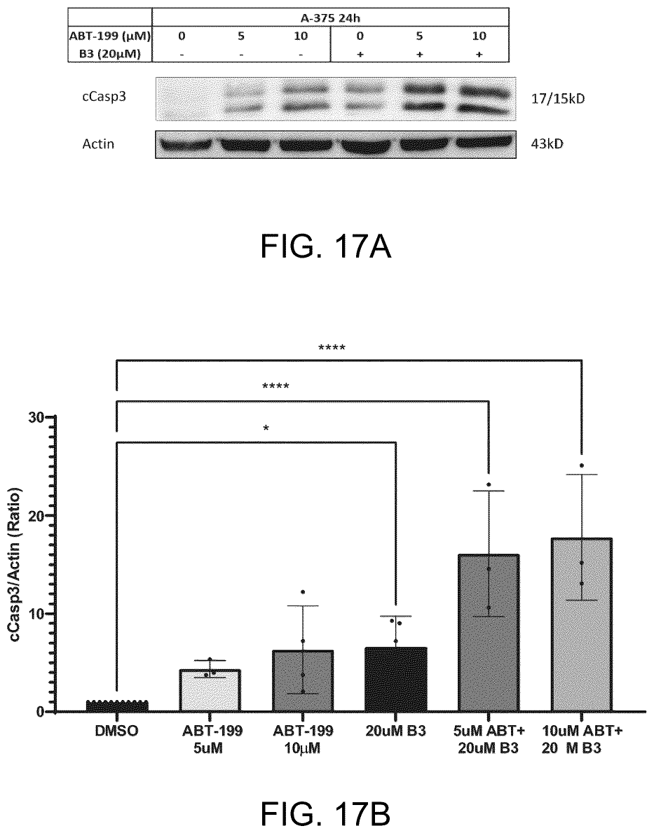

Still further, as shown in FIG. 13A-13E, analysis of A-375 μMelanoma cell line treated for 2, 6 and 24 hrs with ABT-199 with and without the small molecule B3, showed that Bcl-2 levels were upregulated upon treatment with ABT-199 alone. Six hours of B3 treatment decreased Bcl-2 levels. The combined treatment of ABT-199 with B3 decreased Bcl-2 and XIAP levels while cPARP levels were greatly upregulated after 24 hrs. Furthermore, the combined treatment of ABT-199 with increasing levels of B3, in A-375 cells, indicates that Caspas3 cleavage is maximal at 5 μM ABT-199+20 μM B3 (FIG. 16A), and that cell death is maximal at 5 μM ABT-199+20 μM B3 and the killing effect depends on B3 levels (FIG. 16B, 16C). Similar results are also shown by FIG. 17A, 17B.

The inventors further showed that A-375 cells treated with 10 μM or 20 μM ABT199 together with 20 μM B3 showed even better synergistic effect on apoptosis. Significant increase in cCaspase 3 and cPARP levels, and decrease in Bcl2 and XIAP levels in comparison to untreated cells or cells treated with each compound separately (FIG. 24A-24B). The same apoptotic effect was observed in different cell lines from different origins as shown in FIG. 22 and FIG. 25.

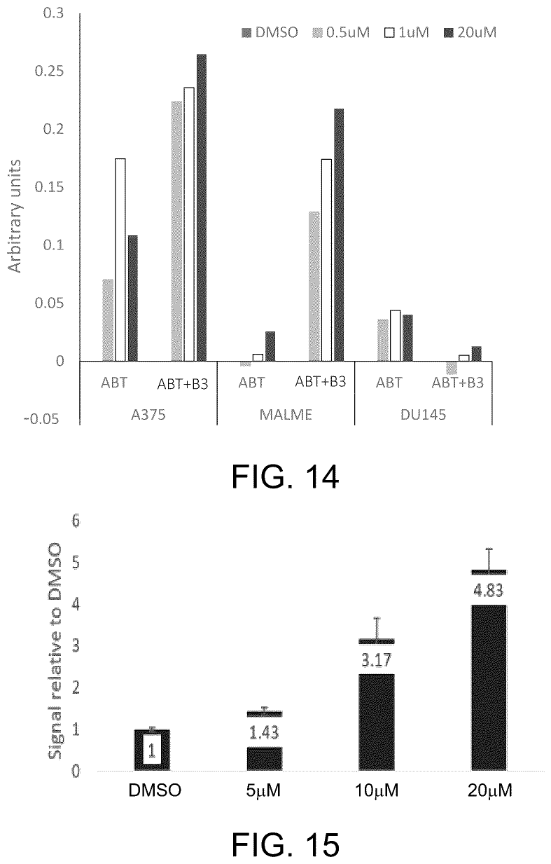

Next, cell death was measured by XTT assay. As shown by FIG. 14, combined treatment of ABT+B3 increased cell death relative to ABT-199 alone in both A375 and MALME in melanoma cell lines.

Significantly, though treatment of B3 alone is sufficient to induce apoptosis in these cells, the combined treatment of ABT-199 with B3 significantly reduced both Bcl-2 and XIAP expression. This culminates in a substantial increase in the ABT-199/B3 induced cancer cell death. Thus, the combined treatment of ABT-199 with B3 rescues the effect of ABT-199 on these resistant cancer cells and restores their sensitivity to this drug. Furthermore, we show that B3 restores the sensitivity to ABT-199 by promoting the degradation of Bcl-2 and XIAP. These results provide an alternative and complementary approach to treatment of cancers that developed resistance to BH3 mimetics.

### Example 5

B3 and ABT-199 promote the binding of XIAP to Bcl-2

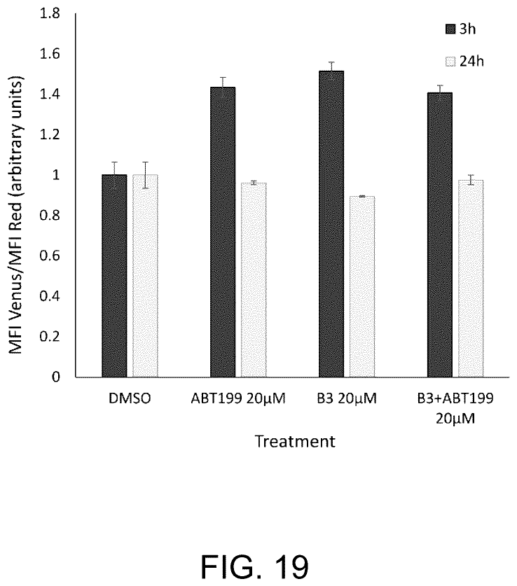

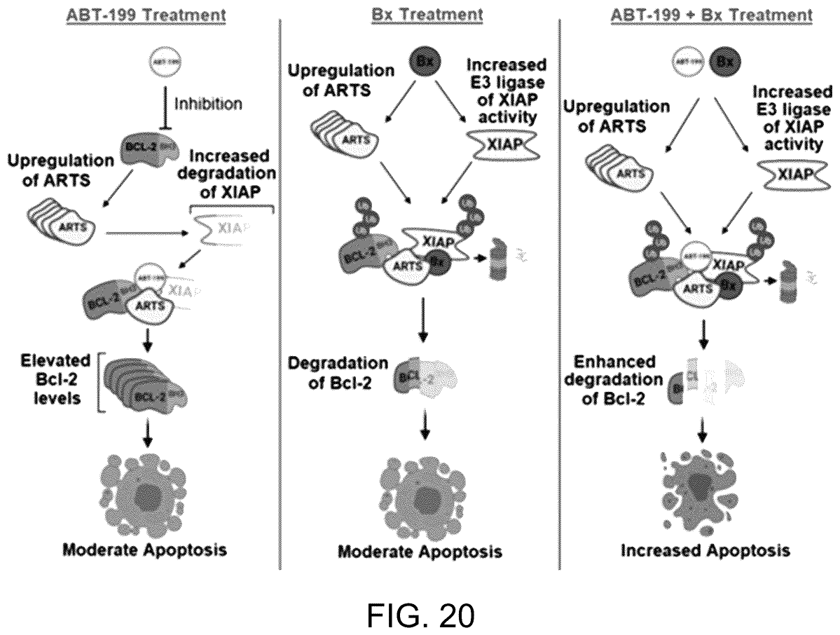

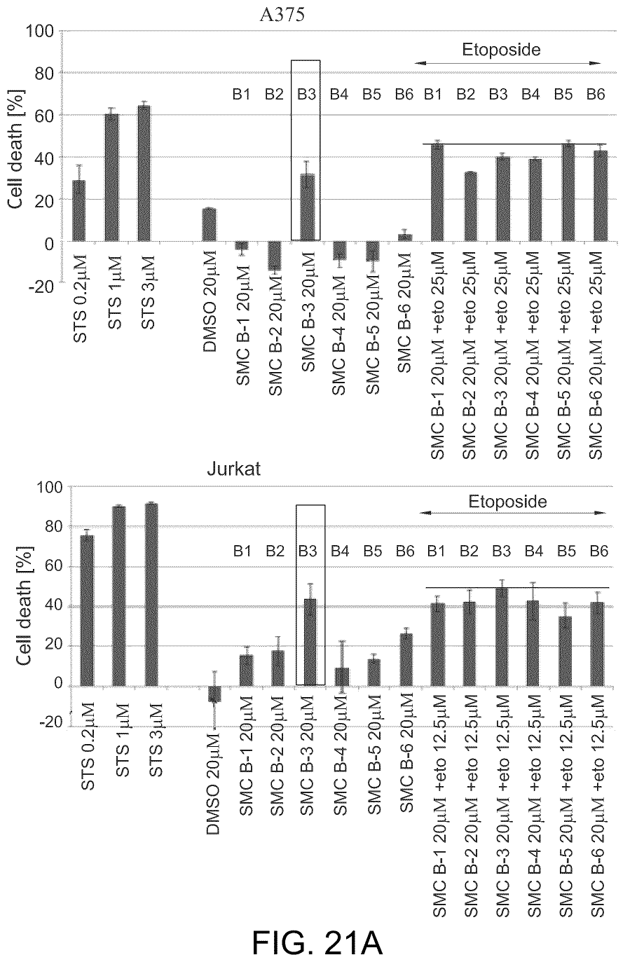

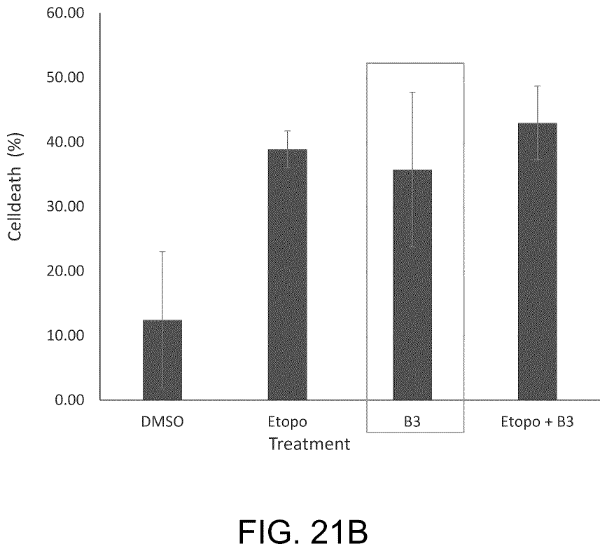

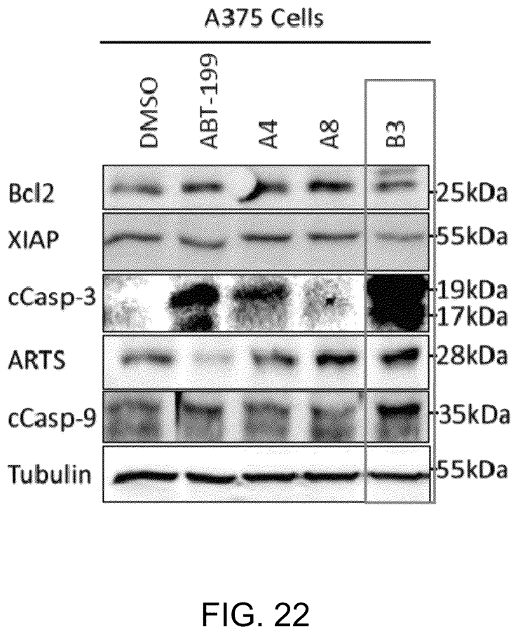

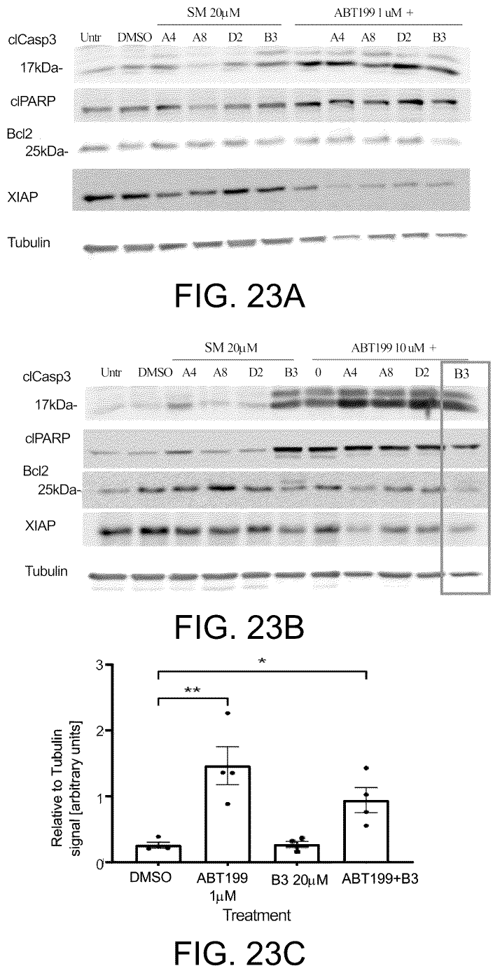

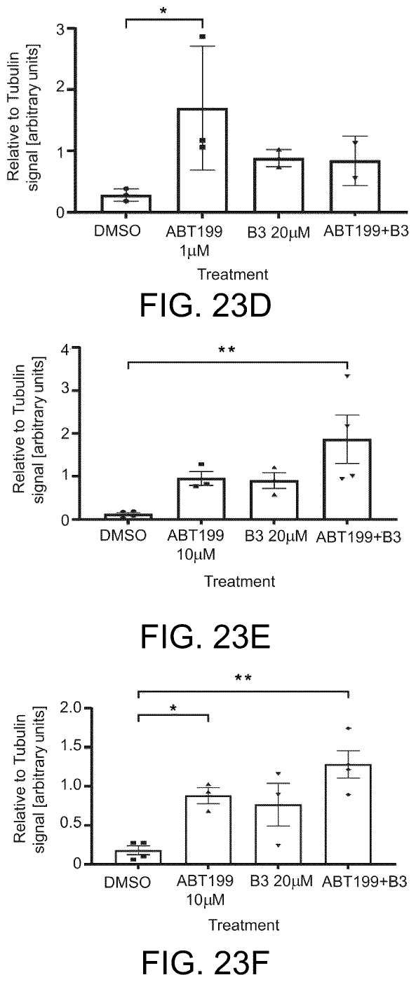

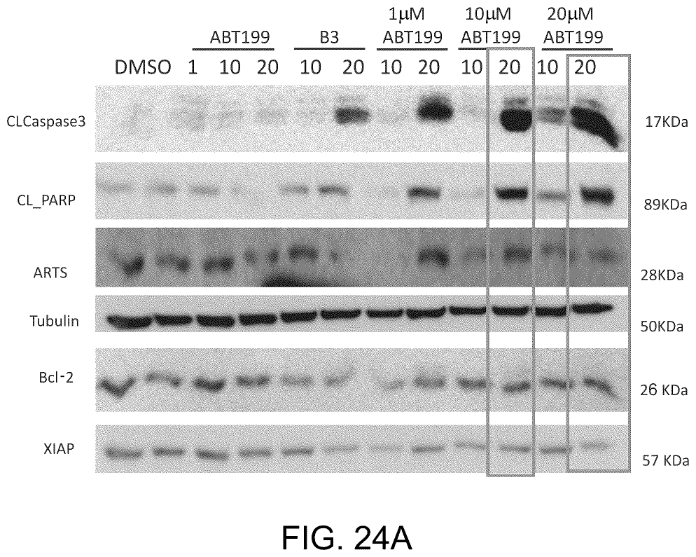

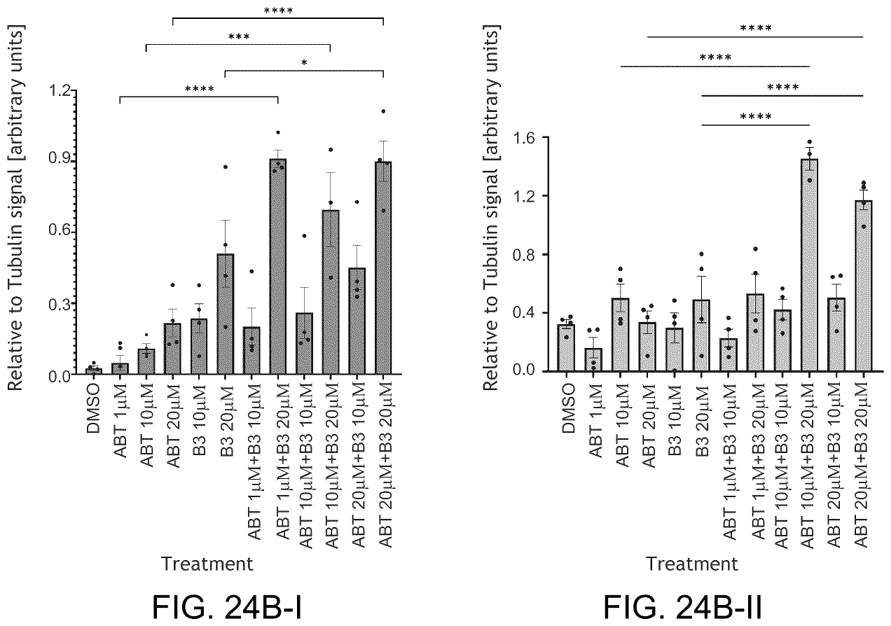

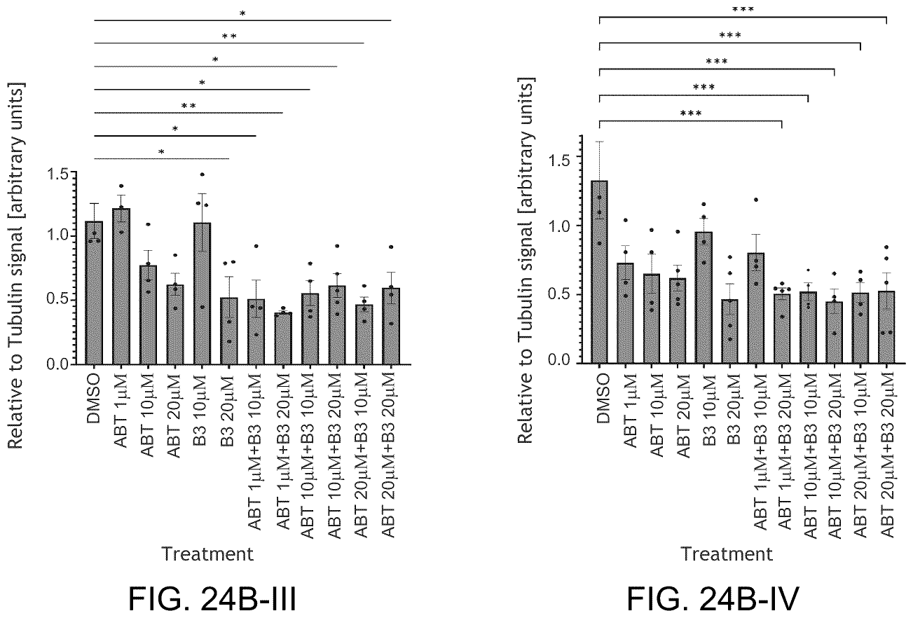

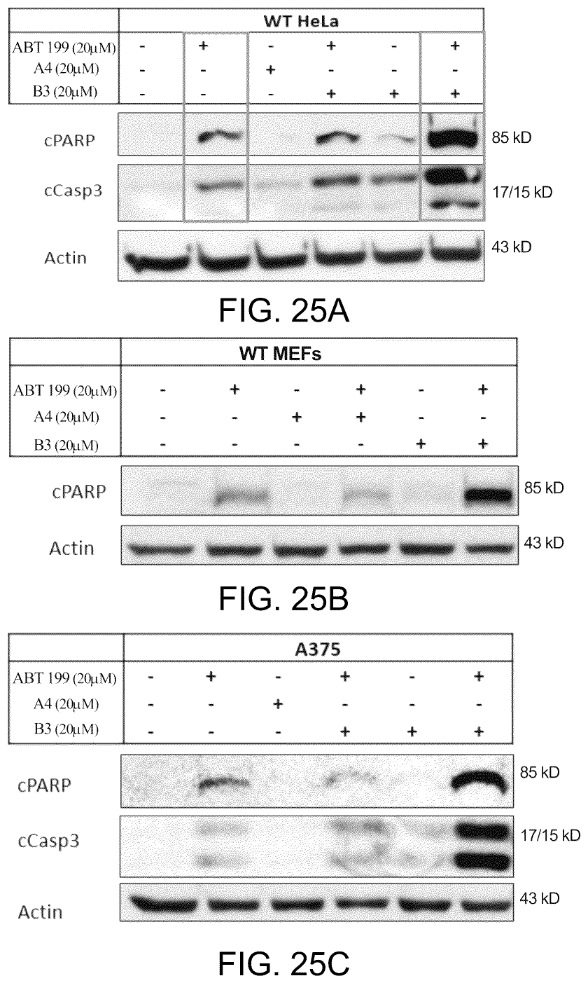

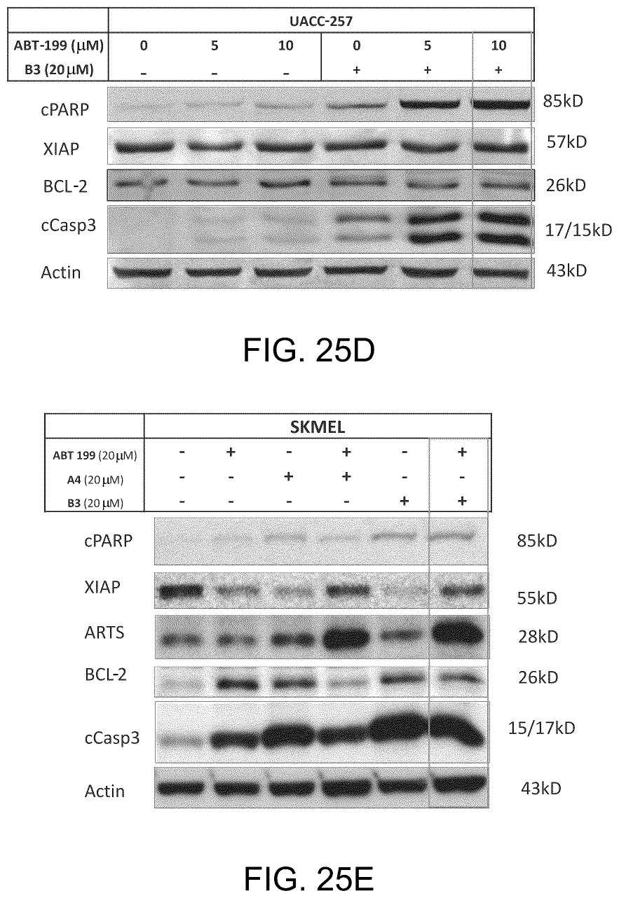

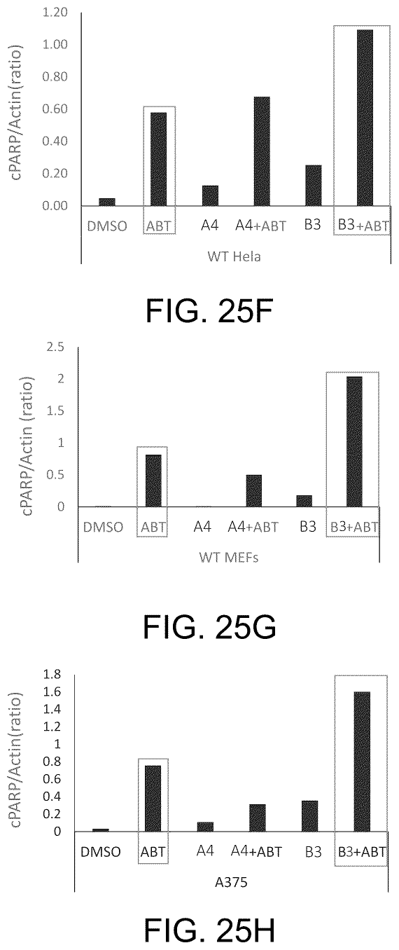

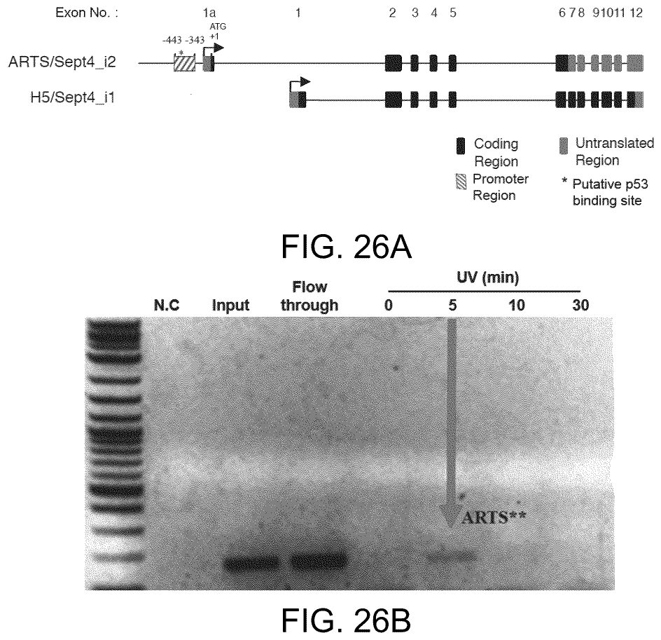

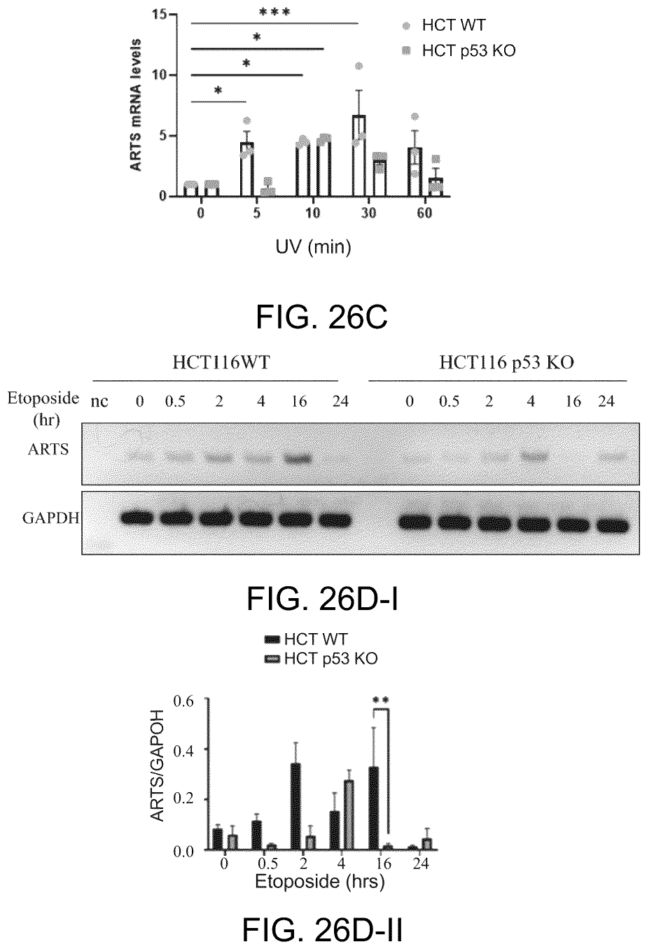

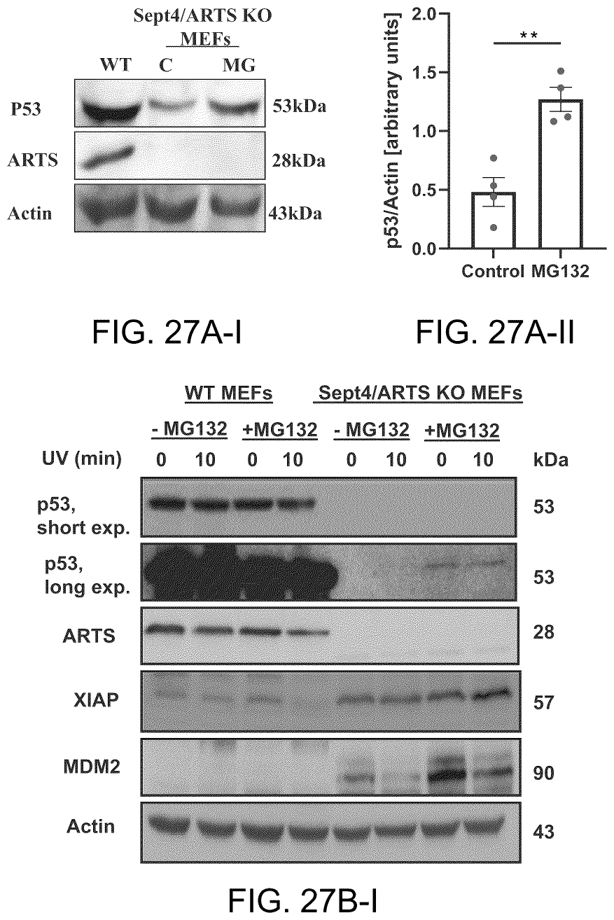

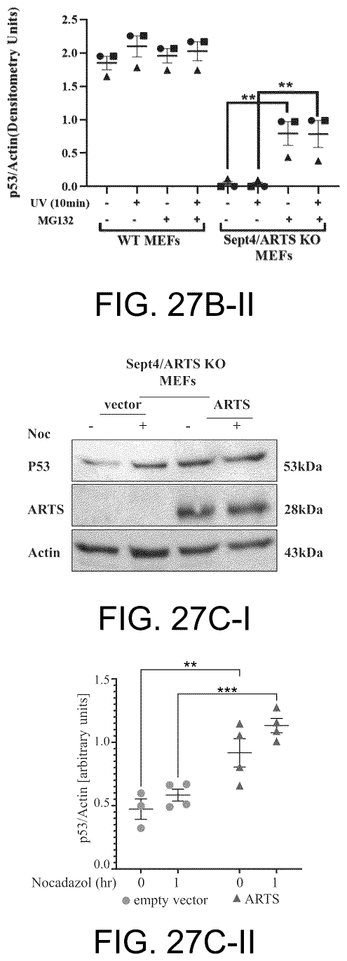

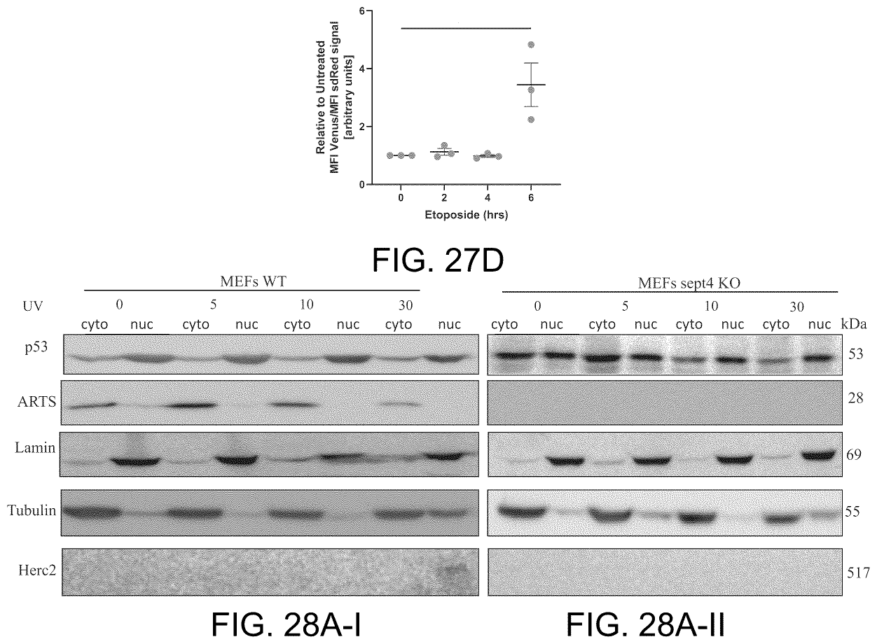

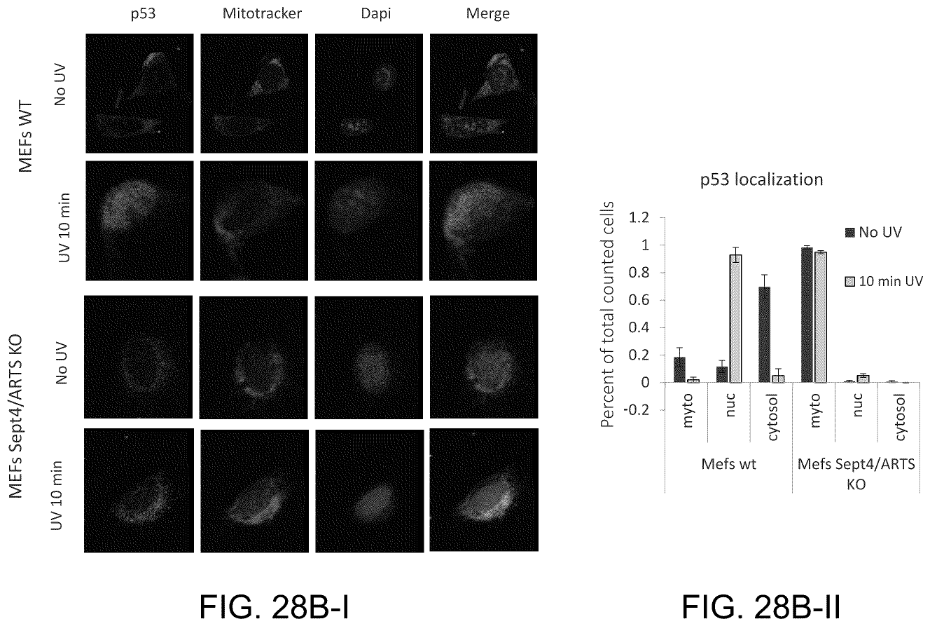

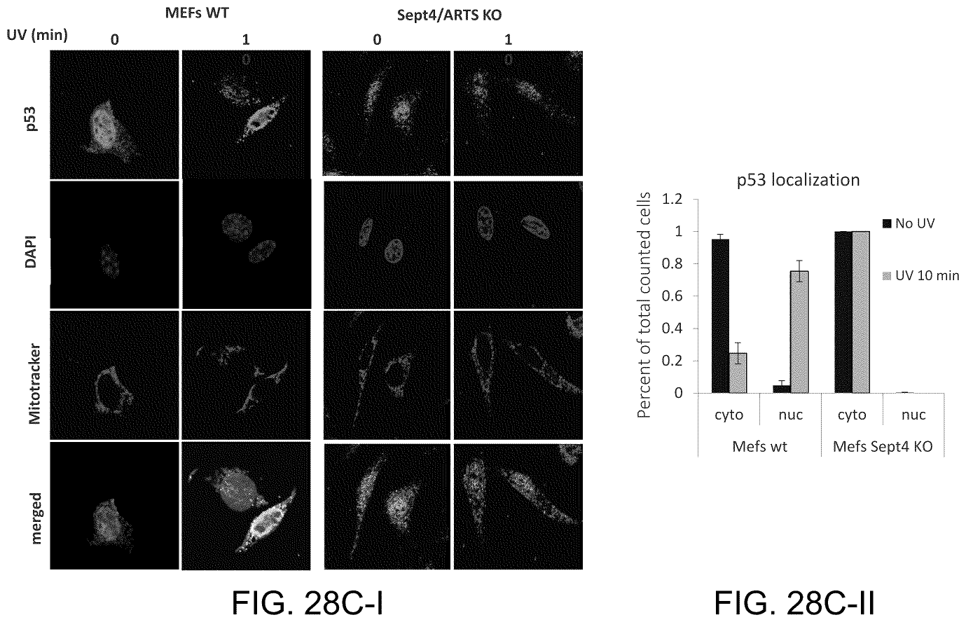

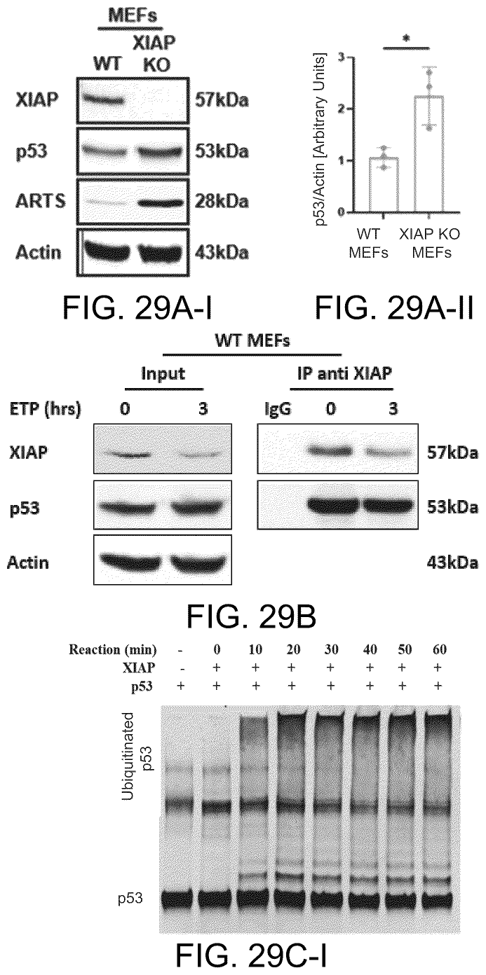

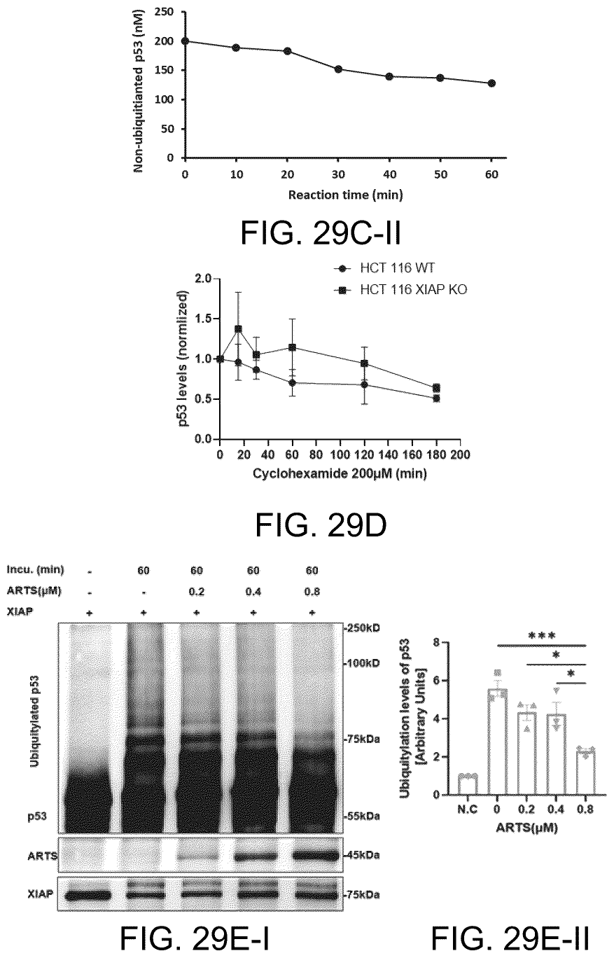

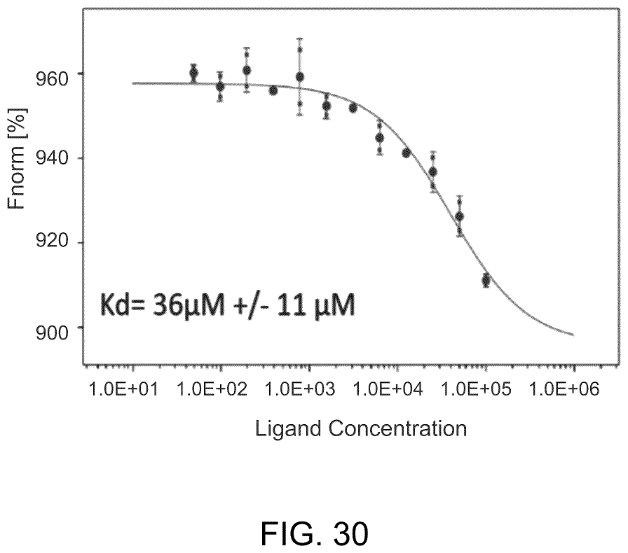

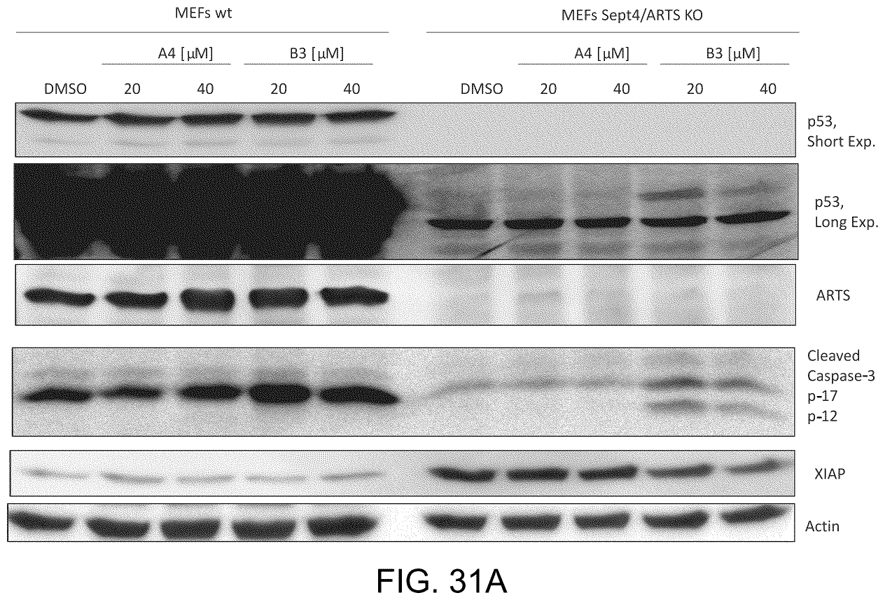

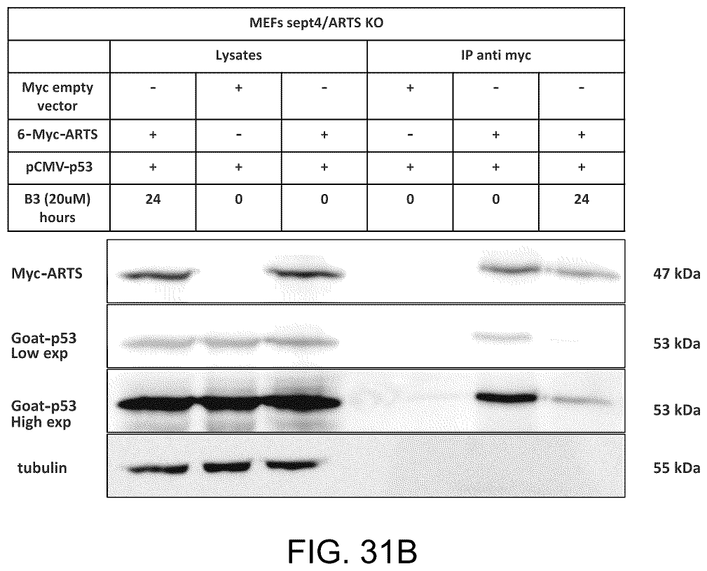

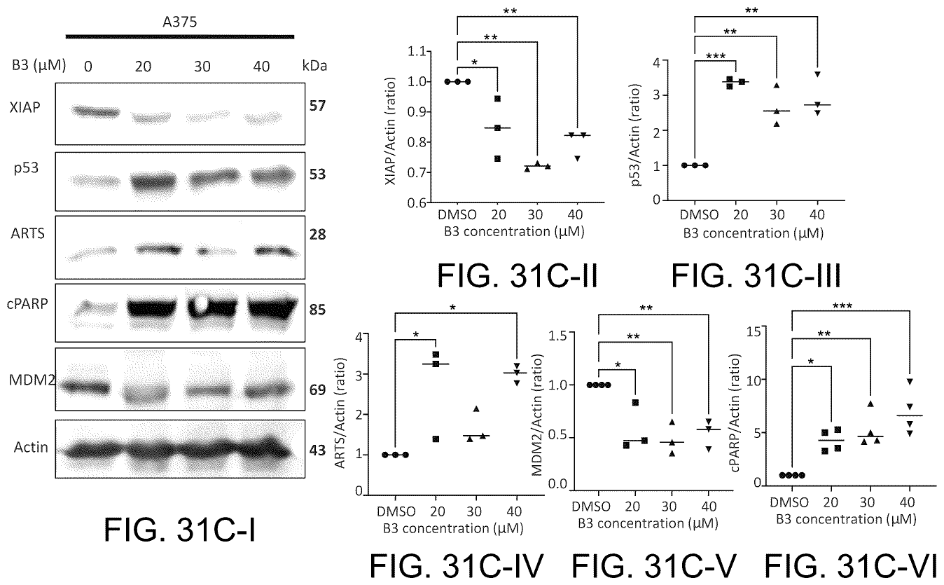

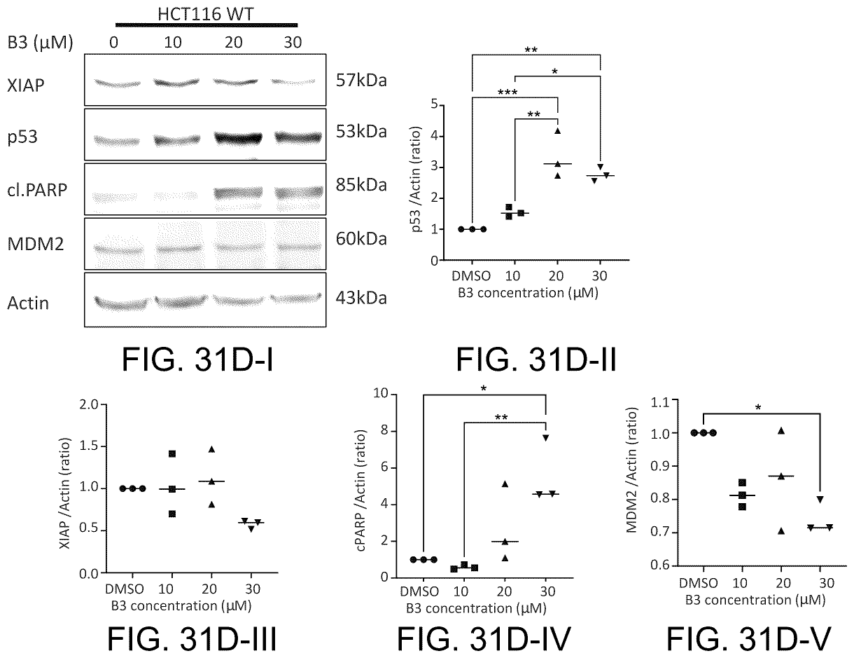

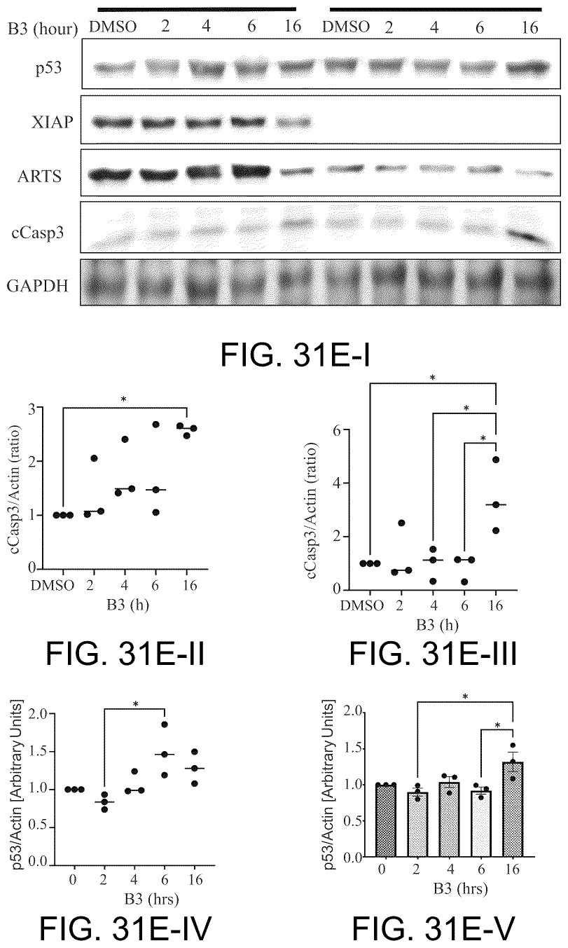

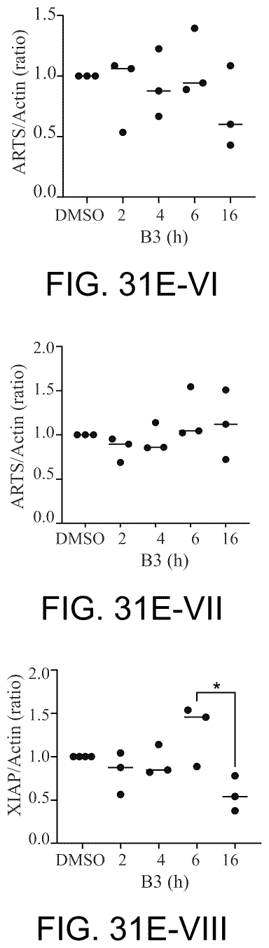

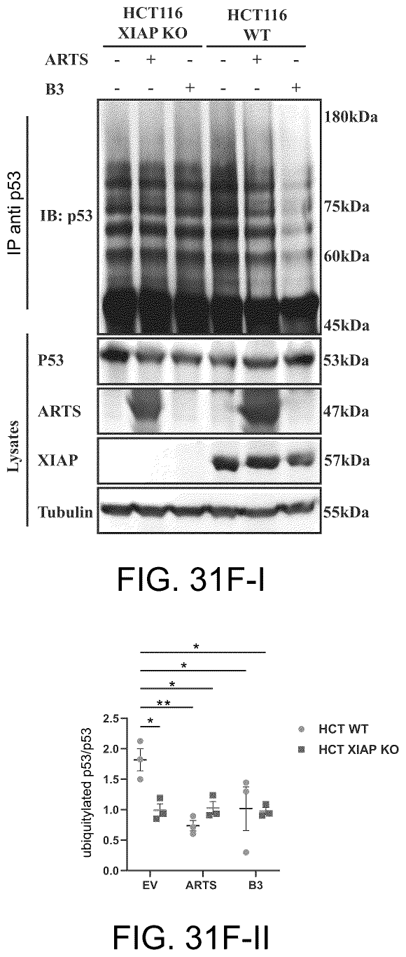

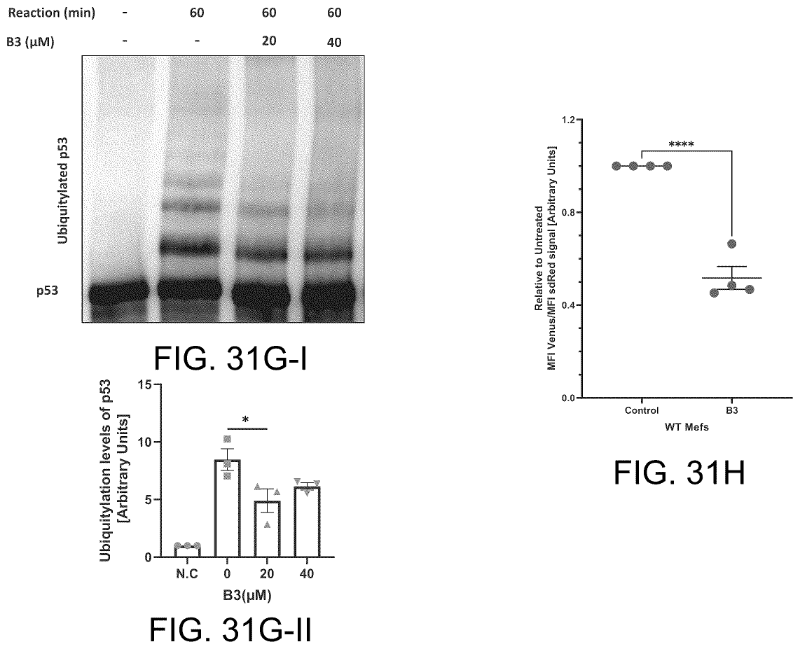

To further investigate the molecular mechanism that is activated when cells are treated with ABT-199 and B3, the investors determine the ability of B3 to directly bind to XIAP and Bcl-2 using nano-DSF method (Crelux) and MicroScaleThermophoresis (MST) respectively. Indeed, binding curves of B3 to XIAP and Bcl-2 suggest it binds with high affinity to both proteins (FIG. 18A.I, A.II) These results suggest that B3, like ARTS, acts as a scaffold bringing XIAP in-close proximity to Bcl-2 to enable its ubiquitylation and degradation by XIAP. To test this hypothesis, the investors performed co-immunoprecipitation assays using XIAP- and Bcl-2-transfected cells that were treated with B3. The results indicate that B3 significantly increase complex formation between XIAP and Bcl-2 (FIG. 18B). Following, the investors determine interaction between the components of this complex in response to ABT-199 using Bimolecular Fluorescence Complementation (BiFC) assay. Results show that binding of XIAP to Bcl-2 is increased following 24 h treatment with 20 μM B3 (FIG. 18C(I), FIG. 18C(II)). Importantly, results of the BiFC assay performed between ARTS and Bcl-2, demonstrate that ABT-199 significantly increased the binding of ARTS to Bcl-2 just after 3 hours of treatment, while after 24 hours of treatment was not noticed (FIG. 19). Together, these results suggest that both ABT-199 and B3 act similarly by inducing the binding of ARTS to Bcl-2, thereby allowing formation of a ternary complex XIAP-ARTS-Bcl-2, which promotes the killing of cancer cells.

Without being bound by any theory, the investors propose a working model that summarizes the molecular mechanism by which ABT-199 and B3 induce apoptosis through regulation of Bcl-2 levels (FIG. 20). According to this model, treatment with ABT-199 and with B3 upregulates ARTS, which promotes the formation of protein complex between Bcl-2-ARTS and XIAP. This results in regulation of Bcl-2 levels. ABT-199-induced upregulation of ARTS promotes the degradation of XIAP, which can lead to elevated levels of Bcl-2 (FIG. 20, left column). However, treatment with B3 (FIG. 20, middle column) or the combined treatment (FIG. 20, right column) lead to reduced levels of Bcl-2. It is possible that the elevated levels of endogenous ARTS seen in response to ABT-199, promote a prominent auto-ubiquitylation and degradation of XIAP. Whereas binding of B3 to XIAP, when added alone or in combination with ABT-199, leads to better activation of the E3-ligase function of XIAP, perhaps through allosteric effect of B3 on XIAP. This leads to an enhanced degradation of Bcl-2, which results in a substantial increase in the apoptotic effect, especially when both compounds are used together (FIG. 20, right column).

### Example 6

### ARTS is a Transcriptional Target of p53

P53 induces apoptosis mainly through its transcriptional induction of target genes (Aylon and Oren 2007 Cell 130 597-600, Levine and Oren 2009 Nat Rev Cancer 9 749-758). Sequence-specific DNA binding of p53 is a prerequisite for trans-activating target genes (Aubrey et al. 2018 Cell Death Differ 25 104-113, Aylon and Oren 2007 Cell 130 597-600, Hassin and Oren 2022 Nat Rev Drug Discov 1-18, Levine and Oren 2009 Nat Rev Cancer 9 749-758). ARTS is a splice variant of the human Sept4 μgene located on human chromosome 17q22-23 (Mandel-Gutfreund et al. 2011 Biological Chemistry 392 783). Differential splicing of the Sept4 mRNA generates two significant isoforms, Sept4_i1 and ARTS (Sept4_i2) (FIG. 26A) (Mandel-Gutfreund et al. 2011 Biol Chem 392 783-790). Importantly, the mRNAs for both major isoforms originate from two distinct transcription start sites (TSS), and the expression, tissue distribution, and function of ARTS and Sept4_i1 are very different (Mandel-Gutfreund et al. 2011 Biological Chemistry 392 783). Remarkably, only ARTS (Sept4_i2) functions as a pro-apoptotic protein (Mandel-Gutfreund et al. 2011 Biological Chemistry 392 783). Accordingly, only ARTS expression is selectively lost in leukemia patients, while Sept4_i1 levels remain intact (Elhasid et al. 2004 Oncogene 23 5468-5475). This suggests the existence of two distinct promoters differentially regulated: a proximal promoter giving rise to Sept4_i1 and a more distally located promoter responsible for generating ARTS. Significantly, Kostic and Shaw reported a putative p53-binding site in the Sept4 μgene (Kostic and Shaw 2000 Oncogene 19 3978). Using bioinformatical analysis, the inventors identified a motif between nucleotides −391 and −351 upstream of ARTS TSS (FIG. 26A). This motif contains two half-sites that strongly resemble the consensus DNA sequence sufficient for p53 binding (el-Deiry et al. 1992 Nat Genet 1 45-49). To determine if p53 binds to the specific sequences of ARTS promoter in the Sept4 μgene to induce its transcriptional activation, the inventors performed a chromatin immunoprecipitation assay (ChIP). For that purpose, WT HCT116 cells were exposed to UV irradiation (FIG. 26B). Specific binding of p53 to the ARTS promoter sequences is shown following 5 minutes of UV irradiation (FIG. 26B).

As p53 upregulates the transcription of pro-apoptotic proteins such as Bax (Chipuk et al. 2004 Science 303 1010-1014), the inventors also tested whether p53 upregulates the transcription of ARTS. Therefore, a real-time PCR assays were performed using isolated mRNA from WT HCT 116 and p53 Knockout (KO) HCT 116 (FIG. 26C). Apoptosis was induced by exposing the cell to UV for 5, 10, 30, and 60 min. While all of WT HCT 116 exhibited a significant increase in levels of ARTS mRNA upon induction of apoptosis, the levels of ARTS mRNA in the p53 KO and KD cells remained low (FIG. 26C). Thus, p53 is required for the upregulation of ARTS mRNA and acts as its transcription factor under DNA-damaging conditions.

FIGS. 26D(i) and 26D(ii) show similar increase in ARTS mRNA in the presence of p53, within 2 hrs. RNA levels of ARTS are upregulated during apoptosis in p53-containing cells, while no upregulation of ARTS is seen in P53 KO cells. HCT116 WT cells and HCT116 p53 KO cells were treated with 50 mM Etoposide for the indicated time points. Cells were harvest and RNA was extracted using RNA isolation kit. RT PCR was performed in order to get cDNA, after which PCR was done, using specific primers for ARTS and GAPDH for amplification. GAPDH served as a loading control. The samples were separated using agarose gel. Quantitation of ARTS and GAPDH levels were done using ImageJ analysis software (Quantification of bands from DNA films. The quantification reflects the relative amounts as a ratio of each protein band relative to the lane's loading control). The graphs represent the ratio numbers of ARTS/GAPDH. These results suggest that p53 acts as a transcriptional regulator of ARTS.

### Example 7

### ARTS Regulates p53 Protein Levels

The transcriptional network of p53-responsive genes, upregulates proteins that interact with various cellular pathways resulting in several positive and negative auto-regulatory feedback loops to regulate p53 levels (Harris and Levine 2005 Oncogene 24 2899-2908). The investors have shown that p53 up-regulates ARTS mRNA levels (FIG. 26B-26D). Next, the inventors wanted to test whether in turn, ARTS can affect p53 protein expression. For this purpose, levels of p53 protein were examined in the presence or the absence of ARTS using WT MEFs and Sept4/ARTS KO MEFs (FIG. 27A, 27B and FIG. 27C). The inventors suspected that p53 low levels observed in sept4/ARTS KO MEFs (FIG. 27A(i and ii), FIG. 27B (i and ii) and FIG. 27C (i and ii)) might be a result of excessive degradation via the UPS, and increased levels of XIAP and MDM2 in ARTS KO cells. Therefore, the sept4/ARTS KO MEFs were treated with the proteasome inhibitor MG-132, 20 μM for 6 hours (FIG. 27A and FIG. 27B) or with 200 ng/ml nocodazole for 1 h (FIG. 27C). The treatment with the inhibitors resulted in the accumulation of p53 protein levels (FIG. 27A(i and ii), FIG. 27B(i and ii) and FIG. 27C (i and ii)). Accordingly, p53 low levels in sept4/ARTS KO MEFs result from excessive degradation through the UPS. Subsequently, the inventors wanted to confirm if the absence of ARTS is responsible for the p53 low levels in sept4/ARTS KO MEFs. Reintroducing exogenous ARTS into the sept4/ARTS KO MEFs resulted in the upregulation of p53 levels (FIG. 27C(i and ii)). ARTS solely significantly upregulated p53 protein levels compared to treatment with 200 ng/ml Nocodazole (NOC) for 1 hour (FIG. 27C). This indicates that ARTS regulates p53 levels through the UPS. To test, if ARTS and p53 interact with each other, a bimolecular fluorescence complementary assay (BiFC) was conducted. The inventors found that ARTS and p53 bind each other upon 6 hours of 200 μM of Etoposide treatment (FIG. 27D). In their previous published data (Bornstein et al. 2011 Apoptosis 16 869-881; Edison et al. 2017 Cell Reports 21 442-454), the inventors have shown that ARTS serves as a physiological antagonist of XIAP and that sept4/ARTS KO MEFs exhibit high levels of XIAP. This suggests another link in the regulatory network between ARTS and p53.

### Example 8

### ARTS Accelerates the Translocation of p53 to Nucleus

The cellular localization of p53 has been next examined. FIG. 28A shows that ARTS accelerates the translocation of p53 to nucleus in WT MEFs cells (FIG. 28A(i)) that express ARTS as compared to cells that do not express ARTS (sept4/ARTS KO MEFs) (FIG. 28A(ii)).

Moreover, FIG. 28B shows that ARTS is important for the localization of p53 to the nucleus, since in sept4/ARTS KO MEFs cells, p53 display predominant mitochondrial localization.

FIG. 28C further shows intense nuclear localization of p53 upon exposure to UV for 10 minutes. In cells not expressing ARTS, the localization is cytoplasmic upon UV exposure. P53 translocate from mitochondria to the nucleus upon induction of apoptosis. MEF WT cells were treated with UV radiation for the indicated time points. With no UV treatment, p53 was localized at the mitochondria and translocated to the nucleus upon UV-induced apoptosis. Nuclei were stained blue with Dapi staining, mitochondria were stained red with Mitotracker. Sept4/ARTS KO MEFS show inhibition of p53 translocation to the nucleus. SEPT4/ARTS KO MEFs were treated with UV radiation for the indicated time points. A major fraction of P53 remained in the mitochondria as compared to WT UV-treated cells. Thus, ARTS seems to play a major role in translocation of P53 to the nucleus following UV treatment. Nuclei were stained blue with Dapi staining, mitochondria stained red with Mitotracker and incubated with anti-p53 (green) antibody. Quantitation of the cellular localization of p53. To quantify the number of cells with mitochondrial versus nuclear localization of p53, IF assay was performed. The graphs represent the numbers of cells displaying cytoplasmic/nuclear staining as a percent of total counted cells (mean±S.E, n¼ 300).

### Example 9

### XIAP Acts as an E3 Ligase for p53

XIAP regulates the levels of many pro-apoptotic proteins through the UPS, including ARTS (Abbas and Larisch 2020 Cells 9; Abbas and Larisch 2021 Cells 10). Vice versa, ARTS regulate XIAP levels by bringing XIAP into close proximity with the E3 ligase SIAH and by initiating the auto-Ubiquitylation of XIAP (Bornstein et al. 2011 Apoptosis 16 869-881; Edison et al. 2012 Clin Cancer Res 18 2569-2578; Garrison et al. 2011 μMol Cell 41 107-116). Based on these findings, the inventors examined the possibility that ARTS regulates p53 protein levels through its effect on XIAP. The inventors tested whether in the absence of XIAP (XIAP KO MEFs), higher levels of p53 are observed compared to MEFs WT. Indeed, p53 protein levels are elevated in XIAP KO MEFs compared to WT MEFs (FIG. 29A(i)-(ii)), that express XIAP. Furthermore, immunoprecipitation of XIAP with anti-XIAP antibodies followed by Western blot confirmed that XIAP binds p53, and upon treatment with 200 μM of ETP for 3 hours the levels of XIAP were downregulated, and the levels of p53 were upregulated thereby the binding between XIAP and p53 was affected (FIG. 29B). Next, to assess if XIAP serves as a novel E3 ligase of p53, the inventors collaborated with Dr. Hyoung-Tae Kim (Harvard Medical School), to perform in vitro Ubiquitylation assays using recombinant XIAP and p53 proteins with UbcH5b as E2 (FIG. 29C(i)-(ii)). The results show that XIAP ubiquitylates p53 as early as 10 minutes of co-incubation. FIG. 29D further demonstrate a higher half-life of P53 in HCT XIAP KO as compared to HCT 116 WT. So far, these data prove that p53 upregulates ARTS mRNA levels and, consequently, ARTS protein levels. In turn, ARTS upregulates p53 protein levels. Moreover, the inventors found XIAP to serve as a novel E3 ligase of p53. To test the dynamics between the three proteins, the inventors performed another in vitro Ubiquitylation assay by incubating XIAP, p53, and ARTS together (FIG. 29E(i)-(ii)). A dose-response between ARTS concentration and p53 Ubiquitylation levels was seen (FIG. 29E). Upon adding ARTS to the solution of p53 and XIAP, p53 Ubiquitylation is downregulated. This indicates that ARTS upregulates p53 levels by inhibiting its Ubiquitylation by XIAP (FIG. 29E).

### Example 10

### ARTS Mimetic Small Molecule Binds XIAP-BIR3

The pursuit of p53-targeted therapy began with the identification of compounds capable of restoring/reactivating wild-type p53 functions or by eliminating mutant p53 (Hu et al. 2021 J Hematol Oncol 14 157). However, the search for efficient and selective drugs that will eventually be able to enter the clinic is still ongoing (Duffy et al. 2022 Cancers (Basel) 14; Hassin and Oren 2022 Nat Rev Drug Discov 1-18, Hu et al. 2021 J Hematol Oncol 14 157). Furthermore, many cancers escape apoptosis by overexpressing XIAP (Prehn et al. 1994 Proc Natl Acad Sci USA 91 12599-12603; Thompson 1995 Science 267 1456-1462; Yang and Korsmeyer 1996 Blood 88 386-401). XIAP is overexpressed in leukemia, lung, colon, melanoma, ovarian, bladder, renal, breast, prostate, and thyroid carcinomas (Dubrez et al. 2013 Onco Targets Ther 9 1285-1304; Krajewska et al. 2003 Clin Cancer Res 9 4914-4925; Tamm et al. 2000 Clin Cancer Res 6 1796-1803). Therefore, XIAP has become an attractive target for developing anti-cancer drugs (Abbas and Larisch 2020 Cells 9; Abbas and Larisch 2021 Cells 10; Jost and Vucic 2019 Cold Spring Harb Perspect Biol; Mamriev et al. 2020 Cell Death Dis 11 483). Most of the efforts to target IAPs were focused on developing IBM (Smac) mimetics (Bergmann et al. 2003 Curr Opin Cell Biol 15 717-724; Chai et al. 2000 Nature 406 855-862; Corti et al. 2018 FEBS J 285 3286-3298; Li et al. 2004 Science 305 1471-1474; Oost et al. 2004 J Med Chem 47 4417-4426; Sun et al. 2008 Acc Chem Res 41 1264-1277; Sun et al. 2004 J Am Chem Soc 126 16686-16687). On one hand, these small molecules can bind and degrade cIAPs. On the other hand, they can bind but not degrade XIAP (Amaravadi et al. 2015 μMol Cancer Ther 14 2569-2575; Benetatos et al. 2014 μMol Cancer Ther 13 867-879; Condon et al. 2014 J Med Chem 57 3666-3677; Corti et al. 2018 FEBS J 285 3286-3298; Finlay et al. 2017 F1000Res 6 587). A major goal of the pharmaceutical industry is to develop potent and specific small molecules that selectively degrade XIAP (Bornstein et al. 2012 Int J Biochem Cell Biol 44 489-495; Lai and Crews 2017 Nat Rev Drug Discov 16 101-114). To address this need, the inventors generated small-molecule ARTS mimetics that can bind directly to the unique recognition sequence of ARTS in the BIR3 domain of XIAP (as denoted by SEQ ID NO: 11), but not to clAPs. These compounds promote XIAP ubiquitylation and degradation via the UPS (Mamriev et al. 2020 Cell Death Dis 11 483). The inventors previously showed that small peptides encompassing the binding site of ARTS to XIAP can promote cell death in cancer cells (Edison et al. 2012 Clin Cancer Res 18 2569-2578; Reingewertz et al. 2011 PLoS One 6 e24655). This provides proof of concept that mimicking the function of ARTS to antagonize XIAP can promote apoptosis. Next, the inventors performed a structure-based computational screen analyzing 600,000 compounds to identify candidates predicted to bind the unique binding pocket for ARTS in XIAP/BIR3 (performed by BioSolveIt Ltd.). The inventors identified 100 molecules with the highest affinity scores of dockings to the unique binding site of ARTS in BIR3/XIAP (FIG. 1). The Small molecule, B3, specifically, (S)-N1-(1-hydroxy-3-phenylpropan-2-yl)-N2-(4-(1-methyl-1H-imidazole-2-carbonyl) phenyl) oxalamide, is one of the top-ranked molecules to bind the backbone of the distinct ARTS binding site of XIAP-BIR3 (specifically the bonds to T271 and T274) (FIG. 3). Using microscale thermophoresis (MST) assay, the inventors found that B3 showed relatively high binding affinity to the XIAP-BIR3 domain (Kd=36 μM+/−11 μM) (FIG. 30).

### Example 11

### ARTS Mimetic Small Molecule Upregulates p53 Through its Effect on XIAP, and Promote Apoptosis

To examine whether B3 can mimic the function of ARTS, the inventors tested in melanoma cell line A375, whether B3 can downregulate XIAP levels, upregulate p53 levels and induce apoptosis.

The B3 compound has been shown as inducing apoptosis as reflected by elevation in cleaved PARP and Caspase 3 (FIG. 4), while reducing the levels of the anti-apoptotic proteins XIAP and Bcl-2. As shown in FIG. 31A, ARTS mimetic small molecules B3 and A4, upregulate p53 levels and downregulates XIAP. FIG. 31B specifically demonstrates that the ARTS mimetic compound B3 reduces the binding between ARTS and p53, as shown by Co—IP between ARTS and p53. Moreover, FIGS. 31C-31D show dose response of the ARTS mimetic B3 compound that leads to elevation in p53, and cPARP or caspase3, and reduction in E3 ligases of p53, MDM2 and XIAP in either A375 cells (FIG. 31C(i)-(vi)), HCT116 cells (FIG. 31D), or MEF cells (FIG. 31E (i)-(viii)).

Thus, ARTS and its mimetic small molecules, specifically, B3, act as negative regulators of E3 ligases that act on p53, and thus function as a strong agonist of p53.

Next, the inventors tested the effect of ARTS and B3 on p53 Ubiquitylation levels by using HCT 116 WT and HCT 116 XIAP KO cells. Notably, ARTS overexpression and 20 μM of B3 both managed to inhibit the Ubiquitylation of p53 (FIG. 31F). Moreover, HCT 116 XIAP KO cells and the overexpression of ARTS in HCT 116 WT show the same Ubiquitylation levels of p53 (FIG. 31F). Importantly, ARTS overexpression and B3 treatment had no effect on p53 Ubiquitylation levels in HCT 116 XIAP KO cells, indicating that ARTS and B3 regulate p53 levels by antagonizing XIAP (FIG. 31F). To further confirm that B3 affects p53 Ubiquitylation levels by antagonizing XIAP, the inventors performed an in vitro Ubiquitylation assay. Indeed, as shown by FIG. 6H(i)-(ii), B3 a dose-dependent inhibition of p53 Ubiquitylation by XIAP. In addition, BiFC assay showed that 20 μM of B3 for 18 hours disrupted the binding between VN-p53 and VC-XIAP (FIG. 31G).

Without being bound by the theory, it appears that ARTS mimetic compounds upregulate p53 through its effect on XIAP.

1. A method for treating, preventing, inhibiting, reducing, eliminating, protecting or delaying the onset of a pathological disorder in a subject in need thereof, and/or for inducing apoptosis in a cell, said method comprises administering to said subject and/or contacting the cell with an effective amount of at least one ARTS mimetic compound or of a composition comprising the same, said ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same; wherein said Formula (I) is: 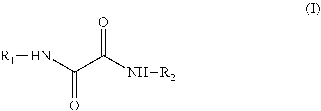
wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L2′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—, each optionally substituted by one or more of C₁-C₅alkoxy, C₁-C₅ carboxylic acid, —(CH₂)ₘ—OH, —(CH₂)ₘ SH, —(CH₂)ₘ—NH₂, —(CH₂)ₘ-halogen; n is an integer selected from any one of 0, 1, 2, 3, 4, 5 and m is an integer selected from 0, 1, 2, 3, 4, 5.

2. The method of claim 1, wherein:
(a) L₁′, L₁″, L₂′ and L₂″ are independently from each other selected from —(CH₂)ₙ—, C(═O) optionally —(CH₂)ₙ— substituted with —(CH₂)ₘ—OH and wherein n is an integer selected from 1, 2, or 3 and m is an integer selected from 1, 2 or 3; or
(b) each one of R₃′, R₃″, R₄′, R₄″ is absent or may be independently from each other an aromatic or a heteroaromatic ring, each is independently from each other optionally substituted with OH, alkyl, halogen, CF₃, NO₂, C(═O).

3. The method of claim 1, having the general formula (II) 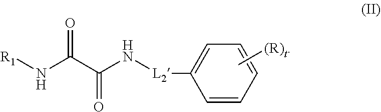
wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5, optionally, wherein L₂′ is (CH₂)ₙ—, or C(═O), optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5.

4. The method of claim 3, having the general formula (IIb): 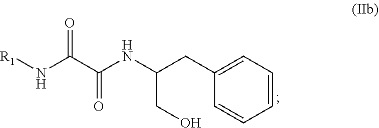
optionally, at least one of:
(a) wherein R₁ is L₁′-R₃′-L1″-R₃″; and/or
(b) wherein R₁ is at least one of
(i) L₁′, L₁″ and R₃″ are each absent and R₃′ is an optionally substituted 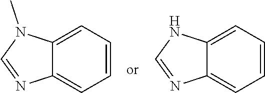
(ii) L₁′, L₁″ and R₃″ are each absent and R₃′ is: 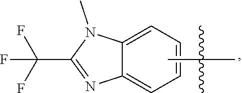
or
(iii) L₁′ is absent R₃′ is an optionally substituted phenyl, R₃″ is an optionally substituted: 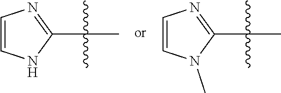
and L1″ is C(═O).

5. The method of claim 1, having the general formula (IIIa) or (IIIb): 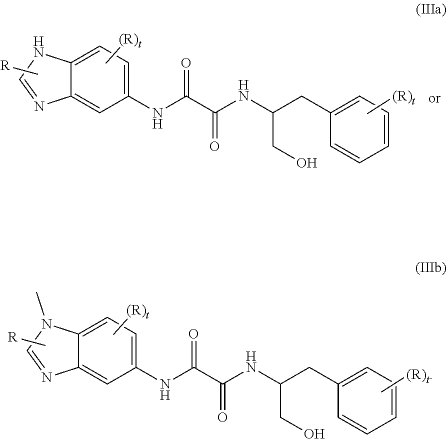
wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

6. The method of claim 1, having the general formula (IIIc), (IIId) or (IIIe): 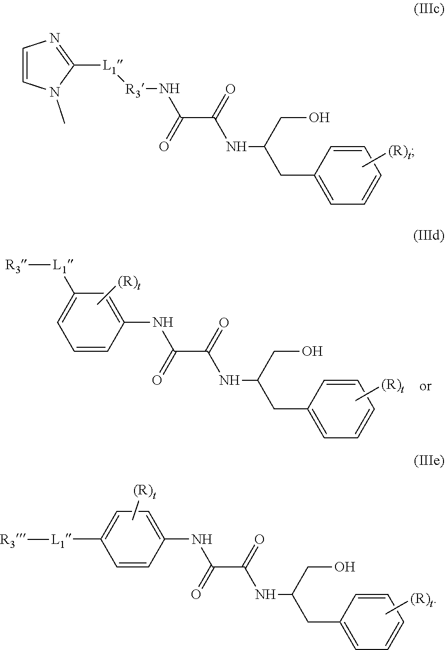
wherein L1″ and R3″ are each as defined above, wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

7. The method of claim 6, having the general formula (IIIc), or (IIIe): 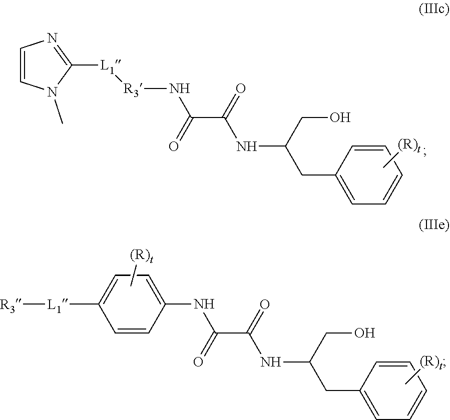
wherein R3′ and R3″ is each independently from the other, an optionally substituted aryl or an optionally substituted heteroaryl, and L1″ is C(═O), wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5.

8. The method of claim 7, having the formula (3.1), (3.2), (3.3); 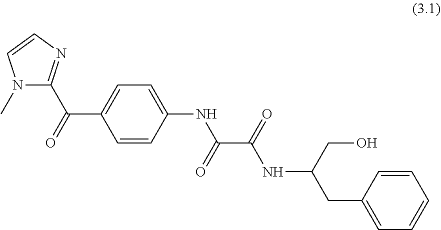 [SMILES: Cn1ccnc1C(=O)c1ccc(NC(=O)C(=O)NC(CO)Cc2ccccc2)cc1]
N1-(1-hydroxy-3-phenylpropan-2-yl)-N2-(4-(1-methyl-1H-imidazole-2-carbonyl)phenyl)oxalamide; 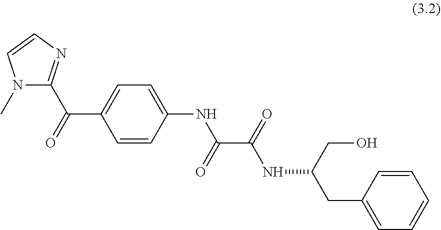
(S)-N1-(1-hydroxy-3-phenylpropan-2-yl)-N2-(4-(1-methyl-1H-imidazole-2-carbonyl)phenyl)oxalamide (denoted herein as “B3”): 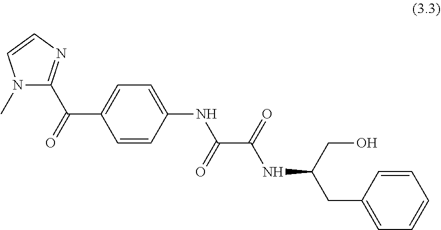 [SMILES: Cn1ccnc1C(=O)c1ccc(NC(=O)C(=O)N[C@@H](CO)Cc2ccccc2)cc1]
(R)-N1-(1-hydroxy-3-phenylpropan-2-yl)-N2-(4-(1-methyl-1H-imidazole-2-carbonyl)phenyl)oxalamide.

9. The method of claim 1, wherein at last one of:
(I) said subject is suffering from a pathologic disorder characterized by at least one of:
(a) over expression of Bcl-2;
(b) over expression of XIAP; and
(c) low or no expression of ARTS; and/or
(II) said ARTS mimetic compound leads to at least one of:
(a) ubiquitin proteasome system (UPS) mediated degradation of at least one of B-cell lymphoma 2 (Bcl-2) and X-linked-Inhibitor of Apoptosis (XIAP) in said cell;
(b) elevation in at least one of c-caspase and c-PARP levels in said cell; and/or
(c) induced apoptosis in a premalignant and/or a malignant cell;
optionally the cell is at least one of an epithelial carcinoma cell, a melanoma cell, a sarcoma cell, and hematological cancer cell.

10. The method according to claim 9, wherein said subject is a subject suffering from at least one proliferative disorder, optionally, said proliferative disorder is at least one of carcinoma, melanoma, sarcoma and/or at least one hematological disorder.

11. An ARTS mimetic compound having the general formula (I) or a pharmaceutically acceptable salt, solvate, hydrate, any stereoisomer thereof or physiologically functional derivative thereof, or any composition thereof or any vehicle, matrix, nano- or micro-particle comprising the same, wherein formula (I) is: 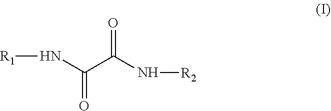
wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5.

12. The compound or composition of claim 11, having the general formula (II) 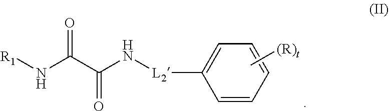
wherein R1 and L2′ is as defined above and wherein R is one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other and t is an integer selected from 1 to 5, optionally, at least one of:
(a) wherein L₂′ is (CH₂)ₙ—, or C(═O), each optionally substituted by one or more of C₁-C₅alkoxy, C₁-C₅ carboxylic acid, —(CH₂)ₘ—OH, —(CH₂)ₘ—SH, —(CH₂)ₘ—NH₂, or —(CH₂)ₘ-halogen, n is an integer selected from any one of 0, 1, 2, 3, 4, 5 and m is an integer selected from 0, 1, 2, 3, 4, 5; and/or
(b) the compound having the general formula (IIb): 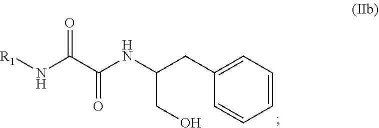
wherein R₁ is L₁′-R₃′-L₁″-R₃″.

13. The compound or composition of claim 11, having the general formula (IIIc), or (IIIe): 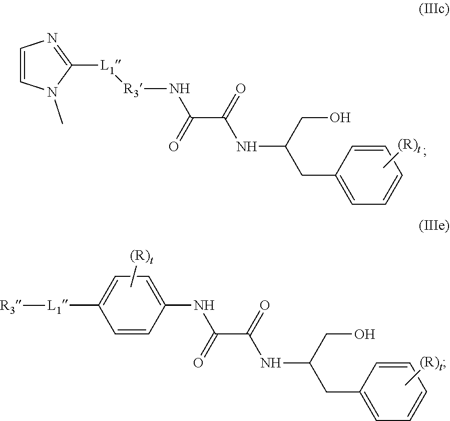
wherein R3′ and R3″ is each independently from the other, an optionally substituted aryl or an optionally substituted heteroaryl, and L1″ is C(═O), wherein R is one or more substituents as defined above, being one or more of H, OH, CF₃, halogen, C(═O), —COOH, —NH₂, alkyl, alkylene, C₁-C₁₂ alkoxy, a ring system containing five to twelve atoms, C₁-C₅ carboxyl, halogen, independently selected from each other.

14. The compound or composition of claim 13, having the formula (3.1), (3.2), (3.3); 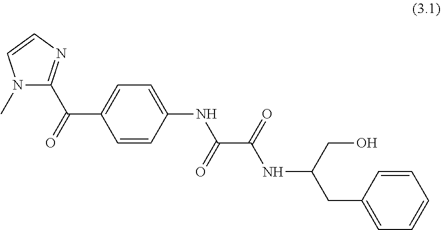 [SMILES: Cn1ccnc1C(=O)c1ccc(NC(=O)C(=O)NC(CO)Cc2ccccc2)cc1]
N1-(1-hydroxy-3-phenylpropan-2-yl)-N2-(4-(1-methyl-1H-imidazole-2-carbonyl)phenyl)oxalamide; 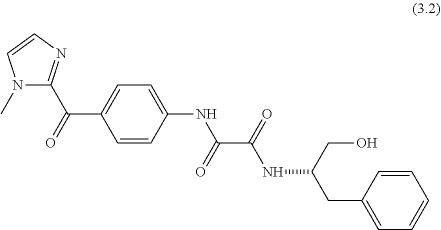
(S)—N1-(1-hydroxy-3-phenylpropan-2-yl)-N2-(4-(1-methyl-1H-imidazole-2-carbonyl)phenyl)oxalamide (denoted herein as “B3”): 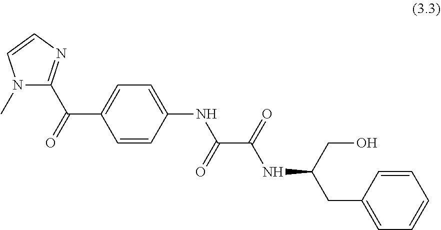 [SMILES: Cn1ccnc1C(=O)c1ccc(NC(=O)C(=O)N[C@@H](CO)Cc2ccccc2)cc1]
(R)-N1-(1-hydroxy-3-phenylpropan-2-yl)-N2-(4-(1-methyl-1H-imidazole-2-carbonyl)phenyl)oxalamide.

15. A combined composition or kit comprising an effective amount of at least one ARTS mimetic compound as defined in claim 11, and at least one and at least one B-cell lymphoma 2 (Bcl-2) Homology 3 (BH3) mimetic compound.

16. A method for treating, preventing, inhibiting, reducing, eliminating, protecting or delaying the onset of a proliferative disorder in a subject in need thereof, comprising administering to said subject a therapeutically effective amount of the combined composition as defined by claim 15.

17. A method for upregulating the levels of p53 in a cell, the method comprising the step of contacting said cell with an effective amount of ARTS, any fragments thereof, or at least one mimetic compound thereof or any vehicle, matrix, nano- or micro-particle comprising the same, or any composition thereof, wherein said ARTS mimetic compound is having the general formula (I), wherein formula (I) is: 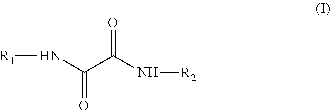
wherein
R₁, R₂ are each, independently from each other, absent or alkyl, alkenyl, alkynyl, alkenylene, a ring system containing five to twelve atoms, or a substituted version of any of these groups; or
R₁ is L₁′-R₃′-L1″-R₃″ and R₂ is alkyl, alkenyl, alkynyl, alkenylene, a ring system containing five to twelve atoms, or a substituted version of any of these groups; or
R₁ is alkyl, alkenyl, alkynyl, alkenylene, a ring system containing five to twelve atoms, or a substituted version of any of these groups and R₂ is L2′-R₄′-L₂″-R₄″; or
R₁ is L₁′-R₃′-L1″-R₃″ and R₂ is L₂′-R₄′-L₂″-R₄″;
wherein R₃′, R₃″, R₄′, R₄″ is each independently from each other, absent or H, alkyl, alkylene, alkenyl, alkenylene, alkynyl, alkynylene, aryl, arylalkyl, heteroaryl, heterocycloalkyl, a ring system containing five to twelve atoms, each optionally substituted;
L1′, L1″, L2′ and L2″ is each independently from each other, absent or —(CH₂)ₙ, —NH—(CH₂)ₙ—, C(═O), C(═O)—(CH₂)ₙ—, —O—, —SO₂—, —S—, —S—S—, —S—(CH₂)ₙ—; n is an integer selected from any one of 0, 1, 2, 3, 4, 5, each one of L1′, L1″, L2′ and L2″ independently from each other, may be optionally substituted by one or more of C₁-C₅alkoxy, C₁-C₅ carboxylic acid, —(CH₂)ₘ—OH, —(CH₂)ₘ—SH, —(CH₂)ₘ—NH₂, —(CH₂)ₘ halogen and m is an integer selected from 0, 1, 2, 3, 4, 5;
optionally, said upregulating p53 levels comprises reduction of p53 ubiquitylation by at least one E3 ligase.

18. The method of claim 17, wherein said compound is having the formula (3.1), (3.2), or (3.3); 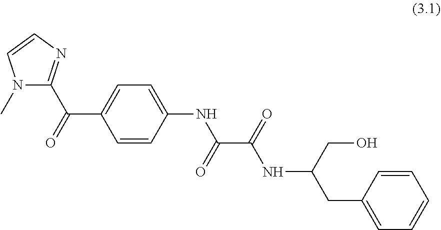 [SMILES: Cn1ccnc1C(=O)c1ccc(NC(=O)C(=O)NC(CO)Cc2ccccc2)cc1]
N1-(1-hydroxy-3-phenylpropan-2-yl)-N2-(4-(1-methyl-1H-imidazole-2-carbonyl)phenyl)oxalamide; 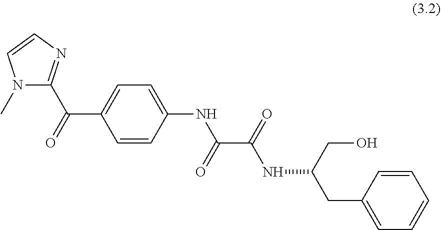
(S)—N1-(1-hydroxy-3-phenylpropan-2-yl)-N2-(4-(1-methyl-1H-imidazole-2-carbonyl)phenyl)oxalamide (denoted herein as “B3”): 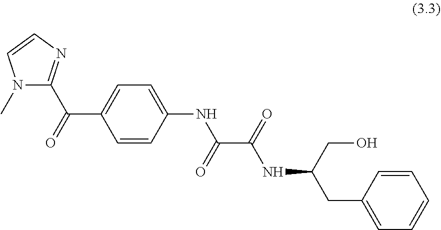 [SMILES: Cn1ccnc1C(=O)c1ccc(NC(=O)C(=O)N[C@@H](CO)Cc2ccccc2)cc1]
(R)—N1-(1-hydroxy-3-phenylpropan-2-yl)-N2-(4-(1-methyl-1H-imidazole-2-carbonyl)phenyl)oxalamide.

19. The method of claim 17, wherein said cell is of a subject suffering from at least one p53-associated disorder, and wherein said contacting step is performed by administering to said subject the effective amount of ARTS, any fragments thereof, or at least one mimetic compound thereof, and wherein said p53-associated disorder comprises at least one of: at least one proliferative disorder and at least one metabolic disorder.

20. The method of claim 17, for treating, preventing, inhibiting, reducing, eliminating, protecting or delaying the onset of a pathological disorder in a subject in need thereof, by upregulating p53 levels in at least one cell of said subject, the method comprising administering to said subject a therapeutically effective amount of at least one of ARTS, any fragments thereof, or at least one mimetic compound thereof or any vehicle, matrix, nano- or micro-particle thereof or of a composition comprising the same, wherein said disorder is at least one p53-associated disorder.


SEQUENCE LISTING (35 sequences)

SEQ ID NO: 1 - protein, 274 aa, Homo sapiens
  source 1..274: note = Amino acid sequence of human ARTS, GenBank Accession No. AF176379
MIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTLMVAGESGLGKSTLVNSLFLTDLYRDRKLLGAEERIMQTVEITKHAVDIEEKGVRLRLTIVDTPGFGDAVNNTECWKPVAEYIDQQFEQYFRDESGLNRKNIQDNRVHCCLYFISPFGHGYGPSLRLLAPPGAVKGTGQEHQGQGCH

SEQ ID NO: 2 - DNA, 1911 bp, Homo sapiens
  source 1..1911: mol_type = genomic DNA; note = Nucleic acid sequence encoding human ARTS GenBank Accession No. AF176379
cggcggtgctgcgaggtcggcgcgcacgtccgccgcgggtcgctcgggcgctgtccaggcggagccggccccgcccgggctgcagccatgatcaagcgtttcctggaggacaccacggatgatggagaactgagcaagttcgtgaaggatttctcaggaaatgcgagctgccacccaccagaggctaagacctgggcatccaggccccaagtcccggagccaaggccccaggccccggacctctatgatgatgacctggagttcagacccccctcgcggccccagtcctctgacaaccagcagtacttctgtgccccagcccctctcagcccatctgccaggccccgcagcccatggggcaagcttgatccctatgattcctctgaggatgacaaggagtatgtgggctttgcaaccctccccaaccaagtccaccgaaagtccgtgaagaaaggctttgactttaccctcatggtggcaggagagtctggcctgggcaaatccacacttgtcaatagcctcttcctcactgatctgtaccgggaccggaaacttcttggtgctgaagagaggatcatgcaaactgtggagatcactaagcatgcagtggacatagaagagaagggtgtgaggctgcggctcaccattgtggacacaccaggttttggggatgcagtcaacaacacagagtgctggaagcctgtggcagaatacattgatcagcagtttgagcagtatttccgagacgagagtggcctgaaccgaaagaacatccaagacaacagggtgcactgctgcctgtacttcatctcacccttcggccatgggtatggtccaagcctgaggctcctggcaccaccgggtgctgtcaagggaacaggccaagagcaccaggggcagggctgccactagcaggtggtcacaggttcctgttccccaggctccggccattggatgttgaattcatgaaggccctgcatcagcgggtcaacatcgtgcctatcctggctaaggcagacacactgacacctcccgaagtggaccacaagaaacgcaaaatccgggaggagattgagcattttggaatcaagatctatcaattcccagactgtgactctgatgaggatgaggacttcaaattgcaggaccaagccctaaaggaaagcatcccatttgcagtaattggcagcaacactgtagtagaggccagagggcggcgagttcggggtcgactctacccctggggcatcgtggaagtggaaaacccagggcactgcgactttgtgaagctgaggacaatgctggtacgtacccacatgcaggacctgaaggatgtgacacgggagacacattatgagaactaccgggcacagtgcatccagagcatgacccgcctggtggtgaaggaacggaatcgcaagtatgaccagaagccaggacaaagctggcagggggagatcccaagcctagccttgggtgagaccaagccctacttttgttcttctataggccctgggctcaatctaagcgggtgctggggtcctcctcgccttatcaacccttttctccctttagcaaactgactcgggaaagtggtaccgacttccccatccctgctgtcccaccagggacagatccagaaactgagaagcttatccgagagaaagatgaggagctgcggcggatgcaggagatgctacacaaaatacaaaaacagatgaaggagaactattaactggctttcagccctggatatttaaatctcctcctcttcttcctgtccatgccggcccctcccagcaccagctctgctcaggccccttcagctactgccacttcgccttacatccctgctgactgcccagagactcagaggaaataaagtttaataaatctgtaggtggctaaaaa

SEQ ID NO: 3 - protein, 239 aa, Homo sapiens
  source 1..239: note = Bcl-2 protein GenBank Accession No. NP_000624
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 4 - protein, 205 aa, Homo sapiens
  source 1..205: note = Bcl-2 protein GenBank Accession No. NP_000648
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 5 - DNA, 6492 bp, Homo sapiens
  source 1..6492: mol_type = genomic DNA; note = Bcl-2 gene of GenBank Accession No. NM_000633
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 6 - DNA, 1207 bp, Homo sapiens
  source 1..1207: mol_type = genomic DNA; note = Bcl-2 gene of GenBank NM_000657 of.
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaggccacaggtccgagatgcgggggttggagtgcgggtgggctcctggggcaatgggaggctgtggagccggcgaaataaaatcagagttgttgcta

SEQ ID NO: 7 - protein, 233 aa, Homo sapiens [sequence omitted]

SEQ ID NO: 8 - DNA, 926 bp, Homo sapiens
  source 1..926: mol_type = genomic DNA; note = Bcl-xL gene as denoted by GenBank Accession No. Z23115
gaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaacccg

SEQ ID NO: 9 - protein, 497 aa, Homo sapiens
  source 1..497: note = human XIAP protein, GenBank Accession Nos. NP_001158, NP_001191330
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 10 - DNA, 8460 bp, Homo sapiens
  source 1..8460: mol_type = genomic DNA; note = XIAP gene GenBank Accession Nos. NM_001167, NM_001204401
gctggggccaagccgcagagcggagttggcatttccagattggggctcgggccgcgcctcctccgggaccctccccttggaccgagccgatcgccgcggggcagttcgggccggctgtcctggcgcgaaaaggtggacaagtcctattttcaagagaagatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaactctatagtaggcatgttatgttgttcttattaccctgattgaatgtgtgatgtgaactgactttaagtaatcaggattgaattccattagcatttgctaccaagtaggaaaaaaaatgtacatggcagtgttttagttggcaatataatctttgaatttcttgatttttcagggtattagctgtattatccattttttttactgttatttaattgaaaccatagactaagaataagaagcatcatactataactgaacacaatgtgtattcatagtatactgatttaatttctaagtgtaagtgaattaatcatctggattttttattcttttcagataggcttaacaaatggagctttctgtatataaatgtggagattagagttaatctccccaatcacataatttgttttgtgtgaaaaaggaataaattgttccatgctggtggaaagatagagattgtttttagaggttggttgttgtgttttaggattctgtccattttcttttaaagttataaacacgtacttgtgcgaattatttttttaaagtgatttgccatttttgaaagcgtatttaatgatagaatactatcgagccaacatgtactgacatggaaagatgtcaaagatatgttaagtgtaaaatgcaagtggcaaaacactatgtatagtctgagccagatcaaagtatgtatgtttttaatatgcatagaacaaaagatttggaaagatatacaccaaactgttaaatgtggtttctcttcggggagggggggattgggggaggggccccagaggggttttataggggccttttcactttctacttttttcattttgttctgttcgaattttttataagtatgtattacttttgtaatcagaatttttagaaagtattttgctgatttaaaggcttaggcatgttcaaacgcctgcaaaactacttatcactcagctttagtttttctaatccaagaaggcagggcagttaacctttttggtgccaatgtgaaatgtaaatgattttatgtttttcctgctttgtggatgaaaaatatttctgagtggtagttttttgacaggtagaccatgtcttatcttgtttcaaaataagtatttctgattttgtaaaatgaaatataaaatatgtctcagatcttccaattaattagtaaggattcatccttaatccttgctagtttaagcctgcctaagtcactttactaaaagatctttgttaactcagtattttaaacatctgtcagcttatgtaggtaaaagtagaagcatgtttgtacactgcttgtagttatagtgacagctttccatgttgagattctcatatcatcttgtatcttaaagtttcatgtgagtttttaccgttaggatgattaagatgtatataggacaaaatgttaagtctttcctctacctacatttgttttcttggctagtaatagtagtagatacttctgaaataaatgttctctcaagatccttaaaacctcttggaaattataaaaatattggcaagaaaagaagaatagttgtttaaatattttttaaaaaacacttgaataagaatcagtagggtataaactagaagtttaaaaatgcttcatagaacgtccagggtttacattacaagattctcacaacaaacctattgtagaggtgagtaaggcatgttactacagaggaaagtttgagagtaaaactgtaaaaaattatatttttgttgtactttctaagagaaagagtattgttatgttctcctaacttctgttgattactactttaagtgatattcatttaaaacattgcaaatttattttatttatttaattttctttttgagatggagtcttgcttgtcacccaggctggagtgcagtggagtgatctctgctcactgcaacctccgccttctgggttcaagcgattctcgtgcctcagcttcctgagtagctggaattacaggcaggtgccaccatgcccgactaatttttttttatttttagtagagacggggtttcaccatgttggccaggctggtatcaaactcctgacctcaagagatccactcgccttgccctcccaaagtgctgggattacaggcttgagccaccacgcccggctaaaacattgcaaatttaaatgagagttttaaaaattaaataatgactgccctgtttctgttttagtatgtaaatcctcagttcttcacctttgcactgtctgccacttagtttggttatatagtcattaacttgaatttggtctgtatagtctagactttaaatttaaagttttctacaaggggagaaaagtgttaaaatttttaaaatatgttttccaggacacttcacttccaagtcaggtaggtagttcaatctagttgttagccaaggactcaaggactgaattgttttaacataaggcttttcctgttctgggagccgcacttcattaaaattcttctaaaacttgtatgtttagagttaagcaagactttttttcttcctctccatgagttgtgaaatttaatgcacaacgctgatgtggctaacaagtttattttaagaattgtttagaaatgctgttgcttcaggttcttaaaatcactcagcactccaacttctaatcaaatttttggagacttaacagcatttgtctgtgtttgaactataaaaagcaccggatcttttccatctaattccgcaaaaattgatcatttgcaaagtcaaaactatagccatatccaaatcttttccccctcccaagagttctcagtgtctacatgtagactattccttttctgtataaagttcactctaggatttcaagtcaccacttattttacattttagtcatgcaaagattcaagtagttttgcaataagtacttatctttatttgtaataatttagtctgctgatcaaaagcattgtcttaatttttgagaactggttttagcatttacaaactaaattccagttaattaattaatagctttatattgcctttcctgctacatttggttttttcccctgtccctttgattacgggctaaggtagggtagagtgggtgtagtgagtgtatataatgtgatttggccctgtgtattatgatattttgttatttttgttgttatattatttacatttcagtagttgttttttgtgtttccattttagtggataaaatttgtattttgaactatgaatggagactaccgccccagcattagtttcacatgatataccctttaaacccgaatcattgttttatttcctgattacacaggtgttgaatggggaaaggggctagtatatcagtaggatatactatgggatgtatatatatcattgctgttagagaaatgaaataaaatggggctgggctcagtggctcacgcctgtaatcccagcactttgggaggctgaggcaggtggatcacgaggtcaggagatcgagaccatcctggctaacacggtgaaaccccgtctctactaaaaaacagaaaattagccgggcgtggtggcgggcgcctgtagtcccagctactcgggaggctgaggcaggagaatggtgtgaacccgggaggcagagcttgcagtgagccgagatctcgccactgcactccagcctgggcaacagagcaagactctgtctcaaaaaaaaaaaaaaaagaaataagaaaatgggaagcaatatttgacatagttctttttagtcaaatctacttgttaaaaaaagggtagcagtttattcatctgtgaaaggaaaataatacttatcttacaaggttgcaagagctcaaggagaccatgtatgtaaagttcctgctgtaaatatgaactcccatcctaatacccttttacctctctgtgggtttgtcttgacctggaaatttgggctaaaacttagaaaaaattcttacatgataactcagtgatgcttactcatagtttttggtgtttctcatagataagatataaatcagctgggcgcggtggctcatgcctgtaatcccagcactttgggaggccgaggcgggcagatcacctgaggtcgggaggtcgagaccagcctgaccaacatggagaaaccccgtctctactaaaaatacaaaattagctgggcgtggtggctcatgcctgtaatcccagctacttgggaggctgaggcaggagaatcgcttgaacccaggaggcggaggttgtggtgagcgaagatcgtgccattgcactccagcctgggcaacaagagcaaaactctgtctcaaaaaaaaaaaaagatataaatcacaataaataaataggtcaatacaaatgttagccaggcgtggtggcacatgcccatagtcgcagctactctggaggcagaggcaggaggatcacttgagcccatgaatttgaggcagcagtgagctatgattgtgccactgtactccagtctgggtgacagagtgagaccccatctctaaataaataggtcaaacccttaaaaatatttaaattcttaaaaaattgaaaagattattcttctcaaatttagttgagctttctaagagaagcaattggctttttcccacttcaataatcattttcagtttgactcatacagttaacacaatgtgaatttcttcctcagcataacagagttatagaatgacagggctggaagtgaccttagagagtatccagttctttcattttacaggtgaggcaactgagactcaaaggtgatgtaatttgtgcaaagattatagctaattagtagcagagccctgactgggacatagtttgaaggtgaaaaacttcaccaagctacctttcttgaaaggtccaaatgtttatgttttcaactactctttccactgtaccataactttcactacatattaaatgacactttataactaatataataggacaatcatcaatgcatatatagccagcccttcatatctgtgggttttgcatccatggattcaaccaaggaggaattgaaaacactgagaaaaaaaaaaaagaccacacaataaaaaaaaaaaatacaaaataatacaaagaaaaagccaaaattgtcatactgttgttaagcaacagtataacaactatttacatagcattaaggttggtgcaaaaatgcaaaaaaaaaaaaagcaattatttttaaaccaacctaatatattgtattaggtattaaagtcatctggacatgaattaaagtatatgatgccagcctggacaaaaggcaaaaccctgtctctacaaaaaatacaaaaattagctgggcatggtggtgtgtgcctgtagtcctggctactccggagcctgaggtgggaggatcgcttgagtctgggaggcagaggctgcattgagctatgatcatggcactgcattccagcctgggtgacagtgcaagaccttgtctcagaataaataaagtatgtgatgaagatgtgcatacattatatgcaaatactgttttttttttttttaatttaaacagtctcactgtgttgcccaggatggagtgcaatggcacaatcttggctcatggcaaactctgcctcgcaagcagctgggactacaggcatgctccacggtgcccagttaattttttttgtattcttagtagagacagggtttcaccatgttggccaggctagtcttgaatttctgacctcaagtgattcatctcccaaagtgctgggattacaggcgtgagccaccacggccggctaatttttgtattttttagtagtgactggtttcgcggtgttgaccaggctggtctcgaactcctgatctcaggtgatctgcctgcctcggcctcacaaagtgctgggattacaggtgtgaaccactgctcccggccttgtgtgattttatctaagggacttaagcgtcctcaggtcctagggggtcgtgaaaccaaaaccccagggatagcaagggacaattgtatcttcaaagtagacaaatggcgccgggcacggtggctcacgcctgtaatcccagcagtttccgaggctgaggcaggcggctcacctgaggtcaggagttggagaccagcctggccaacatgctgaaaccctgtctgtacaaaaatacaaaaatagctgggcatggtggcgcatgcctgtagtcccagctactagagcgactgaggcaggagaattgcttgaacctgggaggcggaggttgcagggagccaagatggcgccaccgcactccagcctaggtgatagagtgagactccctctcaaaaacaaaacaaaacaaaaaaattagacaaatgctacattaatgtttgggtggtcagattctactttgaatctgaagtttgcagatatgcctatagatttttggagtttaccactttcttattctgtatcattaatgtaatattttaaattactatatatgttaccatttttctggatttagtaagaaatttgcagttttggtttgatgtaacaagggttttaatgtaatttatgttagattttgcatttttttcattactgttatattttaacctgactgactgatctaattgtattagtattgtgaataatcatgtgaaatgttttgagacagagtactatatttgtgaatataattttatggtttttttcacttagaacctttctgtgtggaaaactaagaaaattgctttctgctgtataatctggcattcattgtagattaaagcttatttttctgtgaataaaacgtattcaataaaatactattctttaaaattatatcataaaaaaaaaa

SEQ ID NO: 11 - DNA, 23 bp, synthetic construct
  source 1..23: mol_type = other DNA; note = Forward primer for human ARTS
gagacgagagtggcctgaaccga

SEQ ID NO: 12 - DNA, 23 bp, synthetic construct
  source 1..23: mol_type = other DNA; note = Reverse primer for human ARTS; note = Reverse primer for human ARTS
aacaggaacctgtgaccacctgc

SEQ ID NO: 13 - DNA, 19 bp, synthetic construct
  source 1..19: mol_type = other DNA; note = Forward primer for human GAPDH
atggggaaggtgaaggtcg

SEQ ID NO: 14 - DNA, 22 bp, synthetic construct
  source 1..22: mol_type = other DNA; note = Reverse primer for human GAPDH
ggggtcattgatggcaacaata

SEQ ID NO: 15 - DNA, 23 bp, synthetic construct
  source 1..23: mol_type = other DNA; note = Forward PCR primer for Sept4/ARTS gene
gagacgagagtggcctgaaccga

SEQ ID NO: 16 - DNA, 23 bp, synthetic construct
  source 1..23: mol_type = other DNA; note = Reverse PCR primer for Sept4/ARTS gene
aacaggaacctgtgaccacctgc

SEQ ID NO: 17 - DNA, 21 bp, synthetic construct
  source 1..21: mol_type = other DNA; note = Forward PCR primer for p21 gene
gtggctctgattggctttctg

SEQ ID NO: 18 - DNA, 20 bp, synthetic construct
  source 1..20: mol_type = other DNA; note = Reverse PCR primer for p21 gene
ctgaaaacaggcagcccaag

SEQ ID NO: 19 - protein, 393 aa, Homo sapiens [sequence omitted]

SEQ ID NO: 20 - DNA, 1075 bp, Homo sapiens
  source 1..1075: mol_type = genomic DNA; note = Human P53
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagc

SEQ ID NO: 21 - protein, 66 aa, Homo sapiens
  source 1..66: note = Bir3 domain of human XIAP protein
YEARIFTFGTWIYSVNKEQLARAGFYALGEGDKVKCFHCGGGLTDWKPSEDPWEQHAKWYPGCKYL

SEQ ID NO: 22 - protein, 491 aa, Homo sapiens
  source 1..491: note = human MDM2 protein; note = >sp|Q00987|MDM2_HUMAN E3 ubiquitin-protein ligase Mdm2 OS=Homo sapiens OX=9606 GN=MDM2 PE=1 SV=1
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 23 - DNA, 12239 bp, Homo sapiens
  source 1..12239: mol_type = genomic DNA; note = >NM_001145337.3 Homo sapiens MDM2 proto-oncogene (MDM2), transcript variant 3, mRNA; note = human MDM2
agtggcgattggagggtagacctgtgggcacggacgcacgccactttttctctgctgatccaggcaaatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctagttgacctgtctataagagaattatatatttctaactatataaccctaggaatttagacaacctgaaatttattcacatatatcaaagtgagaaaatgcctcaattcacatagatttcttctctttagtataattgacctactttggtagtggaatagtgaatacttactataatttgacttgaatatgtagctcatcctttacaccaactcctaattttaaataatttctactctgtcttaaatgagaagtacttggtttttttttttcttaaatatgtatatgacatttaaatgtaacttattattttttttgagaccgagtcttgctctgttacccaggctggagtgcagtggcgtgatcttggctcactgcaagctctgcctcccgggttcgcaccattctcctgcctcagcctcccaattagcttggcctacagtcatctgccaccacacctggctaattttttgtacttttagtagagacagggtttcaccgtgttagccaggatggtctcgatctcctgacctcgtgatccgcccacctcggcctcccaaagtgctgggattacaggcatgagccaccgcgtccggcctaaatgtcacttagtacctttgatataaagagaaaatgtgtgaaagatttagttttttgtttttttgtttgtttgtttgtttgtttgttttgagatgagtctctctgtcgcccaggctggagtgcagtgtcatgatctagcagtctccgcttcccgggttcaagccattctcctggctcagcctctggagcagctgggattacaggcatgcaccaccatgcccagctaatttttgtatttttagtagagatagggtttcaccatgttggccaggctggtcacgaactcctgacctcaagtgaggtcacccgcctcggcctcccgaagtgctgggattgcagatgtgagccaccatgtccagccaagaattagtatttaaattttagatactctttttttttttttttttttttttttttgagacagagtcttgctccatcacccatgctagagtgcagtggagtgatctcggctcactgcaacttccgccttctgggttcaagctattctcctgcctcagccttccaagtaactgggattacaggcatgtaccaccataccagctgatttttttgtatttttagtaaagacagggtttcaccatgttagccaggctgatcttgaactcctaaactcaagtgatctactcacctcagcctcccaaaatgctgggattacagatgtgaggcacctggcctcagatttttgatactcttaaaccttctgatccttagtttctctctccaaaatactctttctaggttaaaaaaaaaaaggctcttatatttggtgctatgtaaatgaaaatgttttttaggttttcttgatttaacaatagagacagggtctccctgtgttgcccaggctggtctcgaactcctgggctcaagagatcctcctgtcttggcctcgcaaagtgctaagtaggattacaggcgttagccaccacacccggctgtaaaaatgtacttattctccagcctcttttgtataaaccatagtaagggatgggagtaatgatgttatctgtgaaaatagccaccatttacccgtaagacaaaacttgttaaagcctcctgagtctaacctagattacatcaggccctttttcacacacaaaaaaatcctttatgggatttaatggaatctgttgtttccccctaagttgaaaaacaactctaagacactttaaagtaccttcttggcctgggttacatggttcccagcctaggtttcagacttttgcttaaggccagttttagaaacccgtgaattcagaaaagttaattcagaaatttgataaacagaattgttatttaaaaactaactggaaagattgttaagttctttctgaattattcagaaattatgcatcattttccttcaagaatgacagggtcagcatgtggaattccaagatacctcttgacttcctctcaagctccgtgtttggtcagtggaggcccatccgagctcagcactgagaagtgttagtttctttgggacccatctaccctgaccacatcatgatgttcatctgcagctgttgcaaggtgttcagattgtataaacataaatgtcacaaaaactttaaaagaagtgcaattctcaaaaggttaggtggactaaagcattctgtaaagcaactgctaataatgagcttacagtggatttgaatttgaaaaatatagtaacaagcctgtcaaatatctgcaagaactatggaataaaactactgatgcagtgaagacagttgaaaagatcaaacaaatgccaagctatatttataatgaacaaattcaagaaaaaggactacggaaagttcaggacatcaaagaagtcaggcaaaactcatcttgacccctgttgcaggcaaaggaacgcagctggaagaaaagatgatataacagttaacaggatgcagacatggcagaggtttcctaaaaatctcattatctataaccatttctatatttacatttgaaaatctcctttggagacttagaacctctaaattattgacttattttttatataaggtcactccgatgaaaggtgattacaaaatcatctacattgctgtctacaaaacagataatatggatgtttgatcgcatctcattgttaactctttactgatatgtttgtaaatacagaagtgaaatgtggacataaaatagttacgctatttggttaatggtactagacaacatgtaattaatgacattcaaaaatttatggctagtgatatatataaagtaaaattttctttgcagtaaaatatgccctttattatagaagggaggatataaggaaccaacagtttgtatgaaaatagctcaaataatatcttttattttgattttaatatttcttattttggtttattagtgtcttagaacaaaatggccttatataatgaagcctagttatgctggactgttttgatctcttttaattgttctgacagatagttggggatgagagccgaataaggtttgcctgaaataactgacactatataatttctgctttggcaaatactaagttctaacttgtcattcctggtagaacaagctttatttttcgagcctagcaatgatctagaagcagatgttatctcagtgccttttgcaatttgttgtgtgggtttttttttttttaaagccacacaataattttggaaaacaatgtatgggtagaacatgtgtctgttaattgcacacaaaaccacttttaatgggtacagagttaaatttgaaggaataagttctagctgaagtattatgaactccaaataatgctttgaggacctccaaaggtaaaagtactaatccctttggccatttattgagagagagagagagagagagtagggtgactatagttaatgtattgaatgttcttgctacaaataaatgatatttgagctgatgggtgtgctaattacactgatttgatcaatacccattgtatgtgaaacagtacatacaccatatttacaattatgtatttaacatttaaaatttctaatataagtatctctcaaactgtggattaacttcttgatttatatttaaatatgaatcttaagcaaaacagtgaaaataaccatcttgatttagtgtttttctcccatatgtgaattgtatatacttaggtgaagacaataaaatcaactgaactgtaagcttagaataggactgaggtaattctgcacagcaactttactaatggtacattgttgcttcaaaactctctctctctctctctgtctgtctcaataaatggccaaagggattagtagtttacctgtggaggtcctccaagcattatttggagttgataatacttcagctacaaccaagcagaatctcttttttttggaggtcctcgaagcattatttggagttgataatacttcagcttcaatttggagttgataatatttcagctagaacctagtagaatctgtttttttcctttggaggtcctcaaagcattattggagttcataatactgaagctagaaccaagcagaatctgtttttttctgaggagtatcggtagcataaatgtgattataaacatagtacacttgatatatggaggcagtgacagctatttttacaaaatttaaatctgcaaatggattcaacatgtttatgggttattaaaattgtctgatttcttaggttctttatagtacacgtgttgaaaataaatgattaagaattgtttcaagaatgcaattatttgatcttaaatttttatgagttgttaaaatagaaattatttgaatatcatatatttgggtaacaaaaggcacaagtctgaatgtgtttctttttctggaatggccatgcctgcccactttagaaatacaaatatcactgggcagcttgaagcagttgggagcctccaatgagagcaacttgagagaatgatgttgcaagttagtaggagtaagaaatgctgtgttctccctgtcttctcttaggtcacatggcagcctggcctaagtgatcgtgaatggtctataagggaggtagctgggacagggaggggagtttgggctagccaccgtaccacttgtcagcgtgaaaagtaagattgtaattgcctgtttagttttctgcctcatctttgaaagttccaccaagctgggaacctcttgattgtgaggcacaaatgtaagtacatcagaaaaaaacaaaaaaactggctttaaagcaggagcttgtgggcccctaagccagacggggactagcttttggcattatataattaagattttttaaatccttaataagggttttattttatttttatttattttttgagacggagtcttgctctgtggctcaggctggagtacagtggtgcaatcttggctcactgcaacctctgcctcctggctgtgttcaagtggttctgcttcagcctcccaagtagctggggttagagcaccctgtcaccacgccccgctaatttttgtatttctagcagagatgaagtttcactatgttggccaggctgggctcaaactcctgacctcaagtgatctgcccgccttggccccccaaagtgctgtgattacaggcgtgagccgccacgcccagcctaataagggttttaaagataattagtgtgtaggtctgtaggcttatgatggtaaccacaagttgttaatggcattgtgaaaagtttttagttgcgctttatgggtggatgctgaattacattttgatttgatacttataaaaagaaaaagtatttcttcagcttaaaaaattgtttaaaagtttgtgatcatattgtctaccatgtagccagctttcaattatatgtaagagggactttttgacatttacaaataatactttgaggtagatatctgaaagcaccagcacttggaaggtgttcagaagtaacaaattataaaatgagctaacaaacgaaaggcaaaataaaaccgtaaagcaagcagatgggaggcgtgttcagtaacttattcataatgcatctgaaatgattgctgtactcaaatatttaacgttagagtaatagtattttgaatgaaaaccatagttgattgtctttagagagaggagtggtactgaaacatacttgaattcgtattgctttcctttaaagctctcctttaaagactttaaaaaaaattcgataggcattttcatgtttcagtcctacaatcccacatctctgctgccttcagaatgctacaacttagatgtccagccaacagcttaaagccaatttataaaaatagcatctttcctaattccaaaatttcaccttttgtttgttttgttttgagatgcggtcttgctctgtcgcccaggctggaatgcagtgctgcaaacatgactcactacagcctcaacctcctgggctcaagcaatcctcccacctcagcctcccaagtagctggggaccacaggtgcaagccaccaggtccagctaaccttccccccacctcccccgaccccgctgccacccgccctgagacagagtcttgctctttcacccagactggagtgcagtggtgcaatctcggctcattgcaacctccacctcccaggttcaagcaattctgcctcagcctcctgagtagctggaattataggtgctcgccaaccacacctggctaattttttttgtatttttagtagagatggggtttcagcatgttgtccaggctggtctcgaactcctgacctcatgatccgcccacctcggcctcccaaagtactgggattacaggcgtgagccaccgtgcccggcctaatttttgtattttttgtagagacagtttcaccatgttgcccaggctggtctcaaactcctggggtcaagtgatctgcccgtcttggcttcccaaagtgctgagattacaggtgtaagccaccacgccaagcccaaaatttcaccttttaaatttattccatttattttgtaggtgaatttcatttatttctcagggttaaattttagcgcttttgcctgtgtctatttttggttcacacctccccagcccactgtcctgtattaagtaggccttccttcaagatcacagtaatatctccctaaatggatttcactacgtttggtcctatttttggttcagttatagtttacagataccatcatgttgcttccctgatttaaatattgggggaaaaacttgctttttggattttatctctgatgttctactggattattttagacttttagaacactatgaaatgggtgcttgcttttcacttttcaaaccttttttgcttaaacaaagttcataagcttattgtagtgggaagttcaaagtcacagatctggtacttactcgcctagtgaccttagagacaataaatctatagagcctagtatggggtctggcaggtagtaagcacttttaatgaatggaagtttaatattctgtttgcatccactcgaccataatcaccccaaatccagcttaggtagccttgaattaacccctactccaccttgaagttttttatttgttttttgctgagacagggtctcattctgtcacccaggctggagtatagtggcgcaaacatgactcactgcagcctcctgagtagctgggaacacaggcctgttgccaccatgcctggctaatatatatattatatataatatacatatattacatatatatatataatacatatgtgtttatgtatgtatgtgtgtgtacattatatatatatatatactttttttagaggcagagtcctgctatgttgcccaggctggccttgaactcctgggctcaagcaatcctcgtgtcttgaccttgacctctcaaaatgctgggattacaggtgcgagccactgcatctggtgaattaagccttttttatacactcaactctgaggtattctgttcataccaatatgttcctttgtatcttcttgccatagcaggttgtaacatccttttgtatctcctttctttttttttttttttttttgagacggagtcccgctgtttagcccaggccggattgcagtggcgcaatctcggctcactgcaagctccgcctcccaggttcacgccattctcctgcctcagcctcccgagtagctgggactacaggcgcccgccaccgcgcctggctaattttttgtatttttagtagagacggggtttcaccgtgttagccaagatggtctcgatctcctgaccttgtgatccgcccgcctcggcctcccaaagtgctgggattacaggcgtgagccaccgcgcccagccgtatctcctttcaaatgtcaaataaatacattgtgtatacagtagtaaaggatgttgattggcattgaaagcaactatcaagttcttgttgatttagaagacaaaaagacaattttaaaataacatcctgggccaggctctgtgactcacgcctgtaatctcaacactttgggaggccaaggaaggaggatcacttgaggccaggagtttgagatcagcctgggcaacataatgacaccctgtatccaaaaaattgtggtggcatgcatgggagactgaggtgggaggatcacttgagcccaggagtccaatgttacaatgagctatgattgcgccactacactctgcttaggagacagatggagacttgtctcttaaaaaaataaaaaataacagtatgttcattaaacattaatctcactgattgtgattttttgtttgtttttttgagatggcgtcttgctctgttgcccagggtggagtgcaatggcgtgatcttggctcactgcagcctccacctcctgggttcaagtgattctcctgctttagcctcccaagtagctgtgttatttcagctaattttttcatttttagtggagatggggttttgccatgttggccaggctggtctcgaattcctgacctcatgtggtccacctgcctcggcctcccaaagtgctgggattacaggcattgagccactgtgcctggcctgattatgattttttaaagtccccccccaaattattgtttgtgcaataaatggcaaaaaagaagcattctaagaaaaaagtagtcttaaaagttgatatggtgtcagaattgtaagagagagggtcttggctgaaaagcctttttagaaaaaattgctccctacaaaaaaggccttttctaccttttctcacatttgtttctttttataggaggcatctgagaagaccaaatctcttcaaagttaagctttttgtttttttgggggggacgaagtctcgttctgtcacccaggctggagtgtagtggcatgatctcggctcactgcaacctctgcctcctgggttcaagcaattctcctgcctcagcctcccgagtagctggaaattcaggcgcctgccaccatgcccagctaatttttgtatttttaatagaggcagggtttcactatattggccaggctggtctcgaactcctgacctcatgatccgcctgcctctgcctcccaaagtgttgggattacaggcatgagccaccgcacccagcccaaagttaagcattttttaaccacaaggtgggaggtggaagtatctcgttggaagactaaaggtgtccactaacggtagacccttttttttttttttttgagatggagtttcactgtcgcccagactagactgaagtggcgcgatctcggctcactgcaacctctgccccccaggttcaagtgattctcctgcctcagcctctagagtagctaggattacaggggtccaccaccacacctggctagtttttgtattttcagtagagacggggtttcaccatgttggccaggctggtctcgaactcctgacctcaggtgacccacctgctttggcctcccaaagtgctgggattacaggtgtgagccactgcgcctggccgtttttttttttgttgttgttgttgtttgttcgtttgtttgttttgagacagtctctcactccaggttggagtgcagtggcactatctcagctcactgcaaactctgcctcccaaggtcaaatgattctcctgcctcagcctcccagtagttggaattacaggcgcatgccaccatgcatggctaattttttgtatttttagtagagacggggtttcaccatgctagccttgaactcctgaccttgtgatctgcctgcctcggcctcccaaagtgctgggattacagacatgagccaccatgctagccttgaactcctgacctcgtgatctgcctgcctcggcctcccaaagtgctgggattacagacatgagccaccatgcctggcaaatttttgtattttcagtagagatggggttttgctatgttagccaggctagtctcgaactcctgacctcaggtgatccagtcgcctcggcctcccaaagtgctgggattacagatgtgagtcactacgcttggtagactcttaacagcgctgagagaccactccaagagagcagagaaataatagttctattctcagttgtcaaacagcaaacaaaaaacccttggctggggggtgatggctcacccctgtaatcccagcactttgggaggctgaggtgggtggatcacttgaggccaggaattcaagaccaacctgggcaacatggtgcaaccctgtcttaactaaaaatacaaaaattagccaggcgtcatggcatgtgcctgttaattccagctgctcgagaggctgaggcatgagaattacttgaacctgggaggtggaggttgcagtgagccgagatcccaccactgcactccagcctgggtgacagagcgagacttcgtcccaaaaaaaaaaaaaaaagctgcaaatttaaacgttgaagtctaggtaaatataccagggaacttctgcttaagaggcttctatgtaatgaaattctcttgaaaacagagaaactatttcctgtttattttctaaattgagacgtcactttttaaaaattggtacctgtaatttagccatttcctactcagcaatgtctcatttaaactattatttgtttagcgtgtttcaaagagcagatgtaagcttgagcccatcctctgtcctatgactaagtcgatattagcaggggttaggactgttagttttccagttcctactggaggcaaattctttgtttaccactgttctctgtatttcaaaagcacctggaaatcaataaaatttgttattcagaacaaaa

SEQ ID NO: 24 - protein, 27 aa, synthetic construct
  source 1..27: note = ARTS C' terminus
YGPSLRLLAPPGAVKGTGQEHQGQGCH

SEQ ID NO: 25 - protein, 9 aa, synthetic construct
  source 1..9: note = ARTS C' terminal fragment
YGPSLRLLA

SEQ ID NO: 26 - protein, 9 aa, synthetic construct
  source 1..9: note = ARTS C' terminal fragment
PPGAVKGTG

SEQ ID NO: 27 - protein, 9 aa, synthetic construct
  source 1..9: note = ARTS C' terminal fragment
QEHQGQGCH

SEQ ID NO: 28 - protein, 128 aa, synthetic construct
  source 1..128: note = ARTS N' terminal fragment residues 1-128
MIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTL

SEQ ID NO: 29 - protein, 148 aa, synthetic construct
  source 1..148: note = ARTS N' terminal fragment residues 1-148
MIKRFLEDTTDDGELSKFVKDFSGNASCHPPEAKTWASRPQVPEPRPQAPDLYDDDLEFRPPSRPQSSDNQQYFCAPAPLSPSARPRSPWGKLDPYDSSEDDKEYVGFATLPNQVHRKSVKKGFDFTLMVAGESGLGKSTLVNSLFLT

SEQ ID NO: 30 - protein, 43 aa, synthetic construct
  source 1..43: note = ARTS N' terminal fragment residues 106-148
VGFATLPNQVHRKSVKKGFDFTLMVAGESGLGKSTLVNSLFLT

SEQ ID NO: 31 - protein, 28 aa, synthetic construct
  source 1..28: note = ARTS N' terminal fragment residues 106-133
VGFATLPNQVHRKSVKKGFDFTLMVAGE

SEQ ID NO: 32 - protein, 23 aa, synthetic construct
  source 1..23: note = ARTS N' terminal fragment residues 106-128
VGFATLPNQVHRKSVKKGFDFTL

SEQ ID NO: 33 - protein, 37 aa, synthetic construct
  source 1..37: note = ARTS N' terminal fragment residues 112-148
PNQVHRKSVKKGFDFTLMVAGESGLGKSTLVNSLFLT

SEQ ID NO: 34 - protein, 22 aa, synthetic construct
  source 1..22: note = ARTS N' terminal fragment residues 112-133
PNQVHRKSVKKGFDFTLMVAGE

SEQ ID NO: 35 - protein, 17 aa, synthetic construct
  source 1..17: note = ARTS N' terminal fragment 112-128
PNQVHRKSVKKGFDFTL